SEQUENCE LISTING (147 sequences)

SEQ ID NO: 1 - protein, 485 aa, Artificial Sequence
  Anti-human 1928z CAR
MALPVTALLLPLALLLHAEVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKRAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 2 - protein, 466 aa, Artificial Sequence
  Anti-human ROR1 CAR
MLLLVTSLLLCELPHPAFLLIPQEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYADDGALFNIWGPGTLVTISSGGGGSGGGGSGGGGSELVLTQSPSVSAALGSPAKITCTLSSAHKTDTIDWYQQLQGEAPRYLMQVQSDGSYTKRPGVPDRFSGSSSGADRYLIIPSVQADDEADYYCGADYIGGYVFGGGTQLTVTGESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLE

SEQ ID NO: 3 - protein, 608 aa, Artificial Sequence
  HBV-specific TCR
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATWLSGSARQLTFGSGTQLTVLPDIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSGATNFSLLKQAGDVEENPGPMDSWTLCCVSLCILVAKHTDAGVIQSPRHEVTEMGQEVTLRCKPISGHDYLFWYRQTMMRGLELLIYFNNNVPIDDSGMPEDRFSAKMPNASFSTLKIQPSEPRDSAVYFCASSNRASSYNEQFFGPGTRLTVLEDLKNVFPPEVAVFEPSEAEISHTQKATLVCLATGFYPDHVELSWWVNGKEVHSGVCTDPQPLKEQPALNDSRYSLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 4 - DNA, 1458 bp, Artificial Sequence
  Anti-human 1928z CAR
atggctctgcctgtgacagctctgctgctgcctctggctctgcttctgcacgccgaagtgaagctgcagcagtctggcgccgaactcgttagacctggcagcagcgtgaagatcagctgcaaggccagcggctacgccttcagcagctactggatgaactgggtcaagcagaggcctggacagggcctcgaatggatcggccaaatctatcctggcgacggcgacaccaactacaacggcaagttcaaaggccaggccacactgaccgccgacaagtctagcagcacagcctacatgcagctgagcggcctgacaagcgaagatagcgccgtgtacttctgcgccagaaagaccatcagctccgtggtggacttctacttcgactactggggccagggcaccaccgtgacagtttctagcggaggcggaggatctggtggcggaggaagtggcggaggcggctctgatattgagctgacacagagccccaagttcatgagcaccagcgtgggcgatagagtgtccgtgacctgtaaagccagccagaacgtgggcacaaacgtggcctggtatcagcagaagcctggccagtctcctaagcctctgatctacagcgccacctacagaaacagcggcgtgcccgatagattcaccggctctggaagcggcaccgacttcaccctgaccatcaccaacgtgcagagcaaggacctggccgactacttctgccagcagtacaacagatacccctacaccagcggcggaggcaccaagctggaaatcaaaagagccgccgctatcgaagtgatgtaccctcctccttacctggacaacgagaagtccaacggcaccatcatccacgtgaagggcaaacacctgtgtccttctccactgttccccggacctagcaagcctttctgggtgctcgttgttgttggcggcgtgctggcctgttactctctgctggttaccgtggccttcatcatcttttgggtccgaagcaagcggagccggctgctgcacagcgactacatgaacatgacccctagacggcccggaccaaccagaaagcactaccagccttacgctcctcctagagacttcgccgcctaccggtccagagtgaagttcagcagatccgccgagcctcctgcctatcagcagggacagaaccagctgtacaacgagctgaacctggggagaagagaagagtacgacgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagataa

SEQ ID NO: 5 - DNA, 1401 bp, Artificial Sequence
  Anti-human ROR1 (4-1BBz) CAR
atgctgctgctggtgacaagcctgctgctgtgcgagctgccccaccccgcctttctgctgatcccccaggaacagctcgtcgaaagcggcggcagactggtgacacctggcggcagcctgaccctgagctgcaaggccagcggcttcgacttcagcgcctactacatgagctgggtccgccaggcccctggcaagggactggaatggatcgccaccatctaccccagcagcggcaagacctactacgccacctgggtgaacggacggttcaccatctccagcgacaacgcccagaacaccgtggacctgcagatgaacagcctgacagccgccgaccgggccacctacttttgcgccagagacagctacgccgacgacggcgccctgttcaacatctggggccctggcaccctggtgacaatctctagcggcggaggcggatctggtggcggaggaagtggcggcggaggatctgagctggtgctgacccagagcccctctgtgtctgctgccctgggaagccctgccaagatcacctgtaccctgagcagcgcccacaagaccgacaccatcgactggtatcagcagctgcagggcgaggcccccagatacctgatgcaggtgcagagcgacggcagctacaccaagaggccaggcgtgcccgaccggttcagcggatctagctctggcgccgaccgctacctgatcatccccagcgtgcaggccgatgacgaggccgattactactgtggcgccgactacatcggcggctacgtgttcggcggaggcacccagctgaccgtgaccggcgaatctaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagtga

SEQ ID NO: 6 - DNA, 1827 bp, Artificial Sequence
  Anti-HBV-specific TCR (HBcore18-27)
atggaaactctgctgggcgtgtcactggtcattctgtggctgcagctggcaagggtcaatagtcagcagggcgaagaagatcctcaggcactgtccatccaggagggcgaaaatgccaccatgaactgctcatacaagacaagcattaacaacctccagtggtatcggcagaacagcggccgggggctggtgcacctgatcctgattcgatcaaacgagcgggaaaagcatagcgggcggctgagagtgactctggacaccagcaagaaaagctcctctctgctgatcactgcctccagggccgctgataccgcatcttacttttgtgcaacatggctgagcggcagcgcacgacagctgaccttcggcagcggcacacagctgactgtgctgcctgacattcagaatccagatcccgccgtctaccagctgcgggacagcaagagttcagataaatccgtgtgcctgttcacagactttgattctcagactaacgtctctcagagtaaggacagtgatgtgtatatcacagacaaatgtgtcctggatatgcggagcatggacttcaagtcaaatagcgcagtggcctggagcaacaaatccgacttcgcttgcgcaaatgcctttaacaattcaatcattccagaggataccttctttcctagcccagaaagctcctgtgacgtgaagctggtcgagaaaagtttcgaaaccgatacaaatctgaactttcagaatctgtctgtgatcggattccggattctgctgctgaaggtcgccggctttaacctgctgatgaccctgagactgtggtctagtgggtctggagctacaaatttcagtctgctgaaacaggcaggcgatgtggaggaaaaccccgggcctatggacagttggacactgtgctgtgtgtcactgtgcatcctggtcgctaagcacactgacgcaggcgtgattcagagcccaagacatgaagtgactgaaatggggcaggaggtcaccctgaggtgtaaacccatcagcggacacgattacctgttttggtataggcagactatgatgcgcggactggagctgctgatctacttcaacaataacgtgcccattgacgatagcgggatgcccgaagacaggttttccgccaagatgccaaatgcttccttctctaccctgaaaattcagccttccgagccacgagatagcgccgtgtacttttgcgcctcaagcaatcgggcttcctcttataacgagcagttctttggaccagggacccgactgacagtgctggaagacctgaagaacgtcttcccccctgaggtggccgtctttgaacctagtgaggctgaaatctcacacactcagaaagctaccctggtgtgcctggcaaccggcttctacccagatcacgtggagctgtcctggtgggtcaatgggaaggaagtgcattctggagtctgtacagacccacagcccctgaaagagcagcccgccctgaacgattctcggtacagtctgagttcaaggctgcgcgtgagcgctacattttggcagaatcctagaaaccatttcaggtgccaggtgcagttttatgggctgtccgagaacgatgaatggacacaggaccgcgccaagcccgtgactcagatcgtctctgctgaggcatggggacgagctgactgtggcttcaccagtgaatcatatcagcagggcgtgctgagcgccactatcctgtacgagattctgctggggaaggccaccctgtatgctgtgctggtcagcgcactggtgctgatggctatggtgaagcgcaaagatagccgaggctga

SEQ ID NO: 7 - DNA, 734 bp, Artificial Sequence
  anti-CD19 scFv (VH-VL) FMC63
cacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcag

SEQ ID NO: 8 - protein, 245 aa, Artificial Sequence
  anti-CD19 scFv (VH-VL) FMC63
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 9 - DNA, 123 bp, Artificial Sequence
  CD28 effector domain
cggagcaagcggagcagaggcggccacagcgactacatgaacatgacccccagacggcctggccccacccggaagcactaccagccctacgccccacccagggactttgccgcctacagaagc

SEQ ID NO: 10 - DNA, 1476 bp, Artificial Sequence
  P28z CAR
gtggcctcaccgttgacccgctttctgtcgctgaacctgctgctgctgggtgagtcgattatcctggggagtggagaagctgaggtgcagctgcagcagtcaggacctgaactggtgaagcctgggacttcagtgaggatatcctgcaagacttctggatacacattcactgaatataccatacactgggtgaagcagagccatggaaagagccttgagtggattggaaacatcaatcctaacaatggtggtaccacctacaatcagaagttcgaggacaaggccacattgactgtagacaagtcctccagtacagcctacatggagctccgcagcctaacatctgaggattctgcagtctattattgtgcagctggttggaactttgactactggggccaagggaccacggtcaccgtctcctcaggtggaggtggatcaggtggaggtggatctggtggaggtggatctgacattgtgatgacccagtctcacaaattcatgtccacatcagtaggagacagggtcagcatcatctgtaaggccagtcaagatgtgggtactgctgtagactggtatcaacagaaaccaggacaatctcctaaactactgatttattgggcatccactcggcacactggagtccctgatcgcttcacaggcagtggatctgggacagacttcactctcaccattactaatgttcagtctgaagacttggcagattatttctgtcagcaatataacagctatcccctcacgttcggtgctgggaccatgctggacctgaaacgggcggccgcatctactactaccaagccagtgctgcgaactccctcacctgtgcaccctaccgggacatctcagccccagagaccagaagattgtcggccccgtggctcagtgaaggggaccggattggacttcgcctgtgatatttacatctgggcacccttggccggaatctgcgtggcccttctgctgtccttgatcatcactctcatctgctacaatagtagaaggaacagactccttcaaagtgactacatgaacatgactccccggaggcctgggctcactcgaaagccttaccagccctacgcccctgccagagactttgcagcgtaccgccccagagcaaaattcagcaggagtgcagagactgctgccaacctgcaggaccccaaccagctctacaatgagctcaatctagggcgaagagaggaatatgacgtcttggagaagaagcgggctcgggatccagagatgggaggcaaacagcagaggaggaggaacccccaggaaggcgtatacaatgcactgcagaaagacaagatggcagaagcctacagtgagatcggcacaaaaggcgagaggcggagaggcaaggggcacgatggcctttaccagggtctcagcactgccaccaaggacacctatgatgccctgcatatgcagaccctggcccctcgctaa

SEQ ID NO: 11 - protein, 327 aa, Artificial Sequence
  IgG4-Fc
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 229 aa, Artificial Sequence
  Hinge-CH2-CH3
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 119 aa, Artificial Sequence
  Hinge-CH3
ESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 14 - protein, 12 aa, Artificial Sequence
  Hinge
ESKYGPPCPPCP

SEQ ID NO: 15 - protein, 28 aa, Artificial Sequence
  CD28 Transmembrane domain
MFWVLVVVGGVLACYSLLVTVAFIIFWV

SEQ ID NO: 16 - protein, 41 aa, Artificial Sequence
  CD28 Cytoplasmic domain (LL to GG)
RSKRSRGGHSDYMNMTPRRPGPTRKHYQPYAPPRDFAAYRS

SEQ ID NO: 17 - protein, 42 aa, Artificial Sequence
  4-1BB Cytoplasmic domain
KRGRKKLLYIFKQPFMRPVQTTQEEDGCSCRFPEEEEGGCEL

SEQ ID NO: 18 - protein, 112 aa, Artificial Sequence
  CD3-z Cytoplasmic domain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 19 - protein, 24 aa, Artificial Sequence
  T2A
LEGGGEGRGSLLTCGDVEENPGPR

SEQ ID NO: 20 - protein, 356 aa, Artificial Sequence
  tEGFR
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 21 - protein, 8 aa, Artificial Sequence
  Strep tag II
WSHPQFEK

SEQ ID NO: 22 - protein, 10 aa, Artificial Sequence
  Myc tag
EQKLISEEDL

SEQ ID NO: 23 - protein, 14 aa, Artificial Sequence
  V5 tag
GKPIPNPLLGLDST

SEQ ID NO: 24 - protein, 8 aa, Artificial Sequence
  FLAG tag
DYKDDDDK

SEQ ID NO: 25 - protein, 498 aa, Homo sapiens
MNQSIPVAPTPPRRVRLKPWLVAQVNSCQYPGLQWVNGEKKLFCIPWRHATRHGPSQDGDNTIFKAWAKETGKYTEGVDEADPAKWKANLRCALNKSRDFRLIYDGPRDMPPQPYKIYEVCSNGPAPTDSQPPEDYSFGAGEEEEEEEELQRMLPSLSLTEDVKWPPTLQPPTLRPPTLQPPTLQPPVVLGPPAPDPSPLAPPPGNPAGFRELLSEVLEPGPLPASLPPAGEQLLPDLLISPHMLPLTDLEIKFQYRGRPPRALTISNPHGCRLFYSQLEATQEQVELFGPISLEQVRFPSPEDIPSDKQRFYTNQLLDVLDRGLILQLQGQDLYAIRLCQCKVFWSGPCASAHDSCPNPIQREVKTKLFSLEHFLNELILFQKGQTNTPPPFEIFFCFGEEWPDRKPREKKLITVQVVPVAARLLLEMFSGELSWSADSIRLQISNPDLKDRMVEQFKELHHIWQSQQRLQPVAQAPPGAGLGVGQGPWPMHPAGMQ

SEQ ID NO: 26 - protein, 514 aa, Homo sapiens
MNQSIPVAPTPPRRVRLKPWLVAQVNSCQYPGLQWVNGEKKLFCIPWRHATRHGPSQDGDNTIFKAWAKETGKYTEGVDEADPAKWKANLRCALNKSRDFRLIYDGPRDMPPQPYKIYEVCSNGPAPTDSQPPEDYSFGAGEEEEEEEELQRMLPSLSLTDAVQSGPHMTPYSLLKEDVKWPPTLQPPTLRPPTLQPPTLQPPVVLGPPAPDPSPLAPPPGNPAGFRELLSEVLEPGPLPASLPPAGEQLLPDLLISPHMLPLTDLEIKFQYRGRPPRALTISNPHGCRLFYSQLEATQEQVELFGPISLEQVRFPSPEDIPSDKQRFYTNQLLDVLDRGLILQLQGQDLYAIRLCQCKVFWSGPCASAHDSCPNPIQREVKTKLFSLEHFLNELILFQKGQTNTPPPFEIFFCFGEEWPDRKPREKKLITVQVVPVAARLLLEMFSGELSWSADSIRLQISNPDLKDRMVEQFKELHHIWQSQQRLQPVAQAPPGAGLGVGQGPWPMHPAGMQ

SEQ ID NO: 27 - protein, 504 aa, Homo sapiens
MNQSIPVAPTPPRRVRLKPWLVAQVNSCQYPGLQWVNGEKKLFCIPWRHATRHGPSQDGDNTIFKAWAKETGKYTEGVDEADPAKWKANLRCALNKSRDFRLIYDGPRDMPPQPYKIYEVCSNGPAPTDSQPPEDYSFGAGEEEEEEEELQRMLPSLSLTDAVQSGPHMTPYSLLKEDVKWPPTLQPPTLQPPVVLGPPAPDPSPLAPPPGNPAGFRELLSEVLEPGPLPASLPPAGEQLLPDLLISPHMLPLTDLEIKFQYRGRPPRALTISNPHGCRLFYSQLEATQEQVELFGPISLEQVRFPSPEDIPSDKQRFYTNQLLDVLDRGLILQLQGQDLYAIRLCQCKVFWSGPCASAHDSCPNPIQREVKTKLFSLEHFLNELILFQKGQTNTPPPFEIFFCFGEEWPDRKPREKKLITVQVVPVAARLLLEMFSGELSWSADSIRLQISNPDLKDRMVEQFKELHHIWQSQQRLQPVAQAPPGAGLGVGQGPWPMHPAGMQ

SEQ ID NO: 28 - protein, 488 aa, Homo sapiens
MNQSIPVAPTPPRRVRLKPWLVAQVNSCQYPGLQWVNGEKKLFCIPWRHATRHGPSQDGDNTIFKAWAKETGKYTEGVDEADPAKWKANLRCALNKSRDFRLIYDGPRDMPPQPYKIYEVCSNGPAPTDSQPPEDYSFGAGEEEEEEEELQRMLPSLSLTEDVKWPPTLQPPTLQPPVVLGPPAPDPSPLAPPPGNPAGFRELLSEVLEPGPLPASLPPAGEQLLPDLLISPHMLPLTDLEIKFQYRGRPPRALTISNPHGCRLFYSQLEATQEQVELFGPISLEQVRFPSPEDIPSDKQRFYTNQLLDVLDRGLILQLQGQDLYAIRLCQCKVFWSGPCASAHDSCPNPIQREVKTKLFSLEHFLNELILFQKGQTNTPPPFEIFFCFGEEWPDRKPREKKLITVQVVPVAARLLLEMFSGELSWSADSIRLQISNPDLKDRMVEQFKELHHIWQSQQRLQPVAQAPPGAGLGVGQGPWPMHPAGMQ

SEQ ID NO: 29 - protein, 412 aa, Homo sapiens
MNQSIPVAPTPPRRVRLKPWLVAQVNSCQYPGLQWVNGEKKLFCIPWRHATRHGPSQDGDNTIFKAWAKETGKYTEGVDEADPAKWKANLRCALNKSRDFRLIYDGPRDMPPQPYKIYEVCSNGPAPTDSQPPEDYSFGAGEEEEEEEELQRMLPSLSLTVTDLEIKFQYRGRPPRALTISNPHGCRLFYSQLEATQEQVELFGPISLEQVRFPSPEDIPSDKQRFYTNQLLDVLDRGLILQLQGQDLYAIRLCQCKVFWSGPCASAHDSCPNPIQREVKTKLFSLEHFLNELILFQKGQTNTPPPFEIFFCFGEEWPDRKPREKKLITVQVVPVAARLLLEMFSGELSWSADSIRLQISNPDLKDRMVEQFKELHHIWQSQQRLQPVAQAPPGAGLGVGQGPWPMHPAGMQ

SEQ ID NO: 30 - protein, 147 aa, Homo sapiens
MNQSIPVAPTPPRRVRLKPWLVAQVNSCQYPGLQWVNGEKKLFCIPWRHATRHGPSQDGDNTIFKAWAKETGKYTEGVDEADPAKWKANLRCALNKSRDFRLIYDGPRDMPPQPYKIYETPSPLRITLLVQERRRKKRKSCRGCCQA

SEQ ID NO: 31 - protein, 497 aa, Mus musculus
MNHSAPGIPPPPRRVRLKPWLVAQVNSCQYPGLQWVNGEKKLFYIPWRHATRHGPSQDGDNTIFKAWAKETGKYTEGVDEADPAKWKANLRCALNKSRDFQLFYDGPRDMPPQPYKIYEVCSNGPAPTESQPTDDYVLGEEEEEEEEELQRMLPGLSITEPALPGPPNAPYSLPKEDTKWPPALQPPVGLGPPVPDPNLLAPPSGNPAGFRQLLPEVLEPGPLASSQPPTEPLLPDLLISPHMLPLTDLEIKFQYRGRAPRTLTISNPQGCRLFYSQLEATQEQVELFGPVTLEQVRFPSPEDIPSDKQRFYTNQLLDVLDRGLILQLQGQDLYAIRLCQCKVFWSGPCALAHGSCPNPIQREVKTKLFSLEQFLNELILFQKGQTNTPPPFEIFFCFGEEWPDVKPREKKLITVQVVPVAARLLLEMFSGELSWSADSIRLQISNPDLKDHMVEQFKELHHLWQSQQQLQPMVQAPPVAGLDASQGPWPMHPVGMQ

SEQ ID NO: 32 - protein, 325 aa, Homo sapiens
MPITRMRMRPWLEMQINSNQIPGLIWINKEEMIFQIPWKHAAKHGWDINKDACLFRSWAIHTGRYKAGEKEPDPKTWKANFRCAMNSLPDIEEVKDQSRNKGSSAVRVYRMLPPLTKNQRKERKSKSSRDAKSKAKRKSCGDSSPDTFSDGLSSSTLPDDHSSYTVPGYMQDLEVEQALTPALSPCAVSSTLPDWHIPVEVVPDSTSDLYNFQVSPMPSTSEATTDEDEEGKLPEDIMKLLEQSEWQPTNVDGKGYLLNEPGVQPTSVYGDFSCKEEPEIDSPGGDIGLSLQRVFTDLKNMDATWLDSLLTPVRLPSIQAIPCAP

SEQ ID NO: 33 - protein, 427 aa, Homo sapiens
MGTPKPRILPWLVSQLDLGQLEGVAWVNKSRTRFRIPWKHGLRQDAQQEDFGIFQAWAEATGAYVPGRDKPDLPTWKRNFRSALNRKEGLRLAEDRSKDPHDPHKIYEFVNSGVGDFSQPDTSPDTNGGGSTSDTQEDILDELLGNMVLAPLPDPGPPSLAVAPEPCPQPLRSPSLDNPTPFPNLGPSENPLKRLLVPGEEWEFEVTAFYRGRQVFQQTISCPEGLRLVGSEVGDRTLPGWPVTLPDPGMSLTDRGVMSYVRHVLSCLGGGLALWRAGQWLWAQRLGHCHTYWAVSEELLPNSGHGPDGEVPKDKEGGVFDLGPFIVDLITFTEGSGRSPRYALWFCVGESWPQDQPWTKRLVMVKVVPTCLRALVEMARVGGASSLENTVDLHISNSHPLSLTSDQYKAYLQDLVEGMDFQGPGES

SEQ ID NO: 34 - protein, 503 aa, Homo sapiens
MALAPERAAPRVLFGEWLLGEISSGCYEGLQWLDEARTCFRVPWKHFARKDLSEADARIFKAWAVARGRWPPSSRGGGPPPEAETAERAGWKTNFRCALRSTRRFVMLRDNSGDPADPHKVYALSRELCWREGPGTDQTEAEAPAAVPPPQGGPPGPFLAHTHAGLQAPGPLPAPAGDKGDLLLQAVQQSCLADHLLTASWGADPVPTKAPGEGQEGLPLTGACAGGPGLPAGELYGWAVETTPSPGPQPAALTTGEAAAPESPHQAEPYLSPSPSACTAVQEPSPGALDVTIMYKGRTVLQKVVGHPSCTFLYGPPDPAVRATDPQQVAFPSPAELPDQKQLRYTEELLRHVAPGLHLELRGPQLWARRMGKCKVYWEVGGPPGSASPSTPACLLPRNCDTPIFDFRVFFQELVEFRARQRRGSPRYTIYLGFGQDLSAGRPKEKSLVLVKLEPWLCRVHLEGTQREGVSSLDSSSLSLCLSSANSLYDDIECFLMELEQPA

SEQ ID NO: 35 - protein, 426 aa, Homo sapiens
MCDRNGGRRLRQWLIEQIDSSMYPGLIWENEEKSMFRIPWKHAGKQDYNQEVDASIFKAWAVFKGKFKEGDKAEPATWKTRLRCALNKSPDFEEVTDRSQLDISEPYKVYRIVPEEEQKCKLGVATAGCVNEVTEMECGRSEIDELIKEPSVDDYMGMIKRSPSPPEACRSQLLPDWWAQQPSTGVPLVTGYTTYDAHHSAFSQMVISFYYGGKLVGQATTTCPEGCRLSLSQPGLPGTKLYGPEGLELVRFPPADAIPSERQRQVTRKLFGHLERGVLLHSSRQGVFVKRLCQGRVFCSGNAVVCKGRPNKLERDEVVQVFDTSQFFRELQQFYNSQGRLPDGRVVLCFGEEFPDMAPLRSKLILVQIEQLYVRQLAEEAGKSCGAGSVMQAPEEPPPDQVFRMFPDICASHQRSFFRENQQITV

SEQ ID NO: 36 - protein, 329 aa, Mus musculus
MPITRMRMRPWLEMQINSNQIPGLIWINKEEMIFQIPWKHAAKHGWDINKDACLFRSWAIHTGRYKAGEKEPDPKTWKANFRCAMNSLPDIEEVKDQSRNKGSSAVRVYRMLPPLTRNQRKERKSKSSRDTKSKTKRKLCGDVSPDTFSDGLSSSTLPDDHSSYTTQGYLGQDLDMERDITPALSPCVVSSSLSEWHMQMDIIPDSTTDLYNLQVSPMPSTSEAATDEDEEGKIAEDLMKLFEQSEWQPTHIDGKGYLLNEPGTQLSSVYGDFSCKEEPEIDSPRGDIGIGIQHVFTEMKNMDSIMWMDSLLGNSVRLPPSIQAIPCAP

SEQ ID NO: 37 - protein, 419 aa, Mus musculus
METPKPRILPWLVSQLDLGQLEGVAWLDESRTRFRIPWKHGLRQDAQMADFGIFQAWAEASGAYTPGKDKPDVSTWKRNFRSALNRKEVLRLAADNSKDPYDPHKVYEFVTPGARDFVHLGASPDTNGKSSLPHSQENLPKLFDGLILGPLKDEGSSDLAIVSDPSQQLPSPNVNNFLNPAPQENPLKQLLAEEQWEFEVTAFYRGRQVFQQTLFCPGGLRLVGSTADMTLPWQPVTLPDPEGFLTDKLVKEYVGQVLKGLGNGLALWQAGQCLWAQRLGHSHAFWALGEELLPDSGRGPDGEVHKDKDGAVFDLRPFVADLIAFMEGSGHSPRYTLWFCMGEMWPQDQPWVKRLVMVKVVPTCLKELLEMAREGGASSLKTVDLHISNSQPISLTSDQYKAYLQDLVEDMDFQATGNI

SEQ ID NO: 38 - protein, 456 aa, Mus musculus
MAEVRGVQRVLFGDWLLGEVSSGQYEGLQWLNEARTVFRVPWKHFGRRDLDEEDAQIFKAWAVARGRWPPSGVNLPPPEAEAAERRERRGWKTNFRCALHSTGRFILRQDNSGDPVDPHKVYELSRELGSTVGPATENREEVSLSNALPTQGVSPGSFLARENAGLQTPSPLLSSDAGDLLLQVLQYSHILESESGADPVPPQAPGQEQDRVYEEPYAAWQVEAVPSPRPQQPALTERSLGFLDVTIMYKGRTVLQAVVGHPRCVFLYSPMAPAVRTSEPQPVIFPSPAELPDQKQLHYTETLLQHVSPGLQLELRGPSLWALRMGKCKVYWEVGSPMGTTGPSTPPQLLERNRHTPIFDFSTFFRELEEFRARRRQGSPHYTIYLGFGQDLSAGRPKEKTLILVKLEPWVCKAYLEGVQREGVSSLDSSSLGLCLSSTNSLYEDIEHFLMDLQWP

SEQ ID NO: 39 - protein, 525 aa, Mus musculus
MAEVRGVQRVLFGDWLLGEVSSGQYEGLQWLNEARTVFRVPWKHFGRRDLDEEDAQIFKAWAVARGRWPPSGVNLPPPEAEAAERRERRGWKTNFRCALHSTGRFILRQDNSGDPVDPHKVYELSRELGSTVGPATENREEVSLSNALPTQGVSPGSFLARENAGLQTPSPLLSSDAGDLLLQVLQYSHILESESGADPVPPQAPGQEQDRVYEEPYAAWQVEAVPSPRPQQPALTERSLGFLDVTKLFDGLILGPLKDEGSSDLAIVSDPSQQLPSPNVNNFLNPAPQENPLKQLLAEEQWEFEVTAFYRGRQVFQQTLFCPGGLRLVGSTADMTLPWQPVTLPDPEGFLTDKLVKEYVGQVLKGLGNGLALWQAGQCLWAQRLGHSHAFWALGEELLPDSGRGPDGEVHKDKDGAVFDLRPFVADLIAFMEGSGHSPRYTLWFCMGEMWPQDQPWVKRLVMVKVVPTCLKELLEMAREGGASSLKTVDLHIDNDQPIDLDDDQYKAYLQDLVEDMDFQATGNI

SEQ ID NO: 40 - protein, 424 aa, Mus musculus
MCDRNGGRRLRQWLIEQIDSSMYPGLIWENDEKTMFRIPWKHAGKQDYNQEVDASIFKAWAVFKGKFKEGDKAEPATWKTRLRCALNKSPDFEEVTDRSQLDISEPYKVYRIVPEEEQKCKLGVAPAGCMSEVPEMECGRSEIEELIKEPSVDEYMGMTKRSPSPPEACRSQILPDWWVQQPSAGLPLVTGYAAYDTHHSAFSQMVISFYYGGKLVGQATTTCLEGCRLSLSQPGLPKLYGPDGLEPVCFPTADTIPSERQRQVTRKLFGHLERGVLLHSNRKGVFVKRLCQGRVFCSGNAVVCKGRPNKLERDEVVQVFDTNQFIRELQQFYATQSRLPDSRVVLCFGEEFPDTVPLRSKLILVQVEQLYARQLVEEAGKSCGAGSLMPALEEPQPDQAFRMFPDICTSHQRPFFRENQQITV

SEQ ID NO: 41 - protein, 424 aa, Mus musculus
MCDRNGGRRLRQWLIEQIDSSMYPGLIWENDEKTMFRIPWKHAGKQDYNQEVDASIFKAWAVFKGKFKEGDKAEPATWKTRLRCALNKSPDFEEVTDRSQLDISEPYKVYRIVPEEEQKCKLGVAPAGCMSEVPEMECGRSEIEELIKEPSVDEYMGMTKRSPSPPEACRSQILPDWWVQQPSAGLPLVTGYAAYDTHHSAFSQMVISFYYGGKLVGQATTTCLEGCRLSLSQPGLPKLYGPDGLEPVCFPTADTIPSERQRQVTRKLFGHLERGVLLHSNRKGVFVKRLCQGRVFCSGNAVVCKGRPNRLERDEVVQVFDTNQFIRELQQFYATQSRLPDSRVVLCFGEEFPDTVPLRSKLILVQVEQLYARQLVEEAGKSCGAGSLMPALEEPQPDQAFRMFPDICTSHQRPFFRENQQITV

SEQ ID NO: 42 - protein, 756 aa, Homo sapiens
MSWSPSLTTQTCGAWEMKERLGTGGFGNVIRWHNQETGEQIAIKQCRQELSPRNRERWCLEIQIMRRLTHPNVVAARDVPEGMQNLAPNDLPLLAMEYCQGGDLRKYLNQFENCCGLREGAILTLLSDIASALRYLHENRIIHRDLKPENIVLQQGEQRLIHKIIDLGYAKELDQGSLCTSFVGTLQYLAPELLEQQKYTVTVDYWSFGTLAFECITGFRPFLPNWQPVQWHSKVRQKSEVDIVVSEDLNGTVKFSSSLPYPNNLNSVLAERLEKWLQLMLMWHPRQRGTDPTYGPNGCFKALDDILNLKLVHILNMVTGTIHTYPVTEDESLQSLKARIQQDTGIPEEDQELLQEAGLALIPDKPATQCISDGKLNEGHTLDMDLVFLFDNSKITYETQISPRPQPESVSCILQEPKRNLAFFQLRKVWGQVWHSIQTLKEDCNRLQQGQRAAMMNLLRNNSCLSKMKNSMASMSQQLKAKLDFFKTSIQIDLEKYSEQTEFGITSDKLLLAWREMEQAVELCGRENEVKLLVERMMALQTDIVDLQRSPMGRKQGGTLDDLEEQARELYRRLREKPRDQRTEGDSQEMVRLLLQAIQSFEKKVRVIYTQLSKTVVCKQKALELLPKVEEVVSLMNEDEKTVVRLQEKRQKELWNLLKIACSKVRGPVSGSPDSMNASRLSQPGQLMSQPSTASNSLPEPAKKSEELVAEAHNLCTLLENAIQDTVREQDQSFTALDWSWLQTEEEEHSCLEQAS

SEQ ID NO: 43 - protein, 754 aa, Homo sapiens
MFSGGCHSPGFGRPSPAFPAPGSPPPAPRPCRQETGEQIAIKQCRQELSPRNRERWCLEIQIMRRLTHPNVVAARDVPEGMQNLAPNDLPLLAMEYCQGGDLRKYLNQFENCCGLREGAILTLLSDIASALRYLHENRIIHRDLKPENIVLQQGEQRLIHKIIDLGYAKELDQGSLCTSFVGTLQYLAPELLEQQKYTVTVDYWSFGTLAFECITGFRPFLPNWQPVQWHSKVRQKSEVDIVVSEDLNGTVKFSSSLPYPNNLNSVLAERLEKWLQLMLMWHPRQRGTDPTYGPNGCFKALDDILNLKLVHILNMVTGTIHTYPVTEDESLQSLKARIQQDTGIPEEDQELLQEAGLALIPDKPATQCISDGKLNEGHTLDMDLVFLFDNSKITYETQISPRPQPESVSCILQEPKRNLAFFQLRKVWGQVWHSIQTLKEDCNRLQQGQRAAMMNLLRNNSCLSKMKNSMASMSQQLKAKLDFFKTSIQIDLEKYSEQTEFGITSDKLLLAWREMEQAVELCGRENEVKLLVERMMALQTDIVDLQRSPMGRKQGGTLDDLEEQARELYRRLREKPRDQRTEGDSQEMVRLLLQAIQSFEKKVRVIYTQLSKTVVCKQKALELLPKVEEVVSLMNEDEKTVVRLQEKRQKELWNLLKIACSKVRGPVSGSPDSMNASRLSQPGQLMSQPSTASNSLPEPAKKSEELVAEAHNLCTLLENAIQDTVREQDQSFTALDWSWLQTEEEEHSCLEQAS

SEQ ID NO: 44 - protein, 256 aa, Homo sapiens
MSWSPSLTTQTCGAWEMKERLGTGGFGNVIRWHNQETGEQIAIKQCRQELSPRNRERWCLEIQIMRRLTHPNVVAARDVPEGMQNLAPNDLPLLAMEYCQGGDLRKYLNQFENCCGLREGAILTLLSDIASALRYLHENRIIHRDLKPENIVLQQGEQRLIHKIIDLGYAKELDQGSLCTSFVGTLQYLAPELLEQQKYTVTVDYWSFGTLAFECITGFRPFLPNWQPVQCVRMWPGTVAHSCNPSTLGGRGRWIS

SEQ ID NO: 45 - protein, 697 aa, Homo sapiens
MSSDGTIRLTHPNVVAARDVPEGMQNLAPNDLPLLAMEYCQGGDLRKYLNQFENCCGLREGAILTLLSDIASALRYLHENRIIHRDLKPENIVLQQGEQRLIHKIIDLGYAKELDQGSLCTSFVGTLQYLAPELLEQQKYTVTVDYWSFGTLAFECITGFRPFLPNWQPVQWHSKVRQKSEVDIVVSEDLNGTVKFSSSLPYPNNLNSVLAERLEKWLQLMLMWHPRQRGTDPTYGPNGCFKALDDILNLKLVHILNMVTGTIHTYPVTEDESLQSLKARIQQDTGIPEEDQELLQEAGLALIPDKPATQCISDGKLNEGHTLDMDLVFLFDNSKITYETQISPRPQPESVSCILQEPKRNLAFFQLRKVWGQVWHSIQTLKEDCNRLQQGQRAAMMNLLRNNSCLSKMKNSMASMSQQLKAKLDFFKTSIQIDLEKYSEQTEFGITSDKLLLAWREMEQAVELCGRENEVKLLVERMMALQTDIVDLQRSPMGRKQGGTLDDLEEQARELYRRLREKPRDQRTEGDSQEMVRLLLQAIQSFEKKVRVIYTQLSKTVVCKQKALELLPKVEEVVSLMNEDEKTVVRLQEKRQKELWNLLKIACSKVRGPVSGSPDSMNASRLSQPGQLMSQPSTASNSLPEPAKKSEELVAEAHNLCTLLENAIQDTVREQDQSFTALDWSWLQTEEEEHSCLEQAS

SEQ ID NO: 46 - protein, 738 aa, Mus musculus
MSWSPSLPTQTCGAWEMKERLGTGGFGNVIRWHNQATGEQIAIKQCRQELSPKNRDRWCLEIQIMRRLNHPNVVAARDVPEGMQNLAPNDLPLLAMEYCQGGDLRRYLNQFENCCGLREGAVLTLLSDIASALRYLHENRIIHRDLKPENIVLQQGEKRLIHKIIDLGYAKELDQGSLCTSFVGTLQYLAPELLEQQKYTVTVDYWSFGTLAFECITGFRPFLPNWQPVQWHSKVRQKSEVDIVVSEDLNGAVKFSSSLPFPNNLNSVLAERLEKWLQLMLMWHPRQRGTDPQYGPNGCFRALDDILNLKLVHVLNMVTGTVHTYPVTEDESLQSLKTRIQEDTGILETDQELLQEAGLVLLPDKPATQCISDSKTNEGLTLDMDLVFLFDNSKINYETQITPRPQPESVSCILQEPKRNLSFFQLRKVWGQVWHSIQTLKEDCNRLQQGQRAAMMSLLRNNSCLSKMKNAMASTAQQLKAKLDFFKTSIQIDLEKYKEQTEFGITSDKLLLAWREMEQAVEQCGRENDVKHLVERMMALQTDIVDLQRSPMGRKQGGTLDDLEEQARELYRRLREKPRDQRTEGDSQEMVRLLLQAIQSFEKKVRVIYTQLSKTVVCKQKALELLPKVEEVVSLMNEDERTVVRLQEKRQKELWNLLKIACSKVRGPVSGSPDSMNVSRLSHPGQLMSQPSSACDSLPESDKKSEELVAEAHALCSRLESALQDTVKEQDRSFTVTA

SEQ ID NO: 47 - DNA, 1497 bp, Homo sapiens
atgaaccagtccatcccagtggctcccaccccaccccgccgcgtgcggctgaagccctggctggtggcccaggtgaacagctgccagtacccagggcttcaatgggtcaacggggaaaagaaattattctgcatcccctggaggcatgccacaaggcatggtcccagccaggacggagataacaccatcttcaaggcctgggccaaggagacagggaaatacaccgaaggcgtggatgaagccgatccggccaagtggaaggccaacctgcgctgtgcccttaacaagagccgggacttccgcctcatctacgacgggccccgggacatgccacctcagccctacaagatctacgaggtctgctccaatggccctgctcccacagactcccagccccctgaggattactcttttggtgcaggagaggaggaggaagaagaggaagagctgcagaggatgttgccaagcctgagcctcacagaggatgtcaagtggccgcccactctgcagccgcccactctgcggccgcctactctgcagccgcccactctgcagccgcccgtggtgctgggtccccctgctccagaccccagccccctggctcctccccctggcaaccctgctggcttcagggagcttctctctgaggtcctggagcctgggcccctgcctgccagcctgccccctgcaggcgaacagctcctgccagacctgctgatcagcccccacatgctgcctctgaccgacctggagatcaagtttcagtaccgggggcggccaccccgggccctcaccatcagcaacccccatggctgccggctcttctacagccagctggaggccacccaggagcaggtggaactcttcggccccataagcctggagcaagtgcgcttccccagccctgaggacatccccagtgacaagcagcgcttctacacgaaccagctgctggatgtcctggaccgcgggctcatcctccagctacagggccaggacctttatgccatccgcctgtgtcagtgcaaggtgttctggagcgggccttgtgcctcagcccatgactcatgccccaaccccatccagcgggaggtcaagaccaagcttttcagcctggagcattttctcaatgagctcatcctgttccaaaagggccagaccaacaccccaccacccttcgagatcttcttctgctttggggaagaatggcctgaccgcaaaccccgagagaagaagctcattactgtacaggtggtgcctgtagcagctcgactgctgctggagatgttctcaggggagctatcttggtcagctgatagtatccggctacagatctcaaacccagacctcaaagaccgcatggtggagcaattcaaggagctccatcacatctggcagtcccagcagcggttgcagcctgtggcccaggcccctcctggagcaggccttggtgttggccaggggccctggcctatgcacccagctggcatgcaataa

SEQ ID NO: 48 - DNA, 1544 bp, Homo sapiens
atgaaccagtccatcccagtggctcccaccccaccccgccgcgtgcggctgaagccctggctggtggcccaggtgaacagctgccagtacccagggcttcaatgggtcaacggggaaaagaaattattctgcatcccctggaggcatgccacaaggcatggtcccagccaggacggagataacaccatcttcaaggcctgggccaaggagacagggaaatacaccgaaggcgtggatgaagccgatccggccaagtggaaggccaacctgcgctgtgcccttaacaagagccgggacttccgcctcatctacgacgggccccgggacatgccacctcagccctacaagatctacgaggtctgctccaatggccctgctcccacagactcccagccccctgaggattactcttttggtgcaggagaggaggaggaagaagaggaagagctgcagaggatgttgccaagcctgagcctcacagatgcagtgcagtctggcccccacatgacaccctattctttactcaaagaggatgtcaagtggccgcccactctgcagccgcccactctgcggccgcctactctgcagccgcccactctgcagccgcccgtggtgctgggtccccctgctccagaccccagccccctggctcctccccctggcaaccctgctggcttcagggagcttctctctgaggtcctggagcctgggcccctgcctgccagcctgccccctgcaggcgaacagctcctgccagacctgctgatcagcccccacatgctgcctctgaccgacctggagatcaagtttcagtaccgggggcggccaccccgggccctcaccatcagcaacccccatggctgccggctcttctacagccagctggaggccacccaggagcaggtggaactcttcggccccataagcctggagcaagtgcgcttccccagccctgaggacatccccagtgacaagcagcgcttctacacgaaccagctgctggatgtcctggaccgcgggctcatcctccagctacagggccaggacctttatgccatccgcctgtgtcagtgcaaggtgttctggagcgggccttgtgcctcagcccatgactatgccccaaccccatccagcgggaggtcaagaccaagcttttcagcctggagcattttctcaatgagctcatcctgttccaaaagggccagaccaacaccccaccacccttcgagatcttcttctgctttggggaagaatggcctgaccgcaaaccccgagagaagaagctcattactgtacaggtggtgcctgtagcagctcgactgctgctggagatgttctcaggggagctatcttggtcagctgatagtatccggctacagatctcaaacccagacctcaaagaccgcatggtggagcaattcaaggagctccatcacatctggcagtcccagcagcggttgcagcctgtggcccaggcccctcctggagcaggccttggtgttggccaggggccctggcctatgcacccagctggcatgcaataa

SEQ ID NO: 49 - DNA, 1514 bp, Homo sapiens
atgaaccagtccatcccagtggctcccaccccaccccgccgcgtgcggctgaagccctggctggtggcccaggtgaacagctgccagtacccagggcttcaatgggtcaacggggaaaagaaattattctgcatcccctggaggcatgccacaaggcatggtcccagccaggacggagataacaccatcttcaaggcctgggccaaggagacagggaaatacaccgaaggcgtggatgaagccgatccggccaagggaaggccaacctgcgctgtgcccttaacaagagccgggacttccgcctcatctacgacgggccccgggacatgccacctcagccctacaagatctacgaggtctgctccaatggccctgctcccacagactcccagccccctgaggattactcttttggtgcaggagaggaggaggaagaagaggaagagctgcagaggatgttgccaagcctgagcctcacagatgcagtgcagtctggcccccacatgacaccctattctttactcaaagaggatgtcaagtggccgcccactctgcagccgcccactctgcagccgcccgtggtgctgggtccccctgctccagaccccagccccctggctcctccccctggcaaccctgctggcttcagggagcttctctctgaggtcctggagcctgggcccctgcctgccagcctgccccctgcaggcgaacagctcctgccagacctgctgatcagcccccacatgctgcctctgaccgacctggagatcaagtttcagtaccgggggcggccaccccgggccctcaccatcagcaacccccatggctgccggctcttctacagccagctggaggccacccaggagcaggtggaactcttcggccccataagcctggagcaagtgcgcttccccagccctgaggacatccccagtgacaagcagcgcttctacacgaaccagctgctggatgtcctggaccgcgggctcatcctccagctacagggccaggacctttatgccatccgcctgtgtcagtgcaaggtgttctggagcgggccttgtgcctcagcccatgactcatgccccaaccccatccagcgggaggtcaagaccaagcttttcagcctggagcattttctcaatgagctcatcctgttccaaaagggccagaccaacaccccaccacccttcgagatcttcttctgctttggggaagaatggcctgaccgcaaaccccgagagaagaagctcattactgtacaggtggtgcctgtagcagctcgactgctgctggagatgttctcaggggagctatcttggtcagctgatagtatccggctacagatctcaaacccagacctcaaagaccgcatggtggagcaattcaaggagctccatcacatctggcagtcccagcagcggttgcagcctgtggcccaggcccctcctggagcaggccttggtgttggccaggggccctggcctatgcacccagctggcatgcaataa

SEQ ID NO: 50 - DNA, 1466 bp, Homo sapiens
atgaaccagtccatcccagtggctcccaccccaccccgccgcgtgcggctgaagccctggctggtggcccaggtgaacagctgccagtacccagggcttcaatgggtcaacggggaaaagaaattattctgcatcccctggaggcatgccacaaggcatggtcccagccaggacggagataacaccatcttcaaggcctgggccaaggagacagggaaatacaccgaaggcgtggatgaagccgatccggccaagtggaaggccaacctgcgctgtgcccttaacaagagccgggacttccgcctcatctacgacgggccccgggacatgccacctcagccctacaagatctacgaggtctgctccaatggccctgctcccacagactcccagccccctgaggattactcttttggtgcaggagaggaggaggaagaagaggaagagctgcagaggatgttgccaagcctgagcctcacagaggatgtcaagtggcgcccactctgcagccgcccactctgcagccgcccgtggtgctgggtccccctgctccagaccccagccccctggctcctccccctggcaaccctgctggcttcagggagcttctctctgaggtcctggagcctgggcccctgcctgccagcctgccccctgcaggcgaacagctcctgccagacctgctgatcagcccccacatgctgcctctgaccgacctggagatcaagtttcagtaccgggggcggccaccccgggccctcaccatcagcaacccccatggctgccggctcttctacagccagctggaggccacccaggagcaggtggaactcttcggccccataagcctggagcaagtgcgcttccccagccctgaggacatccccagtgacaagcagcgcttctacacgaaccagctgctggatgtcctggaccgcgggctcatcctccagctacagggccaggacctttatgccatccgcctgtgtcagtgcaaggtgttctggagcgggccttgtgcctcagcccatgactcatgccccaaccccatccagcgggaggtcaagaccaagcttttcagcctggagcattttctcaatgagctcatcctgttccaaaagggccagaccaacaccccaccacccttcgagatcttcttctgctttggggaagaatggcctgaccgcaaaccccgagagaagaagctcattactgtacaggtggtgcctgtagcagctcgactgctgctggagatgttctcaggggagctatcttggtcagctgatagtatccggctacagatctcaaacccagacctcaaagaccgcatggtggagcaattcaaggagctccatcacatctggcagtcccagcagcggttgcagcctgtggcccaggcccctcctggagcaggccttggtgttggccaggggccctggcctatgcacccagctggcatgcaataa

SEQ ID NO: 51 - DNA, 1239 bp, Homo sapiens
atgaaccagtccatcccagtggctcccaccccaccccgccgcgtgcggctgaagccctggctggtggcccaggtgaacagctgccagtacccagggcttcaatgggtcaacggggaaaagaaattattctgcatcccctggaggcatgccacaaggcatggtcccagccaggacggagataacaccatcttcaaggcctgggccaaggagacagggaaatacaccgaaggcgtggatgaagccgatccggccaagtggaaggccaacctgcgctgtgcccttaacaagagccgggacttccgcctcatctacgacgggccccgggacatgccacctcagccctacaagatctacgaggtctgctccaatggccctgctcccacagactcccagccccctgaggattactcttttggtgcaggagaggaggaggaagaagaggaagagctgcagaggatgttgccaagcctgagcctcacagtgaccgacctggagatcaagtttcagtaccgggggcggccaccccgggccctcaccatcagcaacccccatggctgccggctcttctacagccagctggaggccacccaggagcaggtggaactcttcggccccataagcctggagcaagtgcgcttccccagccctgaggacatccccagtgacaagcagcgcttctacacgaaccagctgctggatgtcctggaccgcgggctcatcctccagctacagggccaggacctttatgccatccgcctgtgtcagtgcaaggtgttctggagcgggccttgtgcctcagcccatgactcatgccccaaccccatccagcgggaggtcaagaccaagcttttcagcctggagcattttctcaatgagctcatcctgttccaaaagggccagaccaacaccccaccacccttcgagatcttcttctgctttggggaagaatggcctgaccgcaaaccccgagagaagaagctcattactgtacaggtggtgcctgtagcagctcgactgctgctggagatgttctcaggggagctatcttggtcagctgatagtatccggctacagatctcaaacccagacctcaaagaccgcatggtggagcaattcaaggagctccatcacatctggcagtcccagcagcggttgcagcctgtggcccaggcccctcctggagcaggccttggtgttggccaggggccctggcctatgcacccagctggcatgcaataa

SEQ ID NO: 52 - DNA, 444 bp, Homo sapiens
atgaaccagtccatcccagtggctcccaccccaccccgccgcgtgcggctgaagccctggctggtggcccaggtgaacagctgccagtacccagggcttcaatgggtcaacggggaaaagaaattattctgcatcccctggaggcatgccacaaggcatggtcccagccaggacggagataacaccatcttcaaggcctgggccaaggagacagggaaatacaccgaaggcgtggatgaagccgatccggccaagtggaaggccaacctgcgctgtgcccttaacaagagccgggacttccgcctcatctacgacgggccccgggacatgccacctcagccctacaagatctacgagactcccagccccctgaggattactcttttggtgcaggagaggaggaggaagaagaggaagagctgcagaggatgttgccaagcctga

SEQ ID NO: 53 - DNA, 1494 bp, Mus musculus
atgaatcatagtgcacccgggatccctcctcctccaagacgagtacgcctcaagccctggttggtagctcaagtcaactcatgccaataccctgggcttcagtgggtgaacggtgagaagaaattgttttatatcccatggcgacacgcaacaagacatggcccatcacaggatggagataacaccatatttaaggcatgggcaaaggaaacaggaaagtacactgagggcgttgatgaggccgatcctgcaaaatggaaagcaaatttgcgatgcgctctcaataaatcacgagatttccaactcttttacgacggcccaagggacatgccaccacaaccttataaaatctacgaggtatgttccaacggtccagccccaactgaatcccagcctactgacgactatgtccttggagaagaggaggaagaagaagaggaggaacttcagcggatgttgcctgggttgtccataactgagcctgccttgccaggaccccctaatgcaccatactcccttcccaaagaagatacaaaatggccccccgcattgcaaccccccgttggtttgggaccacctgtgcccgacccaaatctcttggccccaccaagcggtaacccagccggatttcgacaacttctgcccgaagtccttgagccaggtcccttggcctcttctcagccccctacagaacctctgctccccgatctcttgatatctccccacatgcttcccttgactgatttggagataaaatttcagtatcgcggccgagctcccagaacactgactatatcaaatccccaaggttgccgcctgttttacagtcagttggaggcaactcaggagcaagtagagctctttgggccagttactctggagcaggtgagattccctagtccagaggacataccaagcgataagcaaagattttacacaaatcaacttctggatgtacttgatcgaggtttgatccttcagttgcagggccaagatttgtatgccattcgactctgtcaatgcaaggtattttggagcggcccatgtgcccttgctcatggcagctgccctaatcccatccaaagagaagtaaagactaaacttttcagcctggaacaatttctcaacgaactcattctgtttcaaaaaggtcagaccaacacaccccctcctttcgagattttcttctgcttcggcgaagagtggcctgatgtgaagccccgcgaaaaaaagcttatcaccgttcaagtggtacccgtcgcagccaggctccttcttgaaatgtttagcggtgaactctcatggtccgctgacagtatccggctccaaatatcaaaccctgatcttaaagaccacatggtagaacagtttaaagaactccaccacctgtggcaatcccaacagcagctccagccaatggttcaagctcctccagtcgctgggctggacgcctcacaaggaccctggcccatgcaccccgtcgggatgcagtaa

SEQ ID NO: 54 - DNA, 978 bp, Homo sapiens
atgcccatcactcggatgcgcatgagaccctggctagagatgcagattaattccaaccaaatcccggggctcatctggattaataaagaggagatgatcttccagatcccatggaagcatgctgccaagcatggctgggacatcaacaaggatgcctgtttgttccggagctgggccattcacacaggccgatacaaagcaggggaaaaggagccagatcccaagacgtggaaggccaactttcgctgtgccatgaactccctgccagatatcgaggaggtgaaagaccagagcaggaacaagggcagctcagctgtgcgagtgtaccggatgcttccacctctcaccaagaaccagagaaaagaaagaaagtcgaagtccagccgagatgctaagagcaaggccaagaggaagtcatgtggggattccagccctgataccttctctgatggactcagcagctccactctgcctgatgaccacagcagctacacagttccaggctacatgcaggacttggaggtggagcaggccctgactccagcactgtcgccatgtgctgtcagcagcactctccccgactggcacatcccagtggaagttgtgccggacagcaccagtgatctgtacaacttccaggtgtcacccatgccctccacctctgaagctacaacagatgaggatgaggaagggaaattacctgaggacatcatgaagctcttggagcagtcggagtggcagccaacaaacgtggatgggaaggggtacctactcaatgaacctggagtccagcccacctctgtctatggagactttagctgtaaggaggagccagaaattgacagcccagggggggatattgggctgagtctacagcgtgtcttcacagatctgaagaacatggatgccacctggctggacagcctgctgaccccagtccggttgccctccatccaggccattccctgtgcaccgtag

SEQ ID NO: 55 - DNA, 1283 bp, Homo sapiens
atgggaaccccaaagccacggatcctgccctggctggtgtcgcagctggacctggggcaactggagggcgtggcctgggtgaacaagagccgcacgcgcttccgcatcccttggaagcacggcctacggcaggatgcacagcaggaggatttcggaatcttccaggcctgggccgaggccactggtgcatatgttcccgggagggataagccagacctgccaacctggaagaggaatttccgctctgccctcaaccgcaaagaagggttgcgtttagcagaggaccggagcaaggaccctcacgacccacataaaatctacgagtttgtgaactcaggagttggggacttttcccagccagacacctctccggacaccaatggtggaggcagtacttctgatacccaggaagacattctggatgagttactgggtaacatggtgttggccccactcccagatccgggacccccaagcctggctgtagcccctgagccctgccctcagcccctgcggagccccagcttggacaatcccactcccttcccaaacctggggccctctgagaacccactgaagcggctgttggtgccgggggaagagtgggagttcgaggtgacagccttctaccggggccgccaagtcttccagcagaccatctcctgcccggagggcctgcggctggtggggtccgaagtgggagacaggacgctgcctggatggccagtcacactgccagaccctggcatgtccctgacagacaggggagtgatgagctacgtgaggcatgtgctgagctgcctgggtgggggactggctctctggcgggccgggcagtggctctgggcccagcggctggggcactgccacacatactgggcagtgagcgaggagctgctccccaacagcgggcatgggcctgatgcgaggtccccaaggacaaggaaggaggcgtgtttgacctggggcccttcattgtagatctgattaccttcacggaaggaagcggacgctcaccacgctatgccctctggttctgtgtgggggagtcatggccccaggaccagccgtggaccaagaggctcgtgatggtcaaggttgtgcccacgtgcctcagggccttggtagaaatggcccgggtagggggtgcctcctccctggagaatactgtggacctgcacatttccaacagccacccactctccctcacctccgaccagtacaaggcctacctgcaggacttggtggagggcatggatttccagggccctggggagagctga

SEQ ID NO: 56 - DNA, 1512 bp, Homo sapiens
atggccttggctcctgagagggcagccccacgcgtgctgttcggagagtggctccttggagagatcagcagcggctgctatgaggggctgcagtggctggacgaggcccgcacctgtttccgcgtgccctggaagcacttcgcgcgcaaggacctgagcgaggccgacgcgcgcatcttcaaggcctgggctgtggcccgcggcaggtggccgcctagcagcaggggaggtggcccgccccccgaggctgagactgcggagcgcgccggctggaaaaccaacttccgctgcgcactgcgcagcacgcgtcgcttcgtgatgctgcgggataactcgggggacccggccgacccgcacaaggtgtacgcgctcagccgggagctgtgctggcgagaaggcccaggcacggaccagactgaggcagaggcccccgcagctgtcccaccaccacagggtgggcccccagggccattcctggcacacacacatgctggactccaagccccaggccccctccctgccccagctggtgacaagggggacctcctgctccaggcagtgcaacagagctgcctggcagaccatctgctgacagcgtcatggggggcagatccagtcccaaccaaggctcctggagagggacaagaagggcttcccctgactggggcctgtgctggaggcccagggctccctgctggggagctgtacgggtgggcagtagagacgacccccagccccgggccccagcccgcggcactaacgacaggcgaggccgcggccccagagtccccgcaccaggcagagccgtacctgtcaccctccccaagcgcctgcaccgcggtgcaagagcccagcccaggggcgctggacgtgaccatcatgtacaagggccgcacggtgctgcagaaggtggtgggacacccgagctgcacgttcctatacggccccccagacccagctgtccgggccacagacccccagcaggtagcattccccagccctgccgagctcccggaccagaagcagctgcgctacacggaggaactgctgcggcacgtggcccctgggttgcacctggagcttcgggggccacagctgtgggcccggcgcatgggcaagtgcaaggtgtactgggaggtgggcggacccccaggctccgccagcccctccaccccagcctgcctgctgcctcggaactgtgacacccccatcttcgacttcagagtcttcttccaagagctggtggaattccgggcacggcagcgccgtggctccccacgctataccatctacctgggcttcgggcaggacctgtcagctgggaggcccaaggagaagagcctggtcctggtgaagctggaaccctggctgtgccgagtgcacctagagggcacgcagcgtgagggtgtgtcttccctggatagcagcagcctcagcctctgcctgtccagcgccaacagcctctatgacgacatcgagtgcttccttatggagctggagcagcccgcctag

SEQ ID NO: 57 - DNA, 1281 bp, Homo sapiens
atgtgtgaccggaatggtggtcggcggcttcgacagtggctgatcgagcagattgacagtagcatgtatccaggactgatttgggagaatgaggagaagagcatgttccggatcccttggaaacacgctggcaagcaagattataatcaggaagtggatgcctccatttttaaggcctgggcagtttttaaagggaagtttaaagaaggggacaaagctgaaccagccacttggaagacgaggttacgctgtgctttgaataagagcccagattttgaggaagtgacggaccggtcccaactggacatttccgagccatacaaagtttaccgaattgttcctgaggaagagcaaaaatgcaaactaggcgtggcaactgctggctgcgtgaatgaagttacagagatggagtgcggtcgctctgaaatcgacgagctgatcaaggagccttctgtggacgattacatggggatgatcaaaaggagcccttccccgccggaggcctgtcggagtcagctccttccagactggtgggcgcagcagcccagcacaggcgtgccgctggtgacggggtacaccacctacgacgcgcaccattcagcattctcccagatggtgatcagcttctactatgggggcaagctggtgggccaggccaccaccacctgccccgagggctgccgcctgtccctgagccagcctgggctgcccggcaccaagctgtatgggcccgagggcctggagctggtgcgcttcccgccggccgacgccatccccagcgagcgacagaggcaggtgacgcggaagctgttcgggcacctggagcgcggggtgctgctgcacagcagccggcagggcgtgttcgtcaagcggctgtgccagggccgcgtgttctgcagcggcaacgccgtggtgtgcaaaggcaggcccaacaagctggagcgtgatgaggtggtccaggtcttcgacaccagccagttcttccgagagctgcagcagttctataacagccagggccggcttcctgacggcagggtggtgctgtgctttggggaagagtttccggatatggcccccttgcgctccaaactcattctcgtgcagattgagcagctgtatgtccggcaactggcagaagaggctgggaagagctgtggagccggctctgtgatgcaggcccccgaggagccgccgccagaccaggtcttccggatgtttccagatatttgtgcctcacaccagagatcatttttcagagaaaaccaacagatcaccgtctaa

SEQ ID NO: 58 - DNA, 990 bp, Mus musculus
atgccaatcactcgaatgcggatgagaccctggctagagatgcagattaattccaaccaaatcccagggctgatctggatcaataaagaagagatgatcttccagattccatggaagcacgctgctaagcacggctgggacatcaacaaggatgcctgtctgttccggagctgggccattcacacaggccgatacaaagcaggagaaaaagagccagatcccaagacatggaaggcaaacttccgttgtgccatgaactccctgccagacatcgaggaagtgaaggatcagagtaggaacaagggcagctctgctgtgcgggtgtaccggatgctgccacccctcaccaggaaccagaggaaagagagaaagtccaagtccagccgagacactaagagcaaaaccaagaggaagctgtgtggagatgttagcccggacactttctctgatggactcagcagctctaccctacctgatgaccacagcagttacaccactcagggctacctgggtcaggacttggatatggaaagggacataactccagcactgtcaccgtgtgtcgtcagcagcagtctctctgagtggcatatgcagatggacattataccagatagcaccactgatctgtataacctacaggtgtcacccatgccttccacctccgaagccgcaacagacgaggatgaggaagggaagatagccgaagaccttatgaagctctttgaacagtctgagtggcagccgacacacatcgatggcaagggatacttgctcaatgagccagggacccagctctcttctgtctatggagacttcagctgcaaagaggaaccagagattgacagccctcgaggggacattgggataggcatacaacatgtcttcacggagatgaagaatatggactccatcatgtggatggacagcctgctgggcaactctgtgaggctgccgccctctattcaggccattccttgtgcaccatag

SEQ ID NO: 59 - DNA, 1260 bp, Mus musculus
atggaaaccccgaaaccgcggattttgccctggctggtgtcacagctggacctggggcagctggaaggcgtggcctggctggacgagagccgaacgaggttcaggatcccgtggaagcatggcctacggcaggacgcacagatggctgactttggcatcttccaggcctgggcagaagccagtggtgcctacaccccggggaaggataagccggacgtgtcaacctggaagaggaatttccggtcagccctgaaccggaaagaagtgttgcggttagctgctgacaatagcaaggacccttatgaccctcataaagtgtatgagtttgtgactccaggggcgcgggacttcgtacatctgggtgcctctcctgacaccaatggcaaaagcagcctgcctcactcccaggaaaacctaccgaagttatttgatggcctgatcttggggcccctcaaagatgaggggtcctcagatctggctattgtttctgatccttctcaacaactgccaagccccaatgtgaacaacttcctaaaccctgcaccccaagaaaatccactgaagcagctgctagctgaggaacaatgggagttcgaggtgaccgccttctaccgaggccgccaggtcttccagcagacactcttttgcccggggggcctgcggctggtgggcagcacagctgacatgacactgccctggcagccagtcaccctgcccgatcctgaggggtttctgacggacaagcttgtgaaggagtacgtggggcaggtgctcaaagggctgggcaatgggctggcactgtggcaggctgggcagtgcctctgggcccagcgcctaggccactcccacgccttctgggctctgggggaggagctgcttccagacagtgggcgagggcctgatggagaggtccacaaggacaaggacggagccgtgttcgacctcaggcccttcgtggcagatctgattgccttcatggaaggaagtggacactccccacgctacactctgtggttctgcatgggggaaatgtggccccaggaccagccatgggtcaagaggcttgtgatggtcaaggttgttcctacatgtcttaaggagctgttagagatggcccgggaagggggagcctcttcactgaaaaccgtggacttgcacatctccaacagccagcctatctcccttacctctgaccagtacaaggcctacctccaggacttggtggaggacatggacttccaggccactggaaatatctga

SEQ ID NO: 60 - DNA, 1374 bp, Mus musculus
atggctgaagtgaggggggtccagcgagtgctgtttggagactggctattgggggaggtcagcagcggccagtacgaggggctgcagtggctgaacgaggctcgcacagtcttccgcgtaccctggaagcatttcggtcgtagggatctggatgaagaagatgcacagatcttcaaggcctgggctgtggcccgagggaggtggccacctagtggagttaacctgccacccccagaggctgaggctgctgagcgaagagagcgaagaggctggaagaccaacttccgctgtgcactccacagcacagggcgttttatcttgcgccaagacaattcaggggatccagttgatccgcataaggtgtacgaacttagccgggagcttggatctactgtgggcccagccacggaaaatagggaagaagtgagcctcagcaatgctctgcccacacagggtgtgtccccaggatcatttctggcaagagaaaatgctgggctccaaaccccaagccctctgctttctagtgatgccggggacctcttgcttcaggttctgcagtacagccacatactggaatccgagtctggggcagaccccgtcccaccacaggctcctggccaggagcaagaccgtgtttacgaggaaccctatgcagcatggcaggtggaagctgtccccagtcccaggcctcaacagccagctctcaccgagcgcagccttgggttcctggatgtgaccatcatgtacaagggccgcacagtgctacaggcagtggtggggcaccccagatgcgtgttcctgtacagccccatggccccagcagtaagaacttcagagccccagccggtgatctttcccagtcctgctgagctcccagatcagaagcagctgcactacacagagacgcttctccagcatgtgtctcccggccttcagctggagcttcgaggaccgtcactgtgggccctgcgtatgggcaagtgcaaggtgtactgggaggtaggcagccctatgggcactaccggcccctccaccccaccccagctgctggagcgcaaccgccacacccccatcttcgacttcagcactttcttccgagaactggaggagtttcgggctcggaggcggcaagggtcaccacactacaccatctacctgggttttgggcaagacttgtcagcagggaggcccaaggagaagaccctgatcctggtgaagctggagccatgggtatgcaaggcatacctggagggcgtgcagcgtgagggtgtgtcctccctggacagcagcagtctcggcttgtgcttgtctagcaccaacagtctctacgaagacatcgaacacttcctcatggacctgggtcagtggccttga

SEQ ID NO: 61 - DNA, 1578 bp, Mus musculus
atggccgaagttcgaggagtacagcgcgtgctgtttggggactggttgcttggtgaagtctcttctggtcagtatgaaggcctgcaatggcttaatgaggcacgcacagtttttcgagtgccatggaaacacttcggtaggcgcgatctcgacgaagaggatgcccagattttcaaggcatgggcagtcgcacggggcaggtggcccccttcaggcgtaaatttgccccccccagaggctgaagctgctgaacgcagagaacgccggggatggaagactaactttcgatgtgcccttcacagtacaggcaggttcatcttgcggcaggataatagtggcgaccctgtagacccacacaaggtttatgagctgagccgggagcttggctcaacagtcggtcctgcaaccgagaacagagaagaggtgtccttgtctaacgccctcccaactcagggtgtgtctcccggtagcttcctggcacgcgaaaacgctggactccaaaccccctccccactgttgtccagtgatgccggtgatcttctccttcaggtgctccaatactcccatatactggagagcgagtcaggggctgatcccgtgccccctcaagctcctggacaggaacaagatcgcgtctacgaggagccatatgctgcctggcaggtcgaggctgtgccatcacctcggcctcaacagcccgctctcaccgagcgctcacttgggtttttggatgtcactaaacttttcgacggcctgatacttggcccattgaaggacgagggatcatccgatcttgccatagtaagtgacccatcacagcagttgccctcaccaaacgtcaacaacttcctcaatccagctccccaggagaaccccctcaaacagcttctcgcagaagagcaatgggagtttgaggtgactgctttctatagaggtaggcaggtgttccaacaaactctgttttgccccggaggtctgcgccttgtaggtagcaccgcagacatgacacttccctggcaacctgtgacacttcccgatcctgagggatttctcacagataaactcgttaaggaatatgtggggcaagtactcaaaggtctgggcaatgggttggccctttggcaagctggtcaatgtctctgggctcaacgactcgggcactcacatgctttttgggctcttggcgaggagctgctccccgacagcgggcgcggacctgacggggaggttcataaggacaaagacggcgccgtatttgatcttagacccttcgtggcagatctgatcgctttcatggaaggatcaggtcatagccccaggtacacactttggttttgcatgggtgaaatgtggcctcaggaccaaccttgggtcaagcgcttggtcatggttaaggtggttcccacttgcctcaaagagttgttggagatggctagggaaggtggggcttcctcactgaaaaccgtagatctccacattgataatgatcagcctatagatttggacgacgaccaatacaaagcttatctccaggacctggttgaagatatggactttcaggctacaggtaacatctaa

SEQ ID NO: 62 - DNA, 1275 bp, Mus musculus
atgtgtgacaggaacggcggtagaagactgagacagtggctgatcgagcaaattgacagctcaatgtaccctgggttgatatgggaaaacgatgaaaagacaatgttcagaataccctggaagcacgctggaaagcaggattacaaccaggaagtggacgccagtatttttaaggcttgggctgtcttcaaagggaagtttaaagagggcgacaaagcagagccagcaacctggaaaacccgcttgaggtgtgcactcaataagtcacccgacttcgaggaagtcactgaccgcagtcaattggacatatcagaaccatacaaagtctacaggatagtccccgaagaagagcagaaatgcaaactcggtgtagcacctgctggctgtatgagtgaagtgcctgaaatggaatgcggcagatcagaaatcgaagaactcataaaagaaccaagtgtagatgagtatatgggaatgaccaaaagatccccatcccccccagaagcctgtcggagccaaatcttgcctgactggtgggtacagcaaccctccgccggacttccccttgtgacaggctatgccgcttacgatactcatcacagcgcttttagccagatggttatttccttctattatggaggaaaactggtcggccaagccacaaccacctgcctcgaggggtgtcgcttgagtttgagtcaacccggtcttcccaaactctatggccccgatgggcttgaacctgtctgctttcccactgctgatactattccctcagagagacaacgacaagtcacccgaaaattgtttggccacctcgagaggggagtactcttgcactctaacaggaagggtgtctttgtgaaacgcctctgtcaaggtagggtattctgttctggaaatgcagttgtttgcaaaggcaggcctaacaaactggaacgggatgaagtcgtacaagtgttcgataccaatcagtttattcgggagttgcagcagttttacgctacacaaagtcgcctccctgacagtcgggttgtgttgtgcttcggggaggagtttcccgacactgtacccctccgaagcaaactcatactggtacaggtagaacaactttatgccaggcaactggtggaagaggccggtaagtcctgtggcgcaggatccctgatgccagccctggaagagccccagcctgaccaagcatttaggatgtttcccgacatttgtacctcacaccagaggccttttttccgcgaaaaccagcagataaccgtgtaa

SEQ ID NO: 63 - DNA, 1274 bp, Mus musculus
atgtgtgacaggaacggcggtagaagactgagacagtggctgatcgagcaaattgacagctcaatgtaccctgggttgatatgggaaaacgatgaaaagacaatgttcagaataccctggaagcacgctggaaagcaggattacaaccaggaagtggacgccagtatttttaaggcttgggctgtcttcaaagggaagtttaaagagggcgacaaagcagagccagcaacctggaaaacccgcttgaggtgtgcactcaataagtcacccgacttcgaggaagtcactgaccgcagtcaattggacatatcagaaccatacaaagtctacaggatagtccccgaagaagagcagaaatgcaaactcggtgtagcacctgctggctgtatgagtgaagtgcctgaaatggaatgcggcagatcagaaatcgaagaactcataaaagaaccaagtgtagatgagtatatgggaatgaccaaaagatccccatcccccccagaacctgtcggagccaaatcttgcctgactggtgggtacagcaaccctccgccggacttccccttgtgacaggctatgccgcttacgatactcatcacagcgcttttagccagatggttatttccttctattatggaggaaaactggtcggccaagccacaaccacctgcctcgaggggtgtcgcttgagtttgagtcaacccggtcttcccaaactctatggccccgatgggcttgaacctgtctgctttcccactgctgatactattccctcagagagacaacgacaagtcacccgaaaattgtttggccacctcgagaggggagtactcttgcactctaacaggaagggtgtctttgtgaaacgcctctgtcaaggtagggtattctgttctggaaatgcagttgtttgcaaaggcaggcctaacagactggaacgggatgaagtcgtacaagtgttcgataccaatcagtttattcgggagttgcagcagttttacgctacacaaagtcgcctccctgacagtcgggttgtgttgtgcttcggggaggagtttcccgacactgtacccctccgaagcaaactcatactggtacaggtagaacaactttatgccaggcaactggtggaagaggccggtaagtcctgtggcgcaggatccctgatgccagccctggaagagccccagcctgaccaagcatttaggatgtttcccgacatttgtacctcacaccagaggccttttttccgcgaaaaccagcagataaccgtgtaa

SEQ ID NO: 64 - DNA, 2271 bp, Homo sapiens
atgagctggtcaccttccctgacaacgcagacatgtggggcctgggaaatgaaagagcgccttgggacagggggatttggaaatgtcatccgatggcacaatcaggaaacaggtgagcagattgccatcaagcagtgccggcaggagctcagcccccggaaccgagagcggtggtgcctggagatccagatcatgagaaggctgacccaccccaatgtggtggctgcccgagatgtccctgaggggatgcagaacttggcgcccaatgacctgcccctgctggccatggagtactgccaaggaggagatctccggaagtacctgaaccagtttgagaactgctgtggtctgcgggaaggtgccatcctcaccttgctgagtgacattgcctctgcgcttagataccttcatgaaaacagaatcatccatcgggatctaaagccagaaaacatcgtcctgcagcaaggagaacagaggttaatacacaaaattattgacctaggatatgccaaggagctggatcagggcagtctttgcacatcattcgtggggaccctgcagtacctggccccagagctactggagcagcagaagtacacagtgaccgtcgactactggagcttcggcaccctggcctttgagtgcatcacgggcttccggcccttcctccccaactggcagcccgtgcagtggcattcaaaagtgcggcagaagagtgaggtggacattgttgttagcgaagacttgaatggaacggtgaagttttcaagctctttaccctaccccaataatcttaacagtgtcctggctgagcgactggagaagtggctgcaactgatgctgatgtggcacccccgacagaggggcacggatcccacgtatgggcccaatggctgcttcaaggccctggatgacatcttaaacttaaagctggttcatatcttgaacatggtcacgggcaccatccacacctaccctgtgacagaggatgagagtctgcagagcttgaaggccagaatccaacaggacacgggcatcccagaggaggaccaggagctgctgcaggaagcgggcctggcgttgatccccgataagcctgccactcagtgtatttcagacggcaagttaaatgagggccacacattggacatggatcttgtttttctctttgacaacagtaaaatcacctatgagactcagatctccccacggccccaacctgaaagtgtcagctgtatccttcaagagcccaagaggaatctcgccttcttccagctgaggaaggtgtggggccaggtctggcacagcatccagaccctgaaggaagattgcaaccggctgcagcagggacagcgagccgccatgatgaatctcctccgaaacaacagctgcctctccaaaatgaagaattccatggcttccatgtctcagcagctcaaggccaagttggatttcttcaaaaccagcatccagattgacctggagaagtacagcgagcaaaccgagtttgggatcacatcagataaactgctgctggcctggagggaaatggagcaggctgtggagctctgtgggcgggagaacgaagtgaaactcctggtagaacggatgatggctctgcagaccgacattgtggacttacagaggagccccatgggccggaagcaggggggaacgctggacgacctagaggagcaagcaagggagctgtacaggagactaagggaaaaacctcgagaccagcgaactgagggtgacagtcaggaaatggtacggctgctgcttcaggcaattcagagcttcgagaagaaagtgcgagtgatctatacgcagctcagtaaaactgtggtttgcaagcagaaggcgctggaactgttgcccaaggtggaagaggtggtgagcttaatgaatgaggatgagaagactgttgtccggctgcaggagaagcggcagaaggagctctggaatctcctgaagattgcttgtagcaaggtccgtggtcctgtcagtggaagcccggatagcatgaatgcctctcgacttagccagcctgggcagctgatgtctcagccctccacggcctccaacagcttacctgagccagccaagaagagtgaagaactggtggctgaagcacataacctctgcaccctgctagaaaatgccatacaggacactgtgagggaacaagaccagagtttcacggccctagactggagctggttacagacggaagaagaagagcacagctgcctggagcaggcctcatga

SEQ ID NO: 65 - DNA, 2265 bp, Homo sapiens
atgttttcaggggggtgtcatagccccgggtttggccgccccagccccgccttccccgccccggggagcccgccccctgccccgcgtccctgccgacaggaaacaggtgagcagattgccatcaagcagtgccggcaggagctcagcccccggaaccgagagcggtggtgcctggagatccagatcatgagaaggctgacccaccccaatgtggtggctgcccgagatgtccctgaggggatgcagaacttggcgcccaatgacctgcccctgctggccatggagtactgccaaggaggagatctccggaagtacctgaaccagtttgagaactgctgtggtctgcgggaaggtgccatcctcaccttgctgagtgacattgcctctgcgcttagataccttcatgaaaacagaatcatccatcgggatctaaagccagaaaacatcgtcctgcagcaaggagaacagaggttaatacacaaaattattgacctaggatatgccaaggagctggatcagggcagtctttgcacatcattcgtggggaccctgcagtacctggccccagagctactggagcagcagaagtacacagtgaccgtcgactactggagcttcggcaccctggcctttgagtgcatcacgggcttccggcccttcctccccaactggcagcccgtgcagtggcattcaaaagtgcggcagaagagtgaggtggacattgttgttagcgaagacttgaatggaacggtgaagttttcaagctctttaccctaccccaataatcttaacagtgtcctggctgagcgactggagaagtggctgcaactgatgctgatgtggcacccccgacagaggggcacggatcccacgtatgggcccaatggctgcttcaaggccctggatgacatcttaaacttaaagctggttcatatcttgaacatggtcacgggcaccatccacacctaccctgtgacagaggatgagagtctgcagagcttgaaggccagaatccaacaggacacgggcatcccagaggaggaccaggagctgctgcaggaagcgggcctggcgttgatccccgataagcctgccactcagtgtatttcagacggcaagttaaatgagggccacacattggacatggatcttgtttttctctttgacaacagtaaaatcacctatgagactcagatctccccacggccccaacctgaaagtgtcagctgtatccttcaagagcccaagaggaatctcgccttcttccagctgaggaaggtgtggggccaggtctggcacagcatccagaccctgaaggaagattgcaaccggctgcagcagggacagcgagccgccatgatgaatctcctccgaaacaacagctgcctctccaaaatgaagaattccatggcttccatgtctcagcagctcaaggccaagttggatttcttcaaaaccagcatccagattgacctggagaagtacagcgagcaaaccgagtttgggatcacatcagataaactgctgctggcctggagggaaatggagcaggctgtggagctctgtgggcgggagaacgaagtgaaactcctggtagaacggatgatggctctgcagaccgacattgtggacttacagaggagccccatgggccggaagcaggggggaacgctggacgacctagaggagcaagcaagggagctgtacaggagactaagggaaaaacctcgagaccagcgaactgagggtgacagtcaggaaatggtacggctgctgcttcaggcaattcagagcttcgagaagaaagtgcgagtgatctatacgcagctcagtaaaactgtggtttgcaagcagaaggcgctggaactgttgcccaaggtggaagaggtggtgagcttaatgaatgaggatgagaagactgttgtccggctgcaggagaagcggcagaaggagctctggaatctcctgaagattgcttgtagcaaggtccgtggtcctgtcagtggaagcccggatagcatgaatgcctctcgacttagccagcctgggcagctgatgtctcagccctccacggcctccaacagcttacctgagccagccaagaagagtgaagaactggtggctgaagcacataacctctgcaccctgctagaaaatgccatacaggacactgtgagggaacaagaccagagtttcacggccctagactggagctggttacagacggaagaagaagagcacagctgcctggagcaggcctcatga

SEQ ID NO: 66 - DNA, 771 bp, Homo sapiens
atgagctggtcaccttccctgacaacgcagacatgtggggcctgggaaatgaaagagcgccttgggacagggggatttggaaatgtcatccgatggcacaatcaggaaacaggtgagcagattgccatcaagcagtgccggcaggagctcagcccccggaaccgagagcggtggtgcctggagatccagatcatgagaaggctgacccaccccaatgtggtggctgcccgagatgtccctgaggggatgcagaacttggcgcccaatgacctgcccctgctggccatggagtactgccaaggaggagatctccggaagtacctgaaccagtttgagaactgctgtggtctgcgggaaggtgccatcctcaccttgctgagtgacattgcctctgcgcttagataccttcatgaaaacagaatcatccatcgggatctaaagccagaaaacatcgtcctgcagcaaggagaacagaggttaatacacaaaattattgacctaggatatgccaaggagctggatcagggcagtctttgcacatcattcgtggggaccctgcagtacctggccccagagctactggagcagcagaagtacacagtgaccgtcgactactggagcttcggcaccctggcctttgagtgcatcacgggcttccggcccttcctccccaactggcagcccgtgcagtgcgtaagaatgtggccgggtacagtggctcactcctgtaatcccagcactttgggaggccgaggcaggtggatcagttga

SEQ ID NO: 67 - DNA, 2094 bp, Homo sapiens
atgtcatccgatggcacaatcaggctgacccaccccaatgtggtggctgcccgagatgtccctgaggggatgcagaacttggcgcccaatgacctgcccctgctggccatggagtactgccaaggaggagatctccggaagtacctgaaccagtttgagaactgctgtggtctgcgggaaggtgccatcctcaccttgctgagtgacattgcctctgcgcttagataccttcatgaaaacagaatcatccatcgggatctaaagccagaaaacatcgtcctgcagcaaggagaacagaggttaatacacaaaattattgacctaggatatgccaaggagctggatcagggcagtctttgcacatcattcgtggggaccctgcagtacctggccccagagctactggagcagcagaagtacacagtgaccgtcgactactggagcttcggcaccctggcctttgagtgcatcacgggcttccggcccttcctccccaactggcagcccgtgcagtggcattcaaaagtgcggcagaagagtgaggtggacattgttgttagcgaagacttgaatggaacggtgaagttttcaagctctttaccctaccccaataatcttaacagtgtcctggctgagcgactggagaagtggctgcaactgatgctgatgtggcacccccgacagaggggcacggatcccacgtatgggcccaatggctgcttcaaggccctggatgacatcttaaacttaaagctggttcatatcttgaacatggtcacgggcaccatccacacctaccctgtgacagaggatgagagtctgcagagcttgaaggccagaatccaacaggacacgggcatcccagaggaggaccaggagctgctgcaggaagcgggcctggcgttgatccccgataagcctgccactcagtgtatttcagacggcaagttaaatgagggccacacattggacatggatcttgtttttctctttgacaacagtaaaatcacctatgagactcagatctccccacggccccaacctgaaagtgtcagctgtatccttcaagagcccaagaggaatctcgccttcttccagctgaggaaggtgtggggccaggtctggcacagcatccagaccctgaaggaagattgcaaccggctgcagcagggacagcgagccgccatgatgaatctcctccgaaacaacagctgcctctccaaaatgaagaattccatggcttccatgtctcagcagctcaaggccaagttggatttcttcaaaaccagcatccagattgacctggagaagtacagcgagcaaaccgagtttgggatcacatcagataaactgctgctggcctggagggaaatggagcaggctgtggagctctgtgggcgggagaacgaagtgaaactcctggtagaacggatgatggctctgcagaccgacattgtggacttacagaggagccccatgggccggaagcaggggggaacgctggacgacctagaggagcaagcaagggagctgtacaggagactaagggaaaaacctcgagaccagcgaactgagggtgacagtcaggaaatggtacggctgctgcttcaggcaattcagagcttcgagaagaaagtgcgagtgatctatacgcagctcagtaaaactgtggtttgcaagcagaaggcgctggaactgttgcccaaggtggaagaggtggtgagcttaatgaatgaggatgagaagactgttgtccggctgcaggagaagcggcagaaggagctctggaatctcctgaagattgcttgtagcaaggtccgtggtcctgtcagtggaagcccggatagcatgaatgcctctcgacttagccagcctgggcagctgatgtctcagccctccacggcctccaacagcttacctgagccagccaagaagagtgaagaactggtggctgaagcacataacctctgcaccctgctagaaaatgccatacaggacactgtgagggaacaagaccagagtttcacggccctagactggagctggttacagacggaagaagaagagcacagctgcctggagcaggcctcatga

SEQ ID NO: 68 - DNA, 2220 bp, Mus musculus
atgagctggtcaccgtccctcccaacccagacatgtggagcctgggaaatgaaagaacgcctggggaccgggggatttggaaacgtcatccggtggcacaatcaggcgacaggtgaacagatcgccatcaagcaatgccgacaggagctcagcccaaagaacagagaccgctggtgcctcgaaatccagatcatgagaaggctgaaccatcccaatgtggtggctgcccgggatgtcccagaggggatgcagaacctggcacccaatgatttgccactgctggccatggagtactgccaaggaggagatctccgaagatacttgaaccagttcgagaactgctgtggcctgcgggaaggagctgtccttaccctgctgagtgacatcgcatcggctcttagataccttcacgaaaacagaatcatccatcgagacctgaagccagaaaacatcgttctgcagcaaggagagaaaagattaatacacaaaattattgatctaggatatgccaaggagctggatcagggcagtctgtgcacgtcatttgtggggactctgcaatacctggcgccagagcttctggagcagcagaagtacaccgtgaccgttgactactggagcttcggcaccctggccttcgagtgcatcactggcttccggcccttcctccctaactggcagcctgtgcagtggcactccaaagtccggcagaagagcgaagtggacatcgttgttagtgaagacttgaatggagcagtgaagttttcaagttcgctacccttccccaataatcttaacagtgtcttggctgaacggctggagaagtggctgcagctgatgcttatgtggcaccctcggcaaaggggcacggatccccagtatggccccaacggctgcttcagagccctggatgacatcttgaacttgaagctggttcatgtcttgaacatggtcacaggcaccgttcacacataccccgtgacggaggatgagagtctgcagagcttaaaaaccagaatccaggaagacacggggatcctggagacagaccaggagctgctgcaagaggcagggctggtgctgctccctgacaagcctgctactcagtgcatctcagacagcaagacaaacgagggcctcacgttggacatggatcttgtttttctctttgacaacagtaaaatcaactatgagactcagatcaccccccgaccccaaccggaaagtgtcagctgtatccttcaggagcccaagcggaacctctccttcttccagctgaggaaagtgtggggccaagtctggcacagcatccagacgctgaaggaagactgtaaccggctgcagcagggacagcgagcagccatgatgagtctcctccggaataacagctgcctctctaagatgaagaacgccatggcctccacggcccagcagctcaaggccaagctggacttcttcaaaaccagcatccagatcgacctggagaagtataaagagcagaccgagtttgggatcacctcagataaattgctgctggcttggcgggagatggagcaggctgtggagcagtgtgggcgggagaatgacgtgaagcatctagtagagcggatgatggcactgcagactgacattgtggacctgcagaggagcccgatgggtcggaagcaggggggcaccctggatgacctagaggaacaagcgagggagctctaccgaagactcagggagaagccaagagaccaaaggacagaaggtgacagccaggagatggtacggctgctgcttcaggcaatccaaagctttgagaagaaagttcgggtgatttatacacagctcagtaagaccgtggtttgtaagcagaaggcactggagttgctgcccaaggtagaagaggtagtgagccttatgaacgaggacgagaggaccgtggtccggcttcaggagaagcggcagaaggaactctggaacctcctgaagatcgcctgtagcaaagtccgaggtcccgtgagtggaagcccagacagcatgaatgtgtctcgactcagtcaccctggtcagctaatgtcccagccttccagtgcctgtgacagcttacctgaatcagacaagaaaagtgaagaactggtggccgaagcccacgccctctgctcccggctagaaagtgcgctgcaggacactgtgaaggagcaagacagaagcttcacggtaaccgcctgataa

SEQ ID NO: 69 - protein, 9 aa, Artificial Sequence
  anti-CD163 CDRL1
ASQSVSHDV

SEQ ID NO: 70 - protein, 8 aa, Artificial Sequence
  anti-CD163 CDRL3
QDYSSPRT

SEQ ID NO: 71 - protein, 8 aa, Artificial Sequence
  anti-CD163 CDRH1
GYSITSDY

SEQ ID NO: 72 - protein, 12 aa, Artificial Sequence
  anti-CD163 CDRH3
CVSGTYYFDYWG

SEQ ID NO: 73 - protein, 9 aa, Artificial Sequence
  anti-CD163 CDRL1
ASQSVSSDV

SEQ ID NO: 74 - protein, 8 aa, Artificial Sequence
  anti-CD163 CDRL3
QDYTSPRT

SEQ ID NO: 75 - protein, 8 aa, Artificial Sequence
  anti-CD163 CDRH1
GYSITSDY

SEQ ID NO: 76 - protein, 12 aa, Artificial Sequence
  anti- CD163 CDRL3
CVSGTYYFDYWG

SEQ ID NO: 77 - protein, 16 aa, Artificial Sequence
  anti-CD23 CDRL1
RSSKSLLYKDGKTYLN

SEQ ID NO: 78 - protein, 7 aa, Artificial Sequence
  anti-CD23 CDRL2
LMSTRAS

SEQ ID NO: 79 - protein, 9 aa, Artificial Sequence
  anti-CD23CDRL3
QQLVEYPFT

SEQ ID NO: 80 - protein, 5 aa, Artificial Sequence
  anti-CD23 CDRH1
GYWMS

SEQ ID NO: 81 - protein, 19 aa, Artificial Sequence
  anti-CD23 CDRH2
EIRLKSDNYATHYAESVKG

SEQ ID NO: 82 - protein, 8 aa, Artificial Sequence
  anti-CD38 CDRL1
SSNIGDNY

SEQ ID NO: 83 - protein, 10 aa, Artificial Sequence
  anti-CD38 CDRL3
QSYDSSLSGS

SEQ ID NO: 84 - protein, 8 aa, Artificial Sequence
  anti-CD38 CDRH1
GFTFDDYG

SEQ ID NO: 85 - protein, 8 aa, Artificial Sequence
  anti-CD38 CDRH2
ISWNGGKT

SEQ ID NO: 86 - protein, 16 aa, Artificial Sequence
  anti-CD38 CDRH3
ARGSLFHDSSGFYFGH

SEQ ID NO: 87 - protein, 8 aa, Artificial Sequence
  anti-CD38 CDRL1
NSNIGSNT

SEQ ID NO: 88 - protein, 11 aa, Artificial Sequence
  anti-CD38 CDRL3
QSYDSSLSGSR

SEQ ID NO: 89 - protein, 8 aa, Artificial Sequence
  anti-CD38 CDRH1
GFTFNNYG

SEQ ID NO: 90 - protein, 8 aa, Artificial Sequence
  anti-CD38 CDRH2
ISYDGSDK

SEQ ID NO: 91 - protein, 15 aa, Artificial Sequence
  anti-CD38 CDRH3
ARVYYYGFSGPSMDV

SEQ ID NO: 92 - protein, 10 aa, Artificial Sequence
  anti-CD64 scFv CDRL3
QLRSNWPPYT

SEQ ID NO: 93 - protein, 7 aa, Artificial Sequence
  anti-CD38 CDRL2
DASNRAT

SEQ ID NO: 94 - protein, 10 aa, Artificial Sequence
  anti-CD38 CDRL3
QQRSNWPPTF

SEQ ID NO: 95 - protein, 5 aa, Artificial Sequence
  anti-CD38 CDRH1
SFAMS

SEQ ID NO: 96 - protein, 17 aa, Artificial Sequence
  anti-CD38 CDRH2
AISGSGGGTYYADSVKG

SEQ ID NO: 97 - protein, 13 aa, Artificial Sequence
  anti-CD38 CDRH3
DKILWFGEPVFDY

SEQ ID NO: 98 - protein, 11 aa, Artificial Sequence
  CDRL1
RASQSVSSYLA

SEQ ID NO: 99 - protein, 7 aa, Artificial Sequence
  anti-CD64 CDRL2
DASSRAT

SEQ ID NO: 100 - protein, 5 aa, Artificial Sequence
  anti-CD64 CDRH1
GYGMH

SEQ ID NO: 101 - protein, 17 aa, Artificial Sequence
  anti-CD64 CDRH2
VIWYDGSNKYYADSVKG

SEQ ID NO: 102 - protein, 9 aa, Artificial Sequence
  anti-CD64 CDRH3
DTGDRFFDY

SEQ ID NO: 103 - protein, 307 aa, Artificial Sequence
  anti-CD19 scFv
MALPVTALLLPLALLLHAEVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKRAAAIEVMYPPPYLDNEKSNGTIIHVKGKHLCPSPLFPGPSKPFW

SEQ ID NO: 104 - protein, 11 aa, Artificial Sequence
  anti-ROR1 scFv CDRL1
ASGFDFSAYYM

SEQ ID NO: 105 - protein, 7 aa, Artificial Sequence
  anti-ROR1 scFv CDRL2
TIYPSSG

SEQ ID NO: 106 - protein, 9 aa, Artificial Sequence
  anti-ROR1 scFv CDRL3
ADRATYFCA

SEQ ID NO: 107 - protein, 6 aa, Artificial Sequence
  anti-ROR1 scFv CDRH1
DTIDWY

SEQ ID NO: 108 - protein, 16 aa, Artificial Sequence
  anti-ROR1 scFv CDRH2
VQSDGSYTKRPGVPDR

SEQ ID NO: 109 - protein, 8 aa, Artificial Sequence
  anti-ROR1 scFv CDRH3
YIGGYVFG

SEQ ID NO: 110 - protein, 301 aa, Artificial Sequence
  anti-ROR1 scFv
MLLLVTSLLLCELPHPAFLLIPQEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYADDGALFNIWGPGTLVTISSGGGGSGGGGSGGGGSELVLTQSPSVSAALGSPAKITCTLSSAHKTDTIDWYQQLQGEAPRYLMQVQSDGSYTKRPGVPDRFSGSSSGADRYLIIPSVQADDEADYYCGADYIGGYVFGGGTQLTVTGESKYGPPCPPCPMFWVLVVVGGVLACYSLLV

SEQ ID NO: 111 - protein, 750 aa, Homo sapiens
MWNLLHETDSAVATARRPRWLCAGALVLAGGFFLLGFLFGWFIKSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 112 - protein, 123 aa, Homo sapiens
MKAVLLALLMAGLALQPGTALLCYSCKAQVSNEDCLQVENCTQLGEQCWTARIRAVGLLTVISKGCSLNCVDDSQDYYVGKKNITCCDTDLCNASGAHALQPAAAILALLPALGLLLWGPGQL

SEQ ID NO: 113 - protein, 622 aa, Homo sapiens [sequence omitted]

SEQ ID NO: 114 - protein, 556 aa, Homo sapiens [sequence omitted]

SEQ ID NO: 115 - protein, 297 aa, Homo sapiens
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEIIPIQEEEEEETETNFPEPPQDQESSPIENDSSP

SEQ ID NO: 116 - protein, 937 aa, Homo sapiens
MHRPRRRGTRPPLLALLAALLLAARGAAAQETELSVSAELVPTSSWNISSELNKDSYLTLDEPMNNITTSLGQTAELHCKVSGNPPPTIRWFKNDAPVVQEPRRLSFRSTIYGSRLRIRNLDTTDTGYFQCVATNGKEVVSSTGVLFVKFGPPPTASPGYSDEYEEDGFCQPYRGIACARFIGNRTVYMESLHMQGEIENQITAAFTMIGTSSHLSDKCSQFAIPSLCHYAFPYCDETSSVPKPRDLCRDECEILENVLCQTEYIFARSNPMILMRLKLPNCEDLPQPESPEAANCIRIGIPMADPINKNHKCYNSTGVDYRGTVSVTKSGRQCQPWNSQYPHTHTFTALRFPELNGGHSYCRNPGNQKEAPWCFTLDENFKSDLCDIPACDSKDSKEKNKMEILYILVPSVAIPLAIALLFFFICVCRNNQKSSSAPVQRQPKHVRGQNVEMSMLNAYKPKSKAKELPLSAVRFMEELGECAFGKIYKGHLYLPGMDHAQLVAIKTLKDYNNPQQWTEFQQEASLMAELHHPNIVCLLGAVTQEQPVCMLFEYINQGDLHEFLIMRSPHSDVGCSSDEDGTVKSSLDHGDFLHIAIQIAAGMEYLSSHFFVHKDLAARNILIGEQLHVKISDLGLSREIYSADYYRVQSKSLLPIRWMPPEAIMYGKFSSDSDIWSFGVVLWEIFSFGLQPYYGFSNQEVIEMVRKRQLLPCSEDCPPRMYSLMTECWNEIPSRRPRFKDIHVRLRSWEGLSSHTSSTTPSGGNATTQTTSLSASPVSNLSNPRYPNYMFPSQGITPQGQIAGFIGPPIPQNQRFIPINGYPIPPGYAAFPAAHYQPTGPPRVIQHCPPPKSRSPSSASGSTSTGHVTSLPSSGSNQEANIPLLPHMSIPNHPGGMGITVFGNKSQKPYKIDSKQASLLGDANIHGHTESMISAEL

SEQ ID NO: 117 - protein, 168 aa, Homo sapiens
MGHHHHHHHHHHSSGHIEGRHMRRVPGVAPTLVRSASETSEKRPFMCAYPGCNKRYFKLSHLQMHSRKHTGEKPYQCDFKDCERRFFRSDQLKRHQRRHTGVKPFQCKTCQRKFSRSDHLKTHTRTHTGEKPFSCRWPSCQKKFARSDELVRHHNMHQRNMTKLQLAL

SEQ ID NO: 118 - protein, 347 aa, Homo sapiens
DPNFWLQVQESVTVQEGLCVLVPCTFFHPIPYYDKNSPVHGYWFREGAIISRDSPVATNKLDQEVQEETQGRFRLLGDPSRNNCSLSIVDARRRDNGSYFFRMERGSTKYSYKSPQLSVHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVHGAIGGAGVTALLALCLCLIFFIVKTHRRKAARTAVGRNDTHPTTGSASPKHQKKSKLHGPTETSSCSGAAPTVEMDEELHYASLNFHGMNPSKDTSTEYSEVRTQ

SEQ ID NO: 119 - protein, 32 aa, Artificial Sequence
  Viral Antigen Sequence
VGFPVTPQVPLRPMTYKAAVDLSHFLKEKGGL

SEQ ID NO: 120 - protein, 30 aa, Artificial Sequence
  Viral Antigen Sequence
HTQGYFPDWQNYTPGPGVRYPLTFGWLYKL

SEQ ID NO: 121 - protein, 19 aa, Artificial Sequence
  Viral Antigen Sequence
EKIRLRPGGKKKYKLKHIV

SEQ ID NO: 122 - protein, 32 aa, Artificial Sequence
  Viral Antigen Sequence
NPPIPVGEIYKRWIILGLNKIVRMYSPTSILD

SEQ ID NO: 123 - protein, 31 aa, Artificial Sequence
  Viral Antigen Sequence
AIFQSSMTKILEPFRKQNPDIVIYQYMDDLY

SEQ ID NO: 124 - protein, 184 aa, Homo sapiens
MLQMAGQCSQNEYFDSLLHACIPCQLRCSSNTPPLTCQRYCNASVTNSVKGTNAILWTCLGLSLIISLAVFVLMFLLRKINSEPLKDEFKNTGSGLLGMANIDLEKSRTGDEIILPRGLEYTVEECTCEDCIKSKPKVDSDHCFPLPAMEEGATILVTTKTNDYCKSLPAALSATEIEKSISAR

SEQ ID NO: 125 - protein, 345 aa, Homo sapiens
MYKDCIESTGDYFLLCDAEGPWGIILESLAILGIVVTILLLLAFLFLMRKIQDCSQWNVLPTQLLFLLSVLGLFGLAFAFIIELNQQTAPVRYFLFGVLFALCFSCLLAHASNLVKLVRGCVSFSWTTILCIAIGCSLLQIIIATEYVTLIMTRGMMFVNMTPCQLNVDFVVLLVYVLFLMALTFFVSKATFCGPCENWKQHGRLIFITVLFSIIIWVVWISMLLRGNPQFQRQPQWDDPVVCIALVTNAWVFLLLYIVPELCILYRSCRQECPLQGNACPVTAYQHSFQVENQELSRARDSDGAEEDVALTSYGTPIQPQTVDPTQECFIPQAKLSPQQDAGGV

SEQ ID NO: 126 - protein, 300 aa, Homo sapiens
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 127 - protein, 334 aa, Homo sapiens
MAGSPTCLTLIYILWQLTGSAASGPVKELVGSVGGAVTFPLKSKVKQVDSIVWTFNTTPLVTIQPEGGTIIVTQNRNRERVDFPDGGYSLKLSKLKKNDSGIYYVGIYSSSLQQPSTQEYVLHVYEHLSKPKVTMGLQSNKNGTCVTNLTCCMEHGEEDVIYTWKALGQAANESHNGSILPISWRWGESDMTFICVARNPVSRNFSSPILARKLCEGAADDPDSSMVLLCLLLVPLLLSLFVLGLFLWFLKRERQEEYIEEKKRVDICRETPNICPHSGENTEYDTIPHTNRTILKEDPANTVYSTVEIPKKMENPHSLLTMPDTPRLFAYENV

SEQ ID NO: 128 - DNA, 20 bp, Artificial Sequence
  primer
actggggcagttatgacagg

SEQ ID NO: 129 - DNA, 19 bp, Artificial Sequence
  primer
gatgatcggccacaaactg

SEQ ID NO: 130 - DNA, 21 bp, Artificial Sequence
  primer
ttgttcccttctcatctgcat

SEQ ID NO: 131 - DNA, 21 bp, Artificial Sequence
  primer
ccttgaccttattctccacga

SEQ ID NO: 132 - DNA, 19 bp, Artificial Sequence
  primer
cctactcccactcggtcct

SEQ ID NO: 133 - DNA, 21 bp, Artificial Sequence
  primer
ctgatttcttgggtttgctgt

SEQ ID NO: 134 - DNA, 18 bp, Artificial Sequence
  primer
agagctccacagcgcttc

SEQ ID NO: 135 - DNA, 18 bp, Artificial Sequence
  primer
cagcacctgggaggtgaa

SEQ ID NO: 136 - DNA, 26 bp, Artificial Sequence
  primer
tcttaaagaccacatggtagaacagt

SEQ ID NO: 137 - DNA, 17 bp, Artificial Sequence
  primer
agctgctgttgggattg

SEQ ID NO: 138 - DNA, 20 bp, Artificial Sequence
  primer
gctgtgcccttaacaaaagc

SEQ ID NO: 139 - DNA, 18 bp, Artificial Sequence
  primer
ggctgaggtggcatgtct

SEQ ID NO: 140 - DNA, 19 bp, Artificial Sequence
  primer
agccacatcgctcagacac

SEQ ID NO: 141 - DNA, 19 bp, Artificial Sequence
  primer
gcccaatacgaccaaatcc

SEQ ID NO: 142 - protein, 11 aa, Artificial Sequence
  anti-CD19 scFv CDRL1
RASQDISKYLN

SEQ ID NO: 143 - protein, 7 aa, Artificial Sequence
  anti-CD19 scFv CDRL2
SRLHSGV

SEQ ID NO: 144 - protein, 9 aa, Artificial Sequence
  anti-CD19 scFv CDRL3
GNTLPYTFG

SEQ ID NO: 145 - protein, 5 aa, Artificial Sequence
  anti-CD19 scFv CDRH1
DYGVS

SEQ ID NO: 146 - protein, 16 aa, Artificial Sequence
  anti-CD19 scFv CDRH2
VTWGSETTYYNSALKS

SEQ ID NO: 147 - protein, 7 aa, Artificial Sequence
  anti-CD19 scFv CDRH3
YAMDYWG

(19) United States
(12) Patent Application Publication
(10) Pub. No.: US 2021/0128485 A1
(43) Pub. Date: May 6, 2021

(54) NANOPARTICLES FOR GENE EXPRESSION AND USES THEREOF

(71) Applicant: FRED HUTCHINSON CANCER RESEARCH CENTER, Seattle, WA (US)

(72) Inventor: Matthias Stephan, Seattle, WA (US)

(73) Assignee: FRED HUTCHINSON CANCER RESEARCH CENTER, Seattle, WA (US)

(21) Appl. No.: 17/044,779

(22) PCT Filed: May 1, 2019
(86) PCT No.: PCT/US19/30263

§ 371 Date: Oct. 1, 2020

Related U.S. Application Data
(60) Provisional application No. 62/665,280, filed on May 1, 2018.

(51) Int. Cl. A61K 9/51; C07K 16/28; A61K 9/127; A61K 45/06

(52) U.S. Cl. CPC A61K 9/51; C07K 16/2809; B82Y 5/00; A61K 45/06; A61K 9/1271

(57) ABSTRACT

Treatment protocols based on expression of therapeutic proteins by genetically-modified selected cell types in vivo are described. The treatment protocols can additionally utilize cell attractants to attract selected cell types to a treatment site and/or macrophage activation protocols at the treatment site.

### CROSS-REFERENCE TO RELATED APPLICATION

This application claims priority to U.S. Provisional Patent Application No. 62/665,280 filed May 1, 2018, the entire contents of which is incorporated by reference herein in its entirety.

### FIELD OF THE DISCLOSURE

The present disclosure provides treatment protocols based on expression of therapeutic proteins by genetically-modified selected cell types in vivo. The treatment protocols can additionally utilize cell attractants to attract selected cell types to a treatment site and/or macrophage activation protocols at the treatment site.

### BACKGROUND OF THE DISCLOSURE

Many new medical therapies involve genetically modifying the cells of a patient's immune system to fight a disease or an infection. For example, adoptive T-cell therapy is a powerful cancer therapy where T cells are harvested from the patient and genetically modified to target and kill cancer cells. However, the complexity and high costs involved in manufacturing a genetically-engineered T cell product for each patient, rather than preparing a drug in bulk in standardized form, makes it difficult to outcompete current frontline therapy options, such as small molecule drugs or monoclonal antibodies. For example, genetically-modifying T cells for adoptive T-cell therapy generally requires:
(i) Leukapheresis to extract T cells from the patient (i.e., the patient is connected by two intravenous tubes to an apheresis machine for several hours; this is not comfortable for the patient, incurs substantial cost, and ultimately, large-scale adoption of autologous T therapy may become rate limited by availability of apheresis capacity);
(ii) Activation and genetic modification of T cells;
(iii) Expansion of genetically-modified T cells over a two-week period in a cytokine-supplemented tissue culture medium;
(iv) Washing and concentrating the T cells prior to administration (and for T cell products made at central facilities and transported to remote treatment centers, cryopreservation); and
(v) quality control (QC) release assays for each formulated batch of genetically-modified T cell product.
   Further adding to the cost and complexity of manufacturing genetically-modified T cell products, all of these procedures must be conducted under environmentally controlled Good Manufacturing Practice (GMP) conditions which are expensive to maintain and run. Moreover, as each genetically-modified T cell product is made from starting materials (T cells) from the patient to be treated, there are no substantial economies of scale.

Another drawback associated with many genetically-modified cell types is that the cells can persist in patients after administration, sometimes leading to unwanted and/or lingering side effects. Thus, mechanisms to achieve effective, and scalable, yet less permanent, therapies are needed.

### SUMMARY OF THE DISCLOSURE

The current disclosure provides treatment protocols based on expression of nucleic acids and/or protein, such as therapeutic proteins, by genetically-modified selected cell types in vivo. In some embodiments, expression of the therapeutic protein is transient, reducing concerns regarding the potential for lingering side effects are overcome. Moreover, the treatment protocols utilize the nanoparticles that can achieve genetic modification of selected cell types in vivo without the need for all extensive cell processing steps required by adoptive T cell therapies (and similar treatment protocols).

In particular embodiments, a subject who is administered nanoparticles that results in genetic modification of selected cell types to express a therapeutic protein is monitored for levels of therapeutic protein expression. When expression falls below a threshold, a treating physician can determine whether a subsequent dose of nanoparticles should be administered to prolong therapeutic protein expression within the subject. This process can be repeated until a therapeutic objective is achieved, and the physician determines that continued expression of the therapeutic protein within the subject would serve no beneficial clinical purpose.

In particular embodiments, the current disclosure provides administration of nanoparticles that genetically modify selected cell types in vivo to express a nucleic acid or protein, such as a therapeutic protein, for 5-10 days. In particular embodiments, nanoparticle-programmed cells transiently express therapeutic proteins on their surface for an average of seven days following in vivo exposure to the described nanoparticles.

In particular embodiments, the current disclosure provides utilizing cell attractants to attract selected cell types to a treatment site within the body. Following attraction of the selected cell types to the treatment site, nanoparticles that genetically modify the attracted cell types to transiently express a therapeutic protein can be administered locally at the treatment site. In particular embodiments, cell attractants are administered at a treatment site 24 hours before nanoparticle delivery.

In particular embodiments, a cell attractant is administered to the subject within a clinically relevant time window of a nanoparticle, in order to recruit cells to a desired site within the subject. For instance, T cell recruitment to a tumor site can be accomplished by administering a T cell attractant into or near the tumor. A nanoparticle treatment administered within a clinically relevant time window of the T cell attractant can then beneficially target the attracted T cells for expression of a therapeutic protein, for instance directed against the tumor.

Particular embodiments additionally utilize nanoparticles to reprogram the activation state of selected cell types. For example, particular embodiments utilize nanoparticles to activate macrophages at a treatment site.

The disclosure shows that the treatment protocols provide therapeutically effective treatments against, for example, lymphoma, prostate cancer, hepatitis B virus (HBV)-induced hepatocellular carcinoma, ovarian cancer, glioblastoma, and lung cancer.

The treatment protocols described herein result in use of affordable, off-the-shelf reagents for the treatment of patients with malignancies or infections where concerns regarding lingering side effects are overcome. Such products can be made available at the day of diagnosis and as frequently as medically necessary.

### BRIEF DESCRIPTION OF THE FIGURES

At least one of the drawings submitted herewith is better understood in color. Applicant considers the color versions of the drawing(s) as part of the original submission and reserve the right to present color images of the drawings in later proceedings.

FIG. 1: Illustration of a Representative Embodiment. Nanoparticles 100 include a coating 105 surrounding a core including passenger mRNA nucleic acid(s) 110 in association with polymer(s) 120. Embedded in and/or associated with the exterior of the coating 105 are one or more cell targeting ligands 140. Nanoparticle 100 is targeted specifically to target cell 160 (such as a T cell) through interaction between the cell targeting ligand(s) 140 and molecule(s) 150 on the surface of the target cell 160. Upon release of contents of the nanoparticle into the cytoplasm of target cell 160 (e.g., through receptor induced endocytosis), the passenger mRNA nucleic acid (shown as 110′ inside cell 140) is translated to express a protein 170, e.g., on the surface of target cell 160.

FIG. 2: Overview illustrating an embodiment of compositions and methods for reprograming T cells in situ to express disease-specific chimeric antigen receptors (CARs) or T cell receptors (TCRs) using in vitro transcribed (IVT) mRNA carried by polymeric nanoparticles. These nanoparticles are coated with ligands that target them to cytotoxic T cells, so once they are infused into the patient's circulation, they transfer the nucleic acid(s) they carry into the lymphocytes and transiently program them to express a therapeutic protein (e.g., a disease-specific CAR, TCR, or CAR/TCR hybrid) on their surfaces.

FIGS. 3A, 3B: Illustration of additional embodiments and modes of delivery. Scheme to genetically reprogram intraperitoneal (FIG. 3A) and intracranial (FIG. 3B) tumor-associated macrophages (TAMs) into tumoricidal macrophages using targeted mRNA nanoparticles. FIG. 3A illustrates delivery via a catheter (infusion via catheter); FIG. 3B illustrates delivery via direct tumoral injection (intratumoral delivery). Through directed, locally infused delivery such as illustrated here, patients can be spared from systemic toxicities because inflammation induced by treatment remains localized at the treatment site. Locally infused particles target cells in the tumor milieu, (2) deliver nucleotides that (as illustrated) selectively reprogram signaling pathways that control macrophage polarization, and (3) are degradable locally by physiological pathways. The administration routes depicted in FIGS. 3A and 3B can also be used to deliver nanoparticles including nucleic acids that result in expression of a therapeutic protein such as a CAR, TCR, or hybrid CAR/TCR.

FIGS. 4A, 4B: Design and manufacture of lymphocyte-programming nanoparticles. (FIG. 4A) Schematic of a representative T cell-targeted IVT mRNA nanoparticle. To create a reagent that can genetically modify primary T lymphocytes (which are notoriously refractory to non-viral transfection methods) simply by contact, polymeric nanoparticles were bioengineered including four functional components:
(i) surface-anchored targeting ligands that selectively bind the nanoparticles to T cells and initiate rapid receptor-induced endocytosis to internalize them. In representative experiments, anti-CD8 binding domains were used;
(ii) a negatively-charged coating that shields the nanoparticles to minimize off-target binding by reducing the surface charge of the nanoparticles. Polyglutamic acid (PGA) was used to accomplish this in representative experiments;
(iii) a carrier matrix that condenses and protects the nucleic acids from enzymatic degradation while they are in the endosome, but releases them once the particles are transported into the cytoplasm, thereby enabling transcription of the encoded protein. For this representation, a biodegradable poly(β-amino ester) (PBAE) polymer formulation that has a half-life between 1 and 7 hours in aqueous conditions was used; and
(iv) nucleic acids (e.g., IVT mRNA) that are encapsulated within the carrier and produce expression of, for instance, a disease-specific CAR or TCR.
   (FIG. 4B) Diagram describing how the nanoparticles were fabricated. The lyophilization and hydration steps are optional.

FIGS. 5A-5J: IVT mRNA nanoparticles efficiently transfect human T cells with CAR- or TCR encoding nucleic acids. Isolated human CD8+ T cells were stimulated with beads coated with antibodies against TCR/CD3 and co-stimulatory CD28 receptors. 24 h later, beads were removed and CD8-targeted NP containing either mRNA encoding the leukemia-specific 1928z CAR (FIG. 5A-5E) or the HBcore18-27 TCR (FIG. 5F-5J) were mixed into the cell suspension at a concentration of 3 μg of mRNA/10⁶ cells. (FIG. 5A) qPCR measurements of relative 1928z CAR mRNA expression over time after T cells were exposed to 1928z CAR nanoparticles. (FIG. 5B) Flow cytometry of T cells at indicated time point after incubation with nanoparticles bearing 1928z CAR encoding mRNA. (FIG. 5C) Summary plot of in vitro encapsulated nucleic acid transfer efficiencies. (FIG. 5D) In vitro assay comparing cytotoxicity of nanoparticle- vs. retrovirus-transfected T cells against Raji lymphoma cells. The IncuCyte Live Cell Analysis System was used to quantify immune cell killing of Raji NucLight Red cells by 1928z CAR-transfected T cells over time. Data are representative of two independent experiments. Each point represents the mean±s.e.m. pooled from two independent experiments conducted in triplicate. (FIG. 5E) ELISA measurements of IL-2 (at 24 h) and TNF-α and IFN-γ (at 48 h) secretion by transfected cells. (FIG. 5F) qPCR measurements of relative HBcore18-27 TCR mRNA expression over time after T cells were exposed to HBcore18-27 TCR nanoparticles. (FIG. 5G, 5H) Encapsulated nucleic acid transfer efficiencies (FIG. 51) Cell killing of HepG2-core NucLight Red cells by HBcore18-27 TCR-transfected T cells over time (FIG. 5J) ELISA measurements of cytokine secretion by transfected cells.

FIGS. 6A-6E: Nanoparticle-programmed CAR lymphocytes cause leukemia regression with efficacies similar to adoptive T-cell therapy. (FIG. 6A) Time line and nanoparticle dosing regimen. (FIG. 6B) Sequential bioimaging of firefly luciferase-expressing Raji lymphoma cells systemically injected into NSG mice. Five representative mice from each cohort (n=10) are shown. (FIG. 6C) Survival of animals following therapy, depicted as Kaplan-Meier curves. Shown are ten mice per treatment group pooled from three independent experiments. ms, median survival. Statistical analysis between the treated experimental and the untreated control group was performed using the Log-rank test; P<0.05 was considered significant. (FIG. 6D) Flow cytometry of peripheral T cells before and after injection of nanoparticles delivering IVT mRNA that encodes the 1928z CAR. The three profiles for each time point shown here are representative of two independent experiments consisting of ten mice per group. (FIG. 6E) Overview graph displaying the percentages of CAR-transfected CD8+ T cells following repeated infusion of 1928z CAR nanoparticles. Every line represents one animal. Shown are ten animals pooled from two independent experiments.

FIGS. 7A-7G: IVT-mRNA nanoparticles encoding prostate tumor-specific CARs improve survival of mice with established disease. (FIG. 7A) Heat map of PSCA, PSMA and ROR1 antigen expression across a panel of 140 prostate cancer metastases showing the diversity of antigen expression. (FIG. 7B) Heat map representation of flow cytometry data showing variability in PSCA, PSMA and ROR1 expression by LNCap C42 prostate carcinoma cells. The colors indicate expression levels in 350 randomly-chosen cells. (FIG. 7C) 3 weeks post-implantation, LNCap C42 prostate tumors are visualized by in vivo bioluminescent imaging. A representative photo of established tumors in the dorsal lobes of the prostates (white arrows) is shown on the right. (FIG. 7D) Sequential bioimaging of firefly luciferase-expressing LNCaP C42 prostate carcinoma cells orthotopically transplanted into the prostate of NGS mice. Four representative mice from each cohort (n=8) are shown. (FIG. 7E) Time line and nanoparticle dosing regimen. (FIG. 7F) Survival of animals following therapy, depicted as Kaplan-Meier curves. Shown are eight mice per treatment group pooled from three independent experiments. ms, median survival. Statistical analysis between the treated experimental and the untreated control group was performed using the Log-rank test; P<0.05 was considered significant. N.s., non-significant. (FIG. 7G) Flow cytometry quantification of ROR1 antigen expression on LNCaP C42 prostate tumor cells following CAR-T cell therapy or ROR1 4-1BBz CAR NP therapy. Shown are 350 randomly-chosen cells pooled from 5 tumors.

FIG. 8: List of antibodies used in myeloid and lymphoid immunophenotyping panels described in Example 2.

FIGS. 9A-9K: Nanoparticles carrying mRNA encoding IRF5 and IKKβ can imprint a pro-inflammatory M1-like phenotype. (FIG. 9A) Design of macrophage-targeted polymeric NPs formulated with mRNAs encoding key regulators of macrophage polarization. The particles consist of a PbAE-mRNA polyplex core coated with a layer of PGA-Di-mannose, which targets the particles to mannose receptors (CD206) expressed by M2-like macrophages. Also depicted is the synthetic mRNA encapsulated in the NP, which is engineered to encode the reprogramming transcription factors. (FIG. 9B) Transmission electron microscopy of a population of NPs (scale bar 200 nm) and a single NP (inset, scale bar 50 nm). (FIG. 9C) Size distributions of NPs, measured using a NanoSight NS300 instrument. (FIG. 9D) NPs demonstrated high transfection (46%) of bone marrow-derived macrophages (BMDMs) after 1 h exposure. (FIG. 9E) Gene-transfer efficiencies into bone marrow derived macrophages (BMDM) measured by flow cytometry 24 hours after nanoparticle transfection. (FIG. 9F) Relative viability of NP transfected and untransfected macrophages (assessed by staining with Annexin V and PI). N.s.; non-significant. (FIG. 9G) Expression kinetics of codon-optimized IRF5 mRNA (blue, left Y axis) and endogenous IRF5 mRNA (black, right Y axis) measured by qRT-PCR, n=3 for each time point. (FIG. 9H) Timelines depicting NP transfection protocols and culture conditions for the BMDMs used in FIGS. 9I-9K. (FIG. 9I) Gene expression profiles of IRF5/IKKβ. NP-transfected macrophages compared to signature M1 cells stimulated with the Toll-like Receptor 6 agonist MPLA. Results are depicted as a Volcano plot that shows the distribution of the fold changes in gene expression. M1 signature genes are indicated. P value of overlap between IRF5/IKKβ. NP-transfected macrophages and the M1 signature gene set was determined by GSEA. (FIG. 9J) Heat map of M1 signature gene expression in macrophages cultured in IL-4 versus cells cultured in IL-4 and transfected with IRF5/IKKβ. NPs. (FIG. 9K) Box plots showing mean counts for indicated genes and S.E.M.

FIGS. 10A-10J: Repeated intraperitoneal injections of mRNA nanocarriers delivering IRF5 and IKKβ genes into macrophages more than doubles mean survival of mice with disseminated ovarian cancer. (FIG. 10A) Time lines and dosing regimens. Arrows indicate time of I.P. injection. (FIG. 10B) Sequential bioluminescence imaging of tumor growth in control and treated mice. (FIG. 100) Kaplan-Meier survival curves for treated versus control mice. Statistical analysis was performed using the log-rank test. (FIG. 10D) Flow cytometric quantitation of in vivo transfection rates in different immune cell subpopulations 48 hours after a single i.p. dose of D-mannose-coated NPs carrying GFP mRNA as a control: macrophages (CD45+, CD11b+, MHCII+, CD11c, Ly6C−/low, Ly6G−), monocytes (CD45+, CD11b+, MHCII+, CD11c−, Ly6C+, Ly6G−), neutrophils (CD45+, CD11b+, MHCII+, CD11c−, Ly6G+), CD4+ T cells (CD45+, TCR-β chain+, CD4+, CD8), CD8+ T cells (CD45+, TCR-β chain+, CD4−, CD8+), and natural killer (NK) cells (CD45+, TCR-β chain, CD49b+) were measured. (FIG. 10E) Flow cytometric analysis of macrophage phenotypes in the peritoneum of mice with disseminated ID8 ovarian cancer. Animals were either treated with 4 doses of IRF5/IKKβ. NPs or PBS. (FIG. 10F) Box plots summarizing relative percent (left panel) and absolute numbers (right panel) of Ly6C−, F4/80+, and CD206+ (M2-like) macrophages. (FIG. 10G) Corresponding numbers for Ly6C−, F4/80+, and CD206− (M1-like) macrophages. (FIG. 10H) Representative hematoxylin and eosin-stained sections of ovarian tumor-infiltrated mesenteries isolated from PBS controls (top panel) or IRF5/IKKβ. NP-treated animals (bottom panel; scale bar 100 μm). 10-fold magnifications of representative malignant lesions are shown on the right (scale bar 50 μm). (FIG. 10I) Luminex assay measuring cytokines produced by isolated peritoneal macrophages from each treatment group. CD11b+, F4/80+ peritoneal macrophages were isolated by fluorescence activated cell sorting, and cultured ex vivo. After 24 hours, cell culture supernatants were collected. In parallel experiments, FACS-sorted CD11b+, F4/80+ peritoneal macrophages were directly analyzed by pRT-PCR to determine expression levels of four master regulators of the macrophage phenotypes (SerpinB2, Retnla, Ccl11, and Ccl5). Results are summarized as box plots in FIG. 10J.

FIGS. 11A-11F: Macrophage-programming mRNA nanocarriers are highly biocompatible and safe for repeated dosing. (FIG. 11A) In vivo biodistribution of macrophage-targeted IRF5/IKKβ NPs following i.p. administration. NP-delivered (codon optimized) mRNA was detected by qPCR 24 hours after a single injection of particles containing 50 μg mRNA. (FIG. 11B) Schematic representation of the experimental timeline. *Twenty-four hours after the last dose, mice were euthanized by CO₂ inhalation. Blood was collected through retro-orbital bleeding into heparin coated tubes for serum chemistry and complete blood count. Necrospy was performed for histological analysis of liver, spleen, pancreas, mesentery and omentum, stomach, and urinary bladder. (FIG. 11C) Representative hematoxylin and eosin-stained sections of various organs isolated from controls or NP-treated animals. Scale bar, 100 μm. Lesions found in the NP-treated animals are shown and described here based on analysis by a Comparative Pathologist. The relevant findings for each numbered image is: [1] Discrete foci of cellular infiltrates largely composed of mononuclear cells admixed with a few granulocytes; Mild extramedullary hematopoiesis. [2] In a few locally extensive areas, hepatocytes are mild to moderately swollen. [3] Moderate myeloid (predominant), erythroid and megakaryocyte hyperplasia within the red pulp. [4] Mild hypocellularity of the white pulp. [5] Within the mesentery, there are moderate, multifocal infiltrates of macrophages, lymphocytes, plasma cells and granulocytes. [6] Mild to moderate infiltrates of macrophages admixed with lymphocytes, plasma cells and granulocytes; Mild dissociation of the acini and acinar loss; Mild diffuse loss of zymogen granules from the acinar cells. [7] Dense aggregates of lymphocytes admixed with macrophages around fat tissue. [8] Mild multifocal vacuolar degeneration of the chief and parietal cells within the gastric mucosa. (FIG. 11D) Serum chemistry and blood counts. (FIGS. 11E, 11F) Luminex assay measurements of serum IL-6 (FIG. 11E) and TNF-α (FIG. 11F) cytokines 4 or 8 days after a single i.p. injection of IRF5/IKKβ NPs.

FIGS. 12A-121: Intravenously infused IRF5/IKKβ nanoparticles can control tumor metastases in the lung. (FIG. 12A) In vivo biodistribution of macrophage-targeted IRF5/IKKβ NPs following i.v. administration. Codon-optimized mRNA was measured by qPCR 24 hours after a single i.v. injection of particles containing 50 μg mRNA. (FIGS. 12B-12H) C57BL/6 albino mice were injected via tail vein with 1×10⁶ B16F10 firefly luciferase-expressing melanoma cells to establish lung metastases. After 7 days, animals were randomly assigned to either the IRF5/IKKβ NP treatment group, the control GFP NP group, or the PBS control. (FIG. 12B) Time lines and dosing regimens. (FIG. 12C) Confocal microscopy of healthy lungs (left panel) and B16F10 tumor-infiltrated lungs (right panel). Infiltrating macrophage populations fluoresce in green. (FIG. 12D) Sequential bioluminescence tumor imaging. (FIG. 12E) Kaplan-Meier survival curves for each treatment group. ms indicates median survival. Statistical analysis was performed using the log-rank test, and P<0.05 was considered significant. (FIG. 12F) Representative photographs (top row) and micrographs of lungs containing B16F10 melanoma metastases representing each group following 2 weeks of treatment. (FIG. 12G) Counts of lung tumor foci. (FIG. 12H) Phenotypic characterization of monocyte/macrophage populations in bronchoalveolar lavage from each treatment group. (FIG. 12I) Summary of the relative percentages of suppressive and activated macrophages.

FIGS. 13A13F: Macrophage reprogramming improves the outcome of radiotherapy in glioma. (FIG. 13A) T2 MRI scan, and histological staining following initiation of a PDGFβ-driven glioma in RCAS-PDGF-B/Nestin-Tv-a; Ink4a/Arf−/−; Pten−/− transgenic mice on post-induction day 21. (FIG. 13B) Confocal microscopy of CD68+ TAMs infiltrating the glioma margin. Scale bar 300 μm. (FIG. 13C) Flow cytometry analysis of macrophage (F4/80+, CD11b+) populations in healthy brain tissue versus glioma. (FIGS. 13D, 13E) Kaplan-Meier survival curves of mice with established gliomas receiving IRF5/IKKβ treatments as a monotherapy (FIG. 13D) or combined with brain tumor radiotherapy (FIG. 13E). Time lines and dosing regimens are shown on top. Ms, median survival. Statistical analysis was performed using the log-rank test, and P<0.05 was considered statistically significant. (FIG. 13F) Sequential bioluminescence imaging of tumor progression.

FIGS. 14A-14E: IVT mRNA-carrying nanoparticles encoding human IRF5/IKKβ efficiently reprogram human macrophages. (FIG. 14A) Time line and culture conditions to differentiate the human THP-1 monocytic cell line into suppressive M2-like macrophages. (FIG. 14B) Bioluminescent imaging of M2-differentiated THP1-Lucia cells cultured in 24 wells and transfected with indicated concentrations of NPs carrying human IRF5/IKKβ mRNA versus control GFP mRNA. Levels of IRF-induced Lucia luciferase were determined 24 hours after transfection using Quanti-Luc. (FIG. 14C) Summary of bioluminescent counts. (FIGS. 14D, 14E) Differences in IL-1β cytokine secretion (FIG. 14D) and surface expression (FIG. 14E) of the M1-macrophage marker CD80.

FIG. 15. Exemplary supporting sequences: SEQ ID NO: 1: Anti-human 1928z CAR; SEQ ID NO: 2: Anti-human ROR1 CAR; SEQ ID NO: 3: HBV-specific TCR; SEQ ID NO: 4: Anti-human 1928z CAR; SEQ ID NO: 5: Anti-human ROR1 (4-1BBz) CAR; SEQ ID NO: 6: Anti-HBV-specific TCR (HBcore18-27); SEQ ID NO: 7: anti-CD19 scFv (VH-VL) FMC63; SEQ ID NO: 8: anti-CD19 scFv (VH-VL) FMC63; SEQ ID NO: 9: CD28 effector domain; SEQ ID NO: 10: P28z CAR; SEQ ID NO: 11: IgG4-Fc; SEQ ID NO: 12: Hinge-CH2-CH3; SEQ ID NO: 13: Hinge-CH3; SEQ ID NO: 14: Hinge only; SEQ ID NO: 15: CD28 Transmembrane domain; SEQ ID NO: 16: CD28 Cytoplasmic domain (LL to GG); SEQ ID NO: 17: 4-1BB Cytoplasmic domain; SEQ ID NO: 18: CD3-ζ Cytoplasmic domain; SEQ ID NO: 19: T2A; SEQ ID NO: 20: tEGFR; SEQ ID NO: 21: Strep tag II; SEQ ID NO: 22: Myc tag; SEQ ID NO: 23: V5 tag; SEQ ID NO: 24: FLAG tag; SEQ ID NO: 25: Human IRF5 Isoform 1 (UniProt Accession Q13568-1); SEQ ID NO: 26: Human IRF5 Isoform 2 (UniProt Accession Q13568-2); SEQ ID NO: 27: Human IRF5 Isoform 3 (UniProt Accession Q13568-3); SEQ ID NO: 28: Human IRF5 Isoform 4 (UniProt Accession Q13568-4); SEQ ID NO: 29: Human IRF5 Isoform 5 (UniProt Accession Q13568-5); SEQ ID NO: 30: Human IRF5 Isoform 6 (UniProt Accession Q13568-6); SEQ ID NO: 31: Murine IRF5 protein (pI=5.19, Mw=56005, UniProt Accession P56477); SEQ ID NO: 32: Human IRF1 (UniProt Accession P10914); SEQ ID NO: 33: Human IRF3 isoform 1 (UniProt Accession Q14653-1); SEQ ID NO: 34: Human IRF7 isoform A (UniProt Accession Q92985-1); SEQ ID NO: 35: Human IRF8 (UniProt Accession Q02556); SEQ ID NO: 36: Murine IRF1 (UniProt Accession P15314); SEQ ID NO: 37: Murine IRF3 (UniProt Accession P70671); SEQ ID NO: 38: Murine IRF7 (UniProt Accession P70434); SEQ ID NO: 39: Murine IRF7/IRF3 5(D) protein (pI=4.72, MW=58456); SEQ ID NO: 40: Murine IRF8 (UniProt Accession P23611); SEQ ID NO: 41: Murine IRF8 (K310R) protein (pI=6.38, MW=48265); SEQ ID NO: 42: Human IKKβ. isoform 1 (UniProt Accession 014920-1); SEQ ID NO: 43: Human IKKβ. isoform 2 (UniProt Accession 014920-2); SEQ ID NO: 44: Human IKKβ. isoform 3 (UniProt Accession 014920-3); SEQ ID NO: 45: Human IKKβ. isoform 4 (UniProt Accession 014920-4); SEQ ID NO: 46: Murine IKKβ protein (pI=6.20, MW=84387.61, GenBank Accession no. NP_034676.1); SEQ ID NO: 47: Human IRF5 isoform 1 cds; SEQ ID NO: 48: Human IRF5 isoform 2 cds; SEQ ID NO: 49: Human IRF5 isoform 3 cds (GenBank Accession U51127); SEQ ID NO: 50: Human IRF5 isoform 4 cds (GenBank Accession nos. AY504946 or AY504947); SEQ ID NO: 51: Human IRF5 isoform 5 cds; SEQ ID NO: 52: Human IRF5 isoform 6 cds; SEQ ID NO: 53: Murine IFS cds (1494nt); SEQ ID NO: 54: Human IRF1 cds; SEQ ID NO: 55: Human IRF3 isoform 1 cds (NM_001571.5); SEQ ID NO: 56: Human IRF7 isoform A cds (NM_001572.3); SEQ ID NO: 57: Human IRF8 cds; SEQ ID NO: 58: Murine IRF1 cds (NM_001159396.1); SEQ ID NO: 59: Murine IRF3 cds (NM_016849.4); SEQ ID NO: 60: Murine IRF7 cds (NM_016850.3); SEQ ID NO: 61: Murine IRF-7/IRF-3 5(D) cds (1578 nt); SEQ ID NO: 62: Murine IRF8 cds; SEQ ID NO: 63: Murine IRF8 K310R cds (1275 nt); SEQ ID NO: 64: Human IKKβ. isoform 1 cds; SEQ ID NO: 65: Human IKKβ. isoform 2 cds; SEQ ID NO: 66: Human IKKβ. isoform 3 cds; SEQ ID NO: 67: Human IKKβ. isoform 4 cds; SEQ ID NO: 68: Murine IKKβ. cds (2217 nt).

### DETAILED DESCRIPTION

Successful genetic therapies depend on successful gene delivery mechanisms into selected cells of interest.

The current disclosure provides compositions and methods that rapidly and selectively modify cells to achieve therapeutic objectives by providing for expression of one or more nucleic acids that lasts, on average, for seven days. In some cases, transient expression of the nucleic acid or protein results. Transient expression optionally can be extended through one or more repeated applications of the compositions, thus providing repeated (serial) periods of expression that may or may not overlap. Because only transient expression is required to achieve the desired therapeutic effect(s), concerns regarding on-going side effects and/or decreased therapeutic protein expression over time are overcome.

In some embodiments, the compositions and methods disclosed herein demonstrate in vivo therapeutic efficacy as great as, or greater than, ex vivo transduced cells administered by adoptive cell therapy. Advantageously, the compositions and methods of the disclosure achieve at least 20%, at least 30%, at least 40%, at least 50%, at least 60%, at least 70%, at least 80%, or at least 90% of in vivo T cells expressing the therapeutic protein following administration of nanoparticles to a subject; result in eradication of cancer in at least 20%, at least 30%, at least 40%, at least 50%, at least 60% or at least 70% of subjects; result in an average of at least 10 days, at least 15 days, at least 20 days, at least 25 days, at least 30 days, at least 35 days, or at least 37 days improvement in survival of relapsing subjects; result in at least about the same efficacy as transplantation of T cells contacted with the nanocarrier ex vivo; and/or result in at least about the same efficacy as transplantation of ex vivo transduced CAR⁺ T cells.

Specifically contemplated herein are embodiments that include repeated delivery of a nanoparticle composition to a patient, where the nanoparticles target selected cells within the patient and result in transient expression of a therapeutic protein by the selected cells. In particular embodiments, repeated delivery occurs every 5-10 days (e.g., every 7 days).

### Definitions

As used herein, “nanoparticle” and “nanocarrier” are used interchangeably and refer generally to a module for transport of another substance, termed a “cargo,” such as a protein, polynucleotide, or drug. Commonly used nanocarriers include micelles, polymers, carbon-based materials, liposomes and other substances. The nanocarriers of the present disclosure generally include, at least, a positively-charged carrier matrix and a neutrally or negatively-charged coating. The coating is on the outer surface of the of the carrier matrix, optionally with or without interposed intermediate layers. The cargo is generally a polynucleotide either encoding a therapeutic protein (e.g., a chimeric antigen receptor (CAR), T cell receptor (TCR), CAR/TCR hybrid, cell receptor, transcription factor, macrophage activator, or signaling molecule, or encoding a therapeutic polynucleotide (e.g., an mRNA, shRNA, gRNA, or sgRNA).

As used herein, “coating” of a nanocarrier refers to the outermost layer of the nanocarrier, although cell targeting ligands may shield portions of the coating. The coating may include a neutral or negatively-charged coating, such as a negatively-charged polyglutamic acid (PGA), poly(acrylic acid), alginic acid, or cholesteryl hemisuccinate/1,2-dioleoyl-sn-glycero-3-phosphoethanolamine or a neutrally-charged zwitterionic polymer.

As used herein, “carrier matrix” refers the constituents of the nanocarrier that mediate incorporation of the cargo into the nanocarrier, excluding the coating and any intermediate layers. Generally, when the cargo is a polynucleotide, the carrier matrix is a positively-charged carrier matrix, which is suitable for incorporation of polynucleotides into the carrier because polynucleotides are negatively charged. The lipid or polymer may be positively-charged poly(β-amino ester, poly(L-lysine), poly(ethylene imine) (PEI), poly-(amidoamine) dendrimers (PAMAMs), poly(amine-co-esters), poly(dimethylaminoethyl methacrylate) (PDMAEMA), chitosan, poly-(L-lactide-co-L-lysine), poly[α-(4-aminobutyl)-L-glycolic acid] (PAGA), or poly(4-hydroxy-L-proline ester) (PHP); combinations of the foregoing; or equivalents.

As used herein, “extending from the surface of the coating” means that ligand is attached to the coating, directly or indirectly, and extends away from the coating a sufficient distance to permit interaction of the ligand with its target. Attachment may be achieved by chemical coupling, by incorporations of a lipid-binding constituent into the ligand (e.g. gene-fusion of the ligand to a transmembrane domain of a protein), by charge-charge interaction, or by other means.

As used herein, “selected cells” refers to a cell or cell type selected as a target for the nanocarrier composition by the maker or user of the nanocarrier. For example, the selected cells may be immune cells, such as T cells, B cells, or NK cells. The selected cells may also be subsets of the foregoing, such as CD4+ T cells, CD8+ T cells, or T regulatory cells. The selected cells may be further subsets of the foregoing, as in some embodiments multiple targeting ligands are employed to achieve targeting to cells distinguished by multiple cell markers.

As used herein, “disease-specific receptor” refers to a protein that specifically binds to a biomolecule related to the causative agent for a disease or indicative of the disease. For example, a disease-specific receptor for a cancer would include a protein that marks cancerous cells and distinguishes them from non-cancerous cells, such as by overexpression on cancerous cells. A disease-specific receptor for an infectious disease might include, for example, a receptor that specifically binds to the infectious agent directly or a receptor that specifically binds to a biomolecule displayed on the surface of infected cells (e.g. a peptide-MHC complex where the peptide is an infectious-agent specific peptide).

As used herein, “selectively incorporated” means that the nanocarrier is incorporated into the selected cells at higher rates or to a greater maximum incorporated amount than the nanocarrier is incorporated into other cells. “Selectively incorporated” may mean that the nanocarrier is incorporated into selected cells 2-fold, 3-fold, 4-fold, 5-fold, 10-fold, 100-fold, 1,000-fold or more rapidly or effectively than into cells other than selected cells.

As used herein, “selectively binds” means binds to a target with at least 2-fold, 3-fold, 4-fold, 5-fold, 10-fold, 100-fold, 1,000-fold or more higher affinity than to a reference molecule.

As used herein, “express the therapeutic protein” means that selected cells are contacted with the nanocarrier or the nanocarrier is administered to subjects, the selected cells express the therapeutic protein in amounts detectable by conventional methods, such as gel electrophoresis, mass-spectrometry, fluorescence microscopy, flow cytometry, and/or Western blotting. Where the therapeutic protein is expressed endogenously by the selected cells, “express the therapeutic protein” means that the contacting or administering step results in at least 5%, 10%, 15%, 20%, or greater increase in expression of therapeutic protein in the selected cells.

As used herein, “HBV-induced hepatocellular carcinoma” refers to hepatocellular carcinoma known to have been caused by HBV or hepatocellular carcinoma that a medical professional, using reasonable judgment, would understand to have been caused by HBV.

As used herein, “eradication” of cancer refers to complete response (CR).

As used herein, “subject” or “patient” are used interchangeably. A “subject” includes any mammal. The mammal can be e.g., a human or appropriate non-human mammal, such as primate, mouse, rat, dog, cat, cow, horse, goat, camel, sheep or a pig. The subject can also be a bird or fowl. Preferably, the subject is a human. A subject can be male or female. A subject in need thereof can be one who has not been previously diagnosed or identified as having a condition, e.g. an autoimmune disease, infectious disease, cancer or a precancerous condition. A subject in need thereof can be one who has been previously diagnosed or identified as having cancer or a precancerous condition. A subject in need thereof can also be one who is having (suffering from) condition, e.g. an autoimmune disease, infectious disease, cancer or a precancerous condition. Alternatively, a subject in need thereof can be one who has a risk of developing such disorder relative to the population at large (i.e., a subject who is predisposed to developing such disorder relative to the population at large).

Optionally, a subject in need thereof has already undergone, is undergoing or will undergo, at least one therapeutic intervention for the condition.

A subject in need thereof may have a refractory condition, e.g. refractory cancer, on most recent therapy. “Refractory cancer” means cancer that does not respond to a previously-administered treatment. The cancer may be resistant at the beginning of treatment or it may become resistant during treatment. Refractory cancer is also called resistant cancer. In some embodiments, the subject in need thereof has cancer recurrence following remission on most recent therapy. In some embodiments, the subject in need thereof received and failed all known effective therapies for cancer treatment. In some embodiments, the subject in need thereof received at least one prior therapy.

As used herein, “relapsing subjects” refers to subjects that have demonstrated CR, partial response (PR), remission, or prolonged remission after prior treatment followed by re-occurrence of the cancer.

As used herein, “ex vivo” refers to methods directed to cells outside of the body of a subject or a donor.

As used herein, “in vivo” refers to methods directed to cells in the body of the subject.

As used herein, “in vitro” refers to methods directed to cells grown in culture rather to primary cells.

As used herein, “cell targeting ligand” or “selected cell targeting ligand” are used interchangeably and refer to a biomolecule (e.g. a protein or a polynucleotide) that selectively binds a selected cell (e.g. through a marker protein on the surface of the selected cell). Generally a cell targeting ligand selectively targets a nanocarrier to the select cell in vivo. Exemplary cell targeting ligands include antibody fragments such as a single chain variable fragment (scFv), engineered ligand such as rationally engineered binding agents, small-molecules ligands, or aptamers.

### Embodiments

In particular embodiments, transient expression is expression for 12 hours to 15 days; for 18 hours to 12 days; from 20 hours to 14 days; from 24 hours to 10 days, from 24 hours to 8 days, or from 30 hours to 7 days. It is specifically contemplated that transient expression in various embodiments is no longer than 14 days. For instance, in particular embodiments transient expression is detectable expression which lasts no longer than 12 days, no longer than 10 days, no longer than 9 days, no longer than 8 days, or no longer than 7 days. In embodiments where longer expression is desired, a nanoparticle providing transient expression of a therapeutic protein can be delivered to a subject with repeated doses, for instance delivery that occurs every 5-10 days (e.g., every 7 days).

In particular embodiments, subjects can be monitored for expression of the therapeutic protein, and when expression falls below a threshold, a treating physician can determine whether additional nanoparticles resulting in additional expression of the therapeutic protein is warranted.

In particular embodiments, the delivery of nanoparticles can be intravenous or at, to, or near a selected anatomical site (e.g., a tumor site).

In particular embodiments, delivery of nanoparticles can be coordinated with the use of cell attractants at a treatment site. For example, a subject can be administered an agent that attracts a cell type to the anatomical site. In particular embodiments, the attracted cell type can be the same cell type as that targeted for genetic modification to express a nucleic acid or protein, such as a therapeutic protein. For example, if the anatomical site is a tumor site, it can be beneficial to attract T cells to the tumor site, and then modify the attracted T cells to express a nucleic acid or protein, such as a therapeutic protein, such as a chimeric antigen receptor (CAR), a T cell receptor (TCR) or a CAR/TCR hybrid. In particular embodiments, the attracted cell type can be a different cell type from that targeted for genetic modification to express a nucleic acid or protein, such as a therapeutic protein. For example, if the anatomical site is a tumor site, it can be beneficial to attract cells to the tumor site that support the activity of the selected cells modified to transiently express the therapeutic protein. Cells that support the activity of T cells can include subsets of T cells (e.g., T helper), natural killer (NK) cells, and macrophages. In particular embodiments, it can be beneficial to attract more than one cell type to an anatomical site. In particular embodiments, cells can be attracted to an anatomical site before delivery of the nanoparticles (e.g., “preconditioning”).

In particular embodiments, treatment protocols described herein can also include activating macrophages at the treatment site. Activating macrophages at a treatment site can, for example, overcomes tumor suppression of macrophage(s) of the subject being treated.

In particular embodiments, nanoparticles utilized to genetically modify selected cell types in vivo to express a nucleic acid or protein, such as a therapeutic protein include (1) a selected cell targeting ligand; (2) a positively-charged carrier; (3) nucleic acids within the positively-charged carrier; and (4) a neutral or negatively-charged coating.

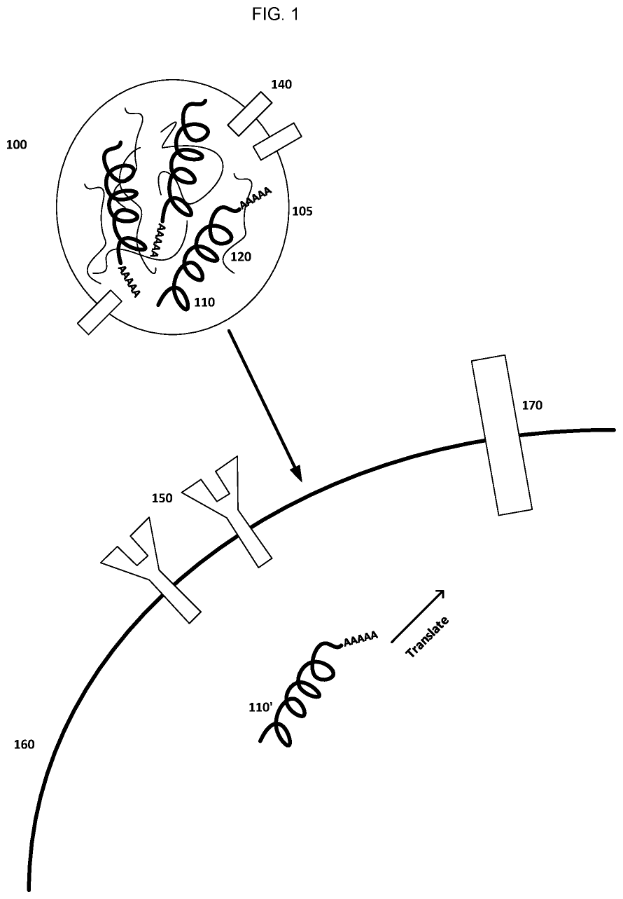

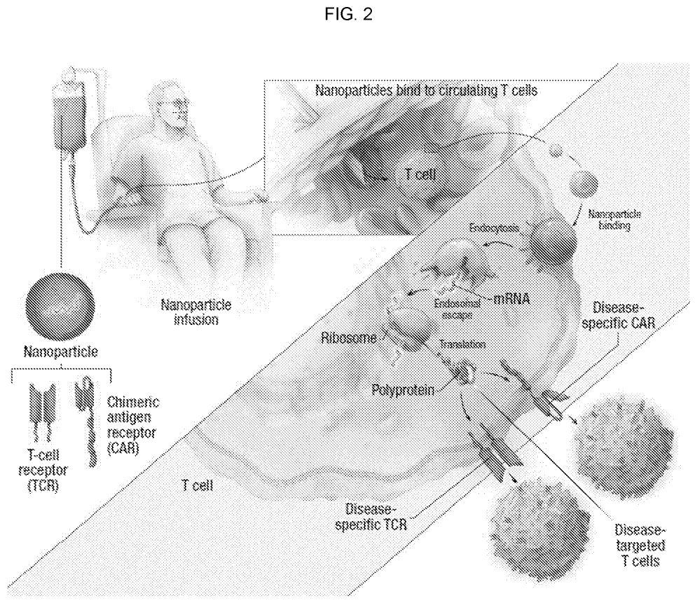

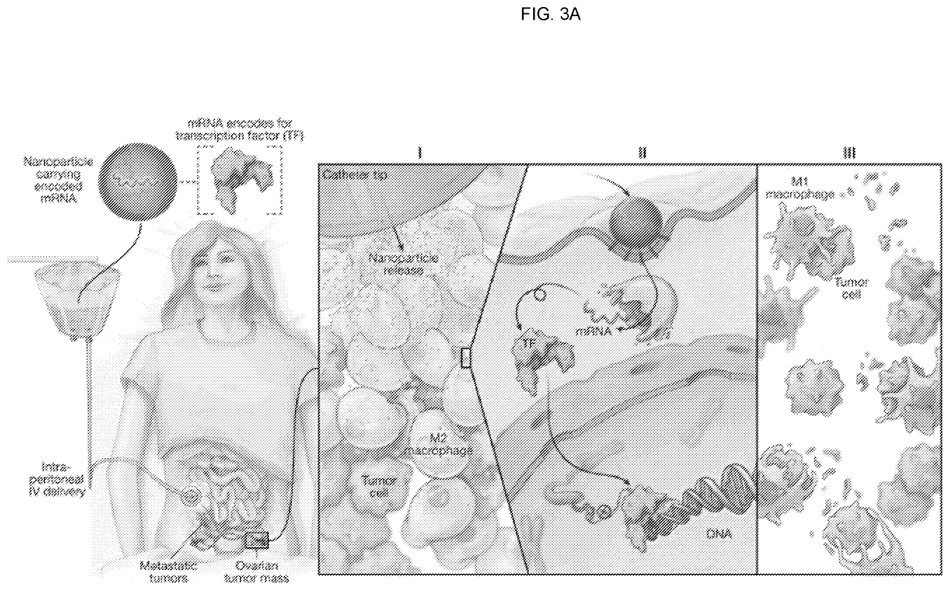

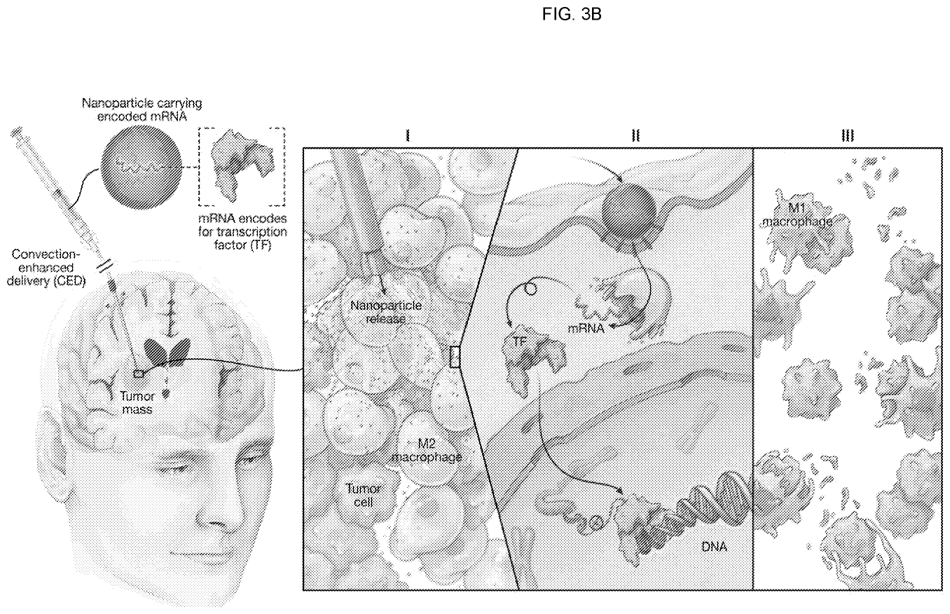

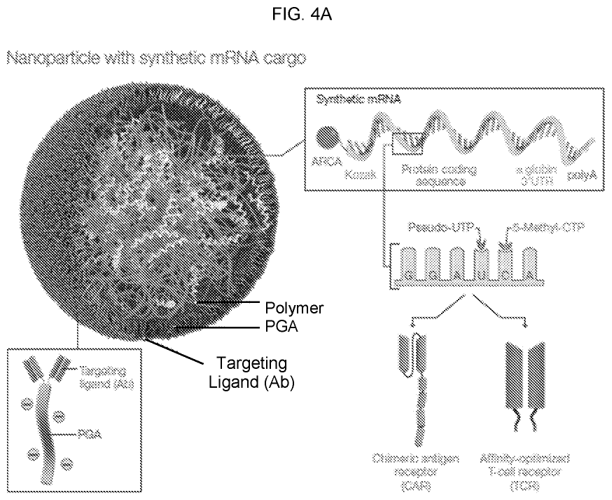

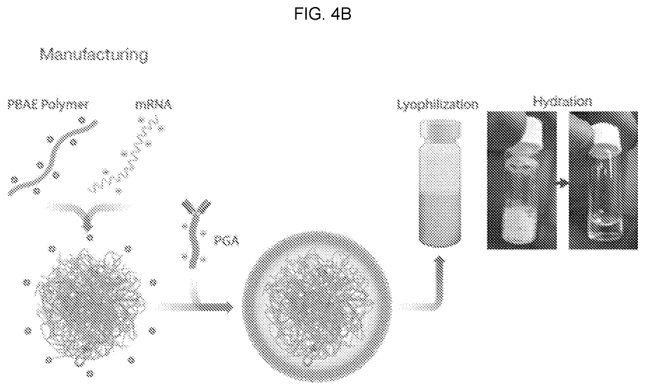

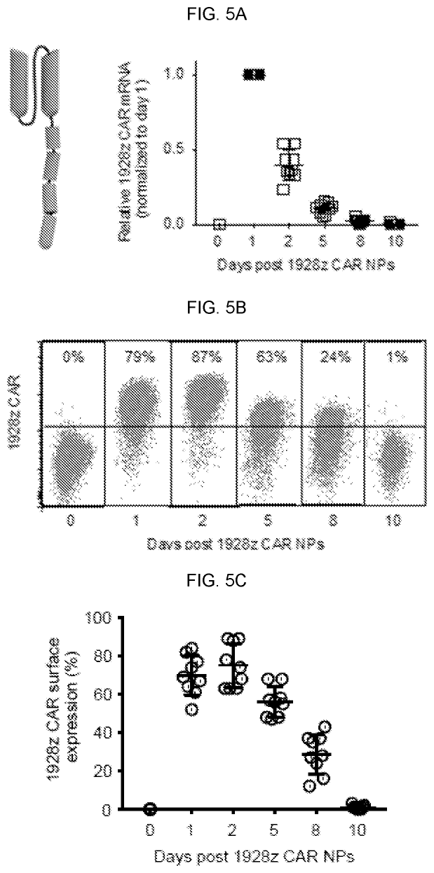

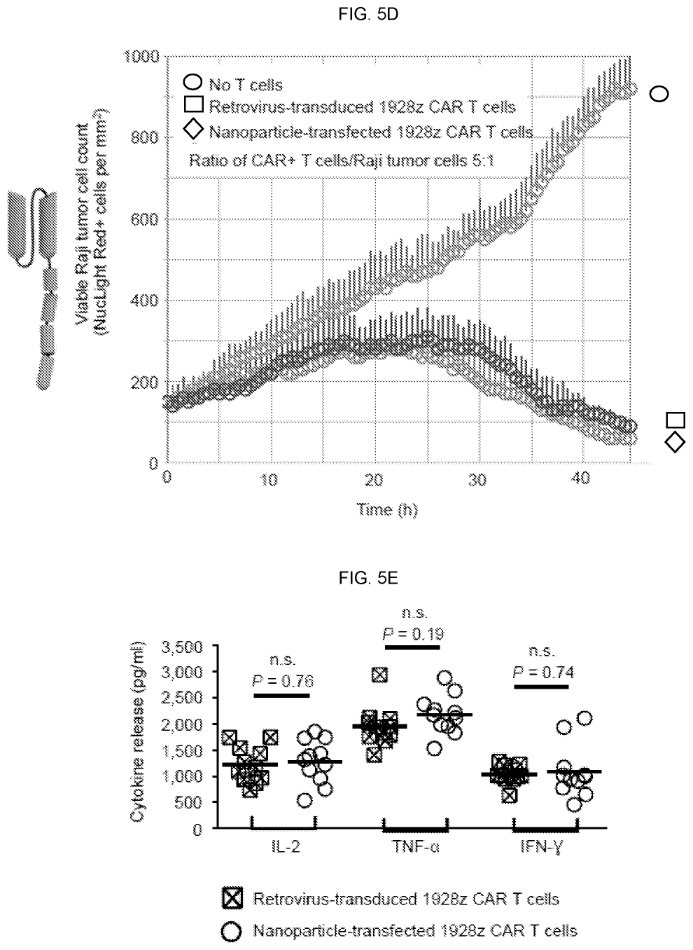

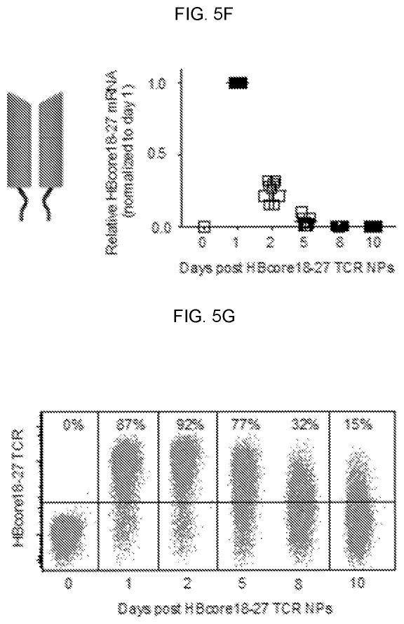

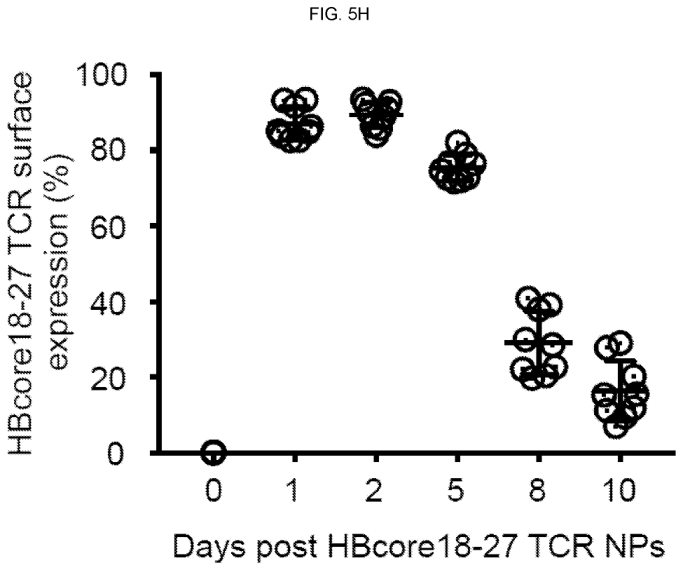

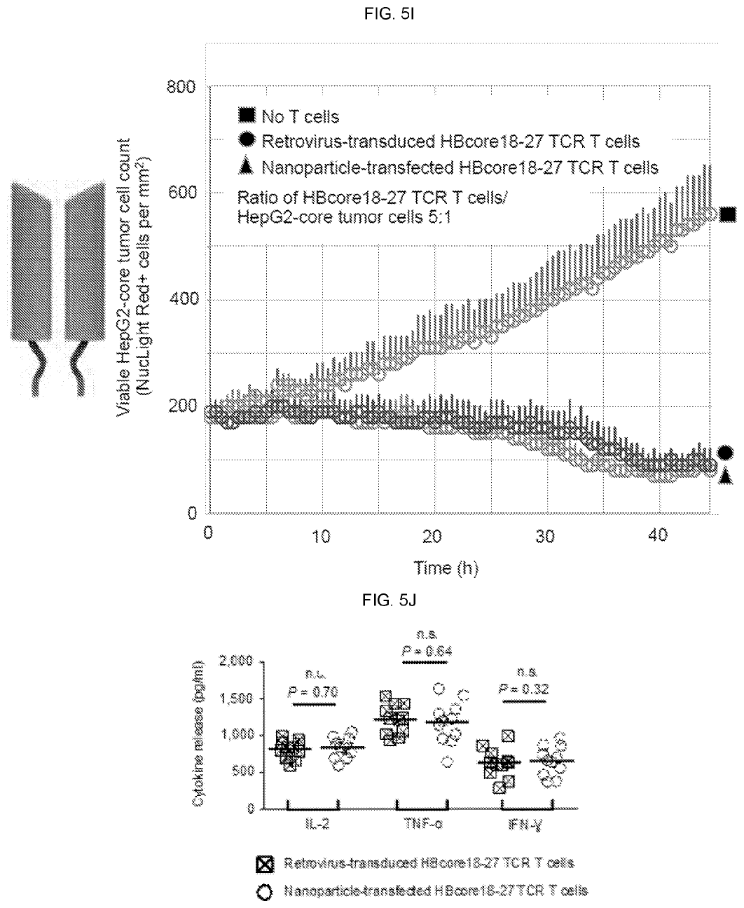

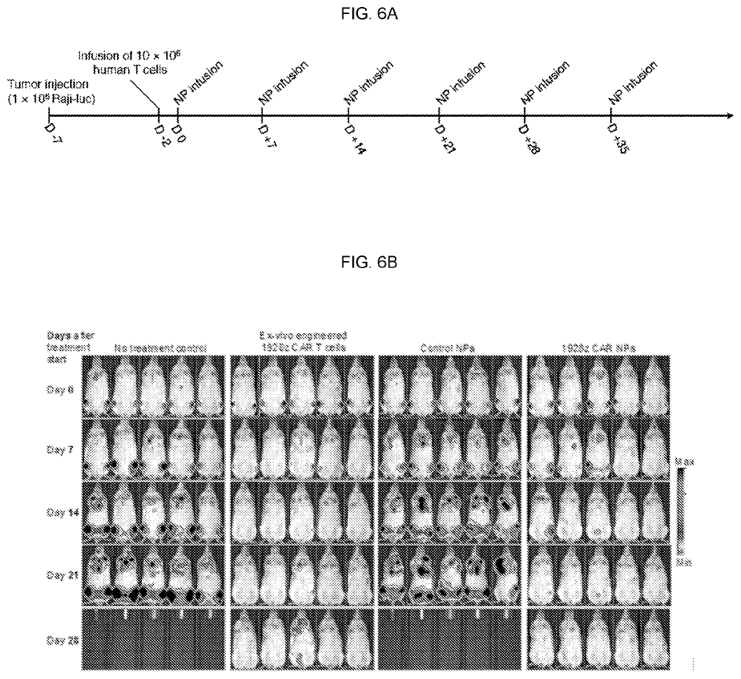

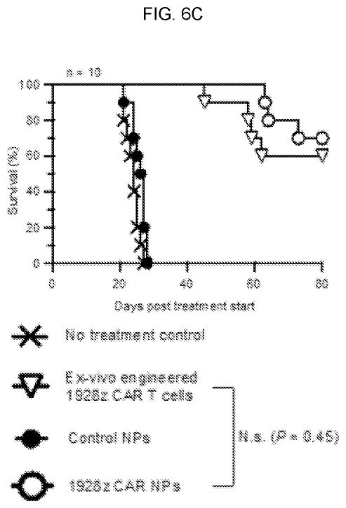

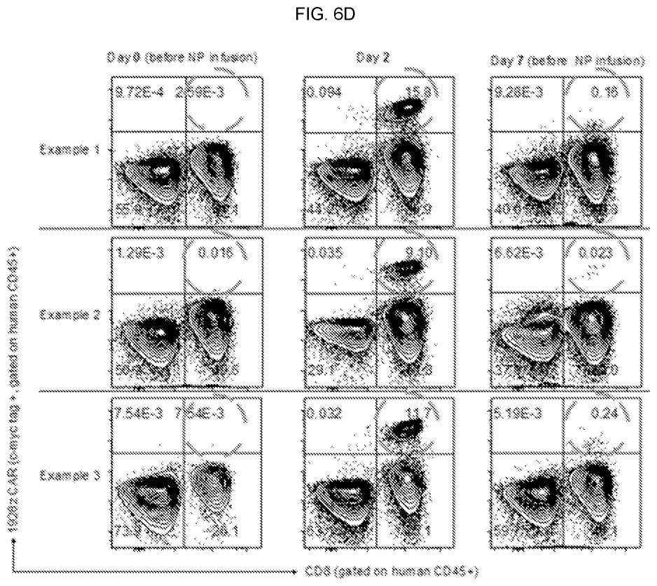

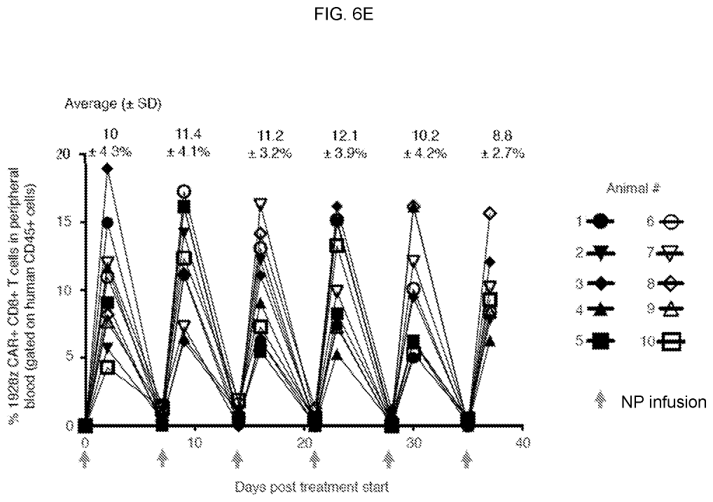

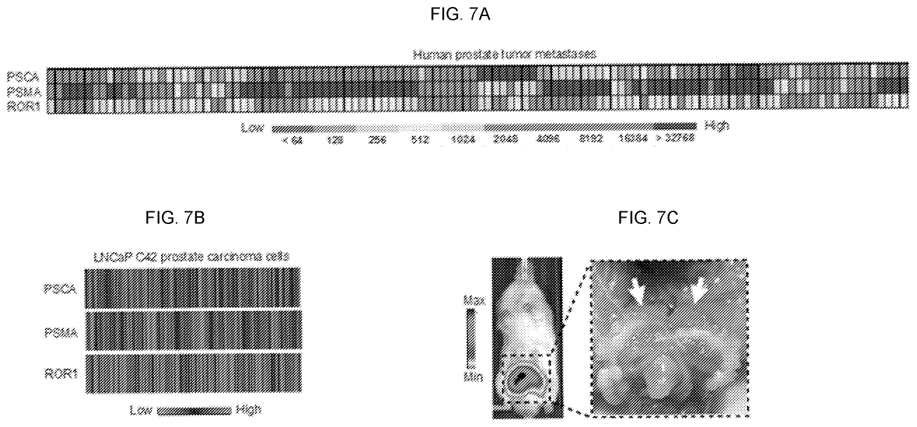

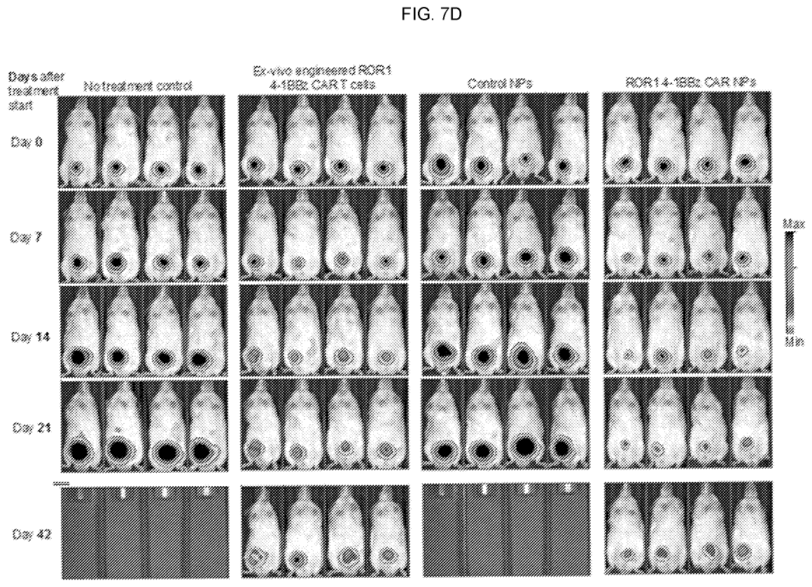

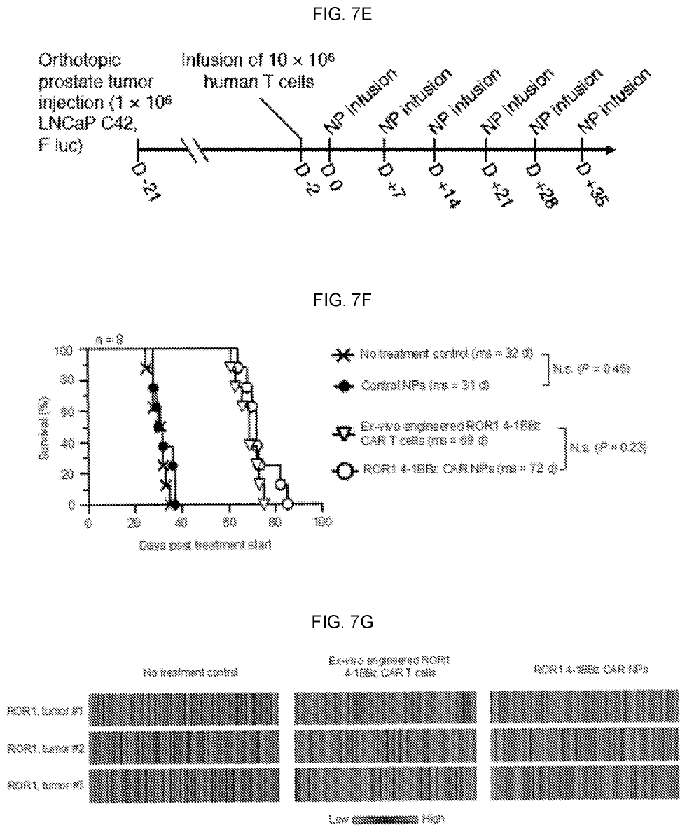

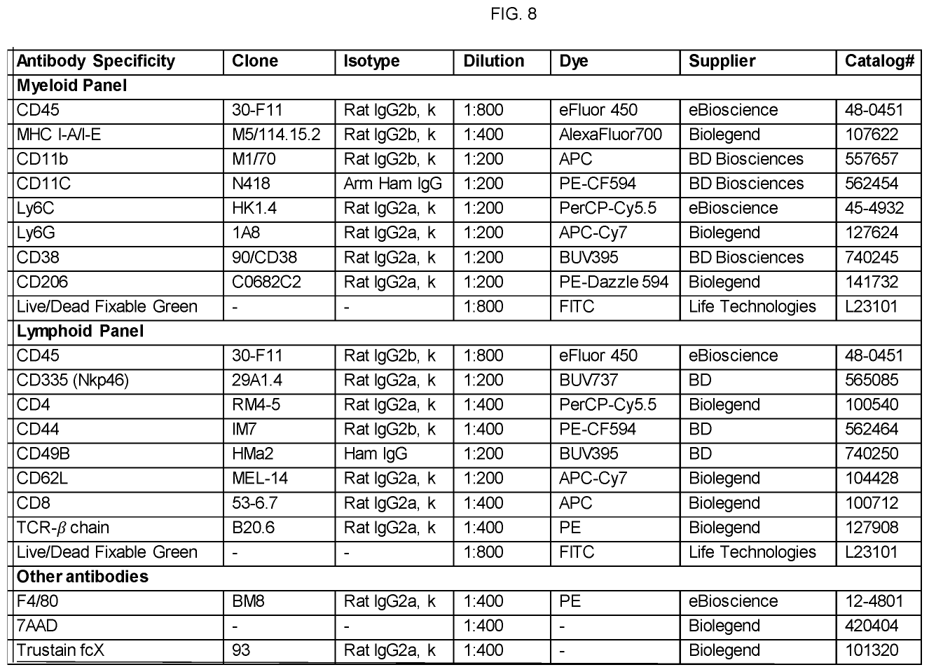

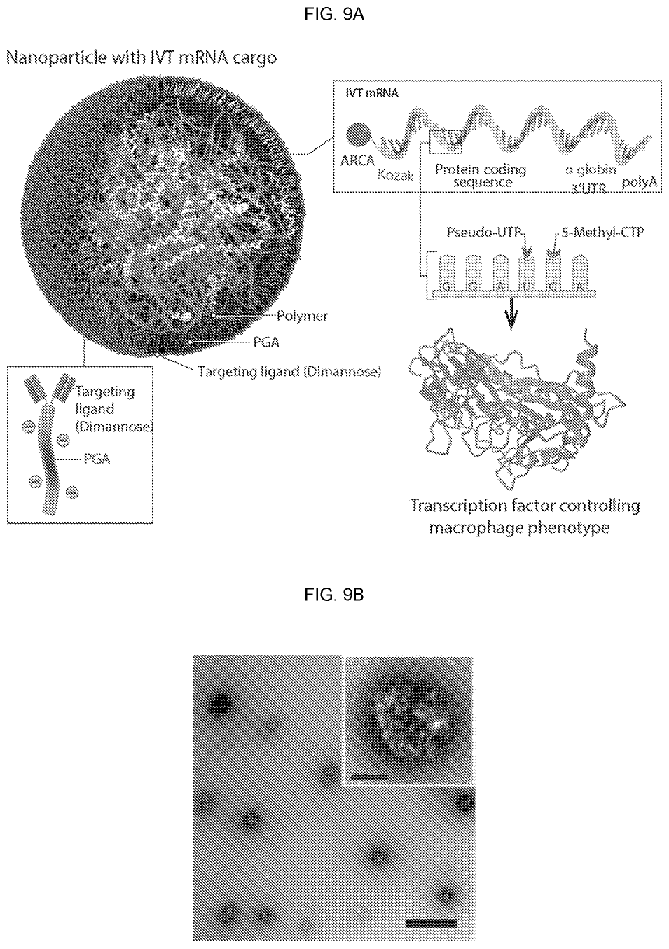

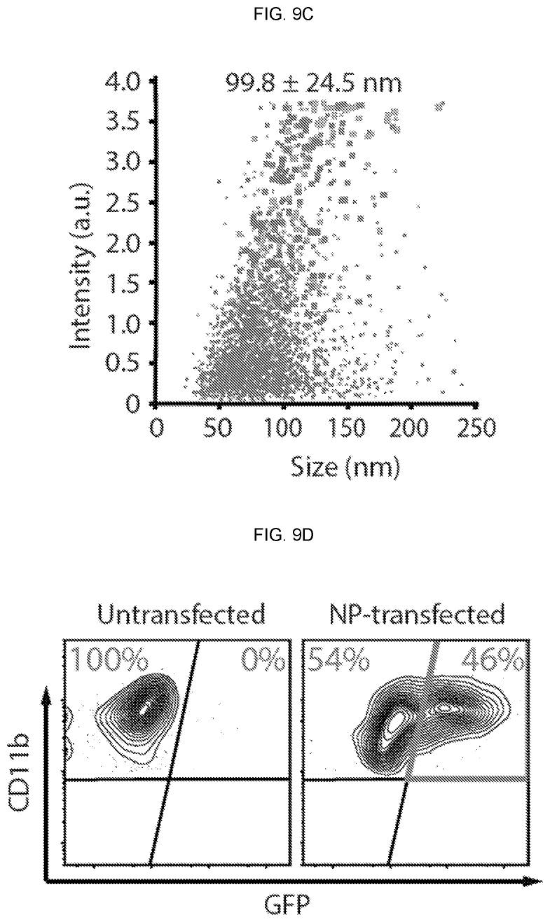

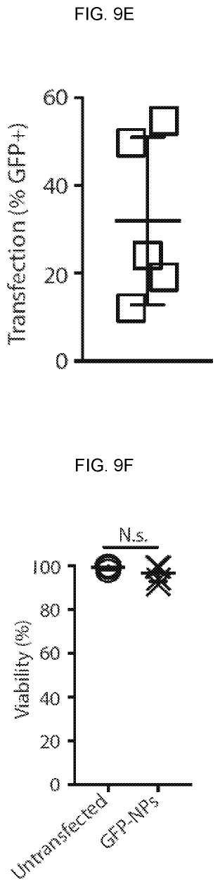

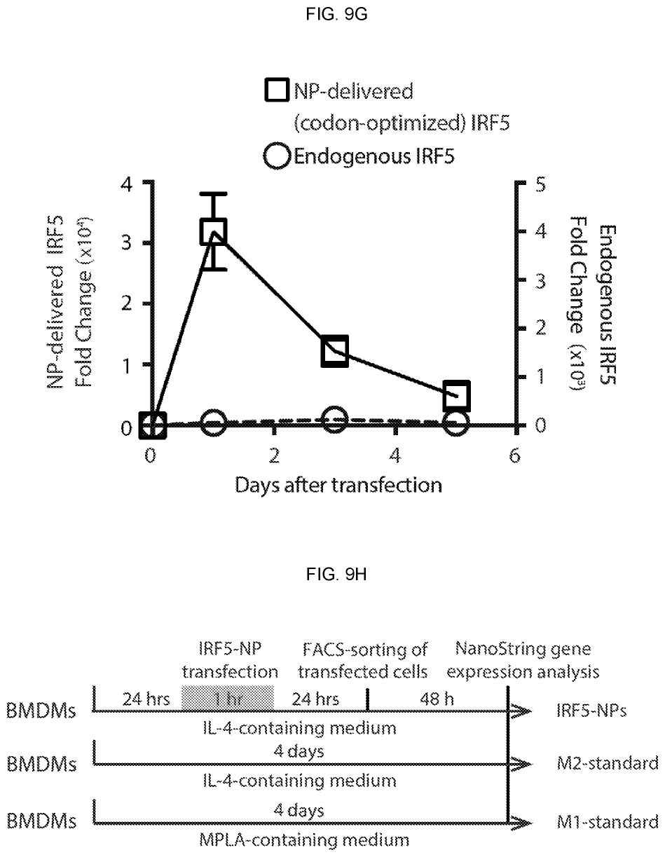

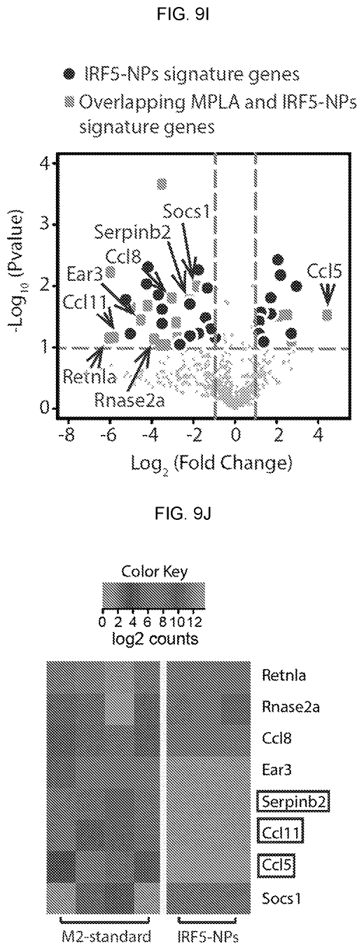

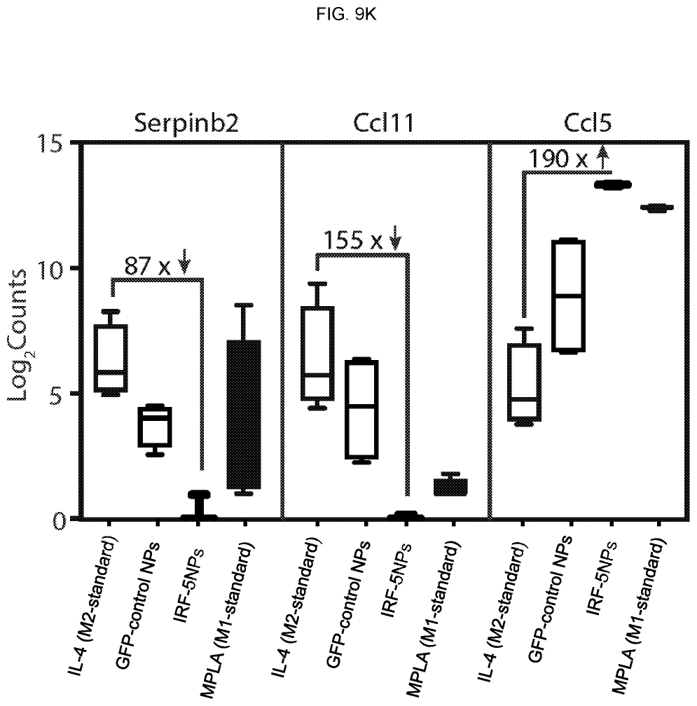

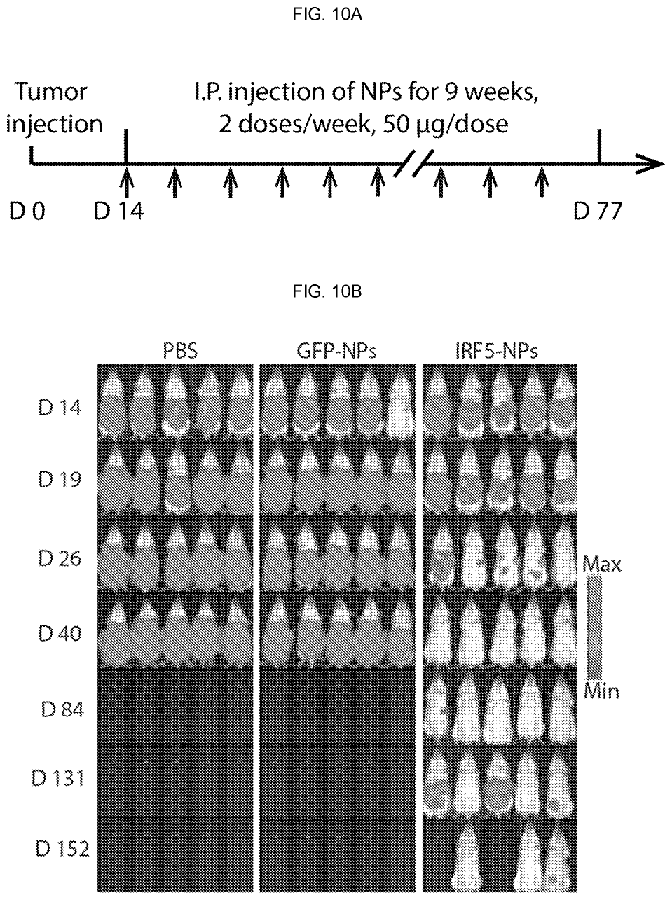

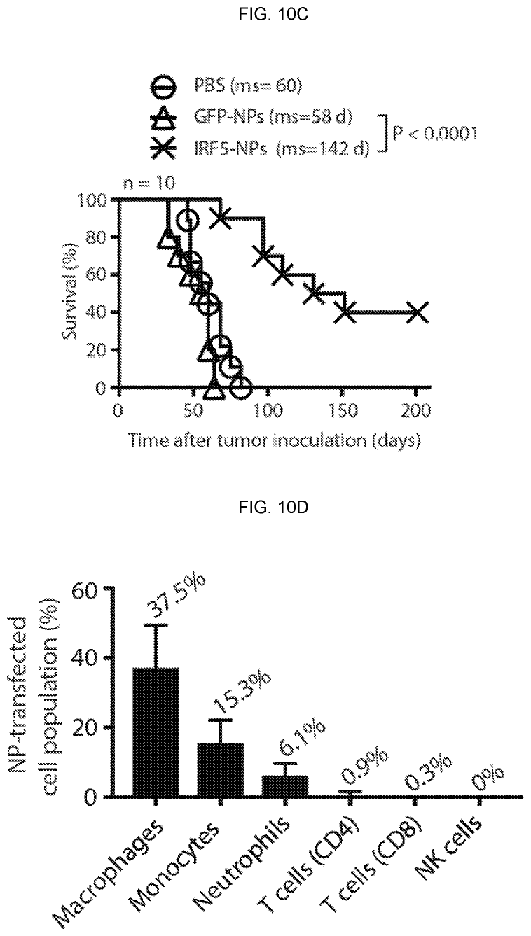

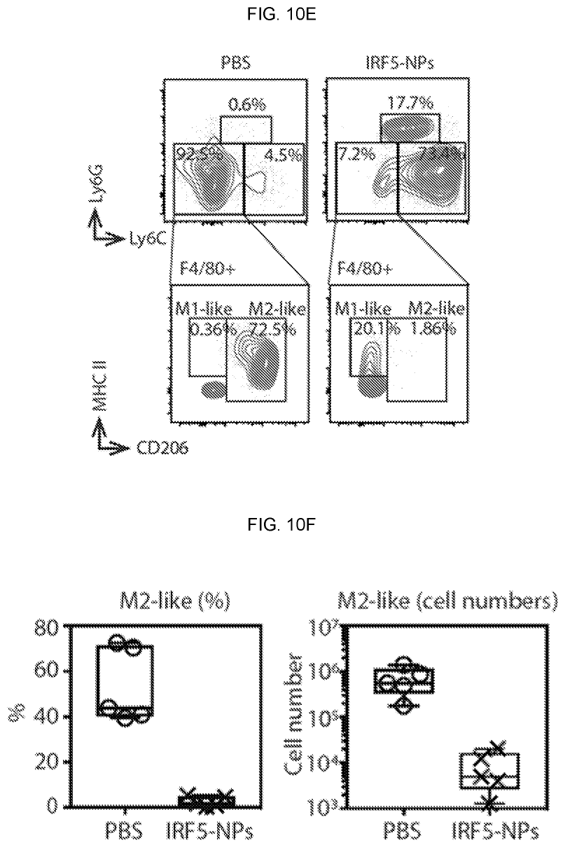

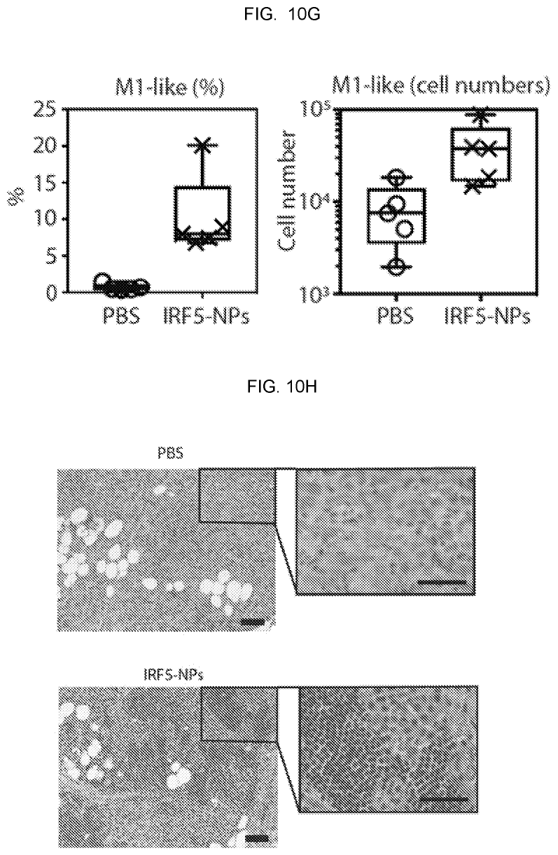

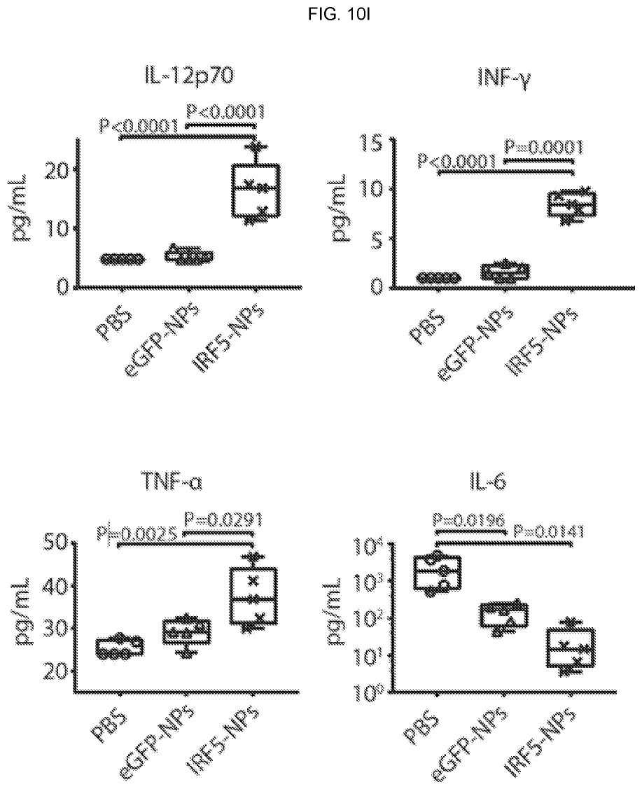

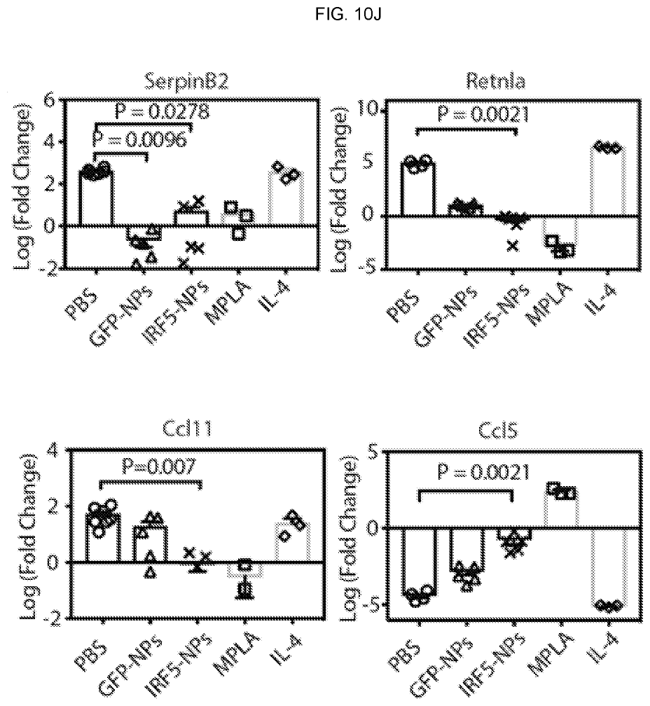

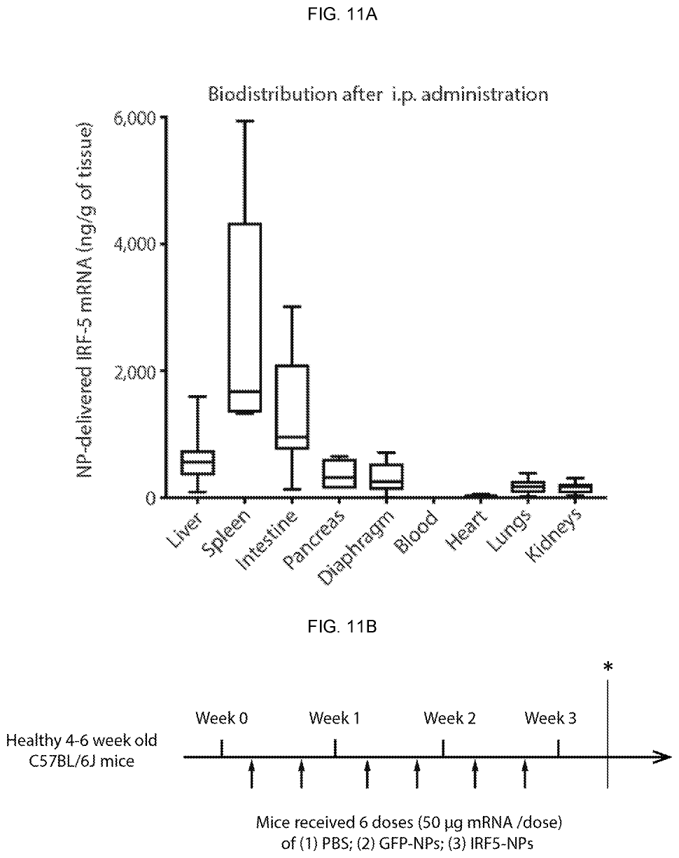

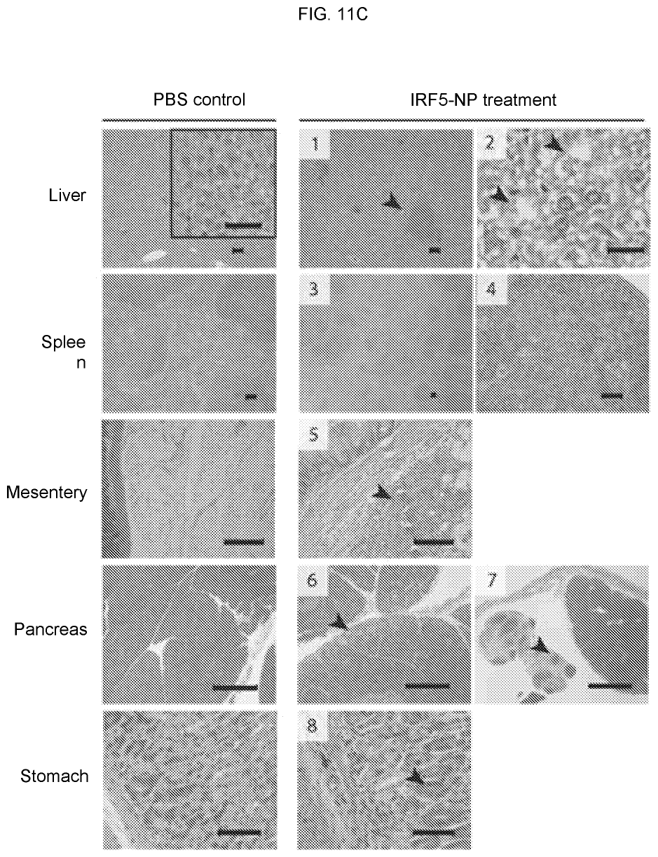

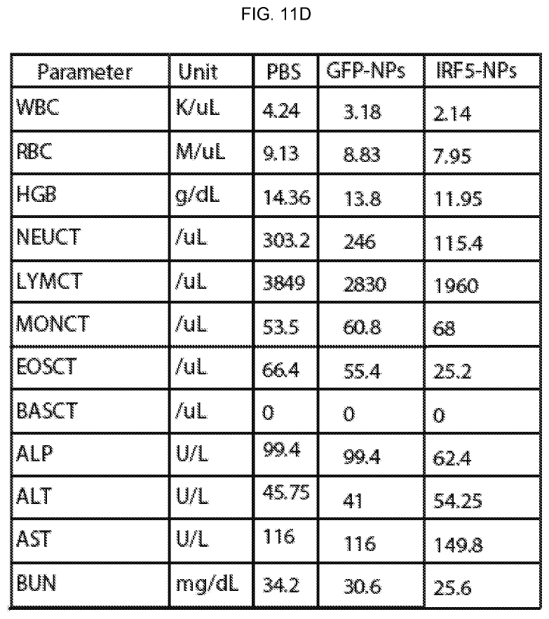

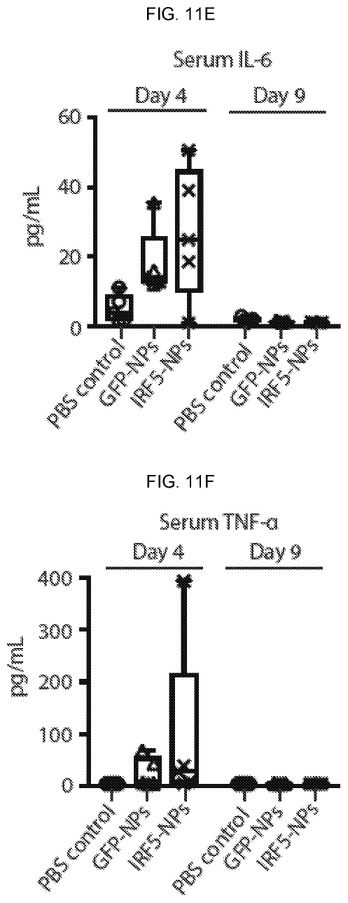

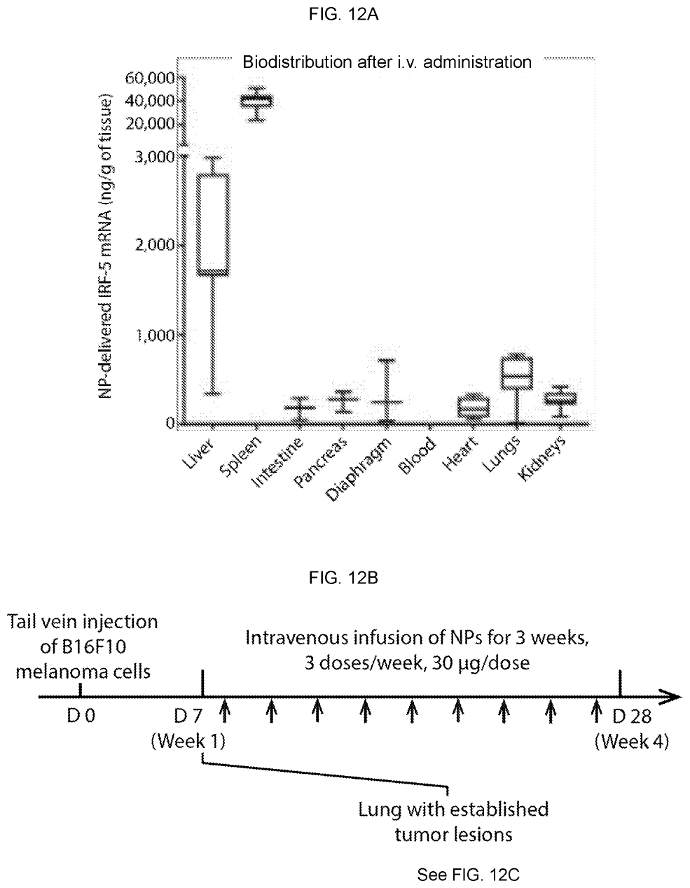

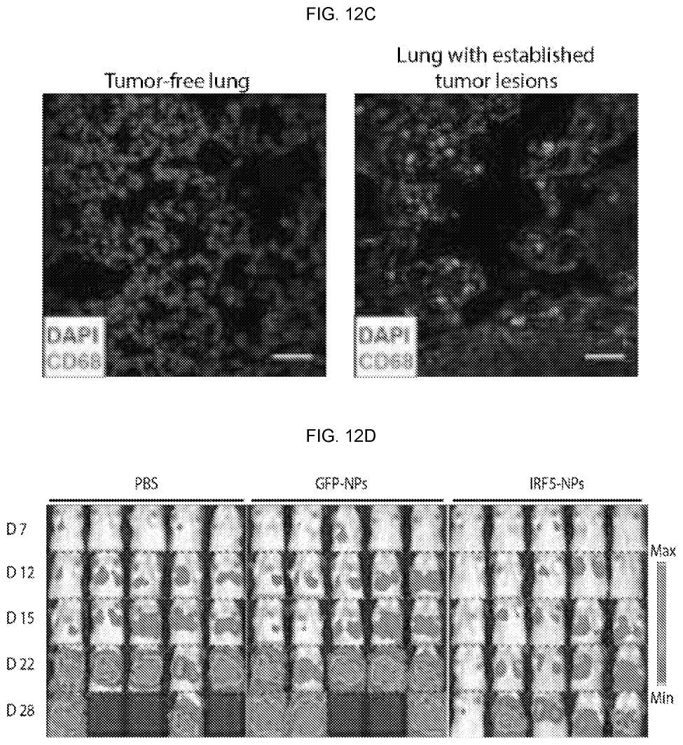

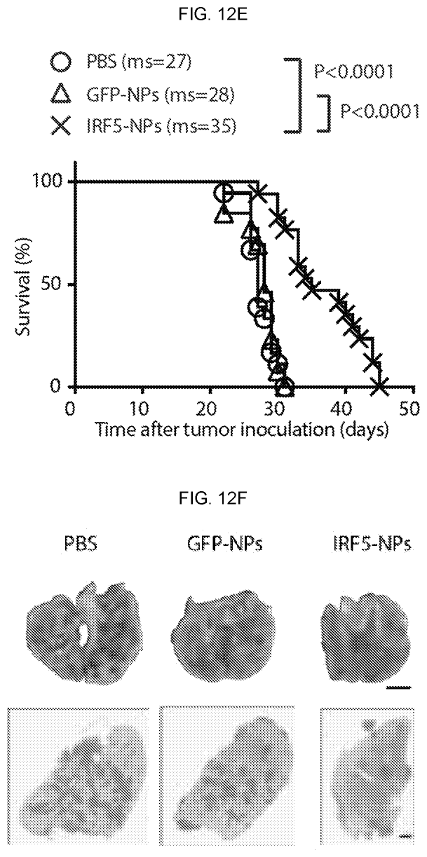

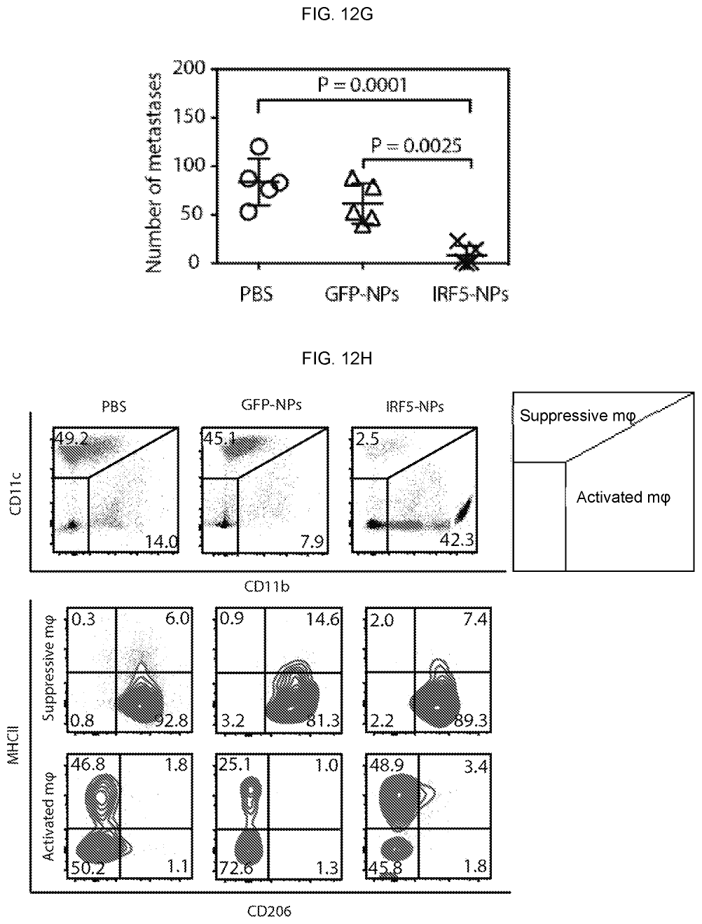

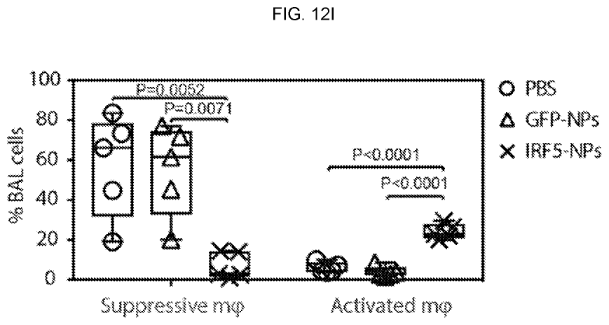

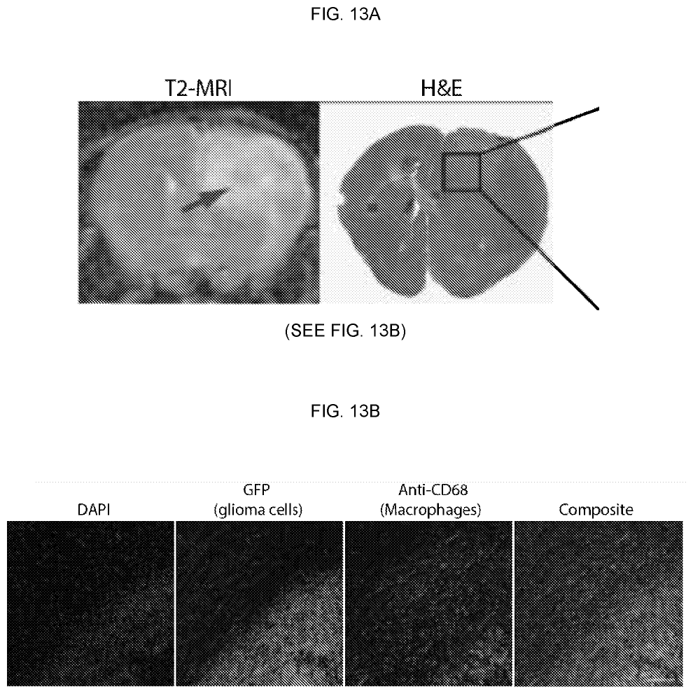

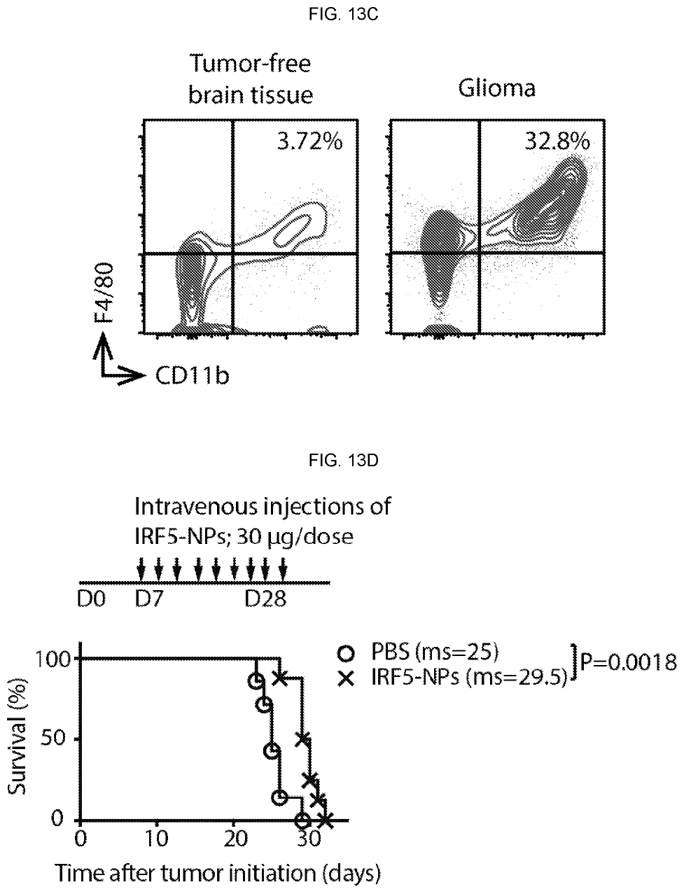

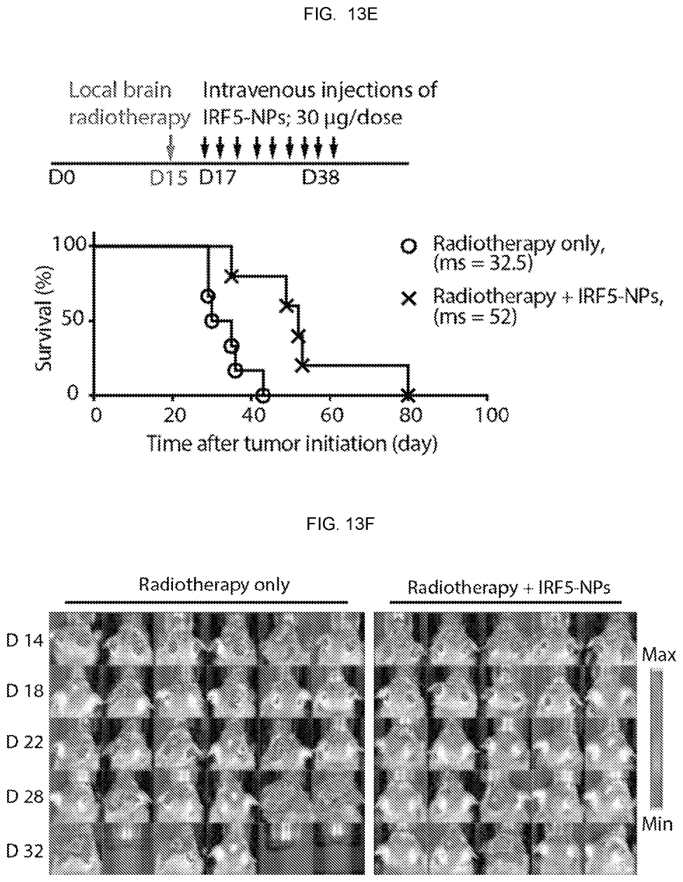

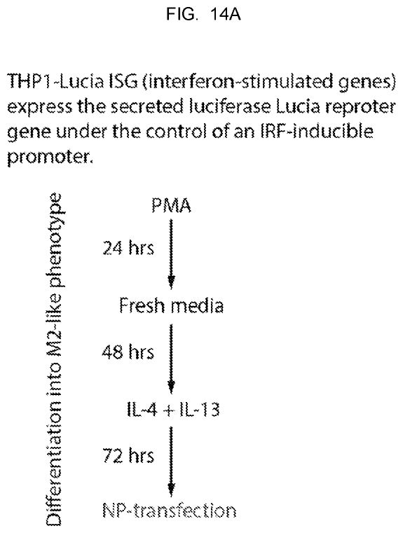

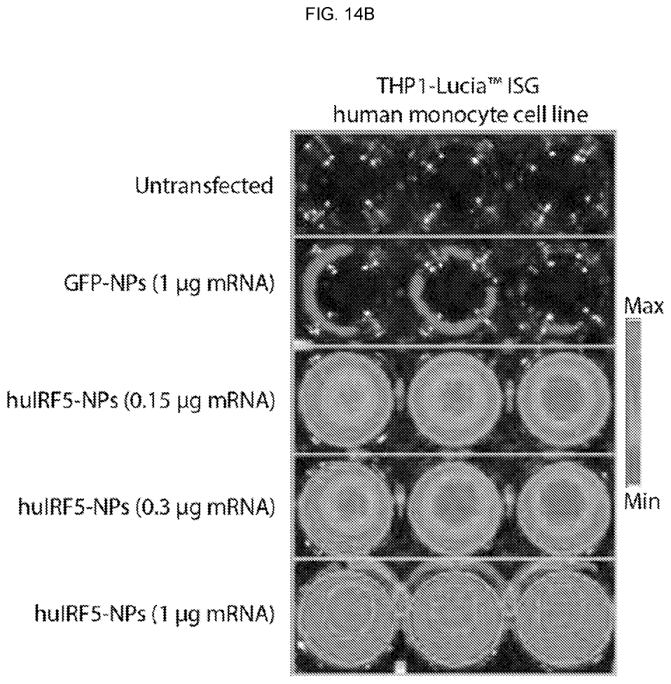

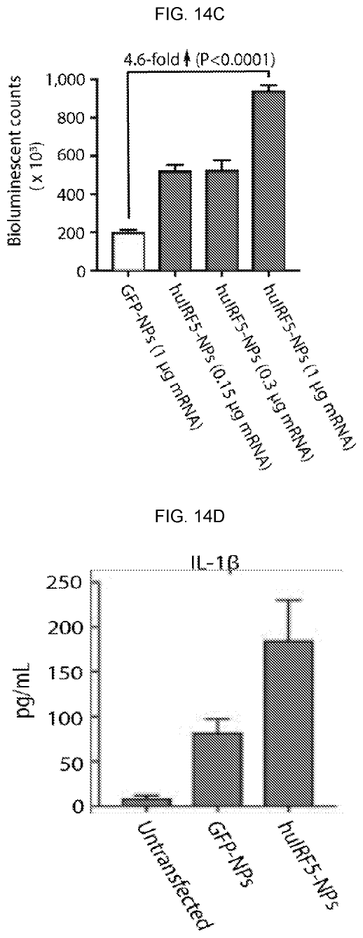

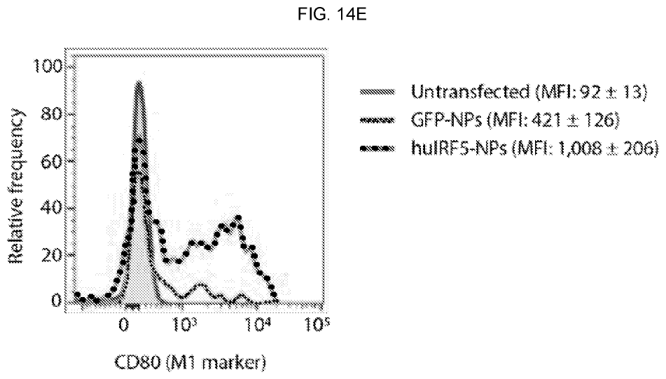

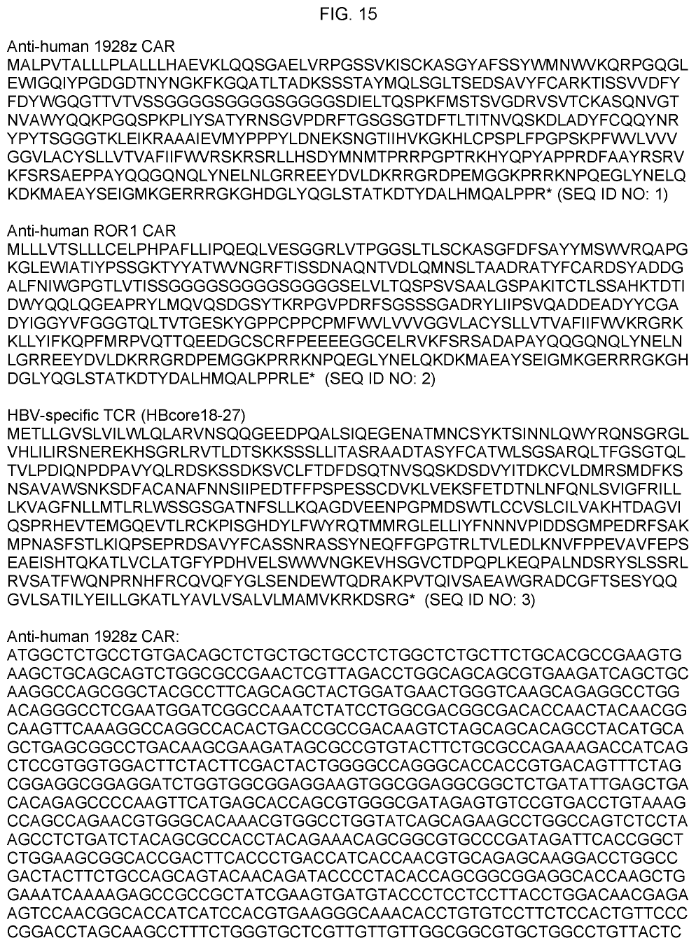

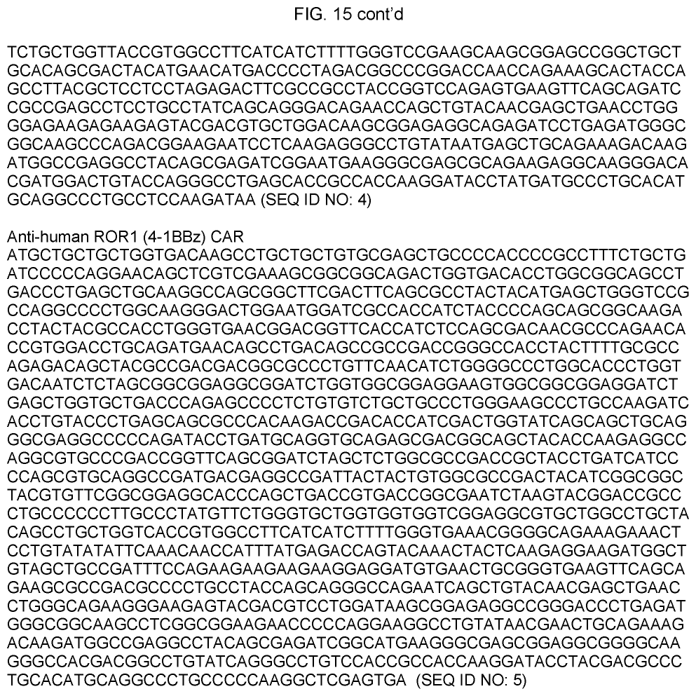

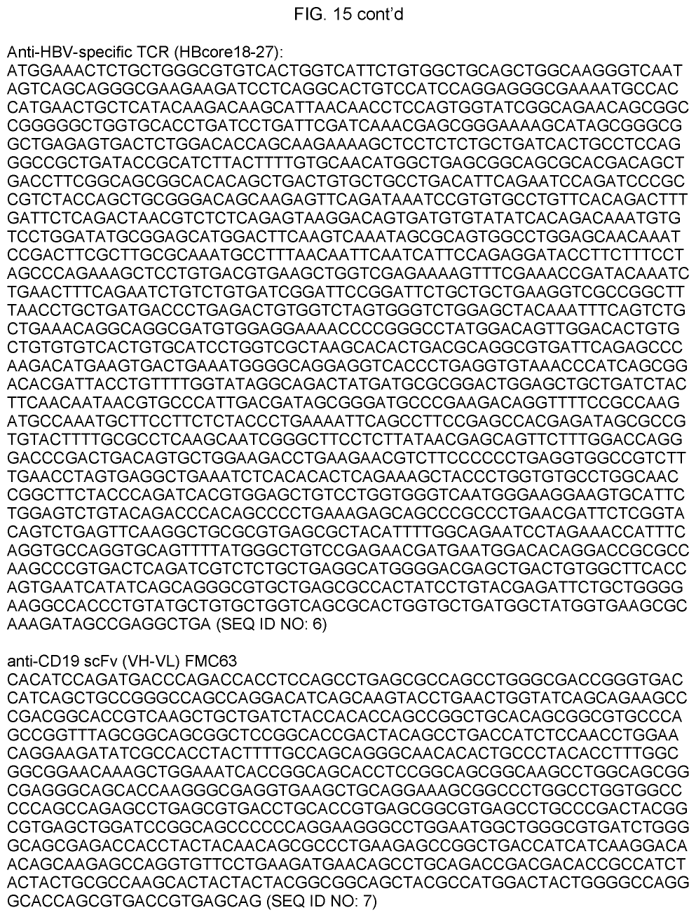

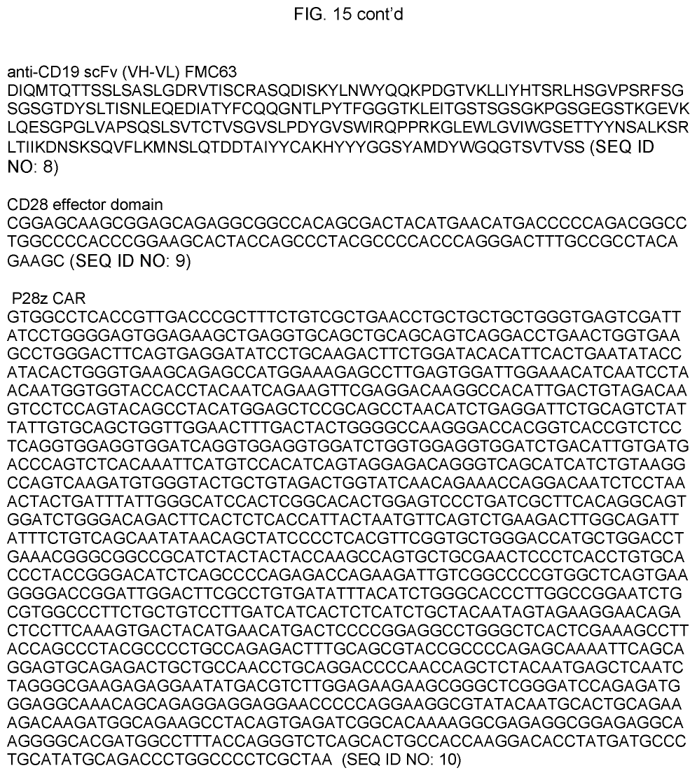

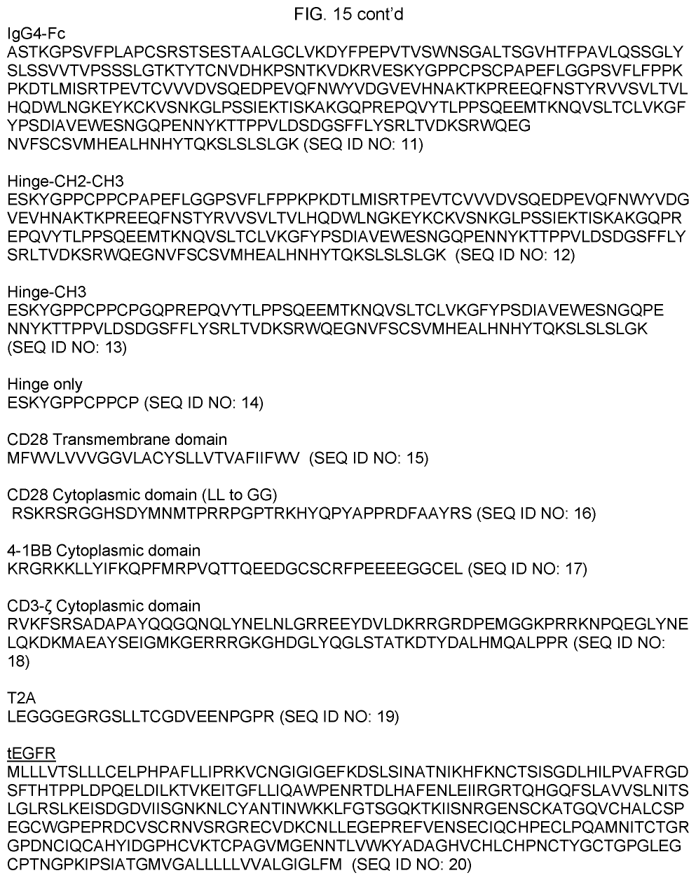

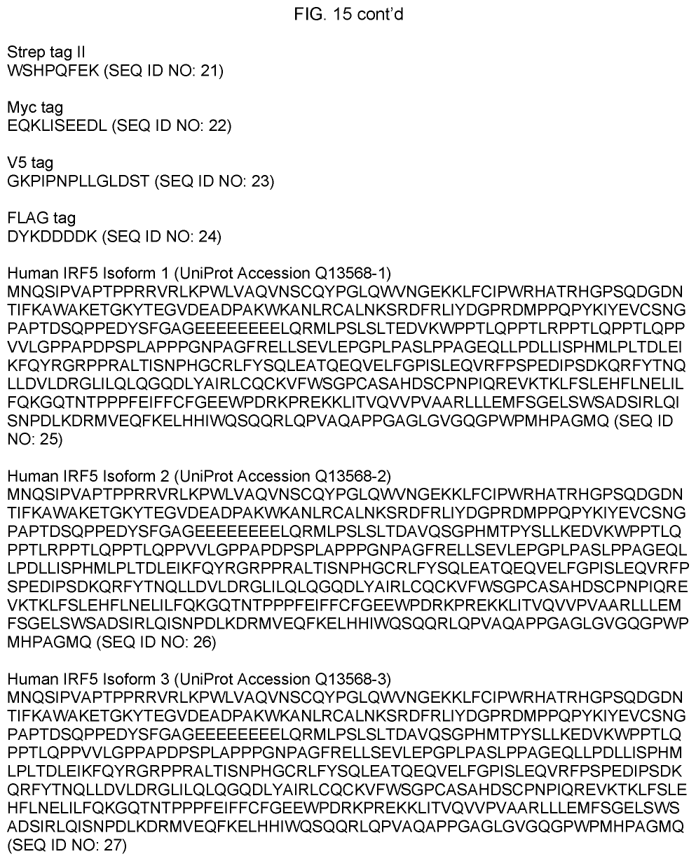

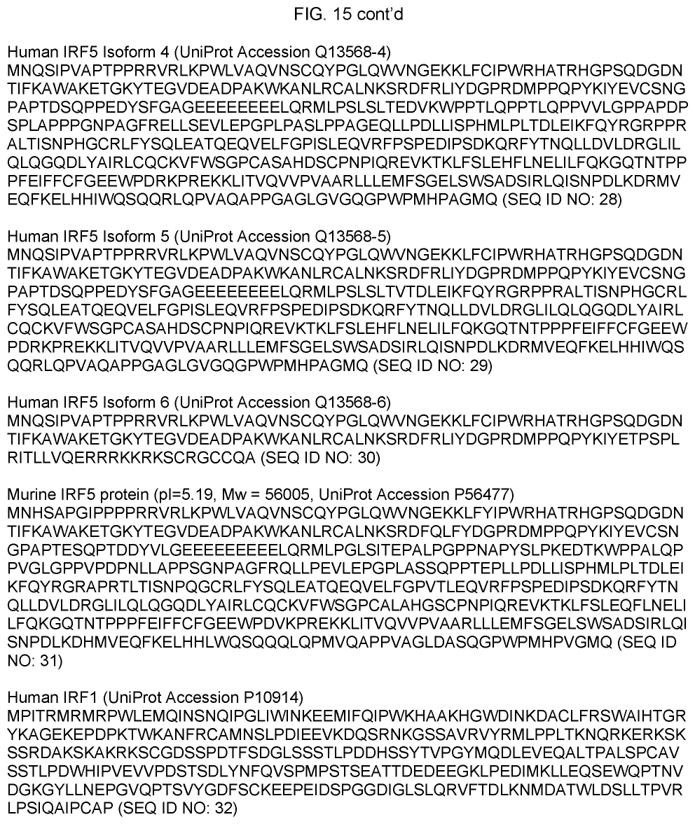

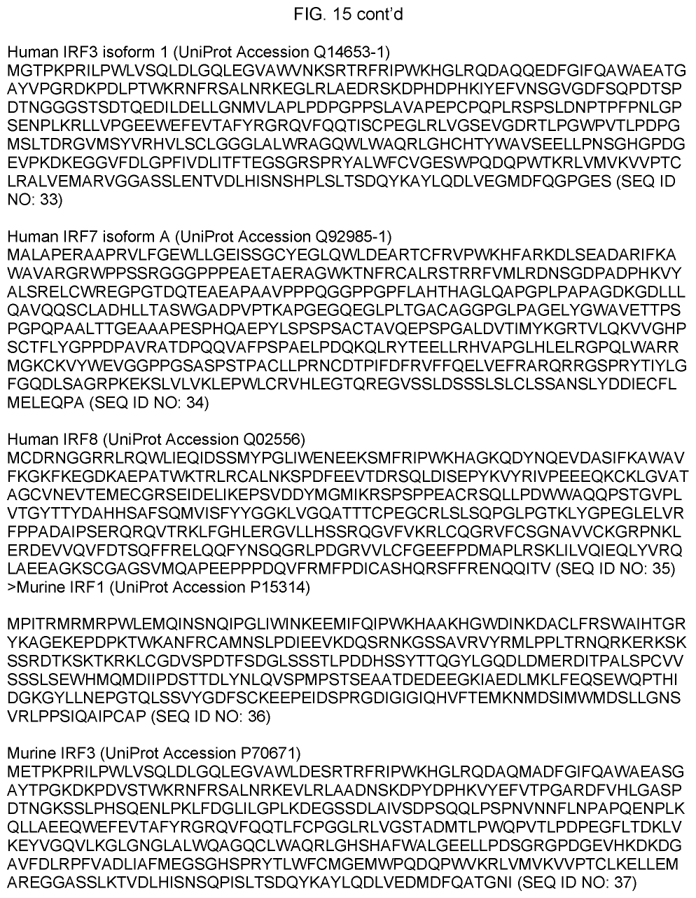

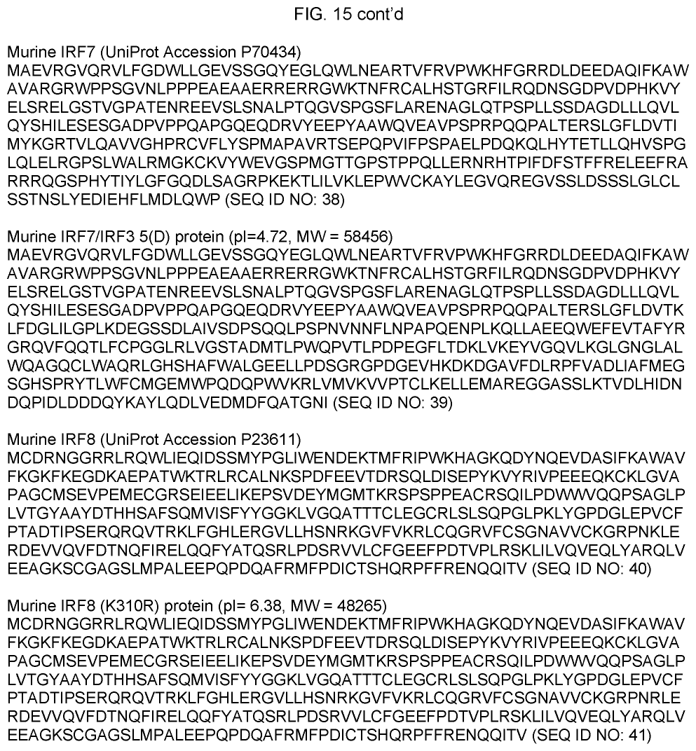

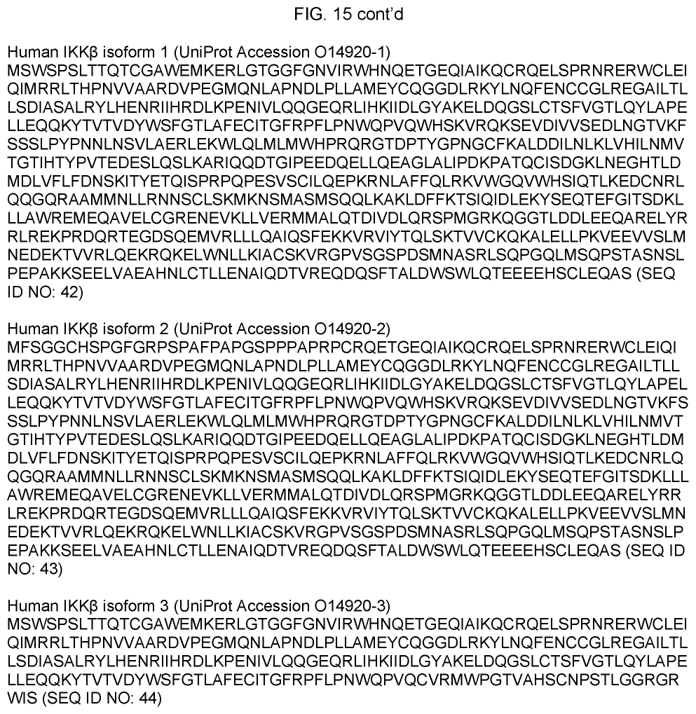

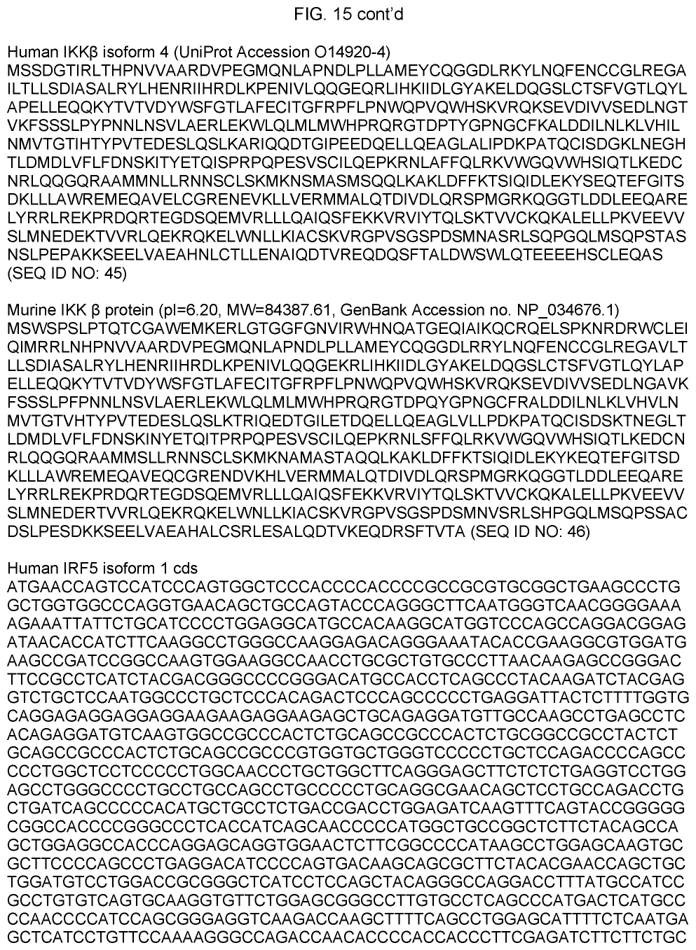

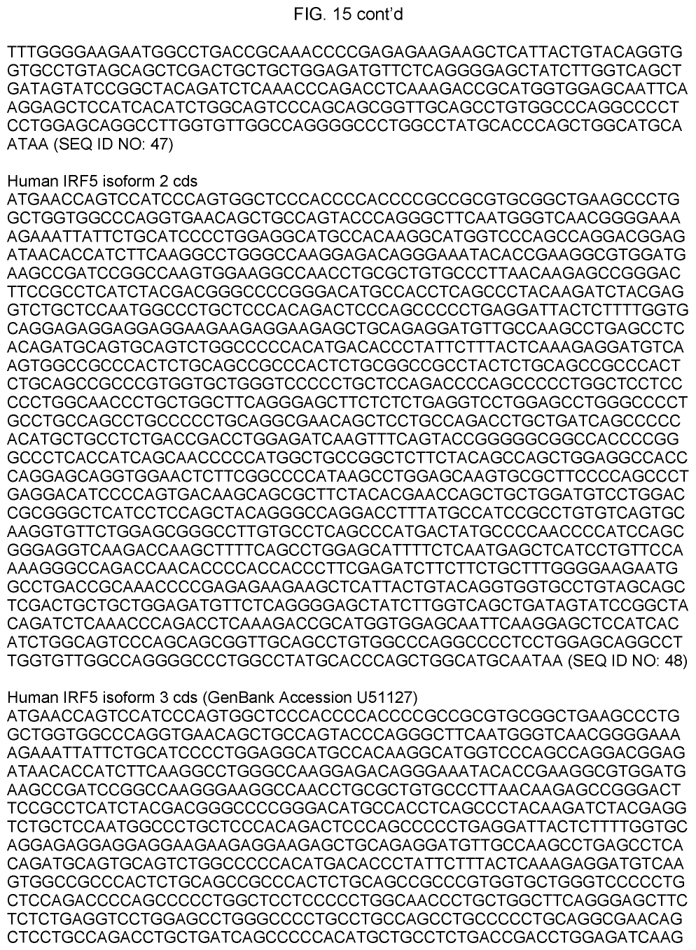

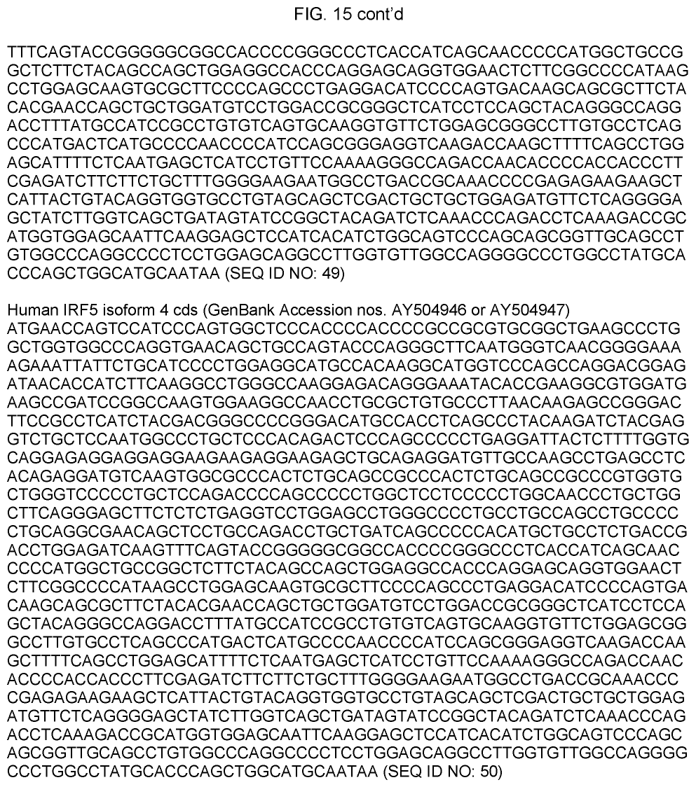

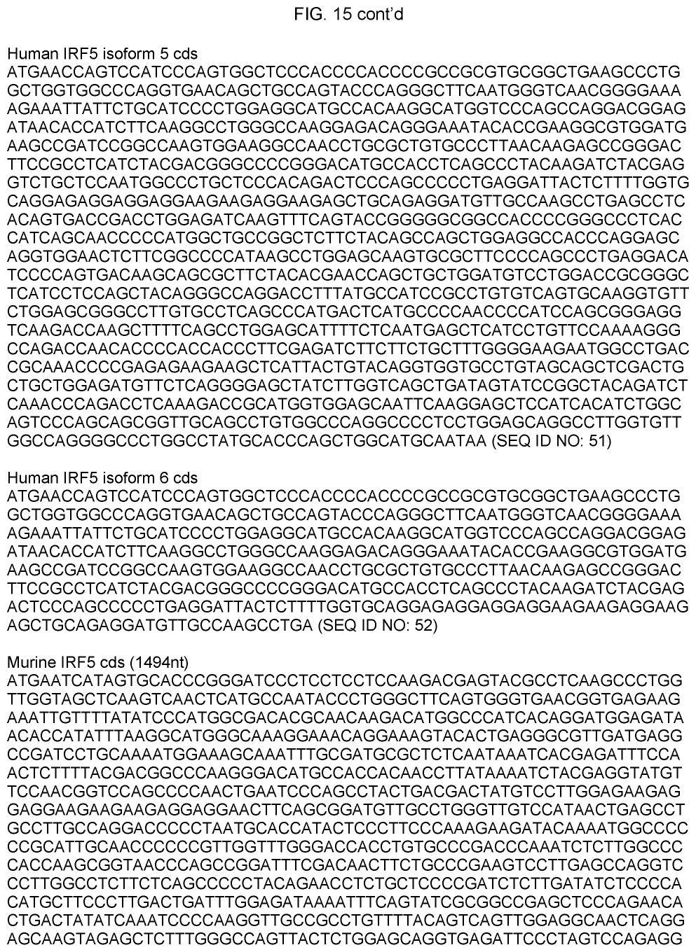

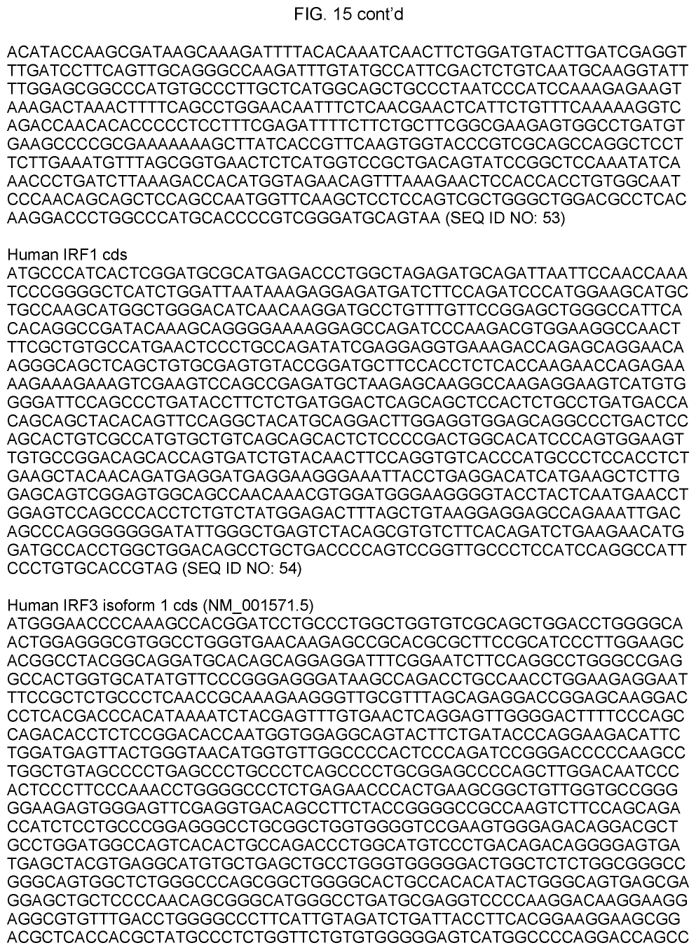

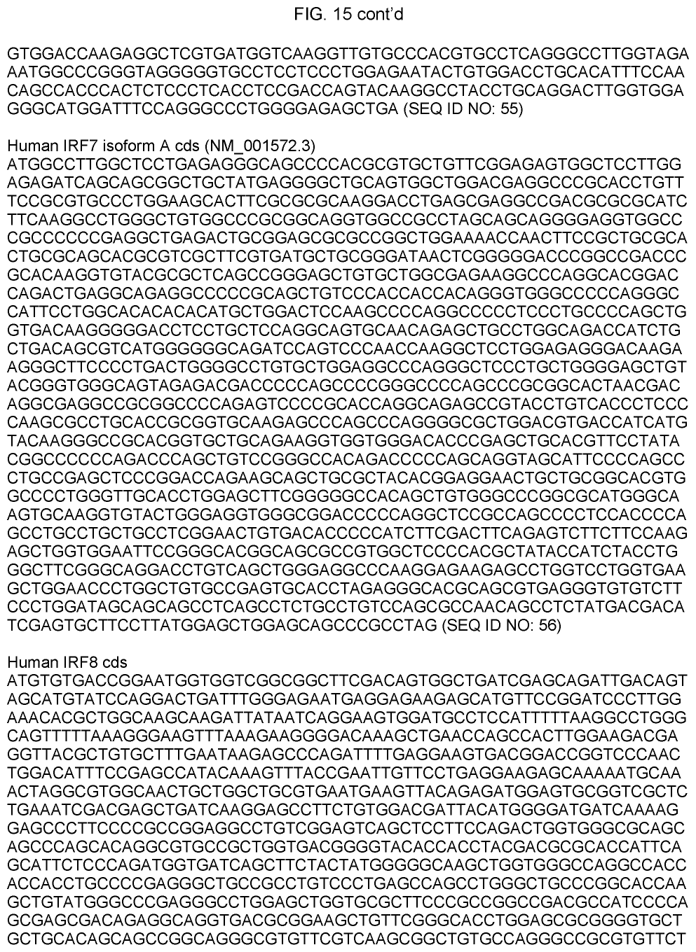

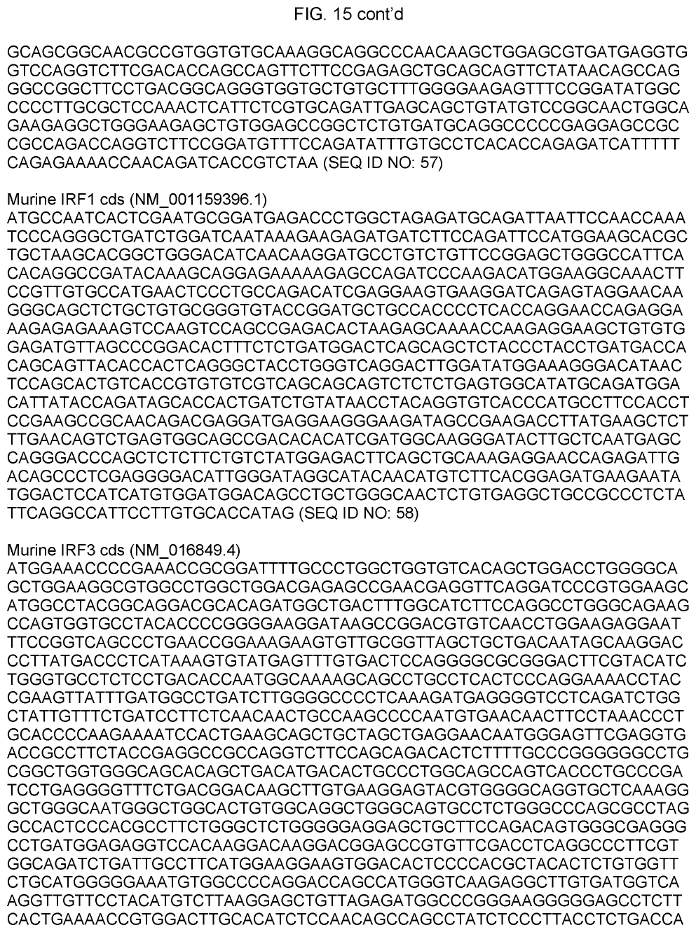

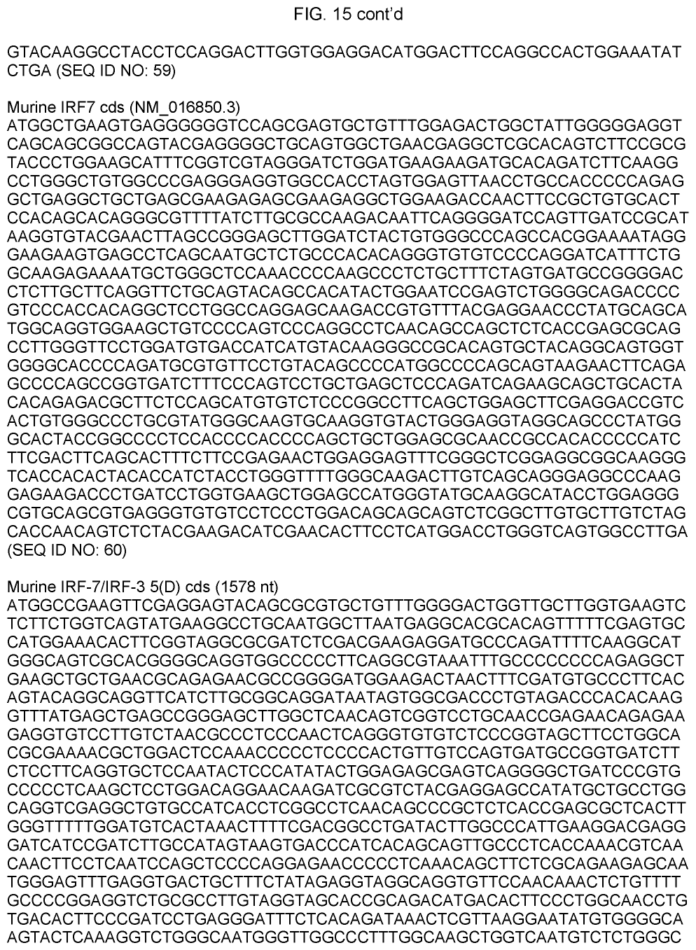

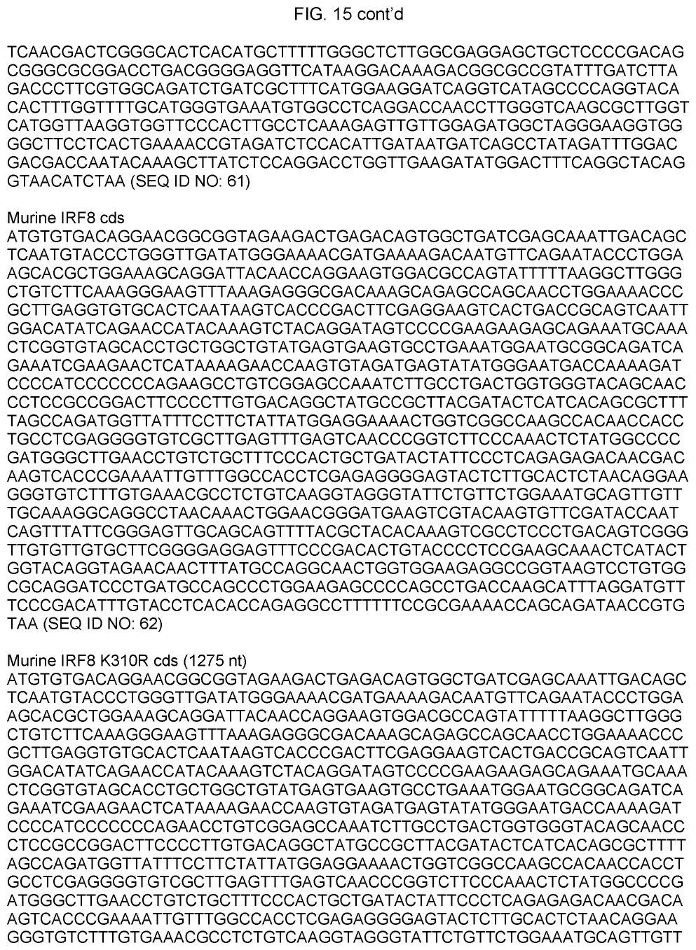

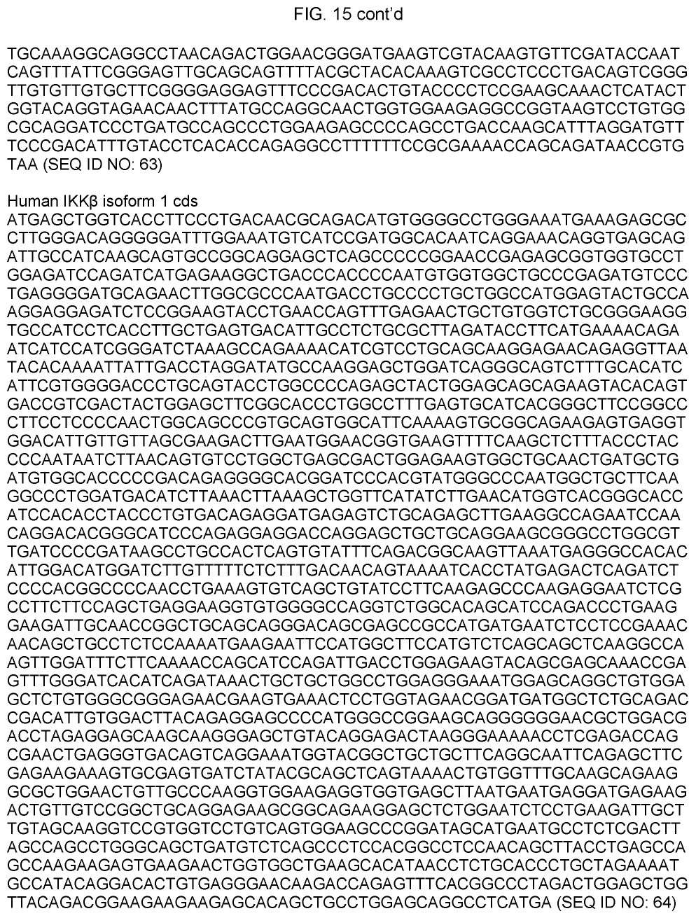

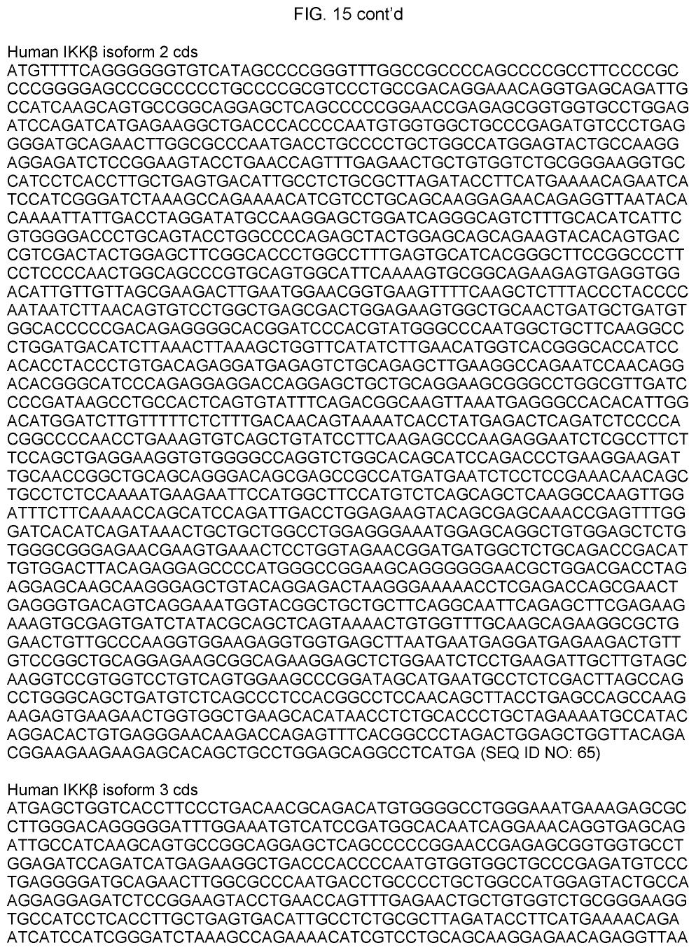

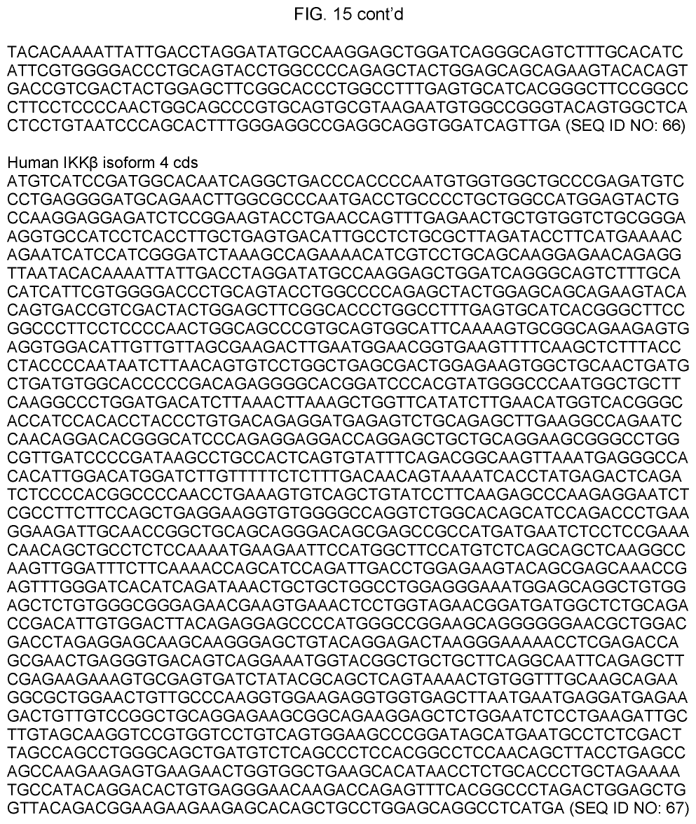

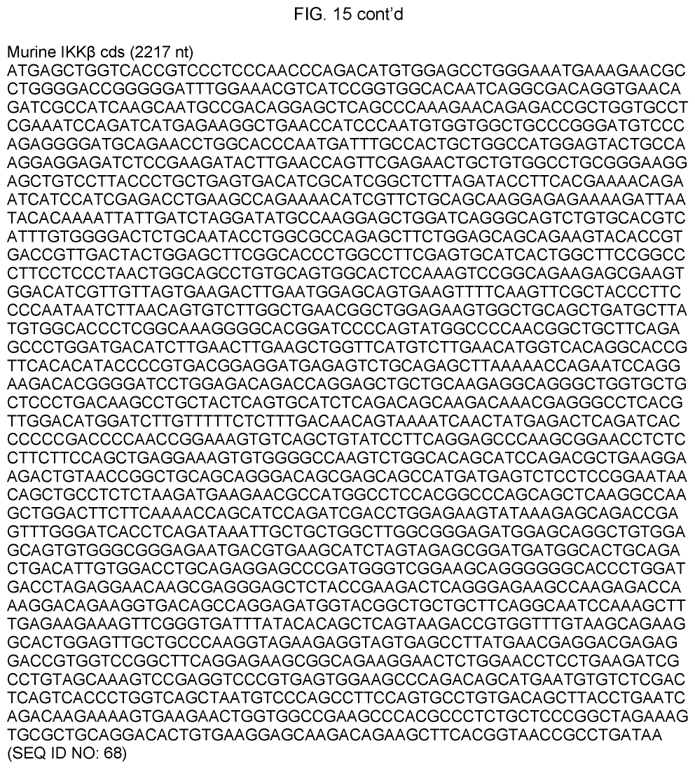

When the disclosed nanoparticles are added to a heterogeneous mixture of cells (e.g., an in vivo environment), the engineered nanoparticles bind to selected cell populations and stimulate receptor-mediated endocytosis; this process provides entry for the nucleic acid (e.g., synthetic mRNA) they carry, and consequently the selected cells begin to express the encoded molecule (FIGS. 1-3B). Because nuclear transport and transcription of the transgene is not required when mRNA is used rather than DNA, this process is, in some cases, rapid and efficient. If required, additional applications of the nanoparticles can be performed until the desired results are achieved. In particular embodiments, the nanoparticles are biodegradable and biocompatible.

In particular embodiments, rapid means that expression of an encoded nucleic acid begins within a selected cell type within 24 hours or within 12 hours of exposure of a heterogeneous sample of cells to nanoparticles disclosed herein. This timeline is possible utilizing nucleic acids such as mRNA which start being transcribed almost immediately (e.g., within minutes) of release into targeted cell cytoplasm.

In particular embodiments, efficient means that encapsulated nucleic acid transfer into targeted cells (e.g., primary human T cells) is >80% and phenotype modification occurs in at least 80% of these cells, at least 90% of these cells or 100% of these cells. In particular embodiments, efficient means that encapsulated nucleic acid transfer into targeted cells is >80% and phenotype modification occurs in at least 25% of these cells, at least 33% of these cells or at least 50% of these cells. In particular embodiments, phenotype modification can occur in 1/3 of selected cells that uptake nanoparticles wherein the delivered nucleic acid encodes a nuclease.

In particular embodiments, the nucleic acids include synthetic mRNA that expresses a therapeutic protein, such as a CAR, TCR, CAR/TCR hybrid or a macrophage activator. Particular embodiments utilize in vitro transcribed (IVT) mRNA (see, e.g., Grudzien-Nogalska et al., Methods Mol. Biol. 969:55-72, 2013), self-amplifying RNA (sa-RNA; Brito et al., Adv Genet. 89:179-233, 2015); or closed-ended DNA (ceDNA; Li et al., PLoS One. 2013 Aug. 1 (doi.org/10.1371/journal.pone.0069879) to transiently express, for example, a leukemia-specific 1928z CAR, a Hepatitis B virus (HBV) core antigen specific HBcore18-27 TCR, a prostate tumor specific anti-ROR1 4-1BBz CAR, or a macrophage activator.

Additional options and embodiments of the disclosure are now described in more detail as follows: (i) Expression of Therapeutic Proteins including (a) CAR, TCR, and CAR/TCR hybrids and (b) Macrophage Activators; (ii) Cell Attractants; (iii) Nanoparticles; (iv) Compositions; (v) Methods of Use; (vi) Kits; (vii) Exemplary Embodiments; and (viii) Experimental Examples.

(i) Expression of Therapeutic Proteins including (a) CAR, TCR, and CAR/TCR hybrids and (b) macrophage activators. In particular embodiments, expression is based on use of mRNA as a nucleic acid within a delivered nanoparticle.

In particular embodiments, nucleic acids include synthetic mRNA. In particular embodiments, synthetic mRNA is engineered for increased intracellular stability using 5′-capping. Multiple distinct 5′-cap structures can be used to generate the 5′-cap of a synthetic mRNA molecule. For example, the Anti-Reverse Cap Analog (ARCA) cap contains a 5′-5′-triphosphate guanine-guanine linkage where one guanine contains an N7 methyl group as well as a 3′-O-methyl group. Synthetic mRNA molecules may also be capped post-transcriptionally using enzymes responsible for generating 5′-cap structures. For example, recombinant Vaccinia Virus Capping Enzyme and recombinant 2′-O-methyltransferase enzyme can create a canonical 5′-5′-triphosphate linkage between the 5′-most nucleotide of an mRNA and a guanine nucleotide where the guanine contains an N7 methylation and the ultimate 5′-nucleotide contains a 2′-O-methyl generating the Cap1 structure. This results in a cap with higher translational-competency and cellular stability and reduced activation of cellular pro-inflammatory cytokines.

Synthetic mRNA or other nucleic acids may also be made cyclic. Synthetic mRNA may be cyclized, or concatemerized, to generate a translation competent molecule to assist interactions between poly-A binding proteins and 5′-end binding proteins. The mechanism of cyclization or concatemerization may occur through at least three different routes: 1) chemical, 2) enzymatic, and 3) ribozyme catalyzed. The newly formed 5′-/3′-linkage may be intramolecular or intermolecular.

In the first route, the 5′-end and the 3′-end of the nucleic acid may contain chemically reactive groups that, when close together, form a new covalent linkage between the 5′-end and the 3′-end of the molecule. The 5′-end may contain an NHS-ester reactive group and the 3′-end may contain a 3′-amino-terminated nucleotide such that in an organic solvent the 3′-amino-terminated nucleotide on the 3′-end of a synthetic mRNA molecule will undergo a nucleophilic attack on the 5′-NHS-ester moiety forming a new 5′-/3′-amide bond.

In the second route, T4 RNA ligase may be used to enzymatically link a 5′-phosphorylated nucleic acid molecule to the 3′-hydroxyl group of a nucleic acid forming a new phosphodiester linkage. In an example reaction, 1 μg of a nucleic acid molecule can be incubated at 37° C. for 1 hour with 1-10 units of T4 RNA ligase (New England Biolabs, Ipswich, Mass.) according to the manufacturer's protocol. The ligation reaction may occur in the presence of a split oligonucleotide capable of base-pairing with both the 5′- and 3′-region in juxtaposition to assist the enzymatic ligation reaction.

In the third route, either the 5′- or 3′-end of a cDNA template encodes a ligase ribozyme sequence such that during in vitro transcription, the resultant nucleic acid molecule can contain an active ribozyme sequence capable of ligating the 5′-end of a nucleic acid molecule to the 3′-end of a nucleic acid molecule. The ligase ribozyme may be derived from the Group I Intron, Group I Intron, Hepatitis Delta Virus, Hairpin ribozyme or may be selected by SELEX (systematic evolution of ligands by exponential enrichment). The ribozyme ligase reaction may take 1 to 24 hours at temperatures between 0 and 37° C.

These nucleic acid sequences include RNA sequences that are translated, in particular embodiments, into protein. The nucleic acid sequences include both the full-length nucleic acid sequences as well as non-full-length sequences derived from the full-length protein. The sequences can also include degenerate codons of the native sequence or sequences that may be introduced to provide codon preference in a specific selected cell type. Gene sequences to encode therapeutic protein are available in publicly available databases and publications. As used herein, the term “encoding” refers to a property of sequences of nucleic acids, such as a plasmid, a gene, cDNA, mRNA, to serve as templates for synthesis of therapeutic protein.

As indicated, nucleic acids are used to drive expression of therapeutic proteins by genetically modified cells, and in particular embodiments, the therapeutic proteins include CAR, TCR, CAR/TCR hybrid or macrophage activators.

(a) CAR, TCR, and CAR/TCR hybrids. CARs refer to synthetically designed receptors including at least a binding domain and an effector domain, and optionally a spacer domain and/or a transmembrane domain. In particular embodiments, a CAR refers to a recombinant polypeptide including an extracellular antigen binding domain in the form of a scFv, a transmembrane domain, and cytoplasmic signaling domains (also referred to herein as “an intracellular signaling domains”) including a functional signaling domain derived from a stimulatory molecule as defined below. In particular embodiments, a central intracellular signaling domain of a CAR is derived from the CD3 zeta chain that is normally found associated with the TCR complex. As described more fully below, the CD3 zeta signaling domain can be fused with one or more functional signaling domains derived from at least one co-stimulatory molecule such as 4-1BB (i.e., CD137), CD27 and/or CD28. Exemplary CARs and CAR architectures useful in the methods and compositions of the present disclosure include those provided by WO2012138475A1, U.S. Pat. No. 9,624,306B2, U.S. Pat. No. 9,266,960B2, US2017017477, EP2694549B1, US20170283504, US20170281766, US20170283500, US20180086846, US20100105136, US20100105136, WO2012079000, WO2008045437, WO2016139487A1, and WO2014039523, each of which is incorporated herein in its entirety.

TCR refer to naturally occurring T cell receptors. CAR/TCR hybrids refer to proteins having an element of a TCR and an element of a CAR. For example, a CAR/TCR hybrid could have a naturally occurring TCR binding domain with an effector domain that the TCR binding domain is not naturally associated with. A CAR/TCR hybrid could have a mutated TCR binding domain and an ITAM signaling domain. A CAR/TCR hybrid could have a naturally occurring TCR with an inserted non-naturally occurring spacer region or transmembrane domain.

Particular CAR/TCR hybrids include TRuC® (T Cell Receptor Fusion Construct) hybrids; TCR² Therapeutics, Cambridge, Mass.]. By way of example, the production of TCR fusion proteins is described in International Patent Publications WO 2018/026953 and WO 2018/067993, and in Application Publication US 2017/0166622, each of which is incorporated by reference herein in its entirety.

In particular embodiments, CAR/TCR hybrids include a “T-cell receptor (TCR) fusion protein” or “TFP”. A TFP includes a recombinant polypeptide derived from the various polypeptides including the TCR that is generally capable of i) binding to a surface antigen on target cells and ii) interacting with other polypeptide components of the intact TCR complex, typically when co-located in or on the surface of a T-cell.

In particular embodiments, a TFP includes an antibody fragment that binds a cancer antigen (e.g., CD19, ROR1) wherein the sequence of the antibody fragment is contiguous with and in the same reading frame as a nucleic acid sequence encoding a TCR subunit or portion thereof. The TFPs are able to associate with one or more endogenous (or alternatively, one or more exogenous, or a combination of endogenous and exogenous) TCR subunits in order to form a functional TCR complex.

Binding domains can particularly include any peptide that specifically binds a marker on a targeted cell. Sources of binding domains include antibody variable regions from various species (which can be in the form of antibodies, sFvs, scFvs, Fabs, scFv-based grababody, or soluble VH domain or domain antibodies). These antibodies can form antigen-binding regions using only a heavy chain variable region, i.e., these functional antibodies are homodimers of heavy chains only (referred to as “heavy chain antibodies”) (Jespers et al., Nat. Biotechnol. 22:1161, 2004; Cortez-Retamozo et al., Cancer Res. 64:2853, 2004; Baral et al., Nature Med. 12:580, 2006; and Barthelemy et al., J. Biol. Chem. 283:3639, 2008).

An alternative source of binding domains includes sequences that encode random peptide libraries or sequences that encode an engineered diversity of amino acids in loop regions of alternative non-antibody scaffolds, such as scTCR (see, e.g., Lake et al., Int. Immunol. 11:745, 1999; Maynard et al., J. Immunol. Methods 306:51, 2005; U.S. Pat. No. 8,361,794), fibrinogen domains (see, e.g., Weisel et al., Science 230:1388, 1985), Kunitz domains (see, e.g., U.S. Pat. No. 6,423,498), designed ankyrin repeat proteins (DARPins) (Binz et al., J. Mol. Biol. 332:489, 2003 and Binz et al., Nat. Biotechnol. 22:575, 2004), fibronectin binding domains (adnectins or monobodies) (Richards et al., J. Mol. Biol. 326:1475, 2003; Parker et al., Protein Eng. Des. Selec. 18:435, 2005 and Hackel et al. (2008) J. Mol. Biol. 381:1238-1252), cysteine-knot miniproteins (Vita et al. (1995) Proc. Nat'l. Acad. Sci. (USA) 92:6404-6408; Martin et al. (2002) Nat. Biotechnol. 21:71, 2002 and Huang et al. (2005) Structure 13:755, 2005), tetratricopeptide repeat domains (Main et al., Structure 11:497, 2003 and Cortajarena et al., ACS Chem. Biol. 3:161, 2008), leucine-rich repeat domains (Stumpp et al., J. Mol. Biol. 332:471, 2003), lipocalin domains (see, e.g., WO 2006/095164, Beste et al., Proc. Nat'l. Acad. Sci. (USA) 96:1898, 1999 and Schönfeld et al., Proc. Nat'l. Acad. Sci. (USA) 106:8198, 2009), V-like domains (see, e.g., US Patent Application Publication No. 2007/0065431), C-type lectin domains (Zelensky and Gready, FEBS J. 272:6179, 2005; Beavil et al., Proc. Nat'l. Acad. Sci. (USA) 89:753, 1992 and Sato et al., Proc. Nat'l. Acad. Sci. (USA) 100:7779, 2003), mAb² or Fcab (Fc antigen binding) (see, e.g., PCT Patent Application Publication Nos. WO 2007/098934; WO 2006/072620; Wozniak-Knopp et al., Prot. Eng. Des. Select. 23:4, 289-297, 2010), armadillo repeat proteins (see, e.g., Madhurantakam et al., Protein Sci. 21: 1015, 2012; PCT Patent Application Publication No. WO 2009/040338), affilin (Ebersbach et al., J. Mol. Biol. 372: 172, 2007), affibody, avimers, knottins, fynomers, atrimers, cytotoxic T-lymphocyte associated protein-4 (Weidle et al., Cancer Gen. Proteo. 10:155, 2013) or the like (Nord et al., Protein Eng. 8:601, 1995; Nord et al., Nat. Biotechnol. 15:772, 1997; Nord et al., Euro. J. Biochem. 268:4269, 2001; Binz et al., Nat. Biotechnol. 23:1257, 2005; Boersma and Plückthun, Curr. Opin. Biotechnol. 22:849, 2011).

In particular embodiments, a binding domain is a single chain TCR (scTCR) including V_{α/β} and C_{α/β} chains (e.g., V_{α}-C_{α}, V_{β}-C_{β}, V_{α}-V_{β} or including V_{α}-C_{α}, V_{β}-C_{β}, V_{α}-V_{β} pair specific for a target of interest (e.g., peptide-MHC complex).

In particular embodiments, engineered CAR, TCR, and hybrid CAR/TCR include a sequence that is at least 90%, at least 91%, at least 92%, at least 93%, at least 94%, at least 95%, at least 96%, at least 97%, at least 98%, at least 99%, at least 99.5%, or 100% identical to an amino acid sequence of a known or identified TCR Vα, Vβ, Cα, or Cβ, wherein each CDR includes zero changes or at most one, two, or three changes, from a TCR or fragment or derivative thereof that specifically binds to the target of interest.

In particular embodiments, engineered CAR, TCR, and hybrid CAR/TCR that can be transiently expressed from the nanoparticles include Vα, Vβ, Cα, or Cβ regions derived from or based on a Vα, Vβ, Cα, or Cβ of a known or identified TCR (e.g., a high-affinity TCR) and includes one or more (e.g., 2, 3, 4, 5, 6, 7, 8, 9, 10) insertions, one or more (e.g., 2, 3, 4, 5, 6, 7, 8, 9, 10) deletions, one or more (e.g., 2, 3, 4, 5, 6, 7, 8, 9, 10) amino acid substitutions (e.g., conservative amino acid substitutions or non-conservative amino acid substitutions), or a combination of the above-noted changes, when compared with the Vα, Vβ, Cα, or Cβ of a known or identified TCR. An insertion, deletion or substitution may be anywhere in a Vα, Vβ, Cα, or Cβ region, including at the amino- or carboxy-terminus or both ends of these regions, provided that each CDR includes zero changes or at most one, two, or three changes and provides a target binding domain containing a modified Vα, Vβ, Cα, or Cβ region can still specifically bind its target with an affinity and action similar to wild type.

In particular embodiments, a binding domain V_{H} region of the present disclosure can be derived from or based on a V_{H} of a known monoclonal antibody and can contain one or more (e.g., 2, 3, 4, 5, 6, 7, 8, 9, 10) insertions, one or more (e.g., 2, 3, 4, 5, 6, 7, 8, 9, 10) deletions, one or more (e.g., 2, 3, 4, 5, 6, 7, 8, 9, 10) amino acid substitutions (e.g., conservative amino acid substitutions or non-conservative amino acid substitutions), or a combination of the above-noted changes, when compared with the V_{H} of a known monoclonal antibody. An insertion, deletion or substitution may be anywhere in the V_{H} region, including at the amino- or carboxy-terminus or both ends of this region, provided that each CDR includes zero changes or at most one, two, or three changes and provided a binding domain containing the modified V_{H} region can still specifically bind its target with an affinity similar to the wild type binding domain.

In particular embodiments, a V_{L} region in a binding domain of the present disclosure is derived from or based on a V_{L} of a known monoclonal antibody and contains one or more (e.g., 2, 3, 4, 5, 6, 7, 8, 9, 10) insertions, one or more (e.g., 2, 3, 4, 5, 6, 7, 8, 9, 10) deletions, one or more (e.g., 2, 3, 4, 5, 6, 7, 8, 9, 10) amino acid substitutions (e.g., conservative amino acid substitutions), or a combination of the above-noted changes, when compared with the V_{L} of the known monoclonal antibody. An insertion, deletion or substitution may be anywhere in the V_{L} region, including at the amino- or carboxy-terminus or both ends of this region, provided that each CDR includes zero changes or at most one, two, or three changes and provided a binding domain containing the modified V_{L} region can still specifically bind its target with an affinity similar to the wild type binding domain.

In particular embodiments, a binding domain of a CAR, TCR, or hybrid CAR/TCR includes or is a sequence that is at least 90%, at least 91%, at least 92%, at least 93%, at least 94%, at least 95%, at least 96%, at least 97%, at least 98%, at least 99%, at least 99.5%, or 100% identical to an amino acid sequence of a light chain variable region (V_{L}) or to a heavy chain variable region (V_{H}), or both, wherein each CDR includes zero changes or at most one, two, or three changes, from a monoclonal antibody or fragment or derivative thereof that specifically binds to target of interest.

In particular embodiments, the binding domain can bind PSMA. A number of antibodies specific for PSMA are known to those of skill in the art and can be readily characterized for sequence, epitope binding, and affinity. In particular embodiments, the binding domain can include anti-Mesothelin ligands (associated with treating ovarian cancer, pancreatic cancer, and mesothelioma); anti-WT-1 (associated with treating leukemia and ovarian cancer); anti-HIV-gag (associated with treating HIV infections); or anti-cytomegalovirus (associated with treating CMV diseases such as herpes virus).

In particular embodiments, the binding domain can bind CD19. In particular embodiments, a binding domain is a single chain Fv fragment (scFv) that includes VH and VL regions specific for CD19. In particular embodiments, the V_{H} and V_{L} regions are human. Exemplary V_{H} and V_{L} regions include the segments of anti-CD19 specific monoclonal antibody FMC63. In particular embodiments, the scFv is a human or humanized scFv including a variable light chain including a CDRL1 sequence of RASQDISKYLN, CDRL2 sequence of SRLHSGV, and a CDRL3 sequence of GNTLPYTFG. In particular embodiments, the scFv is a human or humanized ScFv including a variable heavy chain including CDRHI sequence of DYGVS, CDRH2 sequence of VTWGSETTYYNSALKS), and a CDRH3 sequence of YAMDYWG. Other CD19-targeting antibodies such as SJ25C1 and HD37 are known. (SJ25C1: Bejcek et al. Cancer Res 2005, PMID 7538901; HD37: Pezutto et al. JI 1987, PMID 2437199).

In particular embodiments, an scFV sequence that binds human CD19 includes:

(SEQ ID NO: 103)
MALPVTALLLPLALLLHAEVKLQQSGAELVRPGSSVKISCKASGYAFSSY
WMNWVKQRPGQGLEWIGQIYPGDGDTNYNGKFKGQATLTADKSSSTAYMQ
LSGLTSEDSAVYFCARKTISSVVDFYFDYWGQGTTVTVSSGGGGSGGGGS
GGGGSDIELTQSPKFMSTSVGDRVSVTCKASQNVGTNVAWYQQKPGQSPK
PLIYSATYRNSGVPDRFTGSGSGTDFTLTITNVQSKDLADYFCQQYNRYP
YTSGGGTKLEIKRAAAIEVMYPPPYLDNEKSNGTIIHVKGKHLCPSPLFP
GPSKPFW.

In particular embodiments, the binding domain can bind ROR1. In particular embodiments, the scFv is a human or humanized scFv including a variable light chain including a CDRL1 sequence of ASGFDFSAYYM (SEQ ID NO: 104), CDRL2 sequence of TIYPSSG (SEQ ID NO: 105), and a CDRL3 sequence of ADRATYFCA (SEQ ID NO: 106). In particular embodiments, the scFv is a human or humanized scFv including a variable heavy chain including CDRH1 sequence of DTIDWY (SEQ ID NO: 107), CDRH2 sequence of VQSDGSYTKRPGVPDR (SEQ ID NO: 108), and a CDRH3 sequence of YIGGYVFG (SEQ ID NO: 109). A number of antibodies specific for ROR1 are known to those of skill in the art and can be readily characterized for sequence, epitope binding, and affinity.

In particular embodiments, an scFV sequence that binds human ROR1 includes:

(SEQ ID NO: 110)
MLLLVTSLLLCELPHPAFLLIPQEQLVESGGRLVTPGGSLTLSCKASGFD
FSAYYMSWVRQAPGKGLEWIATIYPSSGKTYYATWVNGRFTISSDNAQNT
VDLQMNSLTAADRATYFCARDSYADDGALFNIWGPGTLVTISSGGGGSGG
GGSGGGGSELVLTQSPSVSAALGSPAKITCTLSSAHKTDTIDWYQQLQGE
APRYLMQVQSDGSYTKRPGVPDRFSGSSSGADRYLIIPSVQADDEADYYC
GADYIGGYVFGGGTQLTVTGESKYGPPCPPCPMFWVLVVVGGVLACYSLL
V.

Additional description regarding the production of nanoparticles can be found in PCT/US2018/012507, which is herein incorporated by reference in its entirety.

The particular following cancers can be targeted by including within an extracellular component of a CAR, TCR, and hybrid CAR/TCR a binding domain that binds the associated cellular marker(s) (e.g. a CAR including an scFV specific to any one of the following markers):

| Targeted Cancer | Cellular Marker(s) |
|---|---|
| Prostate Cancer | PSMA, WT1, Prostate Stem |
| | Cell antigen (PSCA), |
| | SV40 T |
| Breast Cancer | HER2, ERBB2, ROR1 |
| Stem Cell Cancer | CD133 |
| Ovarian Cancer | L1-CAM, extracellular domain of MUC16 |
| | (MUC-CD), folate binding protein |
| | (folate receptor), |
| | Lewis Y, ROR1, mesothelin, WT-1 |
| Mesothelioma | mesothelin |
| Renal Cell Carcinoma | carboxy-anhydrase-IX (CAIX); |
| Melanoma | GD2 |
| Pancreatic Cancer | mesothelin, CEA, CD24, ROR1 |
| Lung Cancer | ROR1 |
| HBV-induced | HBV antigens, such as HBV core antigen |
| hepatocellular carcinoma | |
| Multiple Myeloma | BCMA, GPRC5D, CD38, CS-1 |

In particular embodiments, synthetic mRNA (e.g., IVT mRNA) encodes a CAR, TCR, or CAR/TCR hybrid that specifically binds a cellular marker or a fragment thereof.

Without limiting the foregoing, cellular markers also include A33; BAGE; Bcl-2; β-catenin; BCMA; B7H4; BTLA; CA125; CA19-9; CD3, CD5; CD19; CD20; CD21; CD22; CD25; CD28; CD30; CD33; CD37; CD38; CD40; CD52; CD44v6; CD45; CD56; CD79b; CD80; CD81; CD86; CD123; CD134; CD137; CD151; CD171; CD276; CEA; CEACAM6; c-Met; CS-1; CTLA-4; cyclin B1; DAGE; EBNA; EGFR; EGFRvIII, ephrinB2; ErbB2; ErbB3; ErbB4; EphA2; estrogen receptor; FAP; ferritin; α-fetoprotein (AFP); FLT1; FLT4; folate-binding protein; Frizzled; GAGE; G250; GD-2; GHRHR; GHR; GITR; GM2; GPRC5D; gp75; gp100 (Pmel 17); gp130; HLA; HER-2/neu; HPV E6; HPV E7; hTERT; HVEM; IGF1R; IL6R; KDR; Ki-67; Lewis A; Lewis Y; LIFRβ; LRP; LRP5; LTβR; MAGE; MART; mesothelin; MUC; MUC1; MUM-1-B; myc; NYESO-1; O-acetyl GD-2; O-acetyl GD3; OSMRβ; p53; PD1; PD-L1; PD-L2; PRAME; progesterone receptor; PSA; PSMA; PTCH1; RANK; ras; Robo1; RORI; survivin; TCRα; TCRβ; tenascin; TGFBR1; TGFBR2; TLR7; TLR9; TNFR1; TNFR2; TNFRSF4; TWEAK-R; TSTA tyrosinase; VEGF; and WT1.

Particular cancer cell cellular markers include:

| Marker | Sequence |
|---|---|
| PSMA | MWNLLHETDSAVATARRPRWLCAGALVLAGGFFLLGFLFGWFIKSSNEATNI |
| | TPKHNMKAFLDELKAENIKKFLYNFTQIPHLAGTEQNFQLAKQIQSQWKEFGL |
| | DSVELAHYDVLLSYPNKTHPNYISIINEDGNEIFNTSLFEPPPPGYENVSDIVP |
| | PFSAFSPQGMPEGDLVYVNYARTEDFFKLERDMKINCSGKIVIARYGKVFRG |
| | NKVKNAQLAGAKGVILYSDPADYFAPGVKSYPDGWNLPGGGVQRGNILNLN |
| | GAGDPLTPGYPANEYAYRRGIAEAVGLPSIPVHPIGYYDAQKLLEKMGGSAP |
| | PDSSWRGSLKVPYNVGPGFTGNFSTQKVKMHIHSTNEVTRIYNVIGTLRGAV |
| | EPDRYVILGGHRDSWVFGGIDPQSGAAVVHEIVRSFGTLKKEGWRPRRTILF |
| | ASWDAEEFGLLGSTEWAEENSRLLQERGVAYINADSSIEGNYTLRVDCTPL |
| | MYSLVHNLTKELKSPDEGFEGKSLYESWTKKSPSPEFSGMPRISKLGSGND |
| | FEVFFQRLGIASGRARYTKNWETNKFSGYPLYHSVYETYELVEKFYDPMFKY |
| | HLTVAQVRGGMVFELANSIVLPFDCRDYAVVLRKYADKIYSISMKHPQEMKT |
| | YSVSFDSLFSAVKNFTEIASKFSERLQDFDKSNPIVLRMMNDQLMFLERAFID |
| | PLGLPDRPFYRHVIYAPSSHNKYAGESFPGIYDALFDIESKVDPSKAWGEVK |
| | RQIYVAAFTVQAAAETLSEVA (SEQ ID NO: 111) |
| PSCA | MKAVLLALLMAGLALQPGTALLCYSCKAQVSNEDCLQVENCTQLGEQCWTA |
| | RIRAVGLLTVISKGCSLNCVDDSQDYYVGKKNITCCDTDLCNASGAHALQPA |
| | AAILALLPALGLLLWGPGQL (SEQ ID NO: 112) |
| Mesothelin | MALPTARPLLGSCGTPALGSLLFLLFSLGWVQPSRTLAGETGQEAAPLDGVL |
| | ANPPNISSLSPRQLLGFPCAEVSGLSTERVRELAVALAQKNVKLSTEQLRCL |
| | AHRLSEPPEDLDALPLDLLLFLNPDAFSGPQACTHFFSRITKANVDLLPRGAP |
| | ERQRLLPAALACWGVRGSLLSEADVRALGGLACDLPGRFVAESAEVLLPRL |
| | VSCPGPLDQDQQEAARAALQGGGPPYGPPSTWSVSTMDALRGLLPVLGQP |
| | IIRSIPQGIVAAWRQRSSRDPSWRQPERTILRPRFRREVEKTACPSGKKAREI |
| | DESLIFYKKWELEACVDAALLATQMDRVNAIPFTYEQLDVLKHKLDELYPQG |
| | YPESVIQHLGYLFLKMSPEDIRKWNVTSLETLKALLEVNKGHEMSPQVATLID |
| | RFVKGRGQLDKDTLDTLTAFYPGYLCSLSPEELSSVPPSSIWAVRPQDLDTC |
| | DPRQLDVLYPKARLAFQNMNGSEYFVKIQSFLGGAPTEDLKALSQQNVSMD |
| | LATFMKLRTDAVLPLTVAEVQKLLGPHVEGLKAEERHRPVRDWILRQRQDDL |
| | DTLGLGLQGGIPNGYLVLDLSVQEALSGTPCLLGPGPVLTVLALLLASTLA |
| | (SEQ ID NO: 113) |
| CD19 | MPPPRLLFFLLFLTPMEVRPEEPLVVKVEEGDNAVLQCLKGTSDGPTQQLT |
| | WSRESPLKPFLKLSLGLPGLGIHMRPLASWLFIFNVSQQMGGFYLCQPGPP |
| | SEKAWQPGWTVNVEGSGELFRWNVSDLGGLGCGLKNRSSEGPSSPSGKL |
| | MSPKLYVWAKDRPEIWEGEPPCVPPRDSLNQSLSQDLTMAPGSTLWLSCG |
| | VPPDSVSRGPLSWTHVHPKGPKSLLSLELKDDRPARDMWVMETGLLLPRAT |
| | AQDAGKYYCHRGNLTMSFHLEITARPVLWHWLLRTGGWKVSAVTLAYLIFCL |
| | CSLVGILHLQRALVLRRKRKRMTDPTRRFFKVTPPPGSGPQNQYGNVLSLPT |
| | PTSGLGRAQRWAAGLGGTAPSYGNPSSDVQADGALGSRSPPGVGPEEEE |
| | GEGYEEPDSEEDSEFYENDSNLGQDQLSQDGSGYENPEDEPLGPEDEDSF |
| | SNAESYENEDEELTQPVARTMDFLSPHGSAWDPSREATSLGSQSYEDMRGI |
| | LYAAPQLRSIRGQPGPNHEEDADSYENMDNPDGPDPAWGGGGRMGTWST |
| | R (SEQ ID NO: 114) |
| CD20 | MTTPRNSVNGTFPAEPMKGPIAMQSGPKPLFRRMSSLVGPTQSFFMRESKT |
| | LGAVQIMNGLFHIALGGLLMIPAGIYAPICVTVWYPLWGGIMYIISGSLLAATEK |
| | NSRKCLVKGKMIMNSLSLFAAISGMILSIMDILNIKISHFLKMESLNFIRAHTPYI |
| | NIYNCEPANPSEKNSPSTQYCYSIQSLFLGILSVMLIFAFFQELVIAGIVENEW |
| | KRTCSRPKSNIVLLSAEEKKEQTIEIKEEVVGLTETSSQPKNEEDIEIIPIQEEE |
| | EEETETNFPEPPQDQESSPIENDSSP (SEQ ID NO: 115) |
| ROR1 | MHRPRRRGTRPPLLALLAALLLAARGAAAQETELSVSAELVPTSSWNISSEL |
| | NKDSYLTLDEPMNNITTSLGQTAELHCKVSGNPPPTIRWFKNDAPVVQEPRR |
| | LSFRSTIYGSRLRIRNLDTTDTGYFQCVATNGKEVVSSTGVLFVKFGPPPTAS |
| | PGYSDEYEEDGFCQPYRGIACARFIGNRTVYMESLHMQGEIENQITAAFTMI |
| | GTSSHLSDKCSQFAIPSLCHYAFPYCDETSSVPKPRDLCRDECEILENVLCQ |
| | TEYIFARSNPMILMRLKLPNCEDLPQPESPEAANCIRIGIPMADPINKNHKCYN |
| | STGVDYRGTVSVTKSGRQCQPWNSQYPHTHTFTALRFPELNGGHSYCRNP |
| | GNQKEAPWCFTLDENFKSDLCDIPACDSKDSKEKNKMEILYILVPSVAIPLAIA |
| | LLFFFICVCRNNQKSSSAPVQRQPKHVRGQNVEMSMLNAYKPKSKAKELPL |
| | SAVRFMEELGECAFGKIYKGHLYLPGMDHAQLVAIKTLKDYNNPQQVVTEFQ |
| | QEASLMAELHHPNIVCLLGAVTQEQPVCMLFEYINQGDLHEFLIMRSPHSDV |
| | GCSSDEDGTVKSSLDHGDFLHIAIQIAAGMEYLSSHFFVHKDLAARNILIGEQL |
| | HVKISDLGLSREIYSADYYRVQSKSLLPIRWMPPEAIMYGKFSSDSDIWSFGV |
| | VLWEIFSFGLQPYYGFSNQEVIEMVRKRQLLPCSEDCPPRMYSLMTECWNE |
| | IPSRRPRFKDIHVRLRSWEGLSSHTSSTTPSGGNATTQTTSLSASPVSNLSN |
| | PRYPNYMFPSQGITPQGQIAGFIGPPIPQNQRFIPINGYPIPPGYAAFPAAHY |
| | QPTGPPRVIQHCPPPKSRSPSSASGSTSTGHVTSLPSSGSNQEANIPLLPHM |
| | SIPNHPGGMGITVFGNKSQKPYKIDSKQASLLGDANIHGHTESMISAEL (SEQ |
| | ID NO: 116) |
| WT1 | MGHHHHHHHHHHSSGHIEGRHMRRVPGVAPTLVRSASETSEKRPFMCAYP |
| | GCNKRYFKLSHLQMHSRKHTGEKPYQCDFKDCERRFFRSDQLKRHQRRHT |
| | GVKPFQCKTCQRKFSRSDHLKTHTRTHTGEKPFSCRWPSCQKKFARSDEL |
| | VRHHNMHQRNMTKLQLAL (SEQ ID NO: 117) |
| CD33 | DPNFWLQVQESVTVQEGLCVLVPCTFFHPIPYYDKNSPVHGYWFREGAIISR |
| | DSPVATNKLDQEVQEETQGRFRLLGDPSRNNCSLSIVDARRRDNGSYFFRM |
| | ERGSTKYSYKSPQLSVHVTDLTHRPKILIPGTLEPGHSKNLTCSVSWACEQG |
| | TPPIFSWLSAAPTSLGPRTTHSSVLIITPRPQDHGTNLTCQVKFAGAGVTTER |
| | TIQLNVTYVPQNPTTGIFPGDGSGKQETRAGVVHGAIGGAGVTALLALCLCLI |
| | FFIVKTHRRKAARTAVGRNDTHPTTGSASPKHQKKSKLHGPTETSSCSGAA |
| | PTVEMDEELHYASLNFHGMNPSKDTSTEYSEVRTQ (SEQ ID NO: 118) |
| BCMA | MLQMAGQCSQNEYFDSLLHACIPCQLRCSSNTPPLTCQRYCNASVTNSVKG |
| | TNAILWTCLGLSLIISLAVFVLMFLLRKINSEPLKDEFKNTGSGLLGMANIDLEK |
| | SRTGDEIILPRGLEYTVEECTCEDCIKSKPKVDSDHCFPLPAMEEGATILVTTK |
| | TNDYCKSLPAALSATEIEKSISAR |
| GPRC5D | MYKDCIESTGDYFLLCDAEGPWGIILESLAILGIVVTILLLLAFLFLMRKIQDCS |
| | QWNVLPTQLLFLLSVLGLFGLAFAFIIELNQQTAPVRYFLFGVLFALCFSCLLA |
| | HASNLVKLVRGCVSFSWTTILCIAIGCSLLQIIIATEYVTLIMTRGMMFVNMTPC |
| | QLNVDFVVLLVYVLFLMALTFFVSKATFCGPCENWKQHGRLIFITVLFSIIIWV |
| | VWISMLLRGNPQFQRQPQWDDPVVCIALVTNAWVFLLLYIVPELCILYRSCR |
| | QECPLQGNACPVTAYQHSFQVENQELSRARDSDGAEEDVALTSYGTPIQPQ |
| | TVDPTQECFIPQAKLSPQQDAGGV |
| CD38 | MANCEFSPVSGDKPCCRLSRRAQLCLGVSILVLILVVVLAVVVPRWRQQWS |
| | GPGTTKRFPETVLARCVKYTEIHPEMRHVDCQSVWDAFKGAFISKHPCNITE |
| | EDYQPLMKLGTQTVPCNKILLWSRIKDLAHQFTQVQRDMFTLEDTLLGYLAD |
| | DLTWCGEFNTSKINYQSCPDWRKDCSNNPVSVFWKTVSRRFAEAACDVVH |
| | VMLNGSRSKIFDKNSTFGSVEVHNLQPEKVQTLEAWVIHGGREDSRDLCQD |
| | PTIKELESIISKRNIQFSCKNIYRPDKFLQCVKNPEDSSCTSEI |
| CS-1 | MAGSPTCLTLIYILWQLTGSAASGPVKELVGSVGGAVTFPLKSKVKQVDSIV |
| (SLAMF7) | WTFNTTPLVTIQPEGGTIIVTQNRNRERVDFPDGGYSLKLSKLKKNDSGIYYV |
| | GIYSSSLQQPSTQEYVLHVYEHLSKPKVTMGLQSNKNGTCVTNLTCCMEHG |
| | EEDVIYTWKALGQAANESHNGSILPISWRWGESDMTFICVARNPVSRNFSSP |
| | ILARKLCEGAADDPDSSMVLLCLLLVPLLLSLFVLGLFLWFLKRERQEEYIEEK |
| | KRVDICRETPNICPHSGENTEYDTIPHTNRTILKEDPANTVYSTVEIPKKMENP |
| | HSLLTMPDTPRLFAYENVI |

The present disclosure provides methods for treating, preventing or alleviating a symptom of cancer or a precancerous condition. The method includes administering to a subject in need thereof, a therapeutically effective amount of a nanocarrier of the present disclosure, or a pharmaceutically composition thereof. Exemplary cancers that may be treated include prostate cancer, breast cancer, stem cell cancer, ovarian cancer, mesothelioma, renal cell carcinoma melanoma, pancreatic cancer, lung cancer, HBV-induced hepatocellular carcinoma, and multiple myeloma. Further exemplary cancers that may be treated include medulloblastoma, oligodendroglioma, ovarian clear cell adenocarcinoma, ovarian endomethrioid adenocarcinoma, ovarian serous adenocarcinoma, pancreatic ductal adenocarcinoma, pancreatic endocrine tumor, malignant rhabdoid tumor, astrocytoma, atypical teratoid rhabdoid tumor, choroid plexus carcinoma, choroid plexus papilloma, ependymoma, glioblastoma, meningioma, neuroglial tumor, oligoastrocytoma, oligodendroglioma, pineoblastoma, carcinosarcoma, chordoma, extragonadal germ cell tumor, extrarenal rhabdoid tumor, schwannoma, skin squamous cell carcinoma, chondrosarcoma, clear cell sarcoma of soft tissue, ewing sarcoma, gastrointestinal stromal tumor, osteosarcoma, rhabdomyosarcoma, epitheloid sarcoma, renal medullo carcinoma, diffuse large B-cell lymphoma, follicular lymphoma and not otherwise specified (NOS) sarcoma.

The present disclosure further provides the use of a nanocarrier of the present disclosure, or a pharmaceutically composition thereof in the treatment of cancer or precancer, or, for the preparation of a medicament useful for the treatment of such cancer or pre-cancer. Exemplary cancers that may be treated include prostate cancer, breast cancer, stem cell cancer, ovarian cancer, mesothelioma, renal cell carcinoma melanoma, pancreatic cancer, lung cancer, HBV-induced hepatocellular carcinoma, and multiple myeloma. Further exemplary cancers that may be treated include medulloblastoma, oligodendroglioma, ovarian clear cell adenocarcinoma, ovarian endomethrioid adenocarcinoma, ovarian serous adenocarcinoma, pancreatic ductal adenocarcinoma, pancreatic endocrine tumor, malignant rhabdoid tumor, astrocytoma, atypical teratoid rhabdoid tumor, choroid plexus carcinoma, choroid plexus papilloma, ependymoma, glioblastoma, meningioma, neuroglial tumor, oligoastrocytoma, oligodendroglioma, pineoblastoma, carcinosarcoma, chordoma, extragonadal germ cell tumor, extrarenal rhabdoid tumor, schwannoma, skin squamous cell carcinoma, chondrosarcoma, clear cell sarcoma of soft tissue, ewing sarcoma, gastrointestinal stromal tumor, osteosarcoma, rhabdomyosarcoma, epitheloid sarcoma, renal medullo carcinoma, diffuse large B-cell lymphoma, follicular lymphoma and NOS sarcoma.

In any methods disclosed herein, cancer is selected from the group consisting of brain and central nervous system (CNS) cancer, head and neck cancer, kidney cancer, ovarian cancer, pancreatic cancer, leukemia, lung cancer, lymphoma, multiple myeloma, sarcoma, breast cancer, and prostate cancer. In some embodiments, the cancer is selected from the group consisting of medulloblastoma, oligodendroglioma, ovarian clear cell adenocarcinoma, ovarian endomethrioid adenocarcinoma, ovarian serous adenocarcinoma, pancreatic ductal adenocarcinoma, pancreatic endocrine tumor, malignant rhabdoid tumor, astrocytoma, atypical teratoid rhabdoid tumor, choroid plexus carcinoma, choroid plexus papilloma, ependymoma, glioblastoma, meningioma, neuroglial tumor, oligoastrocytoma, oligodendroglioma, pineoblastoma, carcinosarcoma, chordoma, extragonadal germ cell tumor, extrarenal rhabdoid tumor, schwannoma, skin squamous cell carcinoma, chondrosarcoma, clear cell sarcoma of soft tissue, ewing sarcoma, gastrointestinal stromal tumor, osteosarcoma, rhabdomyosarcoma, epitheloid sarcoma, renal medullo carcinoma, diffuse large B-cell lymphoma, follicular lymphoma and NOS sarcoma.

Also contemplated are binding domains specific for infectious disease agents, for instance by binding to an infectious agent antigen. These include for instance viral antigens or other viral markers, for instance which are expressed by virally-infected cells. Exemplary viruses include adenoviruses, arenaviruses, bunyaviruses, coronavirusess, flavirviruses, hantaviruses, hepadnaviruses, herpesviruses, papilomaviruses, paramyxoviruses, parvoviruses, picornaviruses, poxviruses, orthomyxoviruses, retroviruses, reoviruses, rhabdoviruses, rotaviruses, spongiform viruses or togaviruses. In additional embodiments, viral antigen markers include peptides expressed by CMV, cold viruses, Epstein-Barr, flu viruses, hepatitis A, B, and C viruses, herpes simplex, HIV, influenza, Japanese encephalitis, measles, polio, rabies, respiratory syncytial, rubella, smallpox, varicella zoster or West Nile virus.

As further particular examples, cytomegaloviral antigens include envelope glycoprotein B and CMV pp65; Epstein-Barr antigens include EBV EBNAI, EBV P18, and EBV P23; hepatitis antigens include the S, M, and L proteins of HBV, the pre-S antigen of HBV, HBCAG DELTA, HBV HBE, hepatitis C viral RNA, HCV NS3 and HCV NS4; herpes simplex viral antigens include immediate early proteins and glycoprotein D; HIV antigens include gene products of the gag, pol, and env genes such as HIV gp32, HIV gp41, HIV gp120, HIV gp160, HIV P17/24, HIV P24, HIV P55 GAG, HIV P66 POL, HIV TAT, HIV GP36, the Nef protein and reverse transcriptase; influenza antigens include hemagglutinin and neuraminidase; Japanese encephalitis viral antigens include proteins E, M-E, M-E-NS1, NS1, NS1-NS2A and 80% E; measles antigens include the measles virus fusion protein; rabies antigens include rabies glycoprotein and rabies nucleoprotein; respiratory syncytial viral antigens include the RSV fusion protein and the M2 protein; rotaviral antigens include VP7sc; rubella antigens include proteins E1 and E2; and varicella zoster viral antigens include gpI and gpII.

Additional particular exemplary viral antigen sequences include:

| Source | Sequence |
|---|---|
| Nef (66-97): | VGFPVTPQVPLRPMTYKAAVDLSHFLKEKGGL |
| | (SEQ ID NO: 119) |
| Nef (116-145) | HTQGYFPDWQNYTPGPGVRYPLTFGWLYKL |
| | (SEQ ID NO: 120) |
| Gag p17 | EKIRLRPGGKKKYKLKHIV (SEQ ID |
| (17-35) | NO: 121) |
| Gag p17-p24 | NPPIPVGEIYKRWIILGLNKIVRMYSPTSILD |
| (253-284) | (SEQ ID NO: 122) |
| Pol 325-355 | AIFQSSMTKILEPFRKQNPDIVIYQYMDDLY |
| (RT 158-188) | (SEQ ID NO: 123) |

See Fundamental Virology, Second Edition, eds. Fields, B. N. and Knipe, D. M. (Raven Press, New York, 1991) for additional examples of viral antigens.

In particular embodiments disclosed herein, modified immune system cells recognize and destroy virally-infected cells. Alternatively, or in addition, modified monocytes/macrophages can remove viruses from peripheral tissue or the blood stream (extracellular) before cellular infection by a viral particle. B cells can be modified to transiently express broadly neutralizing antibodies. In one example, B cells can be modified to transiently express broadly neutralizing anti-HIV antibodies.

In particular embodiments, the targeting agent targets HIV gag protein, gp120 or the Hepatitis B envelope protein (S domain).

In particular embodiments, markers are expressed by cells associated with bacterial infections. Exemplary bacteria include anthrax; gram-negative bacilli, chlamydia, diphtheria, haemophilus influenza, Helicobacter pylori, malaria, Mycobacterium tuberculosis, pertussis toxin, pneumococcus, rickettsiae, staphylococcus, streptococcus and tetanus.

As particular examples of bacterial antigen markers, anthrax antigens include anthrax protective antigen; gram-negative bacilli antigens include lipopolysaccharides; haemophilus influenza antigens include capsular polysaccharides; diphtheria antigens include diphtheria toxin; Mycobacterium tuberculosis antigens include mycolic acid, heat shock protein 65 (HSP65), the 30 kDa major secreted protein and antigen 85A; pertussis toxin antigens include hemagglutinin, pertactin, FIM2, FIM3 and adenylate cyclase; pneumococcal antigens include pneumolysin and pneumococcal capsular polysaccharides; rickettsiae antigens include rompA; streptococcal antigens include M proteins; and tetanus antigens include tetanus toxin.

Monocytes/macrophages are particularly useful to modify when the therapeutic objective is treatment of a bacterial infection. In one particular embodiment, monocytes/macrophages can be modified with a ligand recognizing the surface component lipoteichoic acid of Staphyloccus aureus or the Staphylococcus aureus clumping factor A (ClfA).

In particular embodiments, immune cells are modified to target multi-drug resistant “superbugs”. Examples of superbugs include Enterococcus faecium, Clostridium difficile, Acinetobacter baumannii, Pseudomonas aeruginosa, and Enterobacteriaceae (including Escherichia coli, Klebsiella pneumoniae, Enterobacter spp.).

In particular embodiments, markers are expressed by cells associated with fungal infections. Exemplary fungi include candida, coccidiodes, cryptococcus, histoplasma, leishmania, plasmodium, protozoa, parasites, schistosomae, tinea, toxoplasma, and Trypanosoma cruzi.

As further particular examples of fungal antigens, coccidiodes antigens include spherule antigens; cryptococcal antigens include capsular polysaccharides; histoplasma antigens include heat shock protein 60 (HSP60); leishmanial antigens include gp63 and lipophosphoglycan; Plasmodium falciparum antigens include merozoite surface antigens, sporozoite surface antigens, circumsporozoite antigens, gametocyte/gamete surface antigens, protozoal and other parasitic antigens including the blood-stage antigen pf 155/RESA; schistosomae antigens include glutathione-S-transferase and paramyosin; tinea fungal antigens include trichophytin; toxoplasma antigens include SAG-1 and p30; and Trypanosoma cruzi antigens include the 75-77 kDa antigen and the 56 kDa antigen.

Monocytes/macrophages are particularly useful to modify when the therapeutic objective is treatment of a fungal infection.

In particular embodiments, markers are expressed by cells associated with autoimmune or allergic conditions. Exemplary autoimmune conditions include acute necrotizing hemorrhagic encephalopathy, allergic asthma, alopecia areata, anemia, aphthous ulcer, arthritis (including rheumatoid arthritis, juvenile rheumatoid arthritis, osteoarthritis, psoriatic arthritis), asthma, autoimmune thyroiditis, conjunctivitis, Crohn's disease, cutaneous lupus erythematosus, dermatitis (including atopic dermatitis and eczematous dermatitis), diabetes, diabetes mellitus, erythema nodosum leprosum, keratoconjunctivitis, multiple sclerosis, myasthenia gravis, psoriasis, scleroderma, Sjogren's syndrome, including keratoconjunctivitis sicca secondary to Sjogren's syndrome, Stevens-Johnson syndrome, systemic lupus erythematosis, ulcerative colitis, vaginitis and Wegener's granulomatosis.

Examples of autoimmune antigens include glutamic acid decarboxylase 65 (GAD 65), native DNA, myelin basic protein, myelin proteolipid protein, acetylcholine receptor components, thyroglobulin, and the thyroid stimulating hormone (TSH) receptor. Examples of allergic antigens include pollen antigens such as Japanese cedar pollen antigens, ragweed pollen antigens, rye grass pollen antigens, animal derived antigens (such as dust mite antigens and feline antigens), histocompatibility antigens, and penicillin and other therapeutic drugs.

Effector Domains. Effector domains are capable of transmitting functional signals to a cell. In particular embodiments, an effector domain will directly or indirectly promote a cellular response by associating with one or more other proteins that directly promote a cellular response. Effector domains can provide for activation of at least one function of a transduced lymphocyte expressing the CAR, TCR, or CAR/TCR hybrid upon binding to the marker expressed on a targeted cell. Activation of the lymphocyte can include one or more of proliferation, differentiation, activation or other effector functions. In particular embodiments, the delivered polynucleotide encodes for the effector domain.

An effector domain may include one, two, three or more receptor signaling domains, intracellular signaling domains, costimulatory domains, or combinations thereof. Any intracellular effector domain, costimulatory domain or both from any of a variety of signaling molecules (e.g., signal transduction receptors) may be used in the CARs, TCRs, or CAR/TCR hybrids of this disclosure.

Exemplary effector domains include those from 4-1BB, CD3ε, CD3δ, CD3ζ, CD27, CD28, CD79A, CD79B, CARD11, DAP10, FcRα, FcRβ, FcRγ, Fyn, HVEM, ICOS, Lck, LAG3, LAT, LRP, NOTCH1, Wnt, NKG2D, OX40, ROR2, Ryk, SLAMF1, Slp76, pTα, TCRα, TCRβ, TRIM, Zap70, PTCH2, or any combination thereof.

T cell activation can be said to be mediated by two distinct classes of cytoplasmic signaling sequence: those that initiate antigen-dependent primary activation and provide a TCR-like signal (primary cytoplasmic signaling sequences) and those that act in an antigen-independent manner to provide a secondary or co-stimulatory signal (secondary cytoplasmic signaling sequences). Primary cytoplasmic signaling sequences that act in a stimulatory manner may contain signaling motifs which are known as receptor tyrosine-based activation motifs or iTAMs. Examples of iTAM containing primary cytoplasmic signaling sequences include those derived from CD3 zeta, FeR gamma, CD3 gamma, CD3 delta, CD3 epsilon, CD5, CD22, CD79a, CD79b, and CD66d.

In particular embodiments, an effector domain includes a cytoplasmic portion that associates with a cytoplasmic signaling protein, wherein the cytoplasmic signaling protein is a lymphocyte receptor or signaling domain thereof, a protein including a plurality of ITAMs, a costimulatory factor, or any combination thereof.

Examples of intracellular signaling domains include the cytoplasmic sequences of the CD3 zeta chain, and/or co-receptors that act in concert to initiate signal transduction following CAR engagement, as well as any derivative or variant of these sequences and any synthetic sequence that has the same functional capability. In particular embodiments, an intracellular signaling domain of a CAR can be designed to include an intracellular signaling domain combined with any other desired cytoplasmic domain(s). For example, the intracellular signaling domain of a CAR can include an intracellular signaling domain and a costimulatory signaling region. The costimulatory signaling region refers to a portion of the CAR including the intracellular domain of a costimulatory molecule. A costimulatory molecule is a cell surface molecule other than the expressed marker ligand that is required for a response of lymphocytes to a marker. Examples of such molecules include CD27, CD28, 4-1BB (CD 137), OX40, CD30, CD40, lymphocyte function-associated antigen-1 (LFA-1), CD2, CD7, LIGHT, NKG2C, B7-H3, and a ligand that specifically binds with CD83.

Spacer Regions. Spacer regions can be customized for individual markers on targets to optimize target recognition. In particular embodiments, a spacer length can be selected based upon the location of a marker epitope, affinity of an antibody for the epitope, and/or the ability of the lymphocytes expressing the CAR, TCR, or CAR/TCR hybrid to proliferate in vitro and/or in vivo in response to marker recognition.

Typically, a spacer region is found between the binding domain and a transmembrane domain of the CAR, TCR, or CAR/TCR hybrid. Spacer regions can provide for flexibility of the binding domain and allows for high expression levels in the modified cells. In particular embodiments, a spacer region can have at least 10 to 250 amino acids, at least 10 to 200 amino acids, at least 10 to 150 amino acids, at least 10 to 100 amino acids, at least 10 to 50 amino acids or at least 10 to 25 amino acids and including any integer between the endpoints of any of the listed ranges. particular embodiments, a spacer region has 250 amino acids or less; 200 amino acids or less, 150 amino acids or less; 100 amino acids or less; 50 amino acids or less; 40 amino acids or less; 30 amino acids or less; 20 amino acids or less; or 10 amino acids or less.

In particular embodiments, spacer regions can be derived from a hinge region of an immunoglobulin like molecule, for example all or a portion of the hinge region from a human IgG1, human IgG2, a human IgG3, or a human IgG4. Hinge regions can be modified to avoid undesirable structural interactions such as dimerization. In particular embodiments, all or a portion of a hinge region can be combined with one or more domains of a constant region of an immunoglobulin. For example, a portion of a hinge region can be combined with all or a portion of a CH2 or CH3 domain or variant thereof.

Transmembrane Domains. CARs, TCRs, or CAR/TCR hybrids disclosed herein can also include transmembrane domains. In particular embodiments, the CAR, TCR, or CAR/TCR hybrid polynucleotide administered within the nanoparticle encodes the transmembrane domain. The transmembrane domain provides for anchoring of the CAR, TCR, or CAR/TCR hybrid in the lymphocyte membrane. The transmembrane domain may be derived either from a natural or a synthetic source. When the source is natural, the domain may be derived from any membrane-bound or transmembrane protein. Transmembrane regions include at least the transmembrane region(s) of) the alpha, beta, or zeta chain of the T-cell receptor, CD28, CD3, CD45, CD4, CDS, CD9, CDI6, CD22; CD33, CD37, CD64, CD80, CD86, CDI34, CDI37, and CD154. In particular embodiments, synthetic or variant transmembrane domains include predominantly hydrophobic residues such as leucine and valine.

Different potential CAR, TCR, or hybrid CAR/TCR nucleic acids that encode different ligand binding domains, different spacer region lengths, different intracellular binding domains and/or different transmembrane domains, can be tested in vivo (in an animal model) and/or in vitro to identify CAR, TCR, or hybrid CAR/TCR with improved function over other CAR, TCR, or hybrid CAR/TCR.

Exemplary CAR. In particular embodiments, the CAR includes a P28z fusion receptor composed of a single-chain antibody (scFv) specific for the extracellular domain of PSMA (J591) combined with CD28 and CD3 cytoplasmic signaling domains. In particular embodiments, the CAR includes a P28z CAR. Particular examples of P28z CAR described herein includes murine components. Amino acid positions 1-797 include the anti-PSMA scFv (J592) whereas positions 797-1477 include the murine CD8 transmembrane domain, murine CD28 signaling domain and the murine CD3zeta signaling domain. Any P28z domain can be individually replaced with optimized domains. In particular embodiments, the transmembrane domain and signaling domains within positions 797-1477 of P28z CAR described herein can be particularly replaced with domains optimized for use in humans or other animals. In particular embodiments, any whole or portion of a binding domain, any whole or portion of an effector domain, any whole or portion of a spacer domain and/or any whole or portion of a transmembrane domain can be optimized for use in humans or other animals. In particular embodiments, the P28z CAR is optimized for use in humans. When optimized for humans, the P28z CAR can have lowered immunogenicity in humans and have a lower number of non-immunogenic epitopes compared to non-human antibodies.

In particular embodiments, ROR1-specific and CD19-specific CARs can be constructed using VL and VH chain segments of the 2A2, R12, and R11 mAbs (ROR1) and FMC63 mAb (CD19). Variable region sequences for R11 and R12 are provided in Yang et al., Plos One 6(6):e21018, Jun. 15, 2011. Each scFV can be linked by a (Gly₄Ser)₃ protein to a spacer domain derived from IgG4-Fc (UniProt Database: P01861) including either ‘Hinge-CH2-CH3’ (229 AA), ‘Hinge-CH3’ (119 AA) or ‘Hinge’ only (12 AA) sequences. All spacers can contain a S→P substitution within the ‘Hinge’ domain located at position 108 of the native IgG4-Fc protein, and can be linked to the 27 AA transmembrane domain of human CD28 (for an exemplary full-length CD28 see UniProt: P10747) and to an effector domain signaling module including either (i) the 41 AA cytoplasmic domain of human CD28 with an LL→GG substitution located at positions 186-187 of the native CD28 protein or (ii) the 42 AA cytoplasmic domain of human 4-1BB (UniProt: Q07011), each of which can be linked to the 112 AA cytoplasmic domain of isoform 3 of human CD3ζ (UniProt: P20963). The construct encodes a T2A ribosomal skip element and a tEGFR sequence downstream of the chimeric receptor. tEGFR can be replaced or supplemented with a tag cassette binding a sequence, such as STREP TAG® II (IBA Gmbh Ltd., Goettingen, DE), Myc tag, V5 tag, FLAG® tag (Sigma-Aldrich Corp., St. Louis, Mo.), His tag, or other peptides or molecules as disclosed herein. Codon-optimized gene sequences encoding each transgene can be synthesized (Life Technologies) and cloned into the epHIV7 lentiviral vector using NheI and Not1 restriction sites. The epHIV7 lentiviral vector can be derived from the pHIV7 vector by replacing the cytomegalovirus promoter of pHIV7 with an EF-1 promoter. Anti-ROR1 chimeric receptor, anti-CD19 chimeric receptor, tEGFR, or tag cassette-encoding lentiviruses can be produced in 293T cells using the packaging vectors pCHGP-2, pCMV-Rev2 and pCMV-G, and CALPHOS™ transfection reagent (Takara Clontech).

HER2-specific chimeric receptors can be constructed using VL and VH chain segments of a HER2-specific mAb that recognizes a membrane proximal epitope on HER2, and the scFVs can be linked to IgG4 hinge/CH2/CH3, IgG4 hinge/CH3, and IgG4 hinge only extracellular spacer domains and to the CD28 transmembrane domain, 4-1BB and CD3 signaling domains.

An anti-CD19 chimeric receptor can include a single chain variable fragment (scFV) corresponding to the sequence of the CD19-specific mAb FMC63 (scFv: VL-VH), a spacer derived from IgG4-Fc including either the ‘Hinge-CH2-CH3’ domain (229 AA, long spacer) or the ‘Hinge’ domain only (12 AA, short spacer), and a signaling module of CD3 with membrane proximal CD28 or 4-1BB costimulatory domains, either alone or in tandem.

(b) Macrophage Activators. “Macrophage activation” refers to the process of altering the phenotype or function of a macrophage from (i) an inactivated state to an activated state; (ii) a non-activated state to an activated state; (iii) an activated state to a more activated state; or (iv) an inactivated state to a non-activated state. An inactivated state means an immunosuppressed phenotype that facilitates tumor growth and metastasis. A non-activated state means that the macrophage neither facilitates tumor growth or metastasis nor promotes the killing of tumor cells. Activated means that the macrophage exhibits tumoricidal activity. In particular embodiments, the activated state results in an M1 phenotype as described more fully below. In particular embodiments, the inactivated state results in an M2 phenotype. “Macrophage inactivation” refers to the process of altering the phenotype or function of a macrophage from (i) an activated state to a less activated state; (ii) an activated state to an non-activated state; (iii) an activated state to a an inactivated state; or (iv) a non-activated state to an inactivated state. In particular embodiments, the inactivated state is M2. In particular embodiments, the activated state is M1.

Administration of a macrophage stimulating nanoparticle composition can alter the immunosuppressive state in a tumor, which renders the tumor more susceptible to companion treatment with a herein described nanoparticle and the therapeutic protein(s) encoded thereby.

Polarization of a macrophage towards an activated or inactivated phenotype results from macrophage interaction with a number of different molecules or environments. For example, M1 macrophage polarization is triggered by stimuli including Toll-like receptor (TLR) ligands (e.g. lipopolysaccharide (LPS), muramyl dipeptide, lipoteichoic acid, imiquimod, CpG), IFNγ, TNFα, and macrophage colony-stimulating factor (GM-CSF). M2 polarized macrophages can be divided into subsets, depending on the stimuli that initiates the polarization: the M2a subtype is elicited by IL-4, IL-13 or fungal and helminth infections; M2b is elicited by IL-1 receptor ligands, immune complexes and LPS; M2c is elicited by IL-10, TGF-β and glucocorticoids; and M2d is elicited by IL-6 and adenosine. M2 macrophage polarization may also be triggered by IL-21, GM-CSF, complement components, and apoptotic cells. Macrophage polarization is also modulated by local microenvironmental conditions such as hypoxia.

The aforementioned molecules and environments affect macrophage polarization by triggering different intracellular signaling pathways involving transcription factors. Transcription factors that are involved in both M1 and M2 polarization include IRFs, signal transducers and activators of transcription (STAT), SOCS3 proteins, and nuclear factor kappa-light-chain-enhancer of activated B cells (NFκB). Mitogen-activated protein kinases (MAPK) also play a role in directing macrophage function towards either the M1 or M2 phenotype.

The IRF/STAT pathways, activated by such stimuli as IFNs and TLR signaling as discussed above, polarize macrophages to the M1 activation state via STAT1. On the other hand, such stimuli as IL-4 and IL-13 skew macrophages toward the M2 activation state via STAT6 (Sica A & Bronte V (2007) J Clin Invest 117: 1155-1166). These signaling events thus result in either the promotion of an inflammatory immune response and tumoricidal activity, as in the case of M1 macrophage polarization, or in the promotion of an immunosuppressive protumor response, as in the case of M2 macrophage polarization.

Some intracellular molecules implicated in the induction of an M1 phenotype include the G-protein coupled receptor, P2Y(2)R, which plays a role in inducing NO via NOS2 (Eun S Y et al. (2014) Int Immunopharmacol 18: 270-276); SOCS3, which activates NFκB/PI-3 kinase pathways to produce NO (Arnold C E et al. (2014) Immunology 141: 96-110); and growth and differentiation factor Activin A, which promotes M1 markers and down-regulates IL-10 (Sierra-Filardi E et al. (2011) Blood 117: 5092-5101).

Other intracellular molecules involved in induction of the M1 phenotype include IRFs. IRFs are a group of transcription factors with diverse roles, including virus-mediated activation of IFN, and modulation of cell growth, differentiation, apoptosis, and immune system activity. Members of the IRF family are characterized by a conserved N-terminal DNA-binding domain containing tryptophan (W) repeats.

IRF5 is a transcription factor that possesses a helix-turn-helix DNA-binding motif and mediates virus- and IFN-induced signaling pathways. It acts as a molecular switch that controls whether macrophages will promote or inhibit inflammation. IRF5 activates type I IFN genes, inflammatory cytokines, including TNF, IL-6, IL-12 and IL-23, and tumor suppressors as well as Th1 and Th17 responses. It is encoded by the human IRF5 gene located at chromosome 7q32 (OMIM ID 607218). It is appreciated that several isoforms/transcriptional variants of IRF5 exist. In particular embodiments, isoforms of human IRF5 include isoform 1 (UniProt Accession Q13568-1, SEQ ID NO: 25), isoform 2 (UniProt Accession Q13568-2, SEQ ID NO: 26), isoform 3 (UniProt Accession Q13568-3, SEQ ID NO: 27), isoform 4 (UniProt Accession Q13568-4, SEQ ID NO: 28), isoform 5 (UniProt Accession Q13568-5, SEQ ID NO: 29) and isoform 6 (UniProt Accession Q13568-6, SEQ ID NO: 30). In particular embodiments, isoforms of human IRF5 include isoform 1 encoded by a nucleotide sequence shown in SEQ ID NO: 47, isoform 2 encoded by a nucleotide sequence shown in SEQ ID NO: 48, isoform 3 encoded by a nucleotide sequence shown in SEQ ID NO: 49, isoform 4 encoded by a nucleotide sequence shown in SEQ ID NO: 50, isoform 5 encoded by a nucleotide sequence shown in SEQ ID NO: 51 and isoform 6 encoded by a nucleotide sequence shown in SEQ ID NO: 52. In particular embodiments, murine IRF5 includes an amino acid sequence shown in SEQ ID NO: 31. In particular embodiments, murine IRF5 is encoded by a nucleotide sequence shown in SEQ ID NO: 53. M1 macrophages have been shown to upregulate IRF5.

IRF1 and IRF8 also play critical roles in the development and function of myeloid cells, including activation of macrophages by proinflammatory signals such as IFN-γ. Dror N et al. (2007) Mol Immunol. 44(4):338-346. In particular embodiments, human IRF1 includes an amino acid sequence shown in SEQ ID NO: 32. In particular embodiments, human IRF1 is encoded by a nucleotide sequence shown in SEQ ID NO: 54. In particular embodiments, murine IRF1 includes an amino acid sequence shown in SEQ ID NO: 36. In particular embodiments, murine IRF1 is encoded by a nucleotide sequence shown in SEQ ID NO: 58. In particular embodiments, human IRF8 includes an amino acid sequence shown in SEQ ID NO: 35. In particular embodiments, human IRF8 is encoded by a nucleotide sequence shown in SEQ ID NO: 57. In particular embodiments, murine IRF8 includes an amino acid sequence shown in SEQ ID NO: 40. In particular embodiments, murine IRF8 is encoded by a nucleotide sequence shown in SEQ ID NO:

IRF3 is a homolog of IRF1 and IRF2. It contains several functional domains including a NES, a DBD, a C-terminal IRF association domain and several regulatory phosphorylation sites. IRF3 is found in an inactive cytoplasmic form that upon serine/threonine phosphorylation forms a complex with CREB Binding Protein, a transcriptional coactivator. This complex translocates to the nucleus and activates the transcription of IFN-α and -β, as well as other interferon-induced genes. In particular embodiments, isoforms of human IRF3 include isoform 1 (UniProt Accession Q14653-1), isoform 2 (UniProt Accession Q14653-2), isoform 3 (UniProt Accession Q14653-3), isoform 4 (UniProt Accession Q14653-4), and isoform 5 (UniProt Accession Q14653-5). In particular embodiments, human IRF3 isoform 1 includes an amino acid sequence shown in SEQ ID NO: 33. In particular embodiments, human IRF3 isoform 1 is encoded by a nucleotide sequence shown in SEQ ID NO: 55. In particular embodiments, murine IRF3 includes an amino acid sequence shown in SEQ ID NO: 37. In particular embodiments, murine IRF3 is encoded by a nucleotide sequence shown in SEQ ID NO: 59.

IRF7 has been shown to play a role in the transcriptional activation of type I IFN genes. In particular embodiments, isoforms of human IRF7 include isoform A (UniProt Accession Q92985-1), isoform B (UniProt Accession Q92985-2), isoform C (UniProt Accession Q92985-3), and isoform D (UniProt Accession Q92985-4). In particular embodiments, human IRF7 isoform A includes an amino acid sequence shown in SEQ ID NO: 34. In particular embodiments, human IRF7 isoform A is encoded by a nucleotide sequence shown in SEQ ID NO: 56. In particular embodiments, murine IRF7 includes an amino acid sequence shown in SEQ ID NO: 38. In particular embodiments, murine IRF7 is encoded by a nucleotide sequence shown in SEQ ID NO: 60.

One or more IRF mutants that contribute to IRF activation may also be used. For example: phosphomimetic mutants of human variant 3/variant 4 of IRF5 (isoform 4, SEQ ID NO: 28) that substitute amino acid residues S425, S427, S430, S436 with residues mimicking phosphorylation, such as aspartic acid residues (Chen W et al. (2008) Nat Struct Mol Biol. 15(11): 1213-1220); phosphomimetic mutants of human variant 5 of IRF5 (isoform 2, SEQ ID NO: 26) that substitute amino acid residues T10, S158, S309, S317, S451, and/or S462 with residues mimicking phosphorylation, such as aspartic acid residues (Chang Foreman H-C et al. infra); mutation of human IRF5 isoform a (variant 1, isoform 3, SEQ ID NO: 27) and isoform b (variant 2, isoform 1, SEQ ID NO: 25) residues S156, S158 and T160 to residues mimicking phosphorylation, such as aspartic acid residues, for constitutive nuclear accumulation of IRF5 (Lin R et al. (2005) J Biol Chem 280(4): 3088-3095); and IRF3 phosphomimetic mutants that substitute amino acid residue S396 of IRF3 with residues mimicking phosphorylation, such as aspartic acid (Chen W et al. infra). In particular embodiments, a fusion protein of murine IRF7/IRF3 includes Asp (D) mutations at four serine and one threonine residues in the IRF3 association domains (SEQ ID NO: 39), conferring constitutive activation and translocation of the fusion protein (Lin R et al. (1998) supra; Lin et al. (2000) Molecular and Cellular Biology 20: 6342-6353). In particular embodiments, a fusion protein of murine IRF7/IRF3 including D mutations at four serine and one threonine residues in the IRF3 association domains is encoded by a nucleotide sequence shown in SEQ ID NO: 61. In particular embodiments, a murine IRF8 mutant includes substitution of Lysine (K) at amino acid residue 310 with Arginine (R) (SEQ ID NO: 41). In particular embodiments, a murine IRF8 mutant including a substitution of K at amino acid residue 310 with R is encoded by a nucleotide sequence shown in SEQ ID NO: 63. Small ubiquitin-like modifiers (SUMO) bound to IRF8 primarily at K310 inhibit activation of IRF8 responsive genes. Sentrin-specific protease 1 (SENP1) targets SUMO 2/3. The activity of SENP1 “deSUMOylates” IRF8 (and other proteins) and causes IRF8 to go from a repressor of M1 macrophage differentiation to an activator (directly and through transactivation activities). Preventing SUMO binding to IRF8 by mutation of the K310 residue increases IRF8 specific gene transcription 2-5 fold (see Chang T-H et al. (2012) supra).

Particular embodiments of the present disclosure include engineered IRF transcription factors. In particular embodiments, engineered IRF transcription factors include IRFs that lack a functioning autoinhibitory domain and are therefore insensitive to feedback inactivation (Thompson et al. (2018) Front Immunol 9: 2622). For example, a human IRF5 with 2-3-fold increase in activity can be obtained by deleting aa 489-539 of the human IRF5 protein (Barnes et al. (2002) Mol Cell Biol 22: 5721-5740). In particular embodiments, an autoinhibitory domain of IRF4, a transcription factor that promotes an M2 phenotype, can be deleted or mutated to generate a more active IRF4 in the context of treating an autoimmune disease. In particular embodiments, an autoinhibitory domain of an IRF is found at the carboxy terminus of the IRF protein. In particular embodiments, engineered IRF transcription factors include IRFs that lack one or more functioning nuclear export signals (NES) to entrap IRFs in the nucleus and therefore enhance transcription. For example, nuclear accumulation of human IRF5 can be achieved by mutating the NES of human IRF5 by replacing two leucine residues with alanine (L157A/L159A) (Lin et al. (2000) Molecular and Cellular Biology 20: 6342-6353). In particular embodiments, engineered IRF transcription factors include fusions of one or more IRFs, fusions of fragments of one or more IRFs, and fusions of mutated IRFs.

NFκB is also a key transcription factor related to macrophage M1 activation. NFκB regulates the expression of a large number of inflammatory genes including TNFα, IL1B, cyclooxygenase 2 (COX-2), IL-6, and IL12p40. NFκB activity is modulated via the activation of the inhibitor of kappa B kinase (IKK) trimeric complex (two kinases, IKKα, IKKβ, and a regulatory protein, IKKγ). When upstream signals converge at the IKK complex, they first activate IKKβ kinase via phosphorylation, and activated IKKβ further phosphorylates the inhibitory molecule, inhibitor of kappa B (I-κB). This results in the proteosomal degradation of I-κB and the release of NFκB p65/p50 heterodimer from the NFκB/I-κB complex. The NFκB p65/p50 heterodimer is then translocated to the nucleus and binds to the promoters of inflammatory genes.

IKKβ is an activating kinase for NFκB as well as other transcription factors such as IRF5. IKKβ similarly phosphorylates several other signaling pathway components including FOXO3, NCOA3, BCL10, IRS1, NEMO/IKBKG, NFκB subunits RELA and NFκB1, as well as the IKK-related kinases TBK1 and IKBKE. In particular embodiments, isoforms of human IKKβ include isoform 1 (UniProt Accession 014920-1, SEQ ID NO: 42), isoform 2 (UniProt Accession 014920-2 SEQ ID NO: 43), isoform 3 (UniProt Accession 014920-3 SEQ ID NO: 44), and isoform 4 (UniProt Accession 014920-4 SEQ ID NO: 45). In particular embodiments, isoforms of human IKKβ include isoform 1 encoded by a nucleotide sequence shown in SEQ ID NO: 64, isoform 2 encoded by a nucleotide sequence shown in SEQ ID NO: 65, isoform 3 encoded by a nucleotide sequence shown in SEQ ID NO: 66, and isoform 4 encoded by a nucleotide sequence shown in SEQ ID NO: 67. In particular embodiments, murine IKKβ includes an amino acid sequence shown in SEQ ID NO: 46. In particular embodiments, murine IKKβ is encoded by a nucleotide sequence shown in SEQ ID NO: 68.

The present disclosure provides for the co-expression of IRF transcription factors with one or more molecules that can activate the IRFs to effect TAM reprogramming to an activated state for tumor killing. In particular embodiments, co-expression strategies include: co-expression of IRF5 and IKKβ; co-expression of IRF5 and TANK-binding kinase-1 (TBK-1), TNF receptor-associated factor 6 (TRAF6) adaptor, receptor interacting protein 2 (RIP2) kinase, and/or NFκB kinase-ε (IKKε) (Chang Foreman H-C et al. (2012) PLoS One 7(3): e33098); co-expression of IRF5 and protein kinase DNA-PK (Ryzhakov G et al. (2015) J of Interferon & Cytokine Res 35(2): 71-78); co-expression of IRF5 and protein kinase tyrosine kinase BCR-ABL (Massimo M et al. (2014) Carcinogenesis 35(5):1132-1143); and co-expression of IRF5 or IRF8 with one or more components of the COP9 signalosome (Korczeniewska J et al. (2013) Mol Cell Biol 33(6):1124-1138; Cohen H et al. (2000) J Biol Chem 275(50):39081-39089).

As indicated, hypoxia also influences macrophage polarization through hypoxia inducible factors HIF-1α and HIF-2α. HIF-1α regulates NOS2 expression and supports emergence of an M1 phenotype while HIF-2α regulates Arg1 expression and supports emergence of an M2 phenotype (Takeda N et al. (2010) Genes Dev 24: 491-501).

TABLE 1
Signaling molecules and genes involved
in macrophage polarization.

| | M1 | M2 |
|---|---|---|
| Signaling | STAT1alpha/ | STAT6 |
| Molecules | beta | KLF-4 |
| | IRF5 | NFκB |
| | Btk | p50 homodimers |
| | P2Y(2)R | PPARγ |
| | SOCS3 | HIF-2α |
| | Activin A | IL-21 |
| | HIF1-α | BMP-7 |
| | | FABP4 |
| | | LXRα |
| Genes | TNFα, | Arg-1, |
| | Cox-2, | Mrc-1, |
| | CCL5, | Fizz1, |
| | NOS2 | PPARγ |

Adapted from Sica A and Mantovani A 2012 (supra) and Chávez-Galán L et al. (2015) Front Immunol 6, 253. Arg-1, arginase-1; Fizz1, resistin-like molecule-alpha (Retnl-alpha); STAT, signal transducers and activators of transcription; IRF, interferon regulatory factor; SOCS3, suppressor of cytokine signaling 3; Btk, Bruton's tyrosine kinase; HIF-1α, hypoxia inducible factor 1; KLF-4, Krüppel-like factor 4; TNFα, tumor necrosis factor-alpha; BMP-7, bone morphogenetic protein 7; P2Y(2)R, P2Y purinoceptor 2; PPARγ, peroxisome proliferator-activated receptor γ; NFκB, nuclear factor-kappa B; FABP4, fatty acid binding protein 4; LXRα; liver X receptor alpha.

In particular embodiments, a nucleotide encoding an IRF is used in combination with one or more additional nucleotides encoding other IRFs. In particular embodiments, a nucleotide encoding an IRF is used in combination with one or more additional nucleotides encoding other IRFs and with a nucleotide encoding a IKKβ. In particular embodiments, a nucleotide encoding an IRF is used in combination with a nucleotide encoding a IKKβ at a ratio of 0.5:1, 1:1, 2:1, 3:1, 4:1, or 5:1. In particular embodiments, a nucleotide encoding an IRF is used in combination with a nucleotide encoding a IKKβ at a ratio of 3:1.

Table 2 provides particular combinations of criteria that can be used to distinguish an M1 phenotype from M2 phenotypes (including sub-phenotypes designated as M2a, M2b, M2c and M2d).

TABLE 2
Exemplary Criteria to Categorize Macrophage Phenotypes.

| | M1 | M2a | M2b | M2c | M2d |
|---|---|---|---|---|---|
| Stimulation/ | IFN-γ | IL-4 | ICs | IL-10 | IL-6 |
| Activation | LPS | IL-13 | IL-1R | TGF-β | LIF |
| | GM-CSF | Fungal and | | GCs | Adenosine |
| | | Helminth | | | |
| | | infection | | | |
| Marker | CD86 | CD163 | CD86 | CD163 | VEGF |
| Expression | CD80 | CD23 | MHC II | TLR1 | |
| | CD68 | MHC II | | TLR8 | |
| | MHC II | SR | | | |
| | IL-1R | MMR/CD206 | | | |
| | TLR2 | CD200R | | | |
| | TLR4 | TGM2 | | | |
| | iNOS | DecoyR | | | |
| | SOCS3 | IL-1R II | | | |
| | CD28 | Mouse only: | | | |
| | Gpr18 | Ym½ | | | |
| | Fpr2 | Fizz1 | | | |
| | CD64 | Arg-1 | | | |
| Cytokine | TNF | IL-10 | IL-1 | IL-10 | IL-10 |
| secretion | IL-1β | TGF-β | IL-6 | TGF-β | IL-12 |
| | IL-6 | IL-1ra | IL-10 | | TNFα |
| | IL-12 | | TNFα | | TGFβ |
| | IL-23 | | | | |
| Chemokine | CCL10 | CCL17 | CCL1 | CCR2 | CCL5 |
| secretion | CCL11 | CCL22 | | | CXCL10 |
| | CCL5 | CCL24 | | | CXCL16 |
| | CCL8 | | | | |
| | CCL9 | | | | |
| | CCL2 | | | | |
| | CCL3 | | | | |
| | CCL4 | | | | |

Adapted from Röszer T (2015) Mediators Inflamm 2015, 816460 and Duluc D et al. (2007) Blood 110: 4319-4330. Arg-1, arginase-1; Fizz1, resistin-like molecule-alpha (Retnl-alpha); GCs, glucocorticoids; ICs, immune complexes; IL1-ra, IL-1 receptor antagonist; LIF, leukocyte inhibitory factor; TGM2, transglutaminase 2; TGF-β, transforming growth factor-beta; TNFα, tumor necrosis factor alpha; TLR, Toll-like receptor; MMR (CD206), macrophage mannose receptor; iNOS, inducible nitric oxide synthase; SR, scavenger receptor; SOCS3, suppressor of cytokine signaling 3; VEGF, vascular endothelial growth factor; Ym1 (also known as chitinase-3-like protein-3 (Chi3l3)).

Assays to assess macrophage phenotype can take advantage of the different molecular signatures particular to the M1 or M2 phenotype. A commonly accepted marker profile for M1 macrophages is CD80+, whereas M2-macrophages can be characterized as CD163+. Thus, flow cytometry can be performed to assess for these markers. Driving macrophages towards a M1 type and away from a M2 type can also be assessed by measuring an increase of the IL-12/IL-10 ratio or the CD163−/CD163+ macrophage ratio. In particular embodiments, M1 versus M2 morphology can be assessed by light microscopy. In particular embodiments, phagocytosis assays may be used in conjunction with other assays to assess whether a macrophage is M1 type or M2 phenotype. Phagocytosis assays of different macrophage populations may be performed by incubating an entity to be phagocytosed with macrophages at a concentration that is consistent with their normalized total surface area per cell. The entity to be phagocytosed may be added to macrophage cultures. The entity to be phagocytosed may be, for example, labeled with a fluorescent label. Phagocytosis index may be determined by the median total fluorescence intensity measured per macrophage. Quantification of phagocytosis may be by, for example, flow cytometry. Tumor cell killing assays may also be utilized. In particular embodiments, an M1 phenotype includes reduced expression of signature M2 macrophage genes including SerpinB2 (inhibitor of urokinase-type plasminogen activator), CCL2 (C-C motif chemokine ligand 2), CCL11 (C-C motif chemokine ligand 11), and Retnla (resistin like alpha; Fizz1). In particular embodiments, an M1 phenotype includes increased expression of M1 differentiation genes including CCL5 (C-C motif chemokine ligand 5).

Gene expression (e.g., M1 expression of CD80, CD86 and/or other genes noted above) can be measured by assays well known to a skilled artisan. Methods to measure gene expression include NanoString nCounter® expression assays (NanoString Technologies, Inc., Seattle, Wash.), Northern blots, dot blots, microarrays, serial analysis of gene expression (SAGE), RNA-seq, and quantitative RT-PCR. Methods to measure gene expression products, e.g., protein level, include ELISA (enzyme linked immunosorbent assay), western blot, FACS, radioimmunological assay (RIA), sandwich assay, fluorescent in situ hybridization (FISH), immunohistological staining, immunoelectrophoresis, immunoprecipitation, and immunofluorescence using detection reagents such as an antibody or protein binding agents.

(ii) Cell Attractants. In particular embodiments, a cell attractant is used to attract cells to an anatomical site (e.g., a tumor site). In particular embodiments, cell attractants can be administered at, to, or near the selected anatomical site. In particular embodiments, cell attractants can be administered with a compound that directs them to the selected anatomical site.

In particular embodiments, the selected anatomical site is a tumor site and T cells are attracted to the tumor site. In particular embodiments, the attracted T cells include selected T cells that have been or will be modified to transiently express a therapeutic protein. In particular embodiments, the attracted cells include cells that support the activity of a selected cell type modified to transiently express a therapeutic protein. For example, when T cells are modified to transiently express a therapeutic protein, one could recruit NK cells or invariant NK (iNKT) cells to support tumor-specific T cells. In particular embodiments, more than one cell type can be attracted to a selected anatomical site.

In particular embodiments, selected cells can be attracted to an anatomical site using preconditioning. Preconditioning refers to recruiting cells that will be reprogrammed by administered nanoparticles to an anatomical site. In particular embodiments, preconditioning includes recruiting T cells to a tumor site and reprogramming the recruited T cells with nanoparticles described herein to transiently express tumor-specific receptors.

Thus, optionally, treatment with a nanoparticle leading to expression of a therapeutic protein by a selected cell type can be in concert with a cell attractant, such as a T cell attractant to stimulate T cell recruitment to a tumor to be treated. Appropriate T cell attractants include CCL21 and IP10. Additional immune cell attractants are known in the art. By way of example, the following cell/attractant pairs are recognized:

| | |
|---|---|
| Monocytes/Macrophages | CCL2, CCL3, CCL5, CCL7, CCL8, CCL13, |
| | CCL17 and CCL22 |
| T-lymphocytes | CCL2, CCL1, CCL22 and CCL17 (recruitment |
| | of T-cells); FN-y inducible chemokines |
| | CXCL9, CXCL10 and CXCL11 (recruitment |
| | of activated T-cells) |
| Mast Cells | CCL2 and CCL5 |
| Eosinophils | CCL24, CCL26, CCL7, CCL13, CCL3, |
| | CCL11 (eotaxin) and CCL5 (RANTES) |
| Neutrophils | CXC chemokines (e.g., IL-8) neutrophil |
| | attractant/activation |
| | protein-1 (NAP1) |

One of ordinary skill in the art will recognize that different cell types can be attracted/recruited by different attractant treatments.

(iii) Nanoparticles. As indicated previously, nanoparticles utilized within the current disclosure can include (a) a selected cell targeting ligand; (b) a positively-charged carrier; (c) nucleic acids within the positively-charged carrier; and (d) a neutral or negatively-charged coating.

(a) Selected cell targeting ligands. In particular embodiments, selected cell targeting ligands can include nanoparticle surface-anchored targeting ligands that selectively bind the nanoparticles to selected cells and initiate rapid receptor-induced endocytosis to internalize them. In particular embodiments, the selected cell targeting ligands are covalently coupled to polymers making up the neutral or negatively-charged coating.

In particular embodiments, selected cell targeting ligands can include antibody binding domains, scFv proteins, DART molecules, peptides, and/or aptamers. Particular embodiments utilize anti-CD8, anti-CD3, and/or anti-CD45 antibody binding domains to transfect human T cells, and antibody binding domains recognizing CD34, CD133, or CD46 to target hematopoietic stem cells (HSCs). Examples of binding domains for other cell types including macrophages are also provided.

In particular embodiments, selected cell targeting ligands of the nanoparticles selectively bind selected cells of interest (such as immune cells, or infectious disease cells, or cells infected for instance with a virus or other infectious agent) within a heterogeneous cell population. For targeting according to the compositions and methods disclosed herein, the selected cells are associated with a marker that is currently known or later discovered.

In particular embodiments, the markers are antigens. Antigens refer to substances capable of either binding to an antigen binding region of an immunoglobulin molecule or of eliciting an immune response, e.g., a T cell-mediated immune response by the presentation of the antigen on Major Histocompatibility Antigen (MHC) cellular proteins. “Antigens” include antigenic determinants, haptens, and immunogens, which may be peptides, small molecules, carbohydrates, lipids, nucleic acids or combinations thereof. When referencing antigens that are processed for presentation to T cells, the term “antigen” refers to those portions of the antigen (e.g., a peptide fragment) that is a T cell epitope presented by MHC to the TCR. When used in the context of a B cell mediated immune response in the form of an antibody that is specific for an “antigen”, the portion of the antigen that binds to the complementarity determining regions of the variable domains of the antibody (light and heavy) is referenced. The bound portion may be a linear or three-dimensional epitope.

In particular embodiments, selected immune cells of interest are lymphocytes. Lymphocytes include T-cells, B cells, NK cells, monocytes/macrophages and HSCs.

Several different subsets of T-cells have been discovered, each with a distinct function. In particular embodiments, selected cell targeting ligands achieve selective direction to particular lymphocyte populations through receptor-mediated endocytosis. For example, a majority of T-cells have a T-cell receptor (TCR) existing as a complex of several proteins. The native T-cell receptor is composed of two separate peptide chains, which are produced from the independent T-cell receptor alpha and beta (TCRα and TCRβ) genes and are called α- and β-TCR chains. Selected cell targeting ligands disclosed herein can bind α- and/or β-TCR chains to achieve selective delivery of nucleic acids to these T cells.

γβ T-cells represent a small subset of T-cells that possess a distinct T-cell receptor (TCR) on their surface. In γβ T-cells, the TCR is made up of one γ-chain and one δ-chain. This group of T-cells is much less common (2% of total T-cells) than the αβ T-cells. Nonetheless, selected cell targeting ligands disclosed herein can bind γ- and/or δ TCR chains to achieve selective delivery of nucleic acids to these T cells.

CD3 is expressed on all mature T cells. Accordingly, selected cell targeting ligands disclosed herein can bind CD3 to achieve selective delivery of nucleic acids to all mature T-cells. Activated T-cells express 4-1BB (CD137), CD69, and CD25. Accordingly, selected cell targeting ligands disclosed herein can bind 4-1BB, CD69 or CD25 to achieve selective delivery of nucleic acids to activated T-cells. CD5 and transferrin receptor are also expressed on T-cells and can be used to achieve selective delivery of nucleic acids to T-cells.

T-cells can further be classified into helper cells (CD4+ T-cells) and cytotoxic T-cells (CTLs, CD8+ T-cells), which include cytolytic T-cells. T helper cells assist other white blood cells in immunologic processes, including maturation of B cells into plasma cells and activation of cytotoxic T-cells and macrophages, among other functions. These cells are also known as CD4+ T-cells because they express the CD4 protein on their surface. Helper T-cells become activated when they are presented with peptide antigens by MHC class II molecules that are expressed on the surface of antigen presenting cells (APCs). Once activated, they divide rapidly and secrete small proteins called cytokines that regulate or assist in the active immune response. Selected cell targeting ligands disclosed herein can bind CD4 to achieve selective delivery of nucleic acids to T helper cells.

Cytotoxic T-cells destroy virally infected cells and tumor cells, and are also implicated in transplant rejection. These cells are also known as CD8+ T-cells because they express the CD8 glycoprotein on their surface. These cells recognize their targets by binding to antigen associated with MHC class I, which is present on the surface of nearly every cell of the body. Selected cell targeting ligands disclosed herein can bind CD8 to achieve selective delivery of nucleic acids to CTL.

“Central memory” T-cells (or “TCM”) as used herein refers to an antigen experienced CTL that expresses CD62L or CCR7 and CD45RO on the surface thereof, and does not express or has decreased expression of CD45RA as compared to naive cells. In particular embodiments, central memory cells are positive for expression of CD62L, CCR7, CD25, CD127, CD45RO, and CD95, and have decreased expression of CD45RA as compared to naive cells. Selected cell targeting ligands disclosed herein can bind CD62L, CCR7, CD25, CD127, CD45RO and/or CD95 to achieve selective delivery of nucleic acids to TCM.

“Effector memory” T-cell (or “TEM”) as used herein refers to an antigen experienced T-cell that does not express or has decreased expression of CD62L on the surface thereof as compared to central memory cells, and does not express or has decreased expression of CD45RA as compared to a naive cell. In particular embodiments, effector memory cells are negative for expression of CD62L and CCR7, compared to naive cells or central memory cells, and have variable expression of CD28 and CD45RA. Effector T-cells are positive for granzyme B and perforin as compared to memory or naive T-cells. Selected cell targeting ligands disclosed herein can bind granzyme B and/or perforin to achieve selective delivery of nucleic acids to TEM.

Regulatory T cells (“TREG”) are a subpopulation of T cells, which modulate the immune system, maintain tolerance to self-antigens, and abrogate autoimmune disease. TREG express CD25, CTLA-4, GITR, GARP and LAP. Selected cell targeting ligands disclosed herein can bind CD25, CTLA-4, GITR, GARP and/or LAP to achieve selective delivery of nucleic acids to naïve TREG.

“Naive” T-cells as used herein refers to a non-antigen experienced T cell that expresses CD62L and CD45RA, and does not express CD45RO as compared to central or effector memory cells. In particular embodiments, naive CD8+ T lymphocytes are characterized by the expression of phenotypic markers of naive T-cells including CD62L, CCR7, CD28, CD127, and CD45RA. Selected cell targeting ligands disclosed herein can bind CD62L, CCR7, CD28, CD127 and/or CD45RA to achieve selective delivery of nucleic acids to naïve T-cells.

NK cells (also known as K cells, and killer cells) are activated in response to interferons or macrophage-derived cytokines. They serve to contain viral infections while the adaptive immune response is generating antigen-specific cytotoxic T cells that can clear the infection. NK cells express CD8, CD16 and CD56 but do not express CD3. Selected cell targeting ligands disclosed herein can bind CD8, CD16 and/or CD56 to achieve selective delivery of nucleic acids to NK cells.

Macrophages (and their precursors, monocytes) reside in every tissue of the body (in certain instances as microglia, Kupffer cells and osteoclasts) where they engulf apoptotic cells, pathogens, and other non-self-components. Because monocytes/macrophages engulf non-self-components, a particular macrophage- or monocyte-directing agent is not required on the nanoparticles described herein for selective uptake by these cells. Alternatively, selected cell targeting ligands disclosed herein can bind CD11b, F4/80; CD68; CD11c; IL-4Rα; and/or CD163 to achieve selective delivery of nucleic acid to monocytes/macrophages. It is recognized that macrophages will not express a TCR, and thus they are not desired targets for nanoparticle particles described herein that include mRNA encoding a TCR protein or CAR/TCR hybrid protein.

Immature dendritic cells (i.e., pre-activation) engulf antigens and other non-self-components in the periphery and subsequently, in activated form, migrate to T-cell areas of lymphoid tissues where they provide antigen presentation to T cells. Thus, like macrophages, the targeting of dendritic cells need not rely on a selected cell targeting ligand. When a selected cell targeting ligand is used to selectively target dendritic cells, it can bind the following CD antigens: CD1a, CD1b, CD1c, CD1d, CD21, CD35, CD39, CD40, CD86, CD101, CD148, CD209, and DEC-205.

B cells can be distinguished from other lymphocytes by the presence of the B cell receptor (BCR). The principal function of B cells is to make antibodies. B cells express CD5, CD19, CD20, CD21, CD22, CD35, CD40, CD52, and CD80. Selected cell targeting ligands disclosed herein can bind CD5, CD19, CD20, CD21, CD22, CD35, CD40, CD52, and/or CD80 to achieve selective delivery of nucleic acids to B-cells. Antibodies targeting the B-cell receptor isotype constant regions (IgM, IgG, IgA, IgE) can also be used to target B-cell subtypes.

Lymphocyte function-associated antigen 1 (LFA-1) is expressed by all T-cells, B-cells, and monocytes/macrophages. Accordingly, selected cell targeting ligands disclosed herein can bind LFA-1 to achieve selective delivery of nucleic acids to T-cells, B-cells, and monocytes/macrophages.

HSCs can also be targeted for selective delivery of nanoparticles disclosed herein. HSCs express CD34, CD46, CD133, Sca-1 and CD117. Selected cell targeting ligands disclosed herein can bind CD34, CD46, CD133, Sca-1 and/or CD117 to achieve selective delivery of nucleic acids to hematopoietic stem cells.

“Selective delivery” means that nucleic acids are delivered and expressed by one or more selected lymphocyte populations. In particular embodiments, selective delivery is exclusive to a selected lymphocyte population. In particular embodiments, at least 65%, 70%, 75%, 80%, 85%, 90%, 95% or 99% of administered nucleic acids are delivered and/or expressed by a selected lymphocyte population. In particular embodiments, selective delivery ensures that non-lymphocyte cells do not express delivered nucleic acids. For example, when the targeting agent is a T-cell receptor (TCR) gene, selectivity is ensured because only T cells have the zeta chains required for TCR expression. Selective delivery can also be based on lack of nucleic acid uptake into unselected cells.

Selected cell targeting ligands can include binding domains for motifs found on lymphocyte cells. Selected cell targeting ligands can also include any selective binding mechanism allowing selective uptake into lymphocytes. In particular embodiments, selected cell targeting ligands include binding domains for T-cell receptor motifs; T-cell α chains; T-cell β chains; T-cell γ chains; T-cell 8 chains; CCR7; CD1a; CD1b; CD1c; CD1d; CD3; CD4; CDS; CD7; CD8; CD11b; CD11c; CD16; CD19; CD20; CD21; CD22; CD25; CD28; CD34; CD35; CD39; CD40; CD45RA; CD45RO; CD46, CD52; CD56; CD62L; CD68; CD80; CD86; CD95; CD101; CD117; CD127; CD133; CD137 (4-1BB); CD148; CD163; F4/80; IL-4Rα; Sca-1; CTLA-4; GITR; GARP; LAP; granzyme B; LFA-1; transferrin receptor; and combinations thereof.

In particular embodiments, binding domains include cell marker ligands, receptor ligands, antibody binding domains, peptides, peptide aptamers, nucleic acids, nucleic acid aptamers, spiegelmers or combinations thereof. Within the context of selected cell targeting ligands, binding domains include any substance that binds to another substance to form a complex capable of mediating endocytosis.

Antibody binding domains include binding fragments of an antibody, e.g., Fv, Fab, Fab′, F(ab′)₂, and single chain Fv fragments (scFvs) or any biologically effective fragments of an immunoglobulin that bind specifically to a motif expressed by a lymphocyte. Antibodies or antigen binding fragments include all or a portion of polyclonal antibodies, monoclonal antibodies, human antibodies, humanized antibodies, synthetic antibodies, chimeric antibodies, bispecific antibodies, mini bodies, and linear antibodies.

Antibodies from human origin or humanized antibodies have lowered or no immunogenicity in humans and have a lower number of non-immunogenic epitopes compared to non-human antibodies. Antibodies and their fragments will generally be selected to have a reduced level or no antigenicity in human subjects.

Antibodies that specifically bind a motif expressed by a lymphocyte can be prepared using methods of obtaining monoclonal antibodies, methods of phage display, methods to generate human or humanized antibodies, or methods using a transgenic animal or plant engineered to produce antibodies as is known to those of ordinary skill in the art (see, for example, U.S. Pat. Nos. 6,291,161 and 6,291,158). Phage display libraries of partially or fully synthetic antibodies are available and can be screened for an antibody or fragment thereof that can bind to a lymphocyte motif. For example, binding domains may be identified by screening a Fab phage library for Fab fragments that specifically bind to a target of interest (see Hoet et al., Nat. Biotechnol. 23:344, 2005). Phage display libraries of human antibodies are also available. Additionally, traditional strategies for hybridoma development using a target of interest as an immunogen in convenient systems (e.g., mice, HuMAb Mouse® (GenPharm Intl, Inc., Mountain View, Calif., TC Mouse™ (Kyowa Hakko Kirin Co., Tokyo, JP; see, e.g., Takauchi et al., J. Periodontol. 2005 May 76(5): 680-5), KM-Mouse® (Medarex, Inc., Princeton, N.J.), llamas, chicken, rats, hamsters, rabbits, etc.) can be used to develop binding domains. In particular embodiments, antibodies specifically bind to motifs expressed by a selected lymphocyte and do not cross react with nonspecific components or unrelated targets. Once identified, the amino acid sequence or nucleic acid sequence coding for the antibody can be isolated and/or determined.

In particular embodiments, binding domains of selected cell targeting ligands include T-cell receptor motif antibodies; T-cell a chain antibodies; T-cell β chain antibodies; T-cell γ chain antibodies; T-cell δ chain antibodies; CCR7 antibodies; CD1a antibodies; CD1b antibodies; CD1c antibodies; CD1d antibodies; CD3 antibodies; CD4 antibodies; CD5 antibodies; CD7 antibodies; CD8 antibodies; CD11b antibodies; CD11c antibodies; CD16 antibodies; CD19 antibodies; CD20 antibodies; CD21 antibodies; CD22 antibodies; CD25 antibodies; CD28 antibodies; CD34 antibodies; CD35 antibodies; CD39 antibodies; CD40 antibodies; CD45RA antibodies; CD45RO antibodies; CD46 antibodies; CD52 antibodies; CD56 antibodies; CD62L antibodies; CD68 antibodies; CD80 antibodies; CD86 antibodies CD95 antibodies; CD101 antibodies; CD117 antibodies; CD127 antibodies; CD133 antibodies; CD137 (4-1BB) antibodies; CD148 antibodies; CD163 antibodies; F4/80 antibodies; IL-4Rα antibodies; Sca-1 antibodies; CTLA-4 antibodies; GITR antibodies; GARP antibodies; LAP antibodies; granzyme B antibodies; LFA-1 antibodies; or transferrin receptor antibodies. These binding domains also can consist of scFv fragments of the foregoing antibodies.

Exemplary antibodies (such as scFvs) useful in the methods and compositions of the present disclosure include those provided in WO2014164553A1, US20170283504, U.S. Pat. No. 7,083,785B2, U.S. Ser. No. 10/189,906B2, U.S. Ser. No. 10/174,095B2, WO2005102387A2, US20110206701A1, WO2014179759A1, US20180037651A1, US20180118822A1, WO2008047242A2, WO1996016990A1, WO2005103083A2, and WO1999062526A2, each of which is incorporated herein by reference.

Peptide aptamers include a peptide loop (which is specific for a target protein) attached at both ends to a protein scaffold. This double structural constraint greatly increases the binding affinity of the peptide aptamer to levels comparable to an antibody. The variable loop length is typically 8 to 20 amino acids (e.g., 8 to 12 amino acids), and the scaffold may be any protein which is stable, soluble, small, and non-toxic (e.g., thioredoxin-A, stefin A triple mutant, green fluorescent protein, eglin C, and cellular transcription factor SpI). Peptide aptamer selection can be made using different systems, such as the yeast two-hybrid system (e.g., Gal4 yeast-two-hybrid system) or the LexA interaction trap system.

Nucleic acid aptamers are single-stranded nucleic acid (DNA or RNA) ligands that function by folding into a specific globular structure that dictates binding to target proteins or other molecules with high affinity and specificity, as described by Osborne et al., Curr. Opin. Chem. Biol. 1:5-9, 1997; and Cerchia et al., FEBS Letters 528:12-16, 2002. In particular embodiments, aptamers are small (15 KD; or between 15-80 nucleotides or between 20-50 nucleotides). Aptamers are generally isolated from libraries including 10¹⁴-10¹⁵ random oligonucleotide sequences by a procedure termed SELEX (systematic evolution of ligands by exponential enrichment; see, for example, Tuerk et al., Science, 249:505-510, 1990; Green et al., Methods Enzymology. 75-86, 1991; and Gold et al., Annu. Rev. Biochem., 64: 763-797, 1995). Further methods of generating aptamers are described in, for example, U.S. Pat. Nos. 6,344,318; 6,331,398; 6,110,900; 5,817,785; 5,756,291; 5,696,249; 5,670,637; 5,637,461; 5,595,877; 5,527,894; 5,496,938; 5,475,096; and 5,270,16. Spiegelmers are similar to nucleic acid aptamers except that at least one β-ribose unit is replaced by β-D-deoxyribose or a modified sugar unit selected from, for example, β-D-ribose, α-D-ribose, β-L-ribose.

In particular embodiments, Egr2 is targeted on M2 macrophages. Commercially available antibodies for Egr2 can be obtained from Thermo Fisher, Waltham, Mass.; Abcam, Cambridge, Mass.; Millipore Sigma, Burlington, Mass.; Miltenyi Biotec, Bergisch Gladbach, Germany; LifeSpan Biosciences, Inc., Seattle, Wash.; and Novus Biologicals, Littleton, Colo. Generation of anti-Egr2 antibodies are discussed, for example, in Murakami K et al. (1993) Oncogene 8(6): 1559-1566. Anti-Egr2 antibodies include: rabbit monoclonal anti-Egr2 antibody clone EPR4004; mouse monoclonal anti-Egr2 antibody clone 1G5; mouse monoclonal anti-Egr2 antibody clone OTI1B12; rabbit polyclonal anti-Egr2 antibody recognizing AA residues 200-300 of human Egr2; rabbit polyclonal anti-Egr2 antibody recognizing AA residues 340-420 of human Egr2; and rabbit polyclonal anti-Egr2 antibody recognizing AA residues 370-420 of human Egr2. Binding domains can be derived from these antibodies and other antibodies disclosed herein.

A number of additional antibodies specific for CD206 are known to those of skill in the art and can be readily characterized for sequence, epitope binding, and affinity. See, for example, WO 2014/140376, WO 2013/174537, and U.S. Pat. No. 7,560,534. Commercially available antibodies for CD206 can be obtained from Thermo Fisher, Waltham, Mass.; Proteintech, Rosemont, Ill.; BioLegend, San Diego, Calif.; R & D Systems, Minneapolis, Minn.; LifeSpan Biosciences, Inc., Seattle, Wash.; Novus Biologicals, Littleton, Colo.; and Bio-Rad, Hercules, Calif. In particular embodiments, an anti-CD206 antibody includes a rat monoclonal anti-mouse CD206 monoclonal antibody clone C068C2 (Cat #141732, Biolegend, San Diego, Calif.).

In particular embodiments, the targeting ligand includes a human or humanized binding domain (e.g., scfv) including a variable light chain including a CDRL1 sequence including ASQSVSHDV (SEQ ID NO: 69), a CDRL2 sequence including YTS, and a CDRL3 sequence including QDYSSPRT (SEQ ID NO: 70). In particular embodiments, the targeting ligand includes a human or humanized binding domain (e.g., scfv) including a variable heavy chain including a CDRH1 sequence including GYSITSDY (SEQ ID NO: 71), a CDRH2 sequence including YSG, and a CDRH3 sequence including CVSGTYYFDYWG (SEQ ID NO: 72). These reflect CDR sequences of the Mac2-48 antibody that bind CD163.

In particular embodiments, the targeting ligand includes a human or humanized binding domain (e.g., scfv) including a variable light chain including a CDRL1 sequence including ASQSVSSDV (SEQ ID NO: 73), a CDRL2 sequence including YAS, and a CDRL3 sequence including QDYTSPRT (SEQ ID NO: 74). In particular embodiments, the targeting ligand includes a human or humanized binding domain (e.g., scfv) including a variable heavy chain including a CDRH1 sequence including GYSITSDY (SEQ ID NO: 75), a CDRH2 sequence including YSG, and a CDRH3 sequence including CVSGTYYFDYWG (SEQ ID NO: 76). These reflect CDR sequences of the Mac2-158 antibody that bind CD163.

A number of additional antibodies or binding domains specific for CD163 are known to those of skill in the art and can be readily characterized for sequence, epitope binding, and affinity. See, for example, WO 2011/039510, WO 2002/032941, WO 2002/076501, and US 2005/0214871. Commercially available antibodies for CD163 can be obtained from Thermo Fisher, Waltham, Mass.; Enzo Life Sciences, Inc., Farmingdale, N.Y.; BioLegend, San Diego, Calif.; R & D Systems, Minneapolis, Minn.; LifeSpan Biosciences, Inc., Seattle, Wash.; and RDI Research Diagnostics, Flanders, N.J. In particular embodiments, anti-CD163 antibodies can include: mouse monoclonal anti-CD163 antibody clone 3D4; mouse monoclonal anti-CD163 antibody clone Ber-Mac3; mouse monoclonal anti-CD163 antibody clone EDHu-1; and mouse monoclonal anti-CD163 antibody clone GHI/61.

In particular embodiments, the targeting ligand includes a human or humanized binding domain (e.g., scfv) including a variable light chain including a CDRL1 sequence including RSSKSLLYKDGKTYLN (SEQ ID NO: 77), a CDRL2 sequence including LMSTRAS (SEQ ID NO: 78), and a CDRL3 sequence including QQLVEYPFT (SEQ ID NO: 79). In particular embodiments, the targeting ligand includes a human or humanized binding domain (e.g., scfv) including a variable heavy chain including a CDRH1 sequence including GYWMS (SEQ ID NO: 80), a CDRH2 sequence including EIRLKSDNYATHYAESVKG (SEQ ID NO: 81), and a CDRH3 sequence including FID. These reflect CDR sequences that bind CD23.

A number of antibodies or binding domains specific for CD23 are known to those of skill in the art and can be readily characterized for sequence, epitope binding, and affinity. See, for example, U.S. Pat. Nos. 7,008,623, 6,011,138 A (antibodies including 5E8, 6G5, 2C8, B3B1 and 3G12), US 2009/0252725, Rector et al. (1985) J. Immunol. 55: 481-488; Flores-Rumeo et al. (1993) Science 241: 1038-1046; Sherr et al. (1989) J. Immunol. 142: 481-489; and Pene et al., (1988) PNAS 85: 6820-6824. Commercially available antibodies for CD23 can be obtained from Thermo Fisher, Waltham, Mass.; Abcam, Cambridge, Mass.; Bioss Antibodies, Inc., Woburn, Mass.; Bio-Rad, Hercules, Calif.; LifeSpan Biosciences, Inc., Seattle, Wash.; and Boster Biological Technology, Pleasanton, Calif. In particular embodiments, anti-CD23 antibodies can include: mouse monoclonal anti-CD23 antibody clone Tu 1; rabbit monoclonal anti-CD23 antibody clone SP23; rabbit monoclonal anti-CD23 antibody clone EPR3617; mouse monoclonal anti-CD23 antibody clone 5B5; mouse monoclonal anti-CD23 antibody clone 1B12; mouse monoclonal anti-CD23 antibody clone M-L23.4; and mouse monoclonal anti-CD23 antibody clone 3A2.

M1 Binding Domains. In particular embodiments, the targeting ligand includes a human or humanized binding domain (e.g., scfv) including a variable light chain including a CDRL1 sequence including SSNIGDNY (SEQ ID NO: 82), a CDRL2 sequence including RDS, and a CDRL3 sequence including QSYDSSLSGS (SEQ ID NO: 83). In particular embodiments, the targeting ligand includes a human or humanized binding domain (e.g., scfv) including a variable heavy chain including a CDRH1 sequence including GFTFDDYG (SEQ ID NO: 84), a CDRH2 sequence including ISWNGGKT (SEQ ID NO: 85), and a CDRH3 sequence including ARGSLFHDSSGFYFGH (SEQ ID NO: 86). These reflect CDR sequences of the Ab79 antibody that bind CD38.

In particular embodiments, the targeting ligand includes a human or humanized binding domain (e.g., scfv) including a variable light chain including a CDRL1 sequence including NSNIGSNT (SEQ ID NO: 87), a CDRL2 sequence including SDS, and a CDRL3 sequence including QSYDSSLSGSR (SEQ ID NO: 88). In particular embodiments, the targeting ligand includes a human or humanized binding domain (e.g., scfv) including a variable heavy chain including a CDRH1 sequence including GFTFNNYG (SEQ ID NO: 89), a CDRH2 sequence including ISYDGSDK (SEQ ID NO: 90), and a CDRH3 sequence including ARVYYYGFSGPSMDV (SEQ ID NO: 91). These reflect CDR sequences of the Ab19 antibody that bind CD38.

In particular embodiments, the targeting ligand includes a human or humanized binding domain (e.g., scfv) including a variable light chain including a CDRL1 sequence including RASQSVSSYLA (SEQ ID NO: 92), a CDRL2 sequence including DASNRAT (SEQ ID NO: 93), and a CDRL3 sequence including QQRSNWPPTF (SEQ ID NO: 94). In particular embodiments, the targeting ligand includes a human or humanized binding domain (e.g., scfv) including a variable heavy chain including a CDRH1 sequence including SFAMS (SEQ ID NO: 95), a CDRH2 sequence including AISGSGGGTYYADSVKG (SEQ ID NO: 96), and a CDRH3 sequence including DKILWFGEPVFDY (SEQ ID NO: 97). These reflect CDR sequences of the daratumumab antibody that bind CD38 described in U.S. Pat. No. 7,829,693.

A number of antibodies specific for CD38 are known to those of skill in the art and can be readily characterized for sequence, epitope binding, and affinity. See, for example, WO 2005/103083, WO 2006/125640, WO 2007/042309, WO 2008/047242, WO 2012/092612, WO 2006/099875, WO 2011/154453, WO 2015/130728, U.S. Pat. No. 7,829,693, and US 2016/0200828. Commercially available antibodies for CD38 can be obtained from Thermo Fisher, Waltham, Mass.; Abcam, Cambridge, Mass.; and Millipore Sigma, Burlington, Mass. In particular embodiments, anti-CD23 antibodies can include: rabbit monoclonal anti-CD38 antibody clone GAD-3; mouse monoclonal anti-CD38 antibody clone HIT2; mouse monoclonal anti-CD38 antibody clone AT1; mouse monoclonal anti-CD38 antibody clone AT13/5; rat monoclonal anti-CD38 antibody clone NIMR-5; and rat monoclonal IgG2a, κ anti-CD38 antibody clone 90/CD38 (Cat #BD Biosciences, San Jose, Calif.).

In particular embodiments, G-protein coupled receptor 18 (Gpr18) is targeted on M1 macrophages. Commercially available antibodies for Gpr18 can be obtained from Assay Biotechnology Company Inc., Sunnyvale, Calif.; Thermo Fisher, Waltham, Mass.; Abcam, Cambridge, Mass.; GeneTex, Inc., Irvine, Calif.; and Novus Biologicals, Littleton, Colo. In particular embodiments, anti-Gpr18 antibodies include: rabbit polyclonal anti-Gpr18 antibody recognizing a portion of amino acids 1-50 of human Gpr18; rabbit polyclonal anti-Gpr18 antibody recognizing a region including amino acids 160-240 of human Gpr18; rabbit polyclonal anti-Gpr18 antibody recognizing a region including amino acids 100-180 of human Gpr18; rabbit monoclonal anti-Gpr18 antibody clone EPR12359; and rabbit polyclonal anti-Gpr18 antibody recognizing a region including amino acids 140-190 of human Gpr18.

In particular embodiments, formyl peptide receptor 2 (Fpr2) is targeted on M1 macrophages. Commercially available antibodies for Fpr2 can be obtained from Atlas Antibodies, Bromma, Sweden; Biorbyt, LLC, San Francisco, Calif.; Cloud-Clone Corp., Katy, Tex.; US Biological Life Sciences, Salem, Mass.; and Novus Biologicals, Littleton, Colo. In particular embodiments, anti-fpr2 antibodies include: mouse monoclonal anti-fpr2 antibody clone GM1D6; mouse monoclonal anti-fpr2 antibody clone 304405; recombinant anti-fpr2 antibody clone REA663; and rabbit polyclonal anti-fpr2 antibody recognizing a region including amino acids 300-350 of fpr2.

In particular embodiments, the targeting ligand includes a human or humanized binding domain (e.g., scfv) including a variable light chain including a CDRL1 sequence including RASQSVSSYLA (SEQ ID NO: 98), a CDRL2 sequence including DASSRAT (SEQ ID NO: 99), and a CDRL3 sequence including QLRSNWPPYT (SEQ ID NO: 92). In particular embodiments, the targeting ligand includes a human or humanized binding domain (e.g., scfv) including a variable heavy chain including a CDRH1 sequence including GYGMH (SEQ ID NO: 100), a CDRH2 sequence including VIWYDGSNKYYADSVKG (SEQ ID NO: 101), and a CDRH3 sequence including DTGDRFFDY (SEQ ID NO: 102). These reflect CDR sequences that bind CD64.

A number of antibodies specific for CD64 are known to those of skill in the art and can be readily characterized for sequence, epitope binding, and affinity. See, for example, U.S. Pat. No. 7,378,504, WO 2006/131953, and WO 2008/074867. Commercially available antibodies for CD64 can be obtained from Ancell, Bayport, Minn.; Thermo Fisher, Waltham, Mass.; Abcam, Cambridge, Mass.; LifeSpan Biosciences, Inc., Seattle, Wash.; and Novus Biologicals, Littleton, Colo. In particular embodiments, anti-CD64 antibodies include: mouse monoclonal anti-CD64 antibody clone 32-2; mouse monoclonal anti-CD64 antibody clone UMAB74; rat monoclonal anti-CD64 antibody clone 290322; mouse monoclonal anti-CD64 antibody clone 10.1; and mouse monoclonal anti-CD64 antibody clone 1D3.

In particular embodiments, CD86 is targeted on M1 macrophages. A number of antibodies specific for CD86 are known to those of skill in the art and can be readily characterized for sequence, epitope binding, and affinity. See, for example, WO 2004/076488, U.S. Pat. No. 8,378,082 (mAb 2D4) and U.S. Pat. No. 6,346,248 (IG10H6D10). Commercially available antibodies for CD86 can be obtained from Thermo Fisher, Waltham, Mass.; Miltenyi Biotec, Bergisch Gladbach, Germany; LifeSpan Biosciences, Inc., Seattle, Wash.; Bio-Rad, Hercules, Calif.; and Novus Biologicals, Littleton, Colo. In particular embodiments, anti-CD86 antibodies include: mouse monoclonal anti-CD86 antibody clone BU63; polyclonal goat anti-CD86 antibody recognizing a region including Ala23 to His244 of human CD86; mouse monoclonal anti-CD86 antibody clone IT2.2; rabbit monoclonal anti-CD86 antibody clone BFF-3; and mouse monoclonal anti-CD86 antibody clone C86/1146.

Other agents that can facilitate internalization by and/or transfection of lymphocytes, such as poly(ethyleneimine)/DNA (PEI/DNA) complexes can also be used.

(b) Positively-Charged Carriers. In particular embodiments, carriers include a carrier molecule that condenses and protects nucleic acids from enzymatic degradation. As disclosed in more detail elsewhere herein, carriers can include positively charged lipids and/or polymers. Particular embodiments utilize poly(β-amino ester) (PBAE (e.g., PBAE 447 and/or with 1-(3-aminopropyl)pyrrolidine end caps). In some embodiments, the molecular weight of the PBAE is between 4 kDa and 6 kDa, between 5 kDa and 7 kDa, between 6 kDa and 8 kDa, between 7 kDa and 9 kDa, between 8 kDa and 10 kDa, or between 9 kDa and 11 kDa. In some embodiments, the molecular weight of the PBAE is 4 kDa, 5 kDa, 6 kDa, 7 kDa, 8 kDa, 9 kDa, or 11 kDa. In some embodiments, the molecular weight of the PBAE is less than 4 kDa or more than 11 kDa. In some embodiments, the PBAE is PBAE 447. In some embodiments, the molecular weight of the PBAE 447 is between 4 kDa and 6 kDa, between 5 kDa and 7 kDa, between 6 kDa and 8 kDa, between 7 kDa and 9 kDa, between 8 kDa and 10 kDa, or between 9 kDa and 11 kDa. In some embodiments, the molecular weight of the PBAE 447 is 4 kDa, 5 kDa, 6 kDa, 7 kDa, 8 kDa, 9 kDa, or 11 kDa. In some embodiments, the molecular weight of the PBAE 447 is less than 4 kDa or more than 11 kDa.

When assessed by gel permeation chromatography (GPC), polymers (e.g., PBAE) can include a range of polymer lengths within the matrix, including for example, an Mn range of 3,000-6,000 or 4,000-5,000; a mW range of 10,000-20,000 or 14,500-21,000; and/or an Mz range of 55,000-77,000 or 60,000-72,000. In particular embodiments, PBAE within a carrier matrix has a molecular weight distribution by GPC of Mn=4,000-5,000; Mw=14,500-21,000; and Mz=60,000-72,000.

In some embodiments, the PBAE is conjugated to one or more molecules. In some embodiments, the PBAE is conjugate to polyethylene glycol (to form PEG-PBAE). End groups can also a play a large role in transfection efficiency, with end caps containing tertiary amines being preferred.

Additional examples of positively charged lipids include esters of phosphatidic acid with an aminoalcohol, such as an ester of dipalmitoyl phosphatidic acid or distearoyl phosphatidic acid with hydroxyethylenediamine. More particular examples of positively charged lipids include 3β-[N—(N′,N′-dimethylaminoethyl)carbamoyl) cholesterol (DC-chol); N,N′-dimethyl-N,N′-dioctacyl ammonium bromide (DDAB); N,N′-dimethyl-N,N′-dioctacyl ammonium chloride (DDAC); 1,2-dioleoyloxypropyl-3-dimethyl-hydroxyethyl ammonium chloride (DORI); 1,2-dioleoyloxy-3-[trimethylammonio]-propane (DOTAP); N-(1-(2,3-dioleyloxy)propyl)-N,N,N-trimethylammonium chloride (DOTMA); dipalmitoylphosphatidylcholine (DPPC); 1,2-dioctadecyloxy-3-[trimethylammonio]-propane (DSTAP); and the cationic lipids described in e.g. Martin et al., Curr. Pharma. Design, 11:375-394, 2005.

Examples of positively charged polymers that can be used as carriers within the current disclosure include polyamines; polyorganic amines (e.g., polyethyleneimine (PEI), polyethyleneimine celluloses); poly(amidoamines) (PAMAM); polyamino acids (e.g., polylysine (PLL), polyarginine); polysaccharides (e.g., cellulose, dextran, DEAE dextran, starch); spermine, spermidine, poly(vinylbenzyl trialkyl ammonium), poly(4-vinyl-N-alkyl-pyridiumiun), poly(acryloyl-trialkyl ammonium), and Tat proteins.

Blends of lipids and polymers in any concentration and in any ratio can also be used. Blending different polymer types in different ratios using various grades can result in characteristics that borrow from each of the contributing polymers. Various terminal group chemistries can also be adopted.

Without limiting the foregoing, particular embodiments disclosed herein can also utilize porous nanoparticles constructed from any material capable of forming a porous network. Exemplary materials include metals, transition metals and metalloids. Exemplary metals, transition metals and metalloids include lithium, magnesium, zinc, aluminum, and silica. In particular embodiments, the porous nanoparticles include silica. The exceptionally high surface area of mesoporous silica (exceeding 1,000 m²/g) enables nucleic acid loading at levels exceeding conventional DNA carriers such as liposomes.

Carrier matrices can be formed in a variety of different shapes, including spheroidal, cuboidal, pyramidal, oblong, cylindrical, toroidal, and the like. The nucleic acids can be included in the pores of the carriers in a variety of ways. For example, the nucleic acids can be encapsulated in the porous nanoparticles. In other aspects, the nucleic acids can be associated (e.g., covalently and/or non-covalently) with the surface or close underlying vicinity of the surface of the porous nanoparticles. In particular embodiments, the nucleic acids can be incorporated in the porous nanoparticles e.g., integrated in the material of the porous nanoparticles. For example, the nucleic acids can be incorporated into a polymer matrix of polymer nanoparticles.

(c) Nucleic Acids within the Positively-Charged Carrier. Nucleic acids resulting in expression of therapeutic proteins are described above in relation to expression of CAR, TCR, CAR/TCR hybrids and macrophage activators.

(d) Neutral or Negatively-Charged Coating. In particular embodiments, the nanoparticles disclosed herein include a coating that shields the encapsulated nucleic acids and reduces or prevents off-target binding. Off-target binding is reduced or prevented by reducing the surface charge of the nanoparticles to neutral or negative. As disclosed in more detail elsewhere herein, coatings can include neutral or negative polymer- and/or liposome-based coatings. Particular embodiments utilize polyglutamic acid (PGA) as a nanoparticle coating. When used, the coating need not necessarily coat the entire nanoparticle. Advantageously, the coating is be sufficient to reduce off-target binding by the nanoparticle. An antibody fragment (e.g., Fab or scFv) can be directly or indirectly linked to the PGA coating. For example, an antibody fragment (e.g., Fab or scFv) can be chemically coupled to the PGA using, for example, PGA-maleimide reacting with a cysteine added to Fab or scFv sequence. In some embodiments, the antibody is coupled through a linker (e.g. a protein or polypeptide linker, or a chemical linker).

In particular embodiments, the coating is a dense surface coating of hydrophilic and/or neutrally charged hydrophilic polymer sufficient to prevent the encapsulated nucleic acids from being exposed to the environment before release into a selected cell. In particular embodiments, the coating covers at least 80% or at least 90% of the surface of the nanoparticle. In particular embodiments, the coating includes PGA.

Examples of neutrally charged polymers that can be used as coating within embodiments of the disclosure include polyethylene glycol (PEG); poly(propylene glycol); and polyalkylene oxide copolymers (PLURONIC®, BASF Corp., Mount Olive, N.J.).

Neutrally charged polymers also include zwitterionic polymers. Zwitterionic refers to the property of overall charge neutrality while having both a positive and a negative electrical charge. Zwitterionic polymers can behave like regions of cell membranes that resist cell and protein adhesion.

Zwitterionic polymers include zwitterionic constitutional units including pendant groups (i.e., groups pendant from the polymer backbone) with zwitterionic groups. Exemplary zwitterionic pendant groups include carboxybetaine groups (e.g., —Ra-N+(Rb)(Rc)-Rd-CO₂—, where Ra is a linker group that covalently couples the polymer backbone to the cationic nitrogen center of the carboxybetaine groups, Rb and Rc are nitrogen substituents, and Rd is a linker group that covalently couples the cationic nitrogen center to the carboxy group of the carboxybetaine group).

Examples of negatively charged polymers include alginic acids; carboxylic acid polysaccharides; carboxymethyl cellulose; carboxymethyl cellulose-cysteine; carrageenan (e.g., Gelcarin® (FMC Corp., Wilmington, Del.) 209, Gelcarin® 379); chondroitin sulfate; glycosaminoglycans; mucopolysaccharides; negatively charged polysaccharides (e.g., dextran sulfate); poly(acrylic acid); poly(D-aspartic acid); poly(L-aspartic acid); poly(L-aspartic acid) sodium salt; poly(D-glutamic acid); poly(L-glutamic acid); poly(L-glutamic acid) sodium salt; poly(methacrylic acid); sodium alginate (e.g., PROTANAL® (FMC Biopolymer, Oslo, Norway) LF 120M, PROTANAL® LF 200M, PROTANAL® LF 200D); sodium carboxymethyl cellulose (CMC); sulfated polysaccharides (heparins, agaropectins); pectin, gelatin and hyalouronic acid.

In particular embodiments, polymers disclosed herein can include “star shaped polymers,” which refer to branched polymers in which two or more polymer branches extend from a core. The core is a group of atoms having two or more functional groups from which the branches can be extended by polymerization.

In particular embodiments, the branches are zwitterionic or negatively-charged polymeric branches. For star polymers, the branch precursors can be converted to zwitterionic or negatively-charged polymers via hydrolysis, ultraviolet irradiation, or heat. The polymers also may be obtained by any polymerization method effective for polymerization of unsaturated monomers, including atom transfer radical polymerization (ATRP), reversible addition-fragmentation chain transfer polymerization (RAFT), photo-polymerization, ring-opening polymerization (ROP), condensation, Michael addition, branch generation/propagation reaction, or other reactions.

Liposomes are microscopic vesicles including at least one concentric lipid bilayer. Vesicle-forming lipids are selected to achieve a specified degree of fluidity or rigidity of the final complex. In particular embodiments, liposomes provide a lipid composition that is an outer layer surrounding a porous nanoparticle.

Liposomes can be neutral (cholesterol) or bipolar and include phospholipids, such as phosphatidylcholine (PC), phosphatidylethanolamine (PE), phosphatidylinositol (PI), and sphingomyelin (SM) and other type of bipolar lipids including dioleoyl phosphatidylethanolamine (DOPE), with a hydrocarbon chain length in the range of 14-22, and saturated or with one or more double C═C bonds. Examples of lipids capable of producing a stable liposome, alone, or in combination with other lipid components are phospholipids, such as hydrogenated soy phosphatidylcholine (HSPC), lecithin, phosphatidylethanolamine, lysolecithin, lysophosphatidylethanolamine, phosphatidylserine, phosphatidylinositol, sphingomyelin, cephalin, cardiolipin, phosphatidic acid, cerebrosides, distearoylphosphatidylethanolamine (DSPE), dioleoylphosphatidylcholine (DOPC), dipalmitoylphosphatidylcholine (DPPC), palmitoyloleoylphosphatidylcholine (POPC), palmitoyloleoylphosphatidylethanolamine (POPE) and dioleoylphosphatidylethanolamine 4-(N-maleimido-methyl)cyclohexane-1-carboxylate (DOPE-mal). Additional non-phosphorous containing lipids that can become incorporated into liposomes include stearylamine, dodecylamine, hexadecylamine, isopropyl myristate, triethanolamine-lauryl sulfate, alkyl-aryl sulfate, acetyl palmitate, glycerol ricinoleate, hexadecyl stereate, amphoteric acrylic polymers, polyethyloxylated fatty acid amides, DDAB, dioctadecyl dimethyl ammonium chloride (DODAC), 1,2-dimyristoyl-3-trimethylammonium propane (DMTAP), DOTAP, DOTMA, DC-Chol, phosphatidic acid (PA), dipalmitoylphosphatidylglycerol (DPPG), dioleoylphosphatidylglycerol, DOPG, and dicetylphosphate. In particular embodiments, lipids used to create liposomes disclosed herein include cholesterol, hydrogenated soy phosphatidylcholine (HSPC) and, the derivatized vesicle-forming lipid PEG-DSPE.

Methods of forming liposomes are described in, for example, U.S. Pat. Nos. 4,229,360; 4,224,179; 4,241,046; 4,737,323; 4,078,052; 4,235,871; 4,501,728; and 4,837,028, as well as in Szoka et al., Ann. Rev. Biophys. Bioeng. 9:467, 1980; and Hope et al., Chem. Phys. Lip. 40:89, 1986.

Further exemplary methods of generating nanoparticles are disclosed in US2018/0030153, US2017/0296676, and WO2018/129270, the disclosures of which are incorporated herein in their entireties for all purposes.

In particular embodiments, the coating is polymer-based with a polymer size of 5-100 kDa. In particular embodiments, the coating is polymer-based with a polymer size of 5, 10, 15, 20, 25, 30, 35, 40, 45, 50, 55, 60, 65, 70, 75, 80, 85, 90, 95, or 100 kDa.

In particular embodiments, PbAE polymers are mixed with nucleotides (e.g., IVT mRNA) in a ratio of 20:1, 30:1, 40:1, 50:1, 60:1, 70:1, 80:1, 90:1, 100:1, or more to generate PbAE-nucleotide polyplexes. In particular embodiments, PbAE polymers are mixed with nucleotides (e.g., IVT mRNA) in a ratio of 60:1 to generate PbAE-nucleotide polyplexes. In particular embodiments, the PbAE-nucleotide polyplexes can be combined with PGA/Di-mannose to form the nanoparticles.

The size of the nanoparticles disclosed herein can vary over a wide range and can be measured in different ways, for example by dynamic light scattering and/or electron microscopy. For example, the nanoparticles of the present disclosure can have a minimum dimension of 100 nm. The nanoparticles of the present disclosure can also have a minimum dimension of equal to or less than 500 nm, less than 150 nm, less than 100 nm, less than 90 nm, less than 80 nm, less than 70 nm, less than 60 nm, less than 50 nm, less than 40 nm, less than 30 nm, less than 20 nm, or less than 10 nm. In particular embodiments, the nanoparticles can have a minimum dimension ranging between 5 nm and 500 nm, between 10 nm and 100 nm, between 20 nm and 90 nm, between 30 nm and 80 nm, between 40 nm and 70 nm, and between 40 nm and 60 nm. In particular embodiments, the dimension is the diameter of nanoparticles or coated nanoparticles. In particular embodiments, a population of nanoparticles of the present disclosure can have a mean minimum dimension of equal to or less than 500 nm, less than 100 nm, less than 90 nm, less than 80 nm, less than 70 nm, less than 60 nm, less than 50 nm, less than 40 nm, less than 30 nm, less than 20 nm, or less than 10 nm. In particular embodiments, a population of nanoparticles in a composition of the present disclosure can have a mean diameter ranging between 5 nm and 500 nm, between 10 nm and 100 nm, between 20 nm and 90 nm, between 30 nm and 80 nm, between 40 nm and 70 nm, and between 40 nm and 60 nm.

(iv) Compositions. The nanoparticles disclosed herein can be formulated into compositions for direct administration to a subject, wherein the selective targeting occurs in vivo. Optionally, more than one nanoparticle—that is, nanoparticles containing different passenger nucleic acids, encoding different therapeutic proteins—can be administered to the same subject in concert, whether sequentially or simultaneously.

In particular embodiments, the nanoparticles are provided as part of composition that can include at least 0.1% w/v or w/w of nanoparticles; at least 1% w/v or w/w of nanoparticles; at least 10% w/v or w/w of nanoparticles; at least 20% w/v or w/w of nanoparticles; at least 30% w/v or w/w of nanoparticles; at least 40% w/v or w/w of nanoparticles; at least 50% w/v or w/w of nanoparticles; at least 60% w/v or w/w of nanoparticles; at least 70% w/v or w/w of nanoparticles; at least 80% w/v or w/w of nanoparticles; at least 90% w/v or w/w of nanoparticles; at least 95% w/v or w/w of nanoparticles; or at least 99% w/v or w/w of nanoparticles.

The compositions disclosed herein can be formulated for administration by, injection, inhalation, infusion, perfusion, lavage, or ingestion. The compositions disclosed herein can further be formulated for infusion via catheter, intravenous, intramuscular, intratumoral, intradermal, intraarterial, intranodal, intralymphatic, intraperitoneal, intralesional, intraprostatic, intravaginal, intrarectal, topical, intrathecal, intravesicular, oral and/or subcutaneous administration and more particularly by intravenous, intradermal, intraarterial, intranodal, intralymphatic, intraperitoneal, intralesional, intravaginal, intrarectal, topical, intrathecal, intratumoral, intramuscular, intravesicular, oral and/or subcutaneous injection.

For injection and infusion, compositions can be formulated as aqueous solutions, such as in buffers including Hanks' solution, Ringer's solution, or physiological saline. The aqueous solutions can contain formulatory agents such as suspending, stabilizing and/or dispersing agents. Alternatively, the formulation can be in lyophilized and/or powder form for constitution with a suitable vehicle, e.g., sterile pyrogen-free water, before use.

For oral administration, the compositions can be formulated as tablets, pills, dragees, capsules, liquids, gels, syrups, slurries, suspensions, and the like. For oral solid formulations such as, for example, powders, capsules and tablets, suitable excipients include binders (gum tragacanth, acacia, cornstarch, gelatin), fillers such as sugars, e.g. lactose, sucrose, mannitol and sorbitol; dicalcium phosphate, starch, magnesium stearate, sodium saccharine, cellulose, magnesium carbonate; cellulose preparations such as maize starch, wheat starch, rice starch, potato starch, gelatin, gum tragacanth, methyl cellulose, hydroxypropylmethyl-cellulose, sodium carboxy-methylcellulose, and/or polyvinylpyrrolidone (PVP); granulating agents; and binding agents. If desired, disintegrating agents can be added, such as corn starch, potato starch, alginic acid, cross-linked polyvinylpyrrolidone, agar, or alginic acid or a salt thereof such as sodium alginate. If desired, solid dosage forms can be sugar-coated or enteric-coated using standard techniques. Flavoring agents, such as peppermint, oil of wintergreen, cherry flavoring, orange flavoring, etc. can also be used.

For administration by inhalation, compositions can be formulated as aerosol sprays from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of gelatin for use in an inhaler or insufflator may be formulated containing a powder mix of the therapeutic and a suitable powder base such as lactose or starch.

Any composition formulation disclosed herein can advantageously include any other pharmaceutically acceptable carriers which include those that do not produce significantly adverse, allergic, or other untoward reactions that outweigh the benefit of administration, whether for research, prophylactic and/or therapeutic treatments. Exemplary pharmaceutically acceptable carriers and formulations are disclosed in Remington's Pharmaceutical Sciences, 18th Ed. Mack Printing Company, 1990. Moreover, formulations can be prepared to meet sterility, pyrogenicity, general safety and purity standards as required by United States FDA Office of Biological Standards and/or other relevant foreign regulatory agencies.

Exemplary generally used pharmaceutically acceptable carriers include any and all bulking agents or fillers, solvents or co-solvents, dispersion media, coatings, surfactants, antioxidants (e.g., ascorbic acid, methionine, vitamin E), preservatives, isotonic agents, absorption delaying agents, salts, stabilizers, buffering agents, chelating agents (e.g., EDTA), gels, binders, disintegration agents, and/or lubricants.

Exemplary buffering agents include citrate buffers, succinate buffers, tartrate buffers, fumarate buffers, gluconate buffers, oxalate buffers, lactate buffers, acetate buffers, phosphate buffers, histidine buffers and/or trimethylamine salts.

Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens such as methyl or propyl paraben, catechol, resorcinol, cyclohexanol and 3-pentanol.

Exemplary isotonic agents include polyhydric sugar alcohols including trihydric or higher sugar alcohols, such as glycerin, erythritol, arabitol, xylitol, sorbitol, or mannitol.

Exemplary stabilizers include organic sugars, polyhydric sugar alcohols, polyethylene glycol; sulfur-containing reducing agents, amino acids, low molecular weight polypeptides, proteins, immunoglobulins, hydrophilic polymers, or polysaccharides.

Compositions can also be formulated as depot preparations. Depot preparations can be formulated with suitable polymeric or hydrophobic materials (for example as an emulsion in an acceptable oil) or ion exchange resins, or as sparingly soluble derivatives, for example, as a sparingly soluble salts.

Additionally, compositions can be formulated as sustained-release systems utilizing semipermeable matrices of solid polymers containing at least one active ingredient. Various sustained-release materials have been established and are well known by those of ordinary skill in the art. Sustained-release systems may, depending on their chemical nature, release active ingredients following administration for two weeks to 1 month. In particular embodiments, a sustained-release system could be utilized, for example, if a human patient were to miss a weekly administration. The half-life of particular embodiments of nanoparticles described herein is 4 hours. To sustain release, the nanoparticles can be encapsulated within a hydrogel or biodegradable polymer that slowly releases the nanoparticles over time. The mRNA itself is stable when condensed, for example, within a PBAE polymer.

(v) Methods of Use. Methods disclosed herein include treating subjects (including humans, veterinary animals, livestock, and research animals) with compositions disclosed herein. As indicated the compositions can treat a variety of different conditions, ranging from cancer to infectious disease.

Therapeutically Effective Treatments. Treating subjects includes delivering therapeutically effective amounts of one or more composition(s). Therapeutically effective amounts can provide effective amounts, prophylactic treatments, and/or therapeutic treatments.

An “effective amount” is the amount of a compound necessary to result in a desired physiological change in the subject. Effective amounts are often administered for research purposes. For example, effective amounts disclosed herein result in expression (e.g., transient expression) of a nucleic acid or protein, such as a therapeutic protein, by a selected cell type following administration to a subject. As a further example, an effective amount of a cell attractant when administered to a subject results in recruitment of a particular cell type (e.g., T cells) to the site of administration.

A “prophylactic treatment” includes a treatment administered to a subject who does not display signs or symptoms of a disease or condition or displays only early signs or symptoms of the disease or condition such that treatment is administered for the purpose of diminishing, preventing, or decreasing the risk of developing the disease or condition further. Thus, a prophylactic treatment functions as a preventative treatment against a disease or disorder. Vaccines are one example of prophylactic treatments.

In particular embodiments, prophylactic treatments are administered to treat viral infections, such as HIV. For example, the compositions can be administered prophylactically in subjects who are at risk of developing a viral infection, or who have been exposed to a virus, to prevent, reduce, or delay the development of viral infection or disease. For example, the compositions can be administered to a subject likely to have been exposed to a virus (e.g., HIV) or to a subject who is at high risk for exposure to a virus.

A “therapeutic treatment” includes a treatment administered to a subject who displays symptoms or signs of a disease or condition and is administered to the subject for the purpose of diminishing or eliminating those signs or symptoms of the disease or condition. A “therapeutic treatment” results in a desired therapeutic benefit in the subject.

Prophylactic and therapeutic treatments need not fully prevent or cure a disease or condition but can also provide a partial benefit.

In the context of cancers, therapeutically effective amounts can decrease the number of tumor cells, decrease the number of metastases, decrease tumor volume, increase life expectancy, induce apoptosis of cancer cells, induce cancer cell death, induce chemo- or radiosensitivity in cancer cells, inhibit angiogenesis near cancer cells, inhibit cancer cell proliferation, inhibit tumor growth, prevent metastasis, prolong a subject's life, reduce cancer-associated pain, reduce the number of metastases, and/or reduce relapse or re-occurrence of the cancer following treatment.

In the context of viruses, therapeutically effective amounts can decrease the number of virally-infected cells, and reduce one or more symptoms associated with the viral infection, such as fever, chills, vomiting, joint pain, etc.

In the context of HIV, therapeutically effective amounts can decrease the number of HIV-infected cells, increase a subject's number of T cells, reduce incidence, frequency, or severity of infections, increase life expectancy, prolong a subject's life, and/or reduce HIV-associated pain or cognitive impairments.

For administration, therapeutically effective amounts (also referred to herein as doses) can be initially estimated based on results from in vitro assays and/or animal model studies. Such information can be used to more accurately determine useful doses in subjects of interest.

The actual dose amount administered to a particular subject can be determined by a physician, veterinarian or researcher taking into account parameters such as physical and physiological factors including target, body weight, severity of condition, type of disease, previous or concurrent therapeutic interventions, idiopathy of the subject and route of administration.

Useful doses of compositions can include from 0.1 to 5 μg/kg, or from 0.5 to 1 μg/kg, or from 1-1000 mg/kg or more.

Therapeutically effective amounts, which obtain a therapeutic goal or effect, can be achieved by administering single or multiple doses during the course of a treatment regimen. Such doses may be administered, for instance, daily, every other day, every 4 days, every 2-8 days, every 3-10 days, every 5-10 days, every 6-9 days, weekly, or every fortnight. Optionally, the time between dosages may vary. In some embodiments, a single dose will provide the desired therapeutic effect; in others, multiple doses will be required. In particular embodiments, the effectiveness of the treatment regimen, and the need for additional dose(s), can be monitored by determining, tracking, or measuring a phenotypic effect mediated by the transiently expressed therapeutic protein or nucleic acid.

In particular embodiments, once the expression level of a protein or nucleic acid (e.g., a therapeutic protein) falls below a threshold, a treating physician can make a determination whether an additional treatment with the nanoparticle is warranted or if a therapeutic objective has been achieved and that an additional treatment with the nanoparticle is not warranted at that time. In particular embodiments, below a threshold can be 50%, 45%, 40%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1%, or less of peak expression levels as measured by quantitative PCR or flow cytometry. In particular embodiments, below a threshold can be 15%, 14%, 13%, 12%, 11%, 10%, 9%, 8%, 7%, 6%, 5%, 4%, 3%, 2%, 1%, or less of peak expression levels as measured by quantitative PCR. In particular embodiments, the threshold can be 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1%, or less of nanoparticle-transfected T cells expressing the protein or nucleic acid as measured by flow cytometry. In particular embodiments, the threshold can be 20%, 19%, 18%, 17%, 16%, 15%, 14%, 13%, 12%, 11%, 10%, 9%, 8%, 7%, 6%, 5%, 4%, 3%, 2%, 1%, or less of CD8+ T cells in peripheral blood expressing the therapeutic protein. In particular embodiments, the threshold can be tumor cell count obtained in in vitro live cell assays to measure the ability of IVT mRNA-transfected T cells to selectively lyse antigen-positive target cells.

In particular embodiments, once the expression level of a protein or nucleic acid (e.g., a therapeutic protein) falls below a detectable limit, a treating physician can make a determination whether an additional treatment with the nanoparticle is warranted or if a therapeutic objective has been achieved and that an additional treatment with the nanoparticle is not warranted at that time.

In particular embodiments, expression of a protein or nucleic acid (e.g., a therapeutic protein) falls below a detectable limit when its expression is not detected by quantitative PCR. In particular embodiments, the detectable limit can be 1%, 0.9%, 0.8%, 0.7%, 0.6%, 0.5%, 0.4%, 0.3%, 0.2%, 0.1%, 0.005%, 0.001%, or less of the subject's T cells expressing the protein or nucleic acid as measured by flow cytometry. In particular embodiments, the detectable limit can be a percentage of CD8+ T cells in peripheral blood expressing the therapeutic protein. In particular embodiments, the detectable limit can be 2%, 1.5%, 1%, 0.5%, 1%, 0.9%, 0.8%, 0.7%, 0.6%, 0.5%, 0.4%, 0.3%, 0.2%, 0.1%, 0.005%, 0.001%, or less of CD8+ T cells in the subject's peripheral blood expressing the protein or nucleic acid.

In particular embodiments, methods of the disclosure result in at least about the same efficacy as transplantation of T cells contacted with a nanocarrier ex vivo. In particular embodiments, at least about the same efficacy includes comparing the function of nanoparticle-transfected T cells (i.e., IVT mRNA-transfected T cells, where the IVT mRNA encodes a therapeutic protein) with that of T cells engineered with the same corresponding therapeutic protein ex vivo. In particular embodiments, the ex vivo engineered T cells are transduced by viral methods. In particular embodiments, the function to compare is cell killing in an in vitro assay. In particular embodiments, at least about the same efficacy includes no statistically significant difference in killing of antigen-positive target cells by nanoparticle-transfected T cells as compared to T cells engineered with the same corresponding therapeutic protein ex vivo. In particular embodiments, the function to compare is cytokine production in an in vitro assay. In particular embodiments, at least about the same efficacy includes no statistically significant difference in level of cytokine production including IL-2, TNF-α, and IFN-γ by nanoparticle-transfected T cells as compared to T cells engineered with the same corresponding therapeutic protein ex vivo. In particular embodiments, the comparison can include tumor size or growth in vivo. In particular embodiments, at least about the same efficacy includes no statistically significant difference in tumor size or growth in subjects transfused with IVT mRNA encoding a therapeutic protein as compared to subjects receiving adoptive T cell therapy including T cells transduced with the same corresponding therapeutic protein. In particular embodiments, the comparison can include survival of subjects. In particular embodiments, at least about the same efficacy includes no statistically significant difference in survival of subjects transfused with IVT mRNA encoding a therapeutic protein as compared to subjects receiving adoptive T cell therapy including T cells transduced with the same corresponding therapeutic protein.

Statistical significance in observations can be determined by a statistical method known to one of ordinary skill in the art. In particular embodiments, no statistically significant difference refers to a p value >0.05 or >0.01.

Therapeutically effective Treatments in concert with selected Cell Attractants. In particular embodiments, nanoparticles delivering a nucleic acid to provide expression of a therapeutic protein by selected cell types can be administered in concert with a cell attractant. “In concert with” means that the nanoparticles and cell attractants are administered within a clinically relevant time window. A “clinically relevant time window” means within a time period where an increased therapeutic effect is seen based on the administration of the nanoparticles and the cell attractants over what is seen based on the administration of the nanoparticles or the cell attractants alone. Usually, a cell attractant is administered before the nanoparticles, but this timing is not necessary if a clinically relevant time window permits administration of the cell attractant after the nanoparticles.

In particular embodiments, a cell attractant is administered (locally or systemically) to the subject at least one hour and up to two weeks before the expression nanoparticle is administered. For instance, the cell attractant is administered at least one hour, at least 3 hours, at least 6 hours, at least 9 hours, at least 12 hours, at least 24 hours, or more than 24 hours before administration of the nanoparticle composition. In certain embodiments, the preconditioning occurs between one and 24 hour before administration of the nanoparticle, or between one hour and seven days before. In particular embodiments, cell attractants can be co-delivered with T-cell programming nanoparticles.

Therapeutically Effective Treatments Administered in Concert with Macrophage Stimulating Compositions. In particular embodiments, delivery of a nanoparticle containing a transiently expressed mRNA can occur in concert with another treatment strategy, such as treatment with a second targeted nanoparticle that expresses (from DNA or mRNA) a different therapeutic protein. By way of example, macrophage stimulating (macrophage activating) nanoparticle composition(s) are used as the exemplified second therapeutic composition. It will be appreciated that co-administration of additional types of targeted nanoparticles, as well as additional non-nanoparticle therapeutics, is also contemplated.

For example, herein described nanoparticles (including an IVT mRNA encoding a therapeutic protein, such as a disease specific receptor) can be administered (concurrently or in sequence) to a subject along with a nanoparticle composition that stimulates macrophages or overcomes tumor suppression of macrophage(s) of the subject being treated. Such macrophage activating compositions may be themselves nanoparticles that include a nucleic acid encoding a therapeutic protein that reverses or reduces immunosuppression of macrophages, for instance a transcription factor. Examples of such macrophage-activating nanoparticles are structured similarly to nanoparticles described herein (e.g., they have a positive core and a neutral or negatively-charged surface, and deliver nucleotide(s) for expression in the targeted cell). Particular embodiments utilize particles to provide cells with nucleotides encoding genes encoding activation regulators such as transcription factors (e.g., Interferon Regulatory Factors (IRFs)) and/or kinases (e.g., IKKβ) that regulate macrophage polarization. Macrophage polarization is a highly dynamic process through which the physiological activity of macrophages changes. In most tumors, TAMs exhibit an immunosuppressed phenotype which can be an “M2” phenotype. By contrast, activated macrophages can exhibit an “M1” phenotype which results in tumor cell killing. Particular embodiments disclosed herein reverse the polarization of tumor-promoting TAMs into tumor-killing macrophages. This effect ameliorates the immunosuppressive milieu within the tumors by inducing inflammatory cytokines, activating other immune cells, and phagocytosing tumor cells.

By way of example, the passenger nucleic acid(s) in a macrophage-stimulating nanoparticle in some embodiments encode (as DNA or IVT mRNA) the transcription factor interferon-regulatory factor 5 (IRF5) in combination with the kinase IKKβ. Such particles can include a tumor-associated macrophage (TAM) targeting ligand to direct more selective uptake of the particles by TAMs. As one example, TAMs express CD206, a cellular surface receptor that can be targeted by including mannose on the surface of the particles. Other TAM cell surface receptors that can be targeted include early growth response protein 2 (Egr2), CD163, CD23, interleukin (IL)27RA, CLEC4A, CD1a, CD1b, CD93, CD226, IL13-Ra1, IL-4r, IL-1R type II, decoy IL-1R type II, IL-10r, macrophage scavenging receptors A and B, Ym-1, Ym-2, Low density receptor-related protein 1 (LRP1), IL-6r, CXCR1/2, and PD-L1.

Particular embodiments include repeated delivery of nanoparticle compositions to a patient that target selected cells within the patient and result in expression of a therapeutic protein or nucleic acid by the selected cells. In this context, transient expression refers to the expression of a therapeutic protein over a short time period following nucleic acid transfer into cell(s). Such expression can be monitored in various art-recognized ways, including by detection and/or quantification of a phenotype of a cell, which phenotype is generated or influenced by the expressed therapeutic protein or nucleic acid. The phenotype of a cell refers to its physical characteristics and/or its location within the body. In particular embodiments, a researcher or clinician selects a nanoparticle for delivery based on a transient expression profile that it provides.

As indicated previously, in particular embodiments, a transient expression profile lasts from 12 hours to 15 days; from 18 hours to 12 days; from 20 hours to 14 days; from 24 hours to 10 days, from 24 hours to 8 days, or from 30 hours to 7 days. It is specifically contemplated that transient expression in various embodiments is no longer than 14 days. For instance, in particular embodiments transient expression is detectable expression which lasts no longer than 12 days, no longer than 10 days, no longer than 9 days, no longer than 8 days, or no longer than 7 days. In embodiments, where longer expression is desired, a nanoparticle providing transient expression of a therapeutic protein can be delivered to a subject with repeated doses, for instance delivery that occurs every 5-10 days (e.g., every 7 days).

(vi) Kits. Combinations of active components can also be provided as kits. Kits can include containers including one or more or more expression nanoparticles as described herein, optionally along with one or more agents for use in combination therapy. For instance, some kits will include at least one expression nanoparticle, along with an amount of at least one macrophage stimulating composition (which itself may be a nanoparticle containing a mRNA or DNA molecule encoding, for instance, an amount of at least one macrophage stimulating protein). Other kits will include an amount of at least one expression nanoparticle along with an amount of at least one cell attractant, such as a T cell attractant. Any active component in a kit may be provided in premeasured dosages, though this is not required; and it is anticipated that certain kits will include more than one dose, including for instance when the kit is used for a method requiring administration of more than one dose of the desired expression nanoparticle.

Generally, a kit that includes two or more active components will include components intended to be used in conjunction in one of the methods described herein. For instance a macrophage activating compound would be provided in a kit containing a nanoparticle designed to provide expression in a tumor or another site that would benefit from the presence of activated macrophage. Similarly, if a kit is provided with a cell attractant, then at least one nanoparticle included in the kit will in some instances target a cell type attracted by that cell attractant.

Kits can also include a notice in the form prescribed by a governmental agency regulating the manufacture, use, or sale of pharmaceuticals or biological products, which notice reflects approval by the agency of manufacture, use, or sale for human administration. The notice may state that the provided active ingredients can be administered to a subject. The kits can include further instructions for using the kit, for example, instructions regarding preparation of polynucleotides (PN) or nanoparticles (NP), for administration; proper disposal of related waste; and the like. The instructions can be in the form of printed instructions provided within the kit or the instructions can be printed on a portion of the kit itself. Instructions may be in the form of a sheet, pamphlet, brochure, CD-ROM, or computer-readable device, or can provide directions to instructions at a remote location, such as a website. In particular embodiments, kits can also include some or all of the necessary medical supplies needed to use the kit effectively, such as syringes, ampules, tubing, facemask, an injection cap, sponges, sterile adhesive strips, Chloraprep, gloves, and the like. Variations in contents of any of the kits described herein can be made. The instructions of the kit will direct use of the active ingredients to effectuate a new clinical use described herein.

### (vii) Exemplary Embodiments

1. A method for treating a subject in need thereof, including administering to the subject:
   a therapeutically effective amount of a nanoparticle including:
      (i) a polynucleotide (e.g., synthetic mRNA, such as in vitro transcribed (IVT) mRNA) encapsulated within a positively-charged carrier matrix, wherein the polynucleotide encodes a protein and/or a nucleic acid;
      (ii) a neutrally or negatively-charged coating; and
      (iii) at least one cell targeting ligand extending from the surface of the coating, which cell targeting ligand is specific for selected cells;
   wherein the nanoparticles are selectively incorporated into the selected cells within the subject such that the selected cells transiently express the protein from the polynucleotide, thereby treating the subject in need thereof.
2. A method of embodiment 1, further including administering to the subject a therapeutically effective amount of a cell attractant.
3. A method of embodiment 2, wherein the cell attractant is a T cell attractant.
4. A method of embodiment 3, wherein the T cell attractant is CCL21 or IP10.
5. A method of any of embodiments 1-4, wherein the protein includes a disease specific receptor including a cell surface receptor.
6. A method of embodiment 5, wherein the disease specific receptor includes a chimeric antigen receptor (CAR), a T cell receptor (TCR), or a combination thereof.
7. A method of embodiment 5 or 6, wherein the therapeutic protein includes a leukemia-specific CAR, a Hepatitis B virus (HBV) core antigen specific HBcore18-27 TCR, or a prostate tumor specific anti-ROR1 CAR.
8. A method of any of embodiments 1-7, wherein the cell targeting ligand selectively binds lymphocytes and initiates receptor-induced endocytosis.
9. A method of any of embodiments 1-8, wherein the expression of the protein is expression for no longer than 14 days, no longer than 12 days, no longer than 10 days, no longer than 9 days, no longer than 8 days, no longer than 7 days, no longer than 6 days, or no longer than 5 days.
10. A method of any of embodiments 1-9, wherein administering the therapeutically effective amount of a nanoparticle to the subject includes administering two or more doses of the nanoparticle.
11. The method of embodiment 10, wherein the two or more doses are administered every 5-10 days, or every 6-8 days, or every 7 days.
12. A method of any of embodiments 1-11, wherein the subject is in need of treatment for cancer or an infectious disease.
13. A method of any of embodiments 1-12, wherein administering the nanoparticle includes injection or infusion via catheter (a) into or proximal to a tumor (intratumoral), (b) into a vein (intravenous), or (c) into the peritoneum (intraperitoneally).
14. A method of any of embodiments 3-13, wherein administering the T cell attractant includes injection or infusion via catheter into or proximal to a tumor (intratumoral), intravenous injection or infusion, or injection or infusion via catheter interperitoneally.
15. A method of any of embodiments 2-14, wherein the cell attractant is administered to the subject before the nanoparticle is administered.
16. A method of any of embodiments 2-14, wherein the cell attractant is administered no more than one hour before, no more than 3 hours before, no more than 6 hours before, no more than 12 hours before, or no more than 24 hours before the nanoparticle is administered.
17. A method of embodiment 16, wherein the cell attractant is administered (a) only after the first dose of the nanoparticle; (b) after each of at least two doses of the nanoparticle; or (c) after each dose of the nanoparticle.
18. A method of any of embodiments 1-17, further including administering a macrophage stimulating composition to the subject.
19. A method of embodiment 18, wherein the macrophage stimulating composition includes s a nanoparticle targeted to macrophage cells and capable of directing expression of transcription factor interferon-regulatory factor 5 (IRF5) in combination with the kinase IKKβ.
20. A method of any of embodiments 1-19, wherein the carrier includes a positively charged lipid or polymer.
21. A method of embodiment 20, wherein the positively charged polymer includes poly(β-amino ester) (PBAE), poly(L-lysine), poly(ethylene imine) (PEI), poly-(amidoamine) dendrimers (PAMAMs), poly(amine-co-esters), poly(dimethylaminoethyl methacrylate) (PDMAEMA), chitosan, poly-(L-lactide-co-L-lysine), poly[α-(4-aminobutyl)-L-glycolic acid] (PAGA), or poly(4-hydroxy-L-proline ester) (PHP).
22. A method of any of embodiments 1-21, wherein the coating includes a neutrally or negatively-charged lipid or polymer.
23. A method of embodiment 22, wherein the neutrally or negatively-charged coating includes polyglutamic acid (PGA), poly(acrylic acid), alginic acid, or cholesteryl hemisuccinate/1,2-dioleoyl-sn-glycero-3-phosphoethanolamine.
24. A method of embodiment 22, wherein the neutrally or negatively-charged coating includes a zwitterionic polymer.
25. A method of any of embodiments 22-24, wherein the neutrally or negatively-charged coating includes a liposome.
26. A method of embodiment 25, wherein the liposome includes 1,2-dioleoyl-3-trimethylammonium-propane (DOTAP), 1,2-di-O-octadecenyl-3-trimethylammonium propane (DOTMA), 3β[N—(N′,N′-dimethylaminoethane)-carbamoyl]cholesterol (DC-Chol), dioctadecyl-amidoglycylspermine (DOGS), cholesterol, 1,2-dioleoyl-sn-glycero-3-phosphoethanolamine (DOPE), or 1,2-dioleoyl-sn-glycero-3-phosphocholine (DOPC).
27. A method of any of embodiments 1-26, wherein the selected cell targeting ligand selectively binds CD4 and/or CD8.
28. A method of any of embodiments 1-27, wherein the selected cell targeting ligand includes a binding domain selected from a CD4 antibody and/or a CD8 antibody.
29. A method of any of embodiments 1-28, wherein the selected cell targeting ligand includes a binding domain selected from an scFv fragment of a CD4 antibody and/or a CD8 antibody.
30. A method of any of embodiments 1-29, wherein the carrier includes PBAE (e.g., PBAE 447 and/or with 1-(3-aminopropyl)pyrrolidine end caps).
31. A method of any of embodiments 1-30, wherein the coating includes PGA.
32. A method of any of embodiments 1-31, wherein the selected cell targeting ligand includes a binding domain selected from a CD4 antibody and/or a CD8 antibody; the carrier includes PBAE (e.g., PBAE 447 and/or with 1-(3-aminopropyl)pyrrolidine end caps)); and the coating includes PGA.
33. A synthetic nanoparticle including:
   (i) a polynucleotide (e.g., synthetic mRNA, such as IVT mRNA) encoding a protein or a nucleic acid and encapsulated within a positively-charged carrier;
   (ii) a neutrally or negatively-charged coating on the outer surface of the carrier; and
   (iii) a selected cell targeting ligand extending from the surface of the coating;

wherein the protein can be selected from a HBV specific TCR, a leukemia-specific anti-CD19 CAR, or a prostate tumor-specific anti-ROR1 CAR, wherein the intracellular domain of the CAR can be 1928z or 4-1BBz.
34. A synthetic nanoparticle of embodiment 33, wherein the carrier includes a positively charged lipid or polymer.
35. A synthetic nanoparticle of embodiment 34, wherein the positively charged lipid or polymer includes PBAE, poly(L-lysine), PEI, PAMAMs, poly(amine-co-esters), PDMAEMA, chitosan, poly-(L-lactide-co-L-lysine), PAGA, or PHP.
36. A synthetic nanoparticle of embodiment 33 or 34, wherein the coating includes a neutrally or negatively-charged lipid or polymer.
37. A synthetic nanoparticle of embodiment 36, wherein the neutrally or negatively-charged coating includes PGA, poly(acrylic acid), alginic acid, or cholesteryl hemisuccinate/1,2-dioleoyl-sn-glycero-3-phosphoethanolamine.
38. A synthetic nanoparticle of embodiment 36, wherein the neutrally or negatively-charged coating includes a zwitterionic polymer.
39. A synthetic nanoparticle of any of embodiments 36-38, wherein the neutrally or negatively-charged coating includes a liposome.
40. A synthetic nanoparticle of embodiment 39 wherein the liposome includes DOTAP, DOTMA, DC-Chol, DOGS, cholesterol, DOPE, or DOPC.
41. A synthetic nanoparticle of any of embodiments 33-40, wherein the selected cell targeting ligand selectively binds CD4 and/or CD8.
42. A synthetic nanoparticle of any of embodiments 33-41, wherein the selected cell targeting ligand includes a binding domain selected from a CD4 antibody and/or a CD8 antibody.
43. A synthetic nanoparticle of any of embodiments 33-42, wherein the selected cell targeting ligand includes a binding domain selected from an scFv fragment of a CD4 antibody and/or a CD8 antibody.
44. A synthetic nanoparticle of any of embodiments 33-43, wherein the carrier includes PBAE (e.g., PBAE 447 and/or with 1-(3-aminopropyl)pyrrolidine end caps)).
45. A synthetic nanoparticle of any of embodiments 33-44, wherein the coating includes PGA.
46. A synthetic nanoparticle of any of embodiments 33-45, wherein the selected cell targeting ligand includes a binding domain selected from a CD4 antibody and/or a CD8 antibody; the carrier includes PBAE (e.g., PBAE 447 and/or with 1-(3-aminopropyl)pyrrolidine end caps)); and the coating includes PGA.
47. A composition including a synthetic nanoparticle of any of embodiments 33-46.
48. A method of treating a subject in need thereof including administering a therapeutically effective amount of a composition of embodiment 47 thereby treating the subject in need thereof.
49. A method of embodiment 48, further including administering to the subject a T cell attractant before administering the nanoparticle or composition.
50. A method for treating a subject in need thereof, including selecting a nanoparticle that results in expression of a protein or nucleic acid by a selected cell type following administration to the subject and administering a therapeutically effective amount of the selected nanoparticle to the subject thereby treating the subject in need thereof wherein the expression of the protein or nucleic acid falls below a detectable limit within 10 days of administration.
51. The method of embodiment 50, wherein the expression of the protein or nucleic acid falls below the detectable limit within 7 days of administration.
52. The method of embodiment 50 or 51, further including administering a second therapeutically effective amount of the selected nanoparticle to the subject.
53. The method of embodiment 52, wherein the administering of the second therapeutically effective amount occurs after expression of the protein or nucleic acid has fallen below the detectable limit.
54. The method of embodiment 52, wherein the administering of the second therapeutically effective amount occurs before expression of the protein or nucleic acid has fallen below the detectable limit.
55. The method of embodiment 52, wherein the first therapeutically effective amount and the second therapeutically effective amount are administered 5 days apart, 6 days apart, 7 days apart, 8 days apart, 9 days a part or 10 days apart.
56. The method of any of embodiments 50-55, wherein the administering includes systemic or local administration.
57. The method of embodiment 56, wherein the administering includes local administration at a tumor site.
58. The method of any of embodiments 50-57, wherein administering includes injection or infusion via catheter (a) into or proximal to a tumor, (b) into a vein, or (c) into the peritoneum.
59. The method of any of embodiments 50-58, wherein the protein includes a disease specific receptor including a cell surface receptor.
60. The method of embodiment 59, wherein the disease specific receptor includes a CAR, a TCR, or a hybrid thereof.
61. The method of any of embodiments 50-60, wherein the therapeutic protein includes a HBV specific TCR, a leukemia-specific anti-CD19 CAR, or a prostate tumor-specific anti-ROR1 CAR, wherein the intracellular domain of the CAR can be 1928z or 4-1BBz.
62. The method of any of embodiments 50-61, wherein the protein includes a macrophage stimulating protein.
63. The method of embodiment 62, wherein the macrophage stimulating protein includes transcription factor IRF5 in combination with the kinase IKKβ.
64. The method of embodiment 62, wherein the macrophage stimulating protein includes one or more IRFs selected from IRF5, IRF1, IRF3, IRF7, IRF8, and/or a fusion of IRF7 and IRF3.
65. The method of embodiment 64, wherein the IRF7/IRF3 fusion protein includes SEQ ID NO: 39.
66. The method of any of embodiments 63-65, wherein the one or more IRFs lack a functional autoinhibitory domain.
67. The method of any of embodiments 63-66, wherein the one or more IRFs lack a functional nuclear export signal (NES).
68. The method of embodiment 64, wherein the one or more IRFs is selected from a sequence having >90%, >95%, or greater than 98% identity to SEQ ID NOs: 25-41.
69. The method of embodiment 64, wherein the one or more IRFs is IRF5 selected from SEQ ID NOs: 25-31.
70. The method of embodiment 69, wherein IRF5 includes SEQ ID NO: 25 or SEQ ID NO: 27 with one or more mutations selected from S156D, S158D and T160D.
71. The method of embodiment 69, wherein IRF5 includes SEQ ID NO: 26 with one or more mutations selected from T10D, S158D, S309D, S317D, S451D, and S462D.
72. The method of embodiment 69, wherein IRF5 includes SEQ ID NO: 28 with one or more mutations selected from S425D, S427D, S430D, and S436D.
73. The method of embodiment 64, wherein the one or more IRFs is IRF1 selected from SEQ ID NOs: 32 and 36.
74. The method of embodiment 64, wherein the one or more IRFs is IRF8 selected from SEQ ID NOs: 35, 40, and 41.
75. The method of embodiment 74, wherein IRF8 includes SEQ ID NO: 35 with a K310R mutation.
76. The method of any of embodiments 63-75, wherein the encoded IKKβ is selected from a sequence having >90%, >95%, or greater than 98% identity to SEQ ID NOs: 42-46.
77. The method of any of embodiments 63-75, wherein the encoded IKKβ is selected from SEQ ID NOs: 42-46.
78. The method of any of embodiments 50-77, wherein the therapeutic protein includes glucocorticoid-induced leuzine zipper (GILZ).
79. The method of any of embodiments 50-78, wherein the selected and administered nanoparticles are <130 nm.
80. The method of any of embodiments 50-79, wherein the selected and administered nanoparticles include:
   (i) a synthetic mRNA encapsulated within a positively-charged carrier matrix, wherein the synthetic mRNA encodes a therapeutic protein;
   (ii) a neutrally or negatively-charged coating; and
   (iii) at least one selected cell targeting ligand extending from the surface of the coating, which selected cell targeting ligand specifically binds a marker on the selected cell type.
81. The method of embodiment 80, wherein the synthetic mRNA includes IVT mRNA.
82. The method of embodiment 80 or 81, wherein the positively-charged carrier matrix includes a positively charged lipid or polymer.
83. The method of embodiment 82, wherein the positively charged lipid or polymer includes PBAE, poly(L-lysine), PEI, PAMAMs, poly(amine-co-esters), PDMAEMA, chitosan, poly-(L-lactide-co-L-lysine), PAGA, or PHP.
84. The method of any of embodiments 80-83, wherein the positively charged polymer includes PBAE (e.g., PBAE 447 and/or with 1-(3-aminopropyl)pyrrolidine end caps).
85. The method of any of embodiments 80-84, wherein the neutrally or negatively-charged coating includes PGA, poly(acrylic acid), alginic acid, or cholesteryl hemisuccinate/1,2-dioleoyl-sn-glycero-3-phosphoethanolamine.
86. The method of any of embodiments 80-85, wherein the neutrally or negatively-charged coating includes PGA.
87. The method of any of embodiments 80-85, wherein the neutrally or negatively-charged coating includes a zwitterionic polymer.
88. The method of any of embodiments 80-85, wherein the neutrally or negatively-charged coating includes a liposome.
89. The method of embodiment 88, wherein the liposome includes DOTAP, DOTMA, DC-Chol, DOGS, cholesterol, DOPE, or DOPC.
90. The method of any of embodiments 80-89, wherein the selected cell targeting ligand selectively binds lymphocytes and initiates receptor-induced endocytosis.
91. The method of any of embodiments 80-90, wherein the selected cell targeting ligand selectively binds CD4 and/or CD8.
92. The method of any of embodiments 80-91, wherein the selected cell targeting ligand includes a binding domain selected from a CD4 antibody and/or a CD8 antibody.
93. The method of any of embodiments 80-92, wherein the selected cell targeting ligand includes a binding domain selected from an scFv fragment of a CD4 antibody and/or a CD8 antibody.
94. The method of any of embodiments 80-93, wherein the selected cell targeting ligand includes a binding domain selected from a CD4 antibody and/or a CD8 antibody; the carrier includes PBAE (e.g., PBAE 447 and/or with 1-(3-aminopropyl)pyrrolidine end caps)); and the coating includes PGA.
95. The method of any of embodiments 80-89, wherein the selected cell targeting binds CD206, CD163, or CD23.
96. The method of any of embodiments 80-89, wherein the selected cell targeting ligand includes di-mannose.
97. The method of any of embodiments 80-89, wherein the selected cell targeting binds CD38, G-protein coupled receptor 18 (Gpr18), formyl peptide receptor 2 (Fpr2), CD64, or CD68.
98. The method of any of embodiments 50-97, further including administering to the subject a therapeutically effective amount of a cell attractant.
99. The method of embodiment 98, wherein the cell attractant includes a T cell attractant.
100. The method of embodiment 99, wherein the T cell attractant includes CCL21 or IP10.
101. The method of embodiment 99 or 100, wherein the T cell attractant includes CCL1, CCL2, CCL17, CCL22, CXCL9, CXCL10 or CXCL11.
102. The method of any of embodiments 98-101, wherein the cell attractant includes a monocyte/macrophage attractant.
103. The method of embodiment 102, wherein the monocyte/macrophage attractant includes CCL2, CCL3, CCLS, CCL7, CCL8, CCL13, CCL17 or CCL22.
104. The method of any of embodiments 98-103, wherein the cell attractant includes a mast cell attractant.
105. The method of embodiment 104, wherein the mast cell attractant includes CCL2 or CCL5.
106. The method of any of embodiments 98-105, wherein the cell attractant includes an eosinophil attractant.
107. The method of embodiment 106, wherein the eosinophil attractant includes CCL3, CCL5, CCL7, CCL11, CCL13, CCL24, or CCL26.
108. The method of any of embodiments 98-107, wherein the cell attractant includes a neutrophil attractant.
109. The method of embodiment 108, wherein the neutrophil attractant includes IL-8 or NAP1.
110. The method of any of embodiments 98-109, wherein the cell attractant is administered to the subject before the first therapeutically effective amount of nanoparticles is administered.
111. The method of any of embodiments 98-109, wherein the cell attractant is administered no more than one hour before, no more than 3 hours before, no more than 6 hours before, no more than 12 hours before, or no more than 24 hours before the first therapeutically effective amount of nanoparticles is administered.
112. The method of any of embodiments 98-109, wherein the cell attractant is administered at least one hour before, at least 3 hours before, at least 6 hours before, at least 12 hours before, or at least 24 hours before the first therapeutically effective amount of nanoparticles is administered.
113. The method of any of embodiments 98-112, wherein the cell attractant is administered (a) only after the first dose of the first therapeutically effective amount of nanoparticles is administered.
114. The method of any of embodiments 50-113, wherein the subject is in need of treatment for cancer or an infectious disease.
115. The method of embodiment 114, wherein the cancer is leukemia, prostate cancer, or hepatitis B-induced hepatocellular carcinoma, ovarian cancer, glioblastoma, or lung cancer.
116. A method of any of the preceding embodiments, wherein a researcher or clinician selects a nanoparticle described in any of the preceding embodiments for administration to a subject due to the selected nanoparticle's transient expression properties.
117. A method of embodiment 116, wherein the researcher or clinician administers the selected nanoparticle to the subject.
118. A method of embodiment 116 or 117 wherein the transient expression properties result in expression of a protein or nucleic acid for no longer than 14 days, no longer than 12 days, no longer than 10 days, no longer than 9 days, no longer than 8 days, no longer than 7 days, no longer than 6 days, or no longer than 5 days.

### (viii) Experimental Examples

### Example 1

Introduction

Gene therapy makes it possible to engineer disease-specific T-cells by chimeric antigen receptors (CARs), TCRs, or CAR/TCR hybrids. However, the elaborate and expensive protocols currently required to manufacture engineered T cells ex vivo are clearly not practical to introduce these transgenes at a scale large enough to address the requirements of the health care system. Here an injectable nanoparticle delivering in vitro transcribed (IVT) mRNA that can transiently reprogram circulating T cells to recognize disease-relevant antigens is disclosed. Repeated infusions of these polymer nanoparticles delivers tumor-specific CARs or virus-specific TCR transgenes into sufficient quantities of host T cells to induce disease regression in leukemia, prostate cancer, and hepatitis B-induced hepatocellular carcinoma at similar levels as bolus infusions of ex vivo engineered lymphocytes. Given the ease of manufacturing, distributing and administration, this new nanotechnology translates into a high-impact therapeutic for a wide range of diseases.

The efficacy of adoptive T-cell therapies, a powerful modality where T cells harvested from the patient or a donor are genetically targeted to cancers or infectious agents, is now undisputed and supported by numerous clinical trials showing impressive clinical benefit. However, the complexity and the high costs involved in manufacturing a T cell product for each patient, rather than preparing a drug in bulk in a standardized form, makes it difficult to outcompete current frontline therapy options, such as small molecule drugs or monoclonal antibodies. Most CAR-T and TCR-engineered T cells are currently made by a cumbersome and bespoke process involving (i) Leukapheresis to extract T cells from a patient who is connected by two intravenous tubes to an apheresis machine for several hours. This is not comfortable for the patient, incurs a substantial cost, and ultimately, large-scale adoption of autologous T therapy may become rate limited by availability of apheresis capacity; (ii) Activation and transduction of T cells; (iii) Expansion of transduced T cells over a two-week period in a cytokine supplemented tissue culture medium; and (iv) Washing and concentrating the T cells prior to administration. For T products made at central facilities and transported to remote treatment centers, cells must be cryopreserved; and (v) quality control (QC) release assays are conducted for each batch of CAR-T product. The entire process must be conducted under environmentally controlled GMP compliant conditions which are expensive to maintain and run. As each CAR-T product is made from starting materials (T cells) from the patient to be treated, there are no substantial economies of scale.

IVT mRNA has recently come into focus as a potential new drug class to deliver genetic information. Such synthetic mRNA medicines can be engineered to transiently express proteins by structurally resembling natural mRNA. They are easily developed, inexpensive to produce, and efficiently scalable for manufacturing purposes. Advances in addressing the inherent challenges of this drug class, particularly related to controlling the translational efficacy and immunogenicity of the IVT mRNA, provide the basis for a broad range of potential applications.

Here, the use of IVT mRNA as an injectable drug to genetically program circulating T cells to transiently express disease specific receptors, thereby bypassing the need to extract and culture lymphocytes from patients (FIGS. 1, 2, 3A, 3B), was explored. To condense and protect the IVT mRNA payload and to precisely target it to T cells, biodegradable polymeric nanoparticles were formulated. It was first demonstrated ex vivo that a single nanoparticle application can routinely transfect >70% of cultured T cells with the CD19-specific 1928z CAR (Yescarta™ approved by the FDA for the treatment of adult patients with relapsed or refractory large B-cell lymphoma) or with the HBcore18-27 TCR specific for the Hepatitis B virus (HBV) core antigen (currently in a Phase I study to treat patients with HBV-related hepatocellular carcinoma). Nanoparticle-transfected T cells transiently express these CAR- or TCR-transgenes on their surface for an average of seven days.

Compared to personalized T-cell therapy, which is an elaborate and costly procedure, nanoparticle drugs are inexpensive and easy to manufacture in bulk (and continuous flow microfluidic instruments designed for scale-up manufacturing of nanoparticles under cGMP conditions are now available). Exemplary methods for microfluidic assembly of nanocarriers are provided in, for example, Wilson et al. (2017) J. Biomed. Mat. Res. A. 6(105):183-1825. In some embodiments, the nanocarriers are manufactured using a micromixer chip. An exemplary micromixer chip compatible with the methods of the disclosure is Dolomite® micromixer chip (Dolomite Microfluidics, Royston, UK (Dolomite TELOS™). The results transform treatment opportunities from ex vivo engineered T-cell products to affordable off-the-shelf reagents for the treatment of patients with malignancies or chronic infections, that are available at the day of diagnosis and as frequently as medically necessary.

Objective.

The objective of this Example was to explore the use of IVT mRNA as an injectable drug to genetically program circulating T cells to transiently express disease specific receptors, thereby bypassing the need to extract and culture lymphocytes from patients. Experiments were performed which demonstrate that, when administered periodically, CAR- or TCR-encoding mRNA particles can program T cells in quantities that are sufficient to bring about tumor regression with efficacies that are similar to conventional infusions of T cells transduced ex vivo with CAR-encoding viral vectors.

Results.

IVT mRNA nanoparticles efficiently transfect human T cells with CAR- or TCR transgenes. To deliver IVT mRNA encoding disease-specific receptor genes into human lymphocytes, a biodegradable poly(β-amino ester) (PBAE) polymer formulation was used as a carrier matrix (FIG. 4A). Cationic PBAE self-assembles into nanocomplexes with anionic nucleic acids via electrostatic interactions (FIG. 4B). The particles were targeted by coupling an anti-CD8 binding domain to polyglutamic acid (PGA) using PGA-maleimide reacting with a cysteine added to Fab sequence, forming a conjugate that was electrostatically adsorbed to the particles. The resulting mRNA nanoparticles can be lyophilized for long-term storage. Prior to use, particles hydrate within seconds following addition of sterile water to restore their original concentration. No significant differences were observed in the physical properties of nanoparticles loaded with CAR transgenes versus the slightly larger TCR transgenes, which encode TCR alpha and beta chains linked by a 2A linker sequence. Exemplary protein sequences are provided in FIG. 8.

Whether adding targeted IVT mRNA nanoparticles to an established culture of human lymphocytes can choreograph robust transfection in them was tested first. To test this approach in clinically-relevant applications, IVT mRNA encoding the leukemia-specific 19-28z CAR was incorporated into nanoparticles (FIGS. 5A-5E). CD19-targeted receptors are the most investigated CAR-T cell product today, with nearly 30 ongoing clinical trials internationally, and two already FDA approved cancer therapies (Sadelain, J Clin Invest 125:3392-3400, 2015).

As a second example, IVT mRNA encoding a high-affinity HBV-specific TCR (FIGS. 5F-5J) was delivered. T-cell therapy of chronic hepatitis B is a novel approach to restore antiviral immunity and cure the infection. The HBcore18-27 TCR specific for the HBV core antigen was isolated from an HLA-A 02.01 donor with resolved HBV infection (Kah et al., J Clin Invest. 2017 Aug. 1; 127(8):3177-3188).

For both constructs, the 1928z CAR and the HBcore18-27 TCR, real-time quantitative PCR and flow cytometry were used to measure their expression levels in human T cells following a single nanoparticle transfection. Transgene expression peaked 24 hours after nanoparticle exposure, followed by a gradual decline of expression in these proliferating T cells (FIGS. 5A, 5F). This translated into high levels of CAR- or TCR-surface expression, with a maximum on day 2 (75%±11% of T cells expressed the 1928z CAR; FIGS. 5B, 5C; and an average 89%±4% of T cells expressed the HBcore18-27TCR; FIGS. 5G, 5H). As expected, receptor expression was transient, and was reduced to 28%±6% for the CAR and 26%±9% for the TCR after 8 days in culture.

The function (killing and cytokine production) of nanoparticle-transfected T cells was next compared with that of T cells engineered with these receptors using viral methods. Using real-time IncuCyte® (Essen Instruments, Inc., Ann Arbor, Mich.) live cell assays, no significant differences were measured in the ability of IVT mRNA-transfected T cells to selectively lyse antigen-positive target cells (Raji lymphoma cells for the 1928z CARs and HepG2 liver cancer cells stably transduced with HBcAg for HBcore18-27 TCRs) (FIGS. 5D, 5I). Also, similar levels of T-cell secreted effector cytokines were measured in nanoparticle-transfected versus virally transduced T cells (FIGS. 5E, 5J).

A. Infusions of Carrier-Delivered mRNA Reprogram Host T Cells to Recognize Leukemia.

It was next examined whether lymphocyte-targeted IVT mRNA nanoparticles can reprogram circulating T cells in quantities large enough to bring about tumor regression with efficacies that are similar to conventional methods. As an in vivo demonstration of efficacy in leukemia, immunodeficient NOD.Cg-Prkdcscid II2rgtm1Wjl/SzJ (NSG) mice were inoculated with 1×10⁶CD19+ Raji cells expressing firefly luciferase. Five days later, mice were reconstituted with 10×10⁶ CD3+ human T cells then received six weekly infusions of nanoparticles loaded with mRNA encoding the 1928z CAR (to generate leukemia specificity) or control particles loaded with mRNA encoding GFP (FIG. 6A). Controls received no treatment. The weekly nanoparticle administration protocol was chosen based on the kinetics of CAR surface expression measured ex vivo with IVT mRNA nanoparticles, which showed relevant receptor expression for up to 8 days (FIGS. 5B, 5C).

To compare the therapeutic efficacy of nanoparticle infusions with conventional adoptive T cell therapy, an additional group of mice was also treated with a single dose of 5×10⁶ T cells transduced ex vivo with lentiviral vectors encoding the 1928z CAR. This quantity is equivalent to the higher doses of CAR T cells used in current clinical studies, where patients have been treated with up to 1.2×10⁷ CAR T cells per kilogram of body weight (Grupp et al., N Engl J Med 368:1509-1518, 2013). Bioluminescence imaging was used to serially quantify tumor growth. Overall survival was also monitored. Survival was greatly improved in mice treated with ex vivo engineered adoptively transferred 1928z CAR-T cells, compared to untreated controls. Tumors were eradicated in six of ten mice, and the others showed substantial tumor regression along with an average 32 day improvement in survival (FIG. 6C). This therapeutic benefit achieved with conventional adoptive T cell therapy was similar to treatments with IVT mRNA nanoparticles programing the same CARs into the lymphocytes in vivo, which achieved tumor eradication in 7/10 mice and an average 37 day improvement in survival of the relapsing animals (FIG. 6C).

Flow cytometry of peripheral blood 2 days after the first dose revealed that 1928z-carrying nanoparticles rapidly and efficiently programed peripheral T cells to recognize leukemia cells (mean 10% CAR⁺ amongst CD8+±4.3%, FIGS. 6D, 6E). These CARs were transiently expressed for up to one week (0.8%±0.4% CAR+CD8+ T cells on day 7). Repeat doses of nanoparticles were as effective as the first injection, and achieved an average of 10.7%±3.6% encapsulated mRNA transfer into host T cells (FIG. 6E). This suggests that, despite its often transient nature, IVT mRNA can serve as a platform to achieve persistent in situ CAR expression in host lymphocytes.

B. Introducing Tumor-Specific CAR Genes into T Cells Via Nanoparticles Results in Regression of Prostate Tumors.

To demonstrate efficacy in solid tumors, the ability of nanoparticles designed to introduce prostate tumor-specific CAR genes into circulating host T cells to induce regression of prostate tumors in mice was demonstrated. Unlike leukemia cells, which universally express high levels of the CD19 target antigen and are easily accessible to circulating T cells, solid cancers are heterogeneous and protected (Meacham & Morrison, Nature 501:328-337, 2013). This means that a portion of the tumor cells will not be recognized by the targeting CAR, and will be surrounded by immune-suppressing defenses that can render T cells dysfunctional. In fact, whole genome/transcriptional profiling has been used in 140 prostate cancer metastases to establish that prostate tumor lesions exhibit heterogeneous expression of three key cell surface proteins (Prostate-Specific Membrane Antigen (PSMA), Prostate Stem Cell Antigen (PSCA), and Receptor tyrosine kinase-like orphan receptor 1 (ROR1)) between patients (FIG. 7A).

To recapitulate human disease, LNCaP C42 prostate carcinoma cells (which exhibit heterogeneous expression of key cell surface proteins, FIG. 7B) were orthotopically transplanted into the dorsal lobe of the prostate gland of NSG mice (FIG. 7C). To serially monitor tumor burden by bioluminescence imaging, tumor cells were genetically tagged with Firefly luciferase (FLuc). Following orthotopic transplantation, all mice reproducibly developed lesions within three weeks (FIG. 7C, right panel) and were randomly assigned to the various treatment or control groups for the experiments (FIG. 7C).

The therapeutic efficacy of systemically injecting tumor-bearing mice with 10⁶ ex vivo transduced CAR⁺ T cells specific for the tumor antigen ROR1 was measured. Even though anti-ROR1 CAR-T cells did not achieve tumor clearance, treated mice exhibited more than doubled survival rates (69 versus 32 days in the no treatment control group; FIGS. 7D, 7F).

Mice were systemically injected weekly with anti-ROR1 CAR transgene-loaded nanoparticles (FIG. 7E). Nanoparticle-induced CAR programming extended survival by an average of 40 days compared to untreated controls, which is similar to the survival benefit achieved with conventional adoptive T-cell therapy (Δmean survival=3 d, N.s., P=0.23; FIGS. 7D, 7F). This demonstrates that in vivo administration of nanocarriers achieves at least as great therapeutic effects as administration of T cells transduced with nanocarriers ex vivo prior to administration to the subject.

The antigen profile of relapsing prostate tumors was phenotyped by flow cytometry. One of the most common escape strategies seen in cancer is a reduction of target antigen expression because of the selective pressure CARs create. This phenomenon has been reported as a cause of failures in both preclinical and clinical studies when adoptively-transferred T cells specific for only single antigens were used to treat heterogeneous tumors (such as metastatic prostate cancer). In direct comparison to untreated LNCaP C42 prostate tumors, which express the ROR1 tumor antigen at various levels, CAR-targeted tumors in both treatment groups (adoptively transferred T cells or nanoparticle-programmed T cells) eventually developed ROR1 low/negative immune-escape variants (FIG. 7G).

Materials & Methods.

PBAE 447 Synthesis.

This polymer was synthesized using a method similar to that described by Mangraviti et al. (ACS Nano 9, 1236-1249, 2015). 1,4-butanediol diacrylate was combined with 4-amino-1-butanol in a 1.1:1 molar ratio of diacrylate to amine monomer. The mixture was heated to 90° C. with stirring for 24 h to produce acrylate-terminated poly(4-amino-1-butanol-co-1,4-butanediol diacrylate). 2.3 g of this polymer was dissolved in 2 ml tetrahydrofuran (THF). To form the piperazine-capped 447 polymer, 786 mg of 1-(3-aminopropyl)-4-methylpiperazine dissolved in 13 ml THF was added to the polymer/THF solution. The resulting mixture was stirred at room temperature for 2 hours, then the capped polymer was precipitated with 5 volumes of diethyl ether. After the solvent was decanted, the polymer was washed with 2 volumes of fresh ether, then the residue was dried under vacuum for 2 days before use to form a stock of 100 mg/ml in DMSO, which was stored at −20° C.

PGA-antibody Conjugation.

15 kD poly-glutamic acid (from Alamanda Polymers) was dissolved in water to form 20 mg/ml and sonicated for 10 minutes. An equal volume of 4 mg/ml 1-ethyl-3-(3-dimethylaminopropyl) carbodiimide hydrochloride (Thermo Fisher) in water was added, and the solution was mixed for 5 minutes at room temperature. The resulting activated PGA was then combined with antibodies at a 4:1 molar ratio in phosphate buffered saline (PBS) and mixed for 6 hours at room temperature. To remove unlinked PGA, the solution was exchanged 3 times against PBS across a 50,000 NMWCO membrane (Millipore). Antibody concentrations were determined using a NanoDrop 2000 spectrophotometer (Thermo Scientific). Anti-CD8 (clone OKT8) antibodies were used for T cell experiments. Clone C1.18.4 was used as a control antibody.

mRNA Synthesis.

Codon-optimized mRNA encoding the anti-human 1928z CAR, the anti-ROR1 (4-1BBz) CAR and the HBV-specific TCR (HBcore18-27 TCR) were used. The codon-optimized DNA sequences are provided in FIG. 8. All constructs were ordered from Trilink Biotechnologies, with the following modifications: modified mRNA transcript with full substitution of pseudo-U and 5-methyl-C; ARCA capped (CapO); polyadenylated (120A); Dnase and phosphatase treatment; silica membrane purification; and packaged as a solution in 1 mM Sodium Citrate, pH 6.4.

Nanoparticle Preparation.

mRNA stocks were diluted to 100 μg/ml in sterile, nuclease-free 25 mM sodium acetate buffer, pH 5.2 (NaOAc). PBAE-447 polymer in DMSO was diluted to 6 mg/ml in NaOAc, and added to mRNA at a 60:1 (w:w) ratio. After the resulting mixture was vortexed for 15 seconds at medium speed, it was incubated for 5 minutes at room temperature so nanoparticles could form. To add targeting elements to the nanoparticles, PGA-linked binding domains were diluted to 250 μg/ml in NaOAc and added at a 2.5:1 (w:w) ratio to the mRNA. The resulting mixture was vortexed for 15 seconds at medium speed, and then incubated for 5 minutes at room temperature to permit binding of PGA-binding domains to the nanoparticles.

The nanoparticles were lyophilized by mixing them with 60 mg/ml D-sucrose as a cryoprotectant, and flash-freezing them in liquid nitrogen, before processing them in a FreeZone 2.5 L Freeze Dry System (Labconco). The lyophilized nanoparticles were stored at −80° C. until use. For application, lyophilized nanoparticles were re-suspended in a volume of sterile water to restore their original concentration.

Cell sorting and Flow Cytometry.

Data were acquired using BD LSRFortessa or FacsCanto II cell analyzers running FACSDIVA software, sorted on the BD FACS ARIA-II, and analyzed with FlowJo v10.1.

CAR T Cell Killing Assay.

Specific cytolysis of CAR target cells was assayed by flow cytometry. Target K562-CD19 cells were labeled with low (0.4 μM), and control K562 with high (4.0 μM) carboxyfluorescein succinimidyl ester (CFSE) for 15 minutes at 37° C. Both samples were washed in complete medium containing serum, mixed at a ratio of 1:1, then co-cultured with 19-41Bζ at the indicated effector:target ratios. To assess specific cytolysis, each condition was stained with anti-CD8 mAbs (BioLegend) to identify T cells and with 7AAD to exclude dead cells, and analyzed by flow cytometry. Specific cell killing was assessed by measuring the ratio of viable CD19+ target cells (low CFSE) to control CD19− K562 cells (high CFSE).

Microscopy.

10⁶ T cells in 400 μl of XFSFM were treated with anti-CD3 targeted nanoparticles containing 3 μg cy5-labeled eGFP mRNA for 1 h at 4° C. for surface binding, followed by a 2-h incubation at 37° C. for internalization. Following these treatments, the cells were washed 3 times with cold PBS, and loaded onto poly-1-lysine (Sigma)-coated slides for 30 minutes at 4° C. The samples were fixed in 2% paraformaldehyde, mounted in ProLong Gold Antifade reagent (Invitrogen), and imaged with a Zeiss LSM 780 NLO laser scanning confocal microscope.

Statistical Analysis.

Unless otherwise stated, graphs show mean±standard error of the mean. Statistical analysis was done with Prism software (Graphpad).

### Example 2

Materials and Methods.

PbAE Synthesis.

The methods used to synthesize the polymer were described previously (Mangraviti A et al. (2015) ACS Nano 9: 1236-1249). 1,4-butanediol diacrylate was combined with 4-amino-1-butanol in a 1:1 molar ratio of diacrylate to amine monomers. Acrylate-terminated poly(4-amino-1-butanol-co-1,4-butanediol diacrylate) was formed by heating the mixture to 90° C. with stirring for 24 hours. 2.3 g of this polymer was dissolved in 2 mL tetrahydrofuran (THF). To form the piperazine-capped 447 polymer, 786 mg of 1-(3-aminopropyl)-4-methylpiperazine in 13 mL THF was added to the polymer/THF solution and stirred at room temperature (RT) for 2 hours. The capped polymer was precipitated with 5 volumes of diethyl ether, washed with 2 volumes of fresh ether, and dried under vacuum for 1 day. Neat polymer was dissolved in dimethyl sulfoxide (DMSO) to a concentration of 100 mg/mL and stored at −20° C.

PGA conjugation to Di-mannose. α-D-mannopyranosyl-(1→2)-α-D-mannopyranose (Di-mannose, Omicron Biochemicals Inc.) was modified into glycosylamine before being conjugated to PGA. First, the Di-mannose (157 mg) was dissolved in 10.5 mL of saturated aqueous ammonium carbonate, then stirred at RT for 24 hours. On the second day, more solid ammonium carbonate was added until the Di-mannose precipitated from the reaction solution. The mixture was stirred until completion, as measured by TLC, followed by lyophilization to remove the excess ammonium carbonate. Complete removal of volatile salt was accomplished by re-dissolving the solid in methanol. These procedures created an amine on the anomeric carbon for future conjugation with PGA.

To conjugate aminated Di-mannose to PGA, the substrate was dissolved in water to 30 mg m/L, then sonicated for 10 minutes. Ethyl-N′-(3-dimethylaminopropyl) carbodiimide.HCl in water (4 mg/mL, 30 equiv.) was added with mixing at RT for 4 min. N-hydroxysulfosuccinimide in water (30 mg/mL, 35 equiv.) was incubated with the PGA/EDC solution for 1 minute. Aminated Dimannose in phosphate-buffered saline (PBS) was combined with the resulting activated PGA in a 44:1 molar ratio and mixed at RT for 6 h. Excess reagents were removed by dialysis against water for 24 hours.

mRNA Synthesis.

Codon-optimized mRNA for eGFP, IRF5, and IKK (TriLink Biotechnologies) were capped with the Anti-Reverse Cap Analog 3″-O-Me-m7G(5′)ppp(5′)G (ARCA), and fully substituted with the modified ribonucleotides pseudouridine (4)) and 5-methylcytidine (m5C).

Nanoparticle Preparation.

IRF5 and IKKβ mRNAs were combined at a 3:1 (w:w) ratio and diluted to 100 μg/mL in 25 mM sodium acetate (NaOAc) buffer (pH=5.2). Poly(β-amino esters)-447 (PbAE-447) polymer in DMSO (prepared as described above) was diluted from 100 μg/μL to 6 μg/μL, also in NaOAc buffer. To form the nanoparticles, PbAE-447 polymers were added to the mRNA at a ratio of 60:1 (w:w) and vortexed immediately for 15 seconds at a medium speed, then the mixture was incubated at RT for 5 min to allow the formation of PbAE-mRNA polyplexes. In the next step, 100 μg/mL PGA/Di-mannose in NaOAc buffer was added to the polyplexes solution, vortexed for 15 seconds at medium speed, and incubated for 5 min at room temperature. In this process, PGA/Di-mannose coated the surfaces of PbAE-mRNA polyplexes to form the final NPs. For long-term storage, D-sucrose (60 mg/mL) was added to the NP solutions as a cryoprotectant. The nanoparticles were snap-frozen in dry ice, then lyophilized. The dried NPs were stored at −20° C. or −80° C. until use. For in vivo experiments, lyophilized NPs were re-suspended in water at a 1:20 (w:v) ratio.

Characterization of Nanoparticle Size Distribution and ζ-Potential.

The physiochemical properties of NPs (including hydrodynamic radius, polydispersity, potential, and stability) were characterized using a Zetapals instrument (Brookhaven Instrument Corporation) at 25° C. To measure the hydrodynamic radius and polydispersity based on dynamic light scattering, NPs were diluted 5-fold into 25 mM NaOAc (pH=5.2). To measure the ζ-potential, NPs were diluted 10-fold in 10 mM PBS (pH=7.0). To assess the stability of NPs, freshly prepared particles were diluted in 10 mM PBS buffer (pH=7.4). The hydrodynamic radius and polydispersity of NPs were measured every 10 minutes for 5 hours, and their sizes and particle concentrations were derived from Particle Tracking Analysis using a Nanosite 300 instrument (Malvern). To characterize the NPs using transmission electron microscopy, previously described protocols were followed (Smith T T et al. (2017) Nat Nanotechnol 12: 813-820). Freshly made NPs (25 μL containing 0.83 μg of mRNA) were deposited on glow discharge-treated 200 mesh carbon/Formvar-coated copper grids. After 30 seconds, the grids were treated sequentially with 50% Karnovsky's fixative, 0.1 M cacodylate buffer, dH2O, then 1% (w/v) uranyl acetate. Samples were imaged with a JEOL JEM-1400 transmission electron microscope operating at 120 kV (JEOL USA).

Bone marrow derived macrophages (BMDMs) and other cell lines. To prepare BMDMs, bone marrow progenitor cells were harvested from mouse femurs following established protocols (Zhang X et al. (2008) Curr Protoc Immunol Chapter 14: Unit 14 11). These cells were cultured in complete medium [DMEM supplemented with 4.5 g/L D-glucose, L-glutamine, 10% heat-inactivated fetal bovine serum (FBS), 100 U/mL penicillin and 100 μg/mL, Glutamax 50 mL/500 mL, supplemented with 20 ng/mL M-CSF (Peprotech, cat#315-02)] at a seeding density of 0.5-1.0 e6/ml. Cells were allowed to differentiate into BMDMs ex vivo for 7 days under 5% CO2 at 37° C. Next, they were conditioned with macrophage-conditioned medium [macrophage complete medium supplemented with 20 ng/mL MPLA (Sigma, cat#L6895) or 20 ng/mL IL4 (eBioscience, cat#34-8041)]. BMDMs were used between 7-21 days ex vivo. The murine ovarian cancer cell line ID8, a gift from Dr. Katherine Roby (University of Kansas Medical Center, Kansas City, Kans.), was cultured in DMEM supplemented with 10% FBS, 100 U/mL penicillin, 5 μg/mL insulin, 5 μg/mL transferrin, and 5 ng/mL sodium selenite (all Sigma-Aldrich). To generate the more aggressive vascular endothelial growth factor (VEGF)-expressing ID8 strain, ID8 tumor cells were transfected with the pUNO1 plasmid (Invivogen) encoding murine VEGF along with the blasticidin-resistance gene. To obtain stable transfectants, tumor cells were cultured in complete medium containing 10 μg/mL blasticidin (Invivogen) for 3 weeks. The B16F10 melanoma cell line (American Type Culture Collection) was cultured in complete RPMI 1640 medium with 10% FBS, 100 U/mL penicillin, 2 mM/L-glutamine, 1.5 g/L sodium bicarbonate, 4.5 g/L glucose, 10 mM HEPES, 1.0 mM sodium pyruvate, and 0.05 mM 2-mercaptoethanol. For in vivo bioluminescent imaging, both ID8-VEGF and B16F10 cell lines were retrovirally transduced with firefly luciferase. The DF-1 cell line carrying RACS-PDGFβ. or RCAS-cre retrovirus was cultured in complete medium supplemented with 10% FBS and 100 U/mL penicillin under 5% CO2 at 39° C.

mRNA Transfection of BMDMs.

One day prior to transfection, BMDMs were reseeded on 24-well plates in macrophage complete medium at a concentration of 250,000/well. Before transfection, the complete medium was replaced with 300 μL unsupplemented DMEM. To transfect these cells, NPs containing 2 μg mRNA were added into the base medium and co-cultured with the BMDMs at 37° C. After 1 hour, medium containing NPs was removed, and the cells were cultured an additional 24 hours before evaluation of transfection efficiency and cell viability.

Transfection of BMDMs for macrophage signature gene analysis. BMDMs were reseeded on 24-well plates in conditioned medium 24 hours prior to transfection, allowing transformation of the cells into their phenotypes. M2-like macrophages were then exposed to either IRF5/IKKβ NPs carrying 25% eGFP mRNA as a reporter, or eGFP NPs (control) containing 2 μg mRNA, following the transfection protocol described above. After 24 hours, the top 10% percent of highly transfected BMDMs (as measured by eGFP expression) were sorted at 24 hours after transfection and were re-challenged in low-dose (10 ng/mL) IL4 medium for another 48 hours before RNA isolation. RNAs extracted from these cells were compared to those from standard M1- or M2-like macrophages so that signature genes associated with IRF5-NP treatment could be identified.

RNA isolation and preparation. To harvest RNAs, BMDMs were lysed in Trizol reagent (Ambion), and total RNAs were extracted and purified using RNeasy® Plus Universal Mini-Kits (QIAGEN) following the manufacturer's instructions. Sample RNA was quantified using a NanoDrop Microvolume Spectrophotometer (Thermo Fisher) and then subjected to quality control performed by the FHCRC Genomics Shared Resource with an Agilent 4200 TapeStation analyzer (Agilent).

Macrophage Signature Gene Analysis by NanoString Technology.

Gene expression values from stimulated BMDM cultures were measured using the nCounter® Myeloid Innate Immunity Panel (NanoString Technologies, Seattle, Wash.), which analyzes 770 genes occurring in 19 different pathways and processes them across 7 different myeloid cell types. The samples were tested using an nCounter Analysis System (NanoString Technologies, Seattle, Wash.). Raw data were processed and checked for quality using the R/Bioconductor NanoStringQCPro software package (Nickles D, Sandmann T, Ziman R and Bourgon R (2018) NanoStringQCPro: Quality metrics and data processing methods for NanoString mRNA gene expression data. R package version 1.10.0.). Expression values were normalized to the geometric mean of housekeeping genes and log 2-transformed using nSolver 4.0 software (NanoString Technologies, Seattle, Wash.). False Discovery Rates for ratio data were calculated from the p-values returned by the t-tests using the Benjamini-Yekutieli method.

Flow Cytometry and Cell Sorting.

Cells obtained from spleen, blood, peritoneal lavage, and bronchoalveolar lavage were analyzed by flow cytometry with myeloid and lymphoid immunophenotyping panels using the anti-mouse antibody probes listed in FIG. 8. Data were collected using a BD LSRFortessa analyzer running FACSDIVA software (Beckton Dickinson). CD11b+ and F4/80+ peritoneal macrophages were sorted using BD FACS ARIA II. All collected data were analyzed using FlowJo 10.0 software.

Cytokine Analysis.

Cytokine levels were evaluated using a Luminex 200 system (Luminex) at the FHCRC Immune Monitoring Shared Resource center. For ex vivo studies, cell culture supernatant was collected for the measurement of IL-6, IL-12p70, INFγ, and TNFα concentrations. For in vivo studies, plasma concentration of GM-CSF, INFγ, IL-12p70, IL-2, IL-6, and TNFα were measured.

qRT-PCR Analysis.

Gene expression levels were determined by qRT-PCR. To measure selected macrophage signature genes (SerpinB2, Retnla, Ccl5, Ccl11, codon-optimized IRF5, endogenous IRF5, and housekeeping GAPD genes), total RNA was isolated with RNeasy mini-columns (Qiagen) according to the manufacturer's instructions. cDNA was synthesized using a qScript cDNA Synthesis Kit (Quanta). For each sample, qRT-PCR was performed in triplicate via PerfeCTa qPCR SuperMix Low ROX (Quanta) using gene-specific probes from the Roche's Universal Probe Library (UPL) and PCR primers optimized by ProbeFinder (Roche): SerpinB2, UPL-049, F-ACTGGGGCAGTTATGACAGG (SEQ ID NO: 103), R-GATGATCGGCCACAAACTG (SEQ ID NO: 104); Retnla, UPL-078, F-TTGTTCCCTTCTCATCTGCAT (SEQ ID NO: 105), R-CCTTGACCTTATTCTCCACGA (SEQ ID NO: 106); Ccl5, UPL-105, F-CCTACTCCCACTCGGTCCT (SEQ ID NO: 107), R-CTGATTTCTTGGGTTTGCTGT (SEQ ID NO: 108); Cc111, UPL-018, F-AGAGCTCCACAGCGCTTC (SEQ ID NO: 109), R-CAGCACCTGGGAGGTGAA (SEQ ID NO: 110); codon-optimized IRF5, UPL-022, F-TCTTAAAGACCACATGGTAGAACAGT (SEQ ID NO: 111), R-AGCTGCTGTTGGGATTGC (SEQ ID NO: 112); endogenous IRF5, UPL-011, F-GCTGTGCCCTTAACAAAAGC (SEQ ID NO: 113), R-GGCTGAGGTGGCATGTCT (SEQ ID NO: 114). Signature gene mRNA levels were normalized based on amplification of GAPD, UPL-060, F-AGCCACATCGCTCAGACAC (SEQ ID NO: 115) and R-GCCCAATACGACCAAATCC (SEQ ID NO: 116). All qRT-PCR reactions were performed using Quant Studio5 RT-PCR machines running QuantStudio6 software (Applied Biosystems). In cases when the amplification plot did not cross the threshold and no Ct value was obtained (“undetermined”), a Ct value equal to the highest cycle number of in the assay (40 cycles) was used for comparisons of relative expression.

Mice and In Vivo Tumor Models.

Except for the brain tumor model-related experiments, the mice used in these experiments were obtained from Jackson Laboratory; the others were bred and housed in the FHCRC animal facility. All of the mice were used in the context of a protocol approved by the center's Institutional Animal Care and Use Committee. To model ovarian tumors, 5×106 vascular epithelial growth factor (VEGFP)-expressing ID8 cells were injected intraperitoneally (i.p.) into 4- to 6-week-old female albino B6 (C57BL/6J-Tyr<c-2J>) mice and allowed to establish for 2 weeks. For survival studies, the animals were treated i.p. with IRF5 NPs/eGFP NPs carrying 50 μg mRNA (two doses per week for 9 weeks, or until health conditions reached euthanizing requirements). For mechanism studies, the treatments for either 1, 2, or 3 weeks, were used followed by euthanization at 48 hours following the last dose. Peritoneal lavage was performed to collect the peritoneal cells. To compare the efficacy of IRF5/IKKβ NPs with status quo macrophage targeting therapies, one group of mice received treatment with IRF5/IKKβ NPs carrying 50 μg mRNA for 3 weeks with 2 doses per week; the second received oral gavage of 15 mg/kg PI3Kγ inhibitor IPI-594 (MedKoo Biosciences Inc) formulated in vehicle (5% 1-methyl-2-pyrrolidinone in polyethylene glycol 400) daily for 3 weeks; the third group received i.p. injection of 30 mg/kg CSF1R inhibitor Pexidartinib (PLX3397, MedKoo Biosciences Inc) formulated in the same vehicle daily for 3 weeks.

To model metastatic lung cancer, 2.5×104 16F10 cells transduced with F-luc and suspended in 200 μL RPMI medium were injected into 4- to 6-week-old female albino B6 (C57BL/6J-Tyr<c-2J>) mice (Jackson Laboratories) and allowed to establish for 1 week. For survival studies, mice were treated retro-orbitally with (or without) IRF5/IKKβ or eGFP NPs carrying 30 μg mRNA suspended in PBS. Mice were treated with 3 doses/wk for 3 weeks or until health conditions reached euthanizing requirements. For mechanism studies, the mice received the same treatments for 2 weeks. Bronchoalveolar lavage was performed to collect alveolar cells for analysis.

Mice bearing glioma were generated following published protocols (Uhrbom L et al. (2004) Nat Med 10: 1257-1260). Avian DF-1 cells producing RCAS-PDGFβ and RCAS-cre retroviruses were injected intracranially into both brain hemispheres (coordinates: 1 mm caudal from bregma, 2 mm lateral, depth of 2 mm from the dural surface) of Nestin-tv-a/Ink4a-arf−/−; Pten−/− mice (C57BL/6) between 4-6 weeks of age. Tumors were allowed to establish for 2 weeks. At day 15, mice received 10 Gy radiation to one hemisphere, while the unirradiated hemisphere was shielded with lead. The next day, mice received retro-orbital injections of IRF5/IKKβ NPs carrying 30 μg mRNA (3 doses/wk for 3 weeks), or were assigned to the PBS control group.

In Vivo Bioluminescence Imaging.

D-Luciferin (Xenogen) in PBS (15 mg/mL) was used as a substrate for firefly luciferase imaging. Bioluminescence images were collected with a Xenogen IVIS Spectrum Imaging System (Xenogen). Mice were anesthetized with 2% isoflurane (Forane, Baxter Healthcare) before and during imaging. For ID8-VEGF ovarian tumors, each mouse was injected i.p. with 300 μg of D-Luciferin, and images were collected 10 minutes later. For B16F10 lung metastatic tumors, mice were injected i.p. with 3 mg of D-Luciferin, and images were collected 15 minutes afterwards. For brain tumor models, the mice received retro-orbital injection of 75 mg/kg body weight D-Luciferin, and images were collected 4 minutes later. Acquisition times ranged from 10 s to 5 min.

Biodistribution Analysis.

To determine the biodistribution of IRF5 NPs in the I D8-VEGF ovarian tumor model, mice in 7-8 groups received an i.p. or retro-orbital dose of NPs carrying 50 μg mRNA. Twenty-four hours after injection, whole blood was collected, and mice were euthanized with CO2 to retrieve organs (liver, spleen, lung, kidney, heart, intestine, pancreases, and diaphragm). All tissues were stabilized with RNAlater, then frozen on dry ice. The codon-optimized IRF5 mRNA levels in each organ were measured using RT-qPCR.

Toxicity Analysis.

To measure potential in vivo toxicities of repeatedly infusing macrophage-targeting NPs, mice were injected (5/group) intravenously with 6 sequential doses of IRF5/IKKβ or eGFP NPs carrying 50 μg mRNA over the course of 3 weeks. Controls received no treatment. Twenty-four hours after the final infusion, mice were anesthetized and blood was collected by retro-orbital bleed to determine the complete blood counts. Blood was also collected for serum chemistry and cytokine profile analyses (performed by Phoenix Central Laboratories, Mukilteo, Wash.). Animals were then euthanized with CO2 to retrieve organs, which were washed with deionized water before fixation in 4% paraformaldehyde. The tissues were processed routinely, and sections were stained with hematoxylin and eosin. The specimens were interpreted by a board-certified staff pathologist, in a blinded fashion.

Cytokine Assays.

Cytokine levels were evaluated using a Luminex 200 system (Luminex) at the FHCRC Immune Monitoring Shared Resources. For ex vivo studies, cell culture supernatant was collected for the measurement of IL-6, IL12p70, INFγ, and TNFα concentrations. For in vivo studies, plasma concentrations of GM-CSF, INFγ, IL-12p70, IL-2, IL-6, and TNFα were measured.

Statistical Analysis.

The statistical significance of observed differences were analyzed using the unpaired, two-tailed one-way ANOVA test. The P values for each measurement are listed in the figure or figure legends. Survival data was characterized using the Log-rank test. All statistical analyses were performed either using GraphPad Prism software version 6.0 or R software.

Results.

Designing NPs to choreograph IVT mRNA transfection of TAMs. A targeted mRNA delivery system was developed that can introduce robust gene expression in the targeted cells by taking advantage of electrostatic interactions between cationic PBAE polymers and anionic mRNA (FIG. 9A). To improve the stability and translation of the mRNA encapsulated in the resulting nanocarriers, synthetic versions of the message were used that incorporate the modified ribonucleotides pseudouridine (ψ) (Kariko K et al. (2008) Mol Ther 16: 1833-1840) and 5-methylcytidine (m5C), and that are capped with ARCA (Anti-Reverse Cap Analog) (Quabius ES et al. (2015) N Biotechnol 32: 229-235). The mRNA is released from the mRNA-PbAE complex intracellularly by hydrolytic cleavage of ester bonds in the PbAE backbone. Efficient in vivo T cell transfection was previously demonstrated using this system (Smith T T et al. (2017) Nat Nanotechnol). To target the nanoparticles to TAMs as well as further stabilize the mRNA-PbAE complexes they contain, Di-mannose moieties were engineered onto their surfaces using PGA as a linker (FIG. 9A). The NPs were manufactured utilizing a simple two-step, charge driven self-assembly process. First, the synthetic mRNA was complexed with a positively charged PBAE polymer, which condenses the mRNA into nano-sized complexes. This step was followed by the addition of PGA functionalized with Di-mannose, which shields the positive charge of the PBAE-mRNA particles and confers macrophage-targeting. The resulting mRNA nanocarriers had a size of 99.8±24.5 nm, a polydispersity of 0.183, and a neutral surface charge (3.40±2.15 mV potential, FIGS. 9B, 9C). The transfection efficiency was first tested in murine bone marrow-derived macrophages (BMDMs) using NPs formulated with green fluorescent protein-encoding mRNA (GFP-NPs). Briefly, 50,000 BMDMs were exposed to NPs containing 1 μg mRNA for 1 hour, followed by flow cytometry measurements of GFP expression the next day. Following a single NP application, 31.9% (±8.5%) of these primary macrophages were routinely transfected without reducing their viability (FIGS. 9E, 9F). Surface modification of particles with Di-mannose was relevant, as transfection rates with untargeted (but PGA-coated) nanocarriers dropped to an average of 25% (±2.1%) in this inherently phagocytic cell type. The NPs selectively targeted the CD11b+, F4/80+ macrophage population, with 46% of macrophages transfected and expressing high levels of eGFP (FIG. 9D). This high transfection efficiency demonstrates the potency of the disclosed systems and methods in targeted delivery of mRNA to TAMs. Based on the results of an in vitro screen for transcription factor candidates that induce macrophage polarization, two mRNAs were selected for inclusion in the NP: the first encodes IRF5, a key member of the IRF family that favors the polarization of macrophages toward the M1 phenotype, and the second encodes IKKβ, a kinase that phosphorylates and activates IRF5.

Programming Immunosuppressive Macrophages into Proinflammatory Phenotypes.

To induce macrophage polarization, two mRNAs were selected for inclusion into the NPs: the first encodes IRF5, a key member of the interferon regulatory factor family that favors the polarization of macrophages toward the M1 phenotype (Krausgruber T et al. (2011) Nat Immunol 12: 231-238); the second encodes IKKβ, a kinase that phosphorylates and activates IRF5 (Ren J et al. (2014) Proc Natl Acad Sci USA 111: 17438-17443). A ratio of 3 IRF5 mRNAs to 1 IKKβ mRNA was used. Using real-time quantitative PCR specific for the NP-delivered (and codon-optimized) IRF5 mRNA, it was found that mRNA expression in macrophages was maximal at day 1, resulting in a 1,500-fold increase in IRF5 relative to endogenous factor levels (FIG. 9A). As expected, gene expression was transient but IRF5 levels remained strongly upregulated through day 3 (581-fold increased) and day 5 (87-fold increased) before returning to baseline.

To determine if IRF5/IKKβ-encoding NPs can reprogram M2 macrophages into the therapeutically desirable anti-cancer M1 phenotype, NanoString gene expression analysis was used. BMDMs were first cultured in the presence of interleukin-4 (IL-4) to induce a suppressive M2 phenotype (FIG. 9H). Following transfection with either control GFP-mRNA nanoparticles or IRF5/IKKβ. mRNA-containing NPs, gene expression profiles were analyzed and compared with inflammatory macrophages, which were generated separately by exposing BMDMs to the TLR4 agonist Monophosphoryl Lipid A. Despite being cultured in suppressive IL-4-containing medium, macrophages transfected with IRF5/IKKβ. mRNA NPs display gene expression profiles similar to inflammatory macrophages (FIG. 9I). Signature M2 macrophage genes, such as Serpinb2 and Ccl2 (Jablonski K et al. (2015) Plos One 10: e0145342; Varga T et al. (2016) J Immunol 196: 4771-4782), were strongly downregulated while key M1 differentiation genes, such as Ccl5 (Sica A et al. (2012) J Clin Invest 122: 787-795), were upregulated (FIGS. 9J, 9K). These data establish that NP-mediated expression of IRF5 and its kinase skews suppressive macrophages toward a proinflammatory phenotype.

### Example 3

Therapeutic effects of NP-delivered pro-M1 genes for disseminated ovarian cancer.

To evaluate this treatment approach in a clinically-relevant in vivo test system, a model that recapitulates late-stage, unresectable ovarian tumors in C57BL/6 mice was used; these animals are injected with ID8 ovarian cancer cells which were tagged with luciferase to enable serial bioluminescent imaging of tumor growth (Liao J B et al. (2015) J Immunother Cancer 3: 16; Stephan S B et al. (2015) Nat Biotechnol 33: 97-101). The tumors were allowed to establish for two weeks. By this stage, the mice have developed nodules throughout the peritoneal wall and in the intestinal mesentery. The animals were divided into 3 groups that received PBS (control), GFPNPs (sham), or IRF5/IKKβ NP treatment at an i.p. dose of 100 μg mRNA/mouse/week for 9 weeks (FIG. 10A). It was observed that in the IRF5/IKKβ NP treated group, the disease regressed and was eventually cleared in 40% of animals (overall 142 d median survival versus 60 d in controls; FIGS. 10B, 10C). To understand the underlying mechanisms of IRF5/IKKβ NP-mediated anti-tumor effects, how exclusively mannose receptor-targeting confined NP interaction to phagocytes was first examined. Flow cytometry of peritoneal lavage fluid collected 24 h after the first dose of NPs targeted with Di-mannose revealed preferential gene transfer into macrophages and monocytes (average 37.1% and 15.3%, respectively, FIG. 10D), while transfection into off-target cells was low or undetectable. A detailed phenotypic and functional analysis of macrophage/monocyte populations in the peritoneum of mice with established ovarian cancer following treatment with IRF5/IKKβ nanoparticles or PBS over a 3-week period (two weekly injections) was conducted next. Flow cytometric analysis revealed that IRF5/IKKβ NPs reduced the population of immune-suppressive macrophages (Ly6C−, F4/80+, CD206+) to an average 2.6%±2.1% versus 43%±15.6% in controls (FIGS. 10E, 10F). Conversely, the fraction of M1-like macrophages increased from 0.5%±0.2% to 10.2%±4.1% (FIG. 10E, 10G). IRF5 gene therapy also affected the population of other immune cells. In particular, inflammatory monocytes (CD11b+, Ly6C+, Ly6G−) were more abundant (73.4%±3.6% compared to 4.5%±1.9% in untreated mice). One interesting finding in all IRF5 NP-treated animals were multifocal dense clusters of lymphocytes present within or surrounding the neoplasms (FIG. 10H), indicating that genetic programming of immune stimulatory macrophages may restore lymphocyte migration and infiltration into solid tumors.

Peritoneal macrophages were isolated by fluorescence-activated cell sorting to analyze their cytokine secretion, and detected a robust increase in the release of pro-inflammatory (anti-tumor) cytokines IL-12 (3.4-fold higher), IFN-g (8.4-fold higher), and TNF-α (1.5-fold higher), whereas the levels of IL-6, a regulatory cytokine associated with differentiation toward alternatively activated (M2-like) macrophages, were reduced by 97-fold; FIG. 10I). Genome expression profiling confirmed differentiation toward an M1-like macrophage phenotype in IRF5/IKKβ nanoparticle-treated mice. Gene expression levels of macrophages cultured ex vivo in MPLA or IL-4 were included to provide reference values for classic M1-like or M2-like macrophages, respectively (FIG. 10J).

Biodistribution and Safety.

The distribution of nanoparticles in various organs 24 h after intraperitoneal injection using RT-qPCR assays designed to detect only nanoparticle-delivered (codon optimized) IRF5 was next quantified. The highest concentrations of IVT mRNA were found in organs located in the peritoneum, including liver, spleen, intestine, pancreas, and diaphragm (FIG. 11A). Small amounts of particle-delivered mRNA in organs that lie outside of the peritoneum (heart, lungs, kidneys) were detected, suggesting that a fraction of i.p. injected nanocarriers entered the systemic circulation. Guided by the distribution data, whether these nanoreagents are biocompatible and safe for repeated dosing was next assessed. Mice were injected with a total of 8 doses of IRF5/IKKβ NPs (two 50 μg mRNA doses/week for 4 weeks, FIG. 11B). They were euthanized 24 h after the final dose, body weight was recorded, blood was collected by retroorbital bleed for serum chemistry, and a complete gross necropsy was performed. There was no difference in body weights between groups. The following tissues were evaluated by a board certified staff pathologist: liver, spleen, mesentery, pancreas, stomach, kidney, heart, and lungs. Histopathological evaluation revealed in all cases multifocal dense clusters of lymphocytes within or surrounding tumor lesions, but no evidence of inflammation or frank necrosis was observed in tissues where neoplastic cells were not present (FIG. 11C). Also, serum chemistry of IRF5/IKKβ NP-treated mice was comparable to that of PBS controls, indicating that systemic toxicities did not occur (FIG. 11D). Because small amounts of IRF5-mRNA were detected systemically in biodistribution studies, parallel experiments were designed to quantitate inflammatory cytokines in the peripheral blood. Following a single i.p. injection of IRF5/IKKβ NPs, moderate and transient increase was measured in serum levels of interleukin-6 (IL-6) to an average of 26.8 μg/mL (FIG. 11E), and tumor necrosis factor-a (TNF-α) to an average 94.7 μg/mL (FIG. 11F). Based on previous reports, these levels are 500-fold lower than those associated with pathological findings and thus can be considered safe Tarrant J. M. (2010) Toxicol Sci 117: 4-16; Copeland S et al. (2005) Clin Diagn Lab Immunol 12: 60-67).

Controlling Systemic Tumor Metastases with Intravenous Infusions of IRF5/IKKβ Nanoparticles.

Based on the therapeutic responses achieved with IRF5/IKKβ NPs administered directly into the peritoneal cavity to treat tumor lesions spread throughout the peritoneum, the next question asked was whether intravenously infused mRNA nanocarriers could program macrophages systemically to control disseminated disease. RT-qPCR biodistribution studies revealed that i.v.-infused nanocarriers preferentially deliver their mRNA cargo to organs with high levels of resident macrophages/phagocytes, mostly the spleen, liver, and lungs (FIG. 12A). To measure anti-tumor responses in a clinically relevant in vivo test system, particles containing IRF5/IKKβ mRNA were administered into mice with disseminated pulmonary melanoma metastases (FIG. 12B). Recent work describes the foundational role of monocytes and macrophages in establishing metastases caused by this disease (Butler K L et al. (2017) Sci Rep 7: 45593; Nielsen S R et al. (2017) Mediators Inflamm 2017: 9624760), and it was confirmed by confocal microscopy that tumor engraftment was coordinate with phagocyte accumulation in the lungs (FIG. 12C). Tumor burdens were determined by bioluminescent imaging, and mice with detectable cancers were sorted into groups that had matching levels. Groups were then randomly assigned to treatment conditions, receiving no therapy (PBS), or intravenous injections of GFP- or IRF5/IKKβ-encapsulating nanoparticles. Only IRF/IKKβ nanoparticle therapy substantially reduced tumor burdens in the lungs; in fact, they improved overall survival by a mean 1.3-fold (FIGS. 12D, 12E). In parallel experiments, mice were sacrificed 22 days after tumor inoculation to validate bioluminescence tumor signals with counts of pulmonary metastases and to assess macrophage polarization by flow cytometry. The total number of metastases in the lungs of IRF5/IKK NP-treated animals was 8.7-fold reduced (average 40±16 metastases) compared to PBS controls (average 419±139 metastases; FIGS. 12F, 12G). Flow cytometry of bronchoalveolar lavage fluid cells revealed a strong shift from immune-suppressive (CD206+, MHCII−, CD11c+, CD11blow) macrophages toward activated (CD206−, MHCII+, CD11c−, CD11b+) phagocytes (FIGS. 12H, 12I).

Programming Tumor-Suppressing Phagocytes to Treat Glioma.

For a third in vivo test system glioma was examined, which is a difficult to manage cancer type where M2-like macrophages represent the majority of non-neoplastic cells and promote tumor growth and invasion (Hambardzumyan D et al. (2016) Nat Neurosci 19: 20-27). Currently, the standard of care for this disease is radiotherapy, which unfortunately offers only a temporary stabilization or reduction of symptoms and extends median survival by 3 months (Mann J et al. (2017) Front Neurol 8: 748). To recapitulate the genetic events and subsequent molecular evolution of the disease, the RCAS-PDGF-B/Nestin-Tv-a; Ink4a/Arf−/−; Pten−/− transgenic mouse model of PDGFβ-driven glioma (PDG mice (Hambardzumyan D et al. (2009) Transl Oncol 2: 89-95; Quail D F et al. (2016) Science 352: aad3018)) was used. Brain tissue was stereotactically injected with a mixture of DF-1 cells transfected with either RCAS-PDGFβ. or RCAS-cre retrovirus (1:1 mixture, 2 μL). Overexpression of the PDGFβ oncogene and the absence of the tumor suppressor genes Ink4a-arf and Pten in glioma progenitors led to the formation of 4-5 mm diameter tumors (FIG. 13A) with a nearly complete penetrance within 21 days (as established previously (Hambardzumyan D et al. (2009) Transl Oncol 2: 89-95)). Using immunofluorescence, the presence of tumor-infiltrating (CD68+) macrophages (FIG. 13B, indicated in third panel from the left) were confirmed in established gliomas (shown in second panel from the left). Flow cytometry revealed that the F4/80+, CD11b+ macrophage population accounted for 32.8% of total cells in the tumor, which is 9-fold higher than seen in age-matched healthy control mice (3.7%) (FIG. 13C). The PDG mice in the experiments express the firefly luciferase gene linked to a key cancer gene promoter. Bioluminescence from this reporter has been demonstrated to be positively correlated with tumor grade (Uhrbom L et al. (2004) Nat Med 10: 1257-1260), so it was used to monitor tumor development every four days after the onset of treatment. IRF/IKKβ. NPs as a monotherapy was first tested: PDG mice received intravenous infusions of 9 doses of NPs loaded with IRF5/IKKβ mRNA, or PBS in the control group (3 doses/week for 3 weeks). IRF/IKKβ. NP treatments only modestly suppressed tumor progression (producing on average only a 5-day survival advantage compared to untreated controls; FIG. 13D). However, combining radiotherapy as the standard-of-care with IRF5/IKKβ. NP injections substantially reduced tumor growth and more than doubled the survival of treated mice compared to the PBS control group (52 d versus 25 days, respectively; FIGS. 13E, 13F).

In conclusion, in vivo results from three preclinical solid tumor models demonstrate that nanoparticles, administered either locally or systemically, can deliver genes encoding master regulators of macrophage polarization to re-program immunosuppressive macrophages into tumor-clearing phenotypes.

Translation from Murine to Human Macrophages.

To confirm that the data acquired in mice has relevance to treat human disease, NPs delivering IVT mRNA encoding human IRF5 and IKKβ. (huIRF5 NPs) were fabricated. The human monocytic cell line THP-1 was used as a well-established M1 and M2 macrophage polarization model to test these nanocarriers (Li C et al. (2016) Sci Rep 6: 21044; Surdziel E et al. (2017) Plos One 12: e0183679). M2-type macrophages were generated by treating THP-1 cells with PMA and polarizing them with IL-4 and IL-13 (FIG. 14A). To confirm that huIRF5 NPs are functional and activate the IRF pathway, THP1-Lucia™ ISG cells were transfected with nanoparticles loaded with huIRF5/IKKβ. or GFP control mRNAs. THP1-Lucia™ ISG cells secrete the fluorescent Lucia reporter under the control of an IRF-inducible promoter. This composite promoter is includes five IFN-stimulated response elements (ISRE) fused to an ISG54 minimal promoter, which is unresponsive to activators of the NF-κB or AP-1 pathways. As a result, THP1-Lucia™ ISG cells allow the monitoring of the IRF pathway by determining the activity of the Lucia luciferase. It was found that huIRF5 NPs strongly upregulated luciferase expression in M2-polarized THP-1 cells, indicating that the mRNA constructs are functional in human cells (FIGS. 14B, 14C). To determine whether IRF5 pathway activation can reprogram M2-polarized THP-1 cells toward an M1-like phenotype, secretion of the pro-inflammatory cytokine IL-1β following NP transfection was measured. Production of IL-1β was significantly increased in THP-1 cells transfected with huIRF5 NPs versus untransfected controls (mean 21-fold; P<0.0001, FIG. 14D), which correlated with a robust upregulation (10.9-fold increased MFI, P<0.0001) of the M1 macrophage cell surface marker CD80 (FIG. 14E).

### Prophetic Example 1: Preconditioning with T Cell Attractants

Recombinant CCL21 (Chemokine (c-c motif) ligand 21) will be injected into subcutaneously established tumors and, one day later, tumors will be injected with nanoparticles that reprogram recruited T cells with tumor specific CARs, TCRs, or CAR/TCR hybrids. CCL21 is known to induce rapid T-cell infiltration (e.g. Riedl et al., Molecular Cancer 2003).

### Prophetic Example 2: Disseminated Ovarian Cancer

Disseminated ovarian cancer will be established in immunocompetent mice. Animals will be injected intraperitoneally (i.p.) with CCL21 followed one day later by i.p. injections of nanoparticles delivering mRNA that encodes a mesothelin (MSLN) specific TCR. Ovarian cancer cells express high levels of MSLN. Reprogramming efficiency (with or without CCL21 preconditioning) will be measured and tumor progression will be serially monitored using bioluminescent imaging.

### Prophetic Example 3: Murine Xenograft Model of HBV-Induced Hepatocellular Carcinoma

HepG2 tumor cells that are stably transfected with the HBcore18-27 antigen will be surgically transplanted into the liver of NSG mice. HepG2 tumor cells are tagged with firefly luciferase so that tumor progression can be noninvasively monitored. Mice will then be reconstituted with human T cells and injected with T-cell targeted nanoparticles delivering mRNA that encodes the Anti-HBV-specific TCR (HBcore18-27), or control GFP. Tumor progression will be compared in TCR nanoparticle treated versus GFP nanoparticle controls. TCR reprogramming in the peripheral blood will also be directly measured by flow cytometry.

Unless otherwise indicated, the practice of the present disclosure can employ conventional techniques of immunology, molecular biology, microbiology, cell biology and recombinant DNA. These methods are described in the following publications. See, e.g., Sambrook, et al. Molecular Cloning: A Laboratory Manual, 2nd Edition (1989); F. M. Ausubel, et al. eds., Current Protocols in Molecular Biology (1987); the series Methods IN Enzymology (Academic Press, Inc.); M. MacPherson, et al., PCR: A Practical Approach, IRL Press at Oxford University Press (1991); MacPherson et al., eds. PCR 2: Practical Approach (1995); Harlow and Lane, eds. Antibodies, A Laboratory Manual (1988); and R. I. Freshney, ed. Animal Cell Culture (1987).

Sequence information provided by public database can be used to identify gene sequences to target and nucleic acid sequences encoding phenotype-altering proteins as disclosed herein. Exemplary sequences are provided in FIG. 15.

Variants of the sequences disclosed and referenced herein are also included. Variants of proteins can include those having one or more conservative amino acid substitutions. As used herein, a “conservative substitution” involves a substitution found in one of the following conservative substitutions groups: Group 1: Alanine (Ala), Glycine (Gly), Serine (Ser), Threonine (Thr); Group 2: Aspartic acid (Asp), Glutamic acid (Glu); Group 3: Asparagine (Asn), Glutamine (Gin); Group 4: Arginine (Arg), Lysine (Lys), Histidine (His); Group 5: Isoleucine (Ile), Leucine (Leu), Methionine (Met), Valine (Val); and Group 6: Phenylalanine (Phe), Tyrosine (Tyr), Tryptophan (Trp).

Additionally, amino acids can be grouped into conservative substitution groups by similar function or chemical structure or composition (e.g., acidic, basic, aliphatic, aromatic, sulfur-containing). For example, an aliphatic grouping may include, for purposes of substitution, Gly, Ala, Val, Leu, and Ile. Other groups containing amino acids that are considered conservative substitutions for one another include: sulfur-containing: Met and Cysteine (Cys); acidic: Asp, Glu, Asn, and Gln; small aliphatic, nonpolar or slightly polar residues: Ala, Ser, Thr, Pro, and Gly; polar, negatively charged residues and their amides: Asp, Asn, Glu, and Gln; polar, positively charged residues: His, Arg, and Lys; large aliphatic, nonpolar residues: Met, Leu, Ile, Val, and Cys; and large aromatic residues: Phe, Tyr, and Trp. Additional information is found in Creighton (1984) Proteins, W.H. Freeman and Company.

As indicated elsewhere, variants of gene sequences can include codon optimized variants, sequence polymorphisms, splice variants, and/or mutations that do not affect the function of an encoded product to a statistically-significant degree.

Variants of the protein, nucleic acid, and gene sequences disclosed herein also include sequences with at least 70% sequence identity, 80% sequence identity, 85% sequence, 90% sequence identity, 95% sequence identity, 96% sequence identity, 97% sequence identity, 98% sequence identity, or 99% sequence identity to the protein, nucleic acid, or gene sequences disclosed herein.

“% sequence identity” refers to a relationship between two or more sequences, as determined by comparing the sequences. In the art, “identity” also means the degree of sequence relatedness between protein, nucleic acid, or gene sequences as determined by the match between strings of such sequences. “Identity” (often referred to as “similarity”) can be readily calculated by known methods, including those described in: Computational Molecular Biology (Lesk, A. M., ed.) Oxford University Press, N Y (1988); Biocomputing: Informatics and Genome Projects (Smith, D. W., ed.) Academic Press, N Y (1994); Computer Analysis of Sequence Data, Part I (Griffin, A. M., and Griffin, H. G., eds.) Humana Press, N J (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR, Inc., Madison, Wis.). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wis.); BLASTP, BLASTN, BLASTX (Altschul, et al., J. Mol. Biol. 215:403-410 (1990); DNASTAR (DNASTAR, Inc., Madison, Wis.); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, N.Y. Within the context of this disclosure it will be understood that where sequence analysis software is used for analysis, the results of the analysis are based on the “default values” of the program referenced. As used herein “default values” will mean any set of values or parameters, which originally load with the software when first initialized.

As will be understood by one of ordinary skill in the art, each embodiment disclosed herein can comprise, consist essentially of or consist of its particular stated element, step, ingredient, or component. As used herein, the transition term “comprise” or “comprises” means includes, but is not limited to, and allows for the inclusion of unspecified elements, steps, ingredients, or components, even in major amounts. The transitional phrase “consisting of” excludes any element, step, ingredient, or component not specified. The transition phrase “consisting essentially of” limits the scope of the embodiment to the specified elements, steps, ingredients, or components and to those that do not materially affect the embodiment. As used herein, a material effect would cause a statistically-significant reduction in expression of a therapeutic protein within 7 days following administration of a disclosed nanoparticle to a subject.

In particular embodiments, reference to CDR sequences are in accordance with Kabat numbering.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to journal articles, publications, patents, and patent applications (including patent application publications) (collectively “citations”) throughout this specification. Each of the above citations are individually incorporated herein by reference for their particular cited purpose and/or teaching.

It is to be understood that the embodiments disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Ed. Anthony Smith, Oxford University Press, Oxford, 2004).

Throughout the disclosure, all references and patent documents cited are incorporated by reference herein in their entireties.

What is claimed is:

1. A method of treating cancer in a subject in need thereof, comprising selecting a nanoparticle that results in transient expression of an anti-ROR1 chimeric antigen receptor (CAR), an anti-CD19 CAR, or a hepatitis B antigen specific T cell receptor (TCR) selectively by T cells following administration to the subject;
administering a therapeutically effective amount of the selected nanoparticle to the subject;
monitoring the subject for expression of the anti-ROR1 CAR, anti-CD19 CAR, or hepatitis B antigen specific TCR;
and administering a second therapeutically effective amount of the selected nanoparticle to the subject when the expression level of the anti-ROR1 CAR, anti-CD19 CAR, or hepatitis B antigen specific TCR falls below a threshold;
wherein the selected nanoparticle comprises
(i) in vitro transcribed (IVT) mRNA encoding the anti-ROR1 CAR, anti-CD19 CAR, or hepatitis B antigen specific TCR encapsulated within a poly(β-amino ester) (PBAE) core;
(ii) a polyglutamic acid (PGA) coating on the outer surface of the PBAE core; and
(iii) CD4 and/or CD8 binding domains covalently linked to the PGA and extending from the surface of the coating
thereby treating cancer in the subject in need thereof.

2. The method of claim 1, wherein the transient expression lasts no more than two weeks.

3. The method of claim 1, further comprising preconditioning the subject with a T cell attractant and/or monocyte/macrophage attractant locally at a cancer site within the subject.

4. The method of claim 3, wherein the T cell attractant comprises CCL21 or IP10.

5. The method of claim 3, wherein the monocyte/macrophage attractant comprises CCL2, CCL3, CCL5, CCL7, CCL8, CCL13, CCL17 or CCL22.

6. The method of claim 1, further comprising
selecting a second nanoparticle that results in expression of a macrophage activator selectively by macrophages following administration to the subject;
administering a therapeutically effective amount of the second selected nanoparticle to the subject;
monitoring the subject for expression of the macrophage activator;
and administering a second therapeutically effective amount of the second selected nanoparticle to the subject when the expression level of the macrophage activator falls below a threshold;
wherein the selected second nanoparticle comprises
(i) IVT mRNA encoding the macrophage activator encapsulated within a PBAE core;
(ii) a PGA coating on the outer surface of the PBAE core; and
(iii) di-mannose extending from the surface of the coating.

7. The method of claim 6, wherein the macrophage activator comprises transcription factor interferon-regulatory factor (IRF) 5 in combination with the kinase IKKβ.

8. A method for treating a subject in need thereof, comprising selecting a nanoparticle that results in expression of a therapeutic protein by a selected cell type following administration to the subject and administering a first therapeutically effective amount of the selected nanoparticle to the subject thereby treating the subject in need thereof wherein the expression of the therapeutic protein falls below a detectable limit within 10 days of administration.

9. The method of claim 8, wherein the expression of the therapeutic protein falls below the detectable limit within 7 days of administration.

10. The method of claim 8, further comprising administering a second therapeutically effective amount of the selected nanoparticle to the subject.

11. The method of claim 10, wherein the administering of the second therapeutically effective amount occurs after expression of the therapeutic protein has fallen below the detectable limit.

12. The method of claim 10, wherein the administering of the second therapeutically effective amount occurs before expression of the therapeutic protein has fallen below the detectable limit.

13. The method of claim 10, wherein the first therapeutically effective amount and the second therapeutically effective amount are administered 5 days apart, 6 days apart, 7 days apart, 8 days apart, 9 days apart or 10 days apart.

14. The method of claim 8, wherein the administering comprises systemic or local administration.

15. The method of claim 14, wherein the administering comprises local administration at a tumor site.

16. The method of claim 8, wherein the administering comprises injection or infusion via catheter (a) into or proximal to a tumor (intratumoral), (b) into a vein (intravenous), or (c) into the peritoneum (intraperitoneally).

17. The method of claim 8, wherein the therapeutic protein comprises a disease specific receptor comprising a cell surface receptor.

18. The method of claim 17, wherein the disease specific receptor comprises a CAR, a TCR, or a hybrid thereof.

19. The method of claim 8, wherein the therapeutic protein comprises a leukemia-specific anti-CD19 CAR with a 1928z or 4-1BBz intracellular domain, a prostate tumor specific anti-ROR1 CAR with a 1928z or 4-1BBz intracellular domain, or a Hepatitis B virus (HBV) core antigen specific HBcore18-27 TCR.

20. The method of claim 8, wherein the therapeutic protein comprises a macrophage stimulating protein.

21. The method of claim 20, wherein the macrophage stimulating protein comprises transcription factor IRF5 in combination with the kinase IKKβ.

22. The method of claim 20, wherein the macrophage stimulating protein comprises one or more IRFs selected from IRF5, IRF1, IRF3, IRF7, IRF8, and/or a fusion of IRF7 and IRF3.

23. The method of claim 22, wherein the IRF7/IRF3 fusion protein comprises SEQ ID NO: 39.

24. The method of claim 22, wherein the one or more IRFs lack a functional autoinhibitory domain.

25. The method of claim 22, wherein the one or more IRFs lack a functional nuclear export signal (NES).

26. The method of claim 22, wherein the one or more IRFs is selected from a sequence having >90%, >95%, or >98% identity to SEQ ID NOs: 25-41.

27. The method of claim 22, wherein the one or more IRFs is IRF5 selected from SEQ ID NOs: 25-31.

28. The method of claim 27, wherein IRF5 comprises SEQ ID NO: 25 or SEQ ID NO: 27 with one or more mutations selected from S156D, S158D and T160D.

29. The method of claim 27, wherein IRF5 comprises SEQ ID NO: 26 with one or more mutations selected from T10D, S158D, S309D, S317D, S451D, and S462D.

30. The method of claim 27, wherein IRF5 comprises SEQ ID NO: 28 with one or more mutations selected from S425D, S427D, S430D, and S436D.

31. The method of claim 22, wherein the one or more IRFs is IRF1 selected from SEQ ID NOs: 32 and 36.

32. The method of claim 22, wherein the one or more IRFs is IRF8 selected from SEQ ID NOs: 35, 40, and 41.

33. The method of claim 32, wherein IRF8 comprises SEQ ID NO: 35 with a K310R mutation.

34. The method of claim 21, wherein the encoded IKKβ is selected from a sequence having >90%, >95%, or >98% identity to SEQ ID NOs: 42-46.

35. The method of claim 21, wherein the encoded IKKβ is selected from SEQ ID NOs: 42-46.

36. The method of claim 8, wherein the therapeutic protein comprises glucocorticoid-induced leuzine zipper (GILZ).

37. The method of claim 8, wherein the selected and administered nanoparticles are <130 nm.

38. The method of claim 8, wherein the selected and administered nanoparticles comprise:
(i) a synthetic mRNA encapsulated within a positively-charged carrier matrix, wherein the synthetic mRNA encodes the therapeutic protein;
(ii) a neutrally or negatively-charged coating on the outer surface of the carrier matrix; and
(iii) at least one selected cell targeting ligand extending from the surface of the coating, which selected cell targeting ligand specifically binds a marker on the selected cell type.

39. The method of claim 38, wherein the synthetic mRNA comprises IVT mRNA.

40. The method of claim 38, wherein the positively-charged carrier matrix comprises a positively charged lipid or polymer.

41. The method of claim 40, wherein the positively charged lipid or polymer comprises PBAE, poly(L-lysine), poly(ethylene imine) (PEI), poly-(amidoamine) dendrimers (PAMAMs), poly(amine-co-esters), poly(dimethylaminoethyl methacrylate) (PDMAEMA), chitosan, poly-(L-lactide-co-L-lysine), poly[α-(4-aminobutyl)-L-glycolic acid] (PAGA), or poly(4-hydroxy-L-proline ester) (PHP).

42. The method of claim 40, wherein the positively charged polymer comprises PBAE.

43. The method of claim 38, wherein the neutrally or negatively-charged coating comprises PGA, poly(acrylic acid), alginic acid, or cholesteryl hemisuccinate/1,2-dioleoyl-sn-glycero-3-phosphoethanolamine.

44. The method of claim 38, wherein the neutrally or negatively-charged coating comprises PGA.

45. The method of claim 38, wherein the neutrally or negatively-charged coating comprises a zwitterionic polymer.

46. The method of claim 38, wherein the neutrally or negatively-charged coating comprises a liposome.

47. The method of claim 46, wherein the liposome comprises 1,2-dioleoyl-3-trimethylammonium-propane (DOTAP), 1,2-di-O-octadecenyl-3-trimethylammonium propane (DOTMA), 3β-[N—(N′,N′-dimethylaminoethane)-carbamoyl]cholesterol (DC-Chol), dioctadecyl-amidoglycylspermine (DOGS), cholesterol, 1,2-dioleoyl-sn-glycero-3-phosphoethanolamine (DOPE), or 1,2-dioleoyl-sn-glycero-3-phosphocholine (DOPC).

48. The method of claim 38, wherein the at least one selected cell targeting ligand selectively binds lymphocytes and initiates receptor-induced endocytosis.

49. The method of claim 38, wherein the at least one selected cell targeting ligand selectively binds CD4 and/or CD8.

50. The method of claim 38, wherein the at least one selected cell targeting ligand comprises a binding domain selected from a CD4 antibody and/or a CD8 antibody.

51. The method of claim 38, wherein the at least one selected cell targeting ligand comprises a binding domain selected from an scFv fragment of a CD4 antibody and/or a CD8 antibody.

52. The method of claim 38, wherein the at least one selected cell targeting ligand comprises a binding domain selected from a CD4 antibody and/or a CD8 antibody; the carrier comprises PBAE; and the coating comprises PGA.

53. The method of claim 38, wherein the at least one selected cell targeting binds CD206, CD163, or CD23.

54. The method of claim 38, wherein the at least one selected cell targeting ligand comprises di-mannose.

55. The method of claim 38, wherein the at least one selected cell targeting binds CD38, G-protein coupled receptor 18 (Gpr18), formyl peptide receptor 2 (Fpr2), CD64, or CD68.

56. The method of claim 8, further comprising administering to the subject a therapeutically effective amount of a cell attractant.

57. The method of claim 56, wherein the cell attractant comprises a T cell attractant.

58. The method of claim 57, wherein the T cell attractant comprises CCL21 or IP10.

59. The method of claim 57, wherein the T cell attractant comprises CCL1, CCL2, CCL17, CCL22, CXCL9, CXCL10 or CXCL11.

60. The method of claim 56, wherein the cell attractant comprises a monocyte/macrophage attractant.

61. The method of claim 60, wherein the monocyte/macrophage attractant comprises CCL2, CCL3, CCLS, CCL7, CCLS, CCL13, CCL17 or CCL22.

62. The method of claim 56, wherein the cell attractant comprises a mast cell attractant.

63. The method of claim 62, wherein the mast cell attractant comprises CCL2 or CCLS.

64. The method of claim 56, wherein the cell attractant comprises an eosinophil attractant.

65. The method of claim 64, wherein the eosinophil attractant comprises CCL3, CCLS, CCL7, CCL11, CCL13, CCL24, or CCL26.

66. The method of claim 56, wherein the cell attractant comprises a neutrophil attractant.

67. The method of claim 66, wherein the neutrophil attractant comprises IL-8 or NAP1.

68. The method of claim 56, wherein the cell attractant is administered to the subject before the first therapeutically effective amount of nanoparticles is administered.

69. The method of claim 56, wherein the cell attractant is administered no more than one hour before, no more than 3 hours before, no more than 6 hours before, no more than 12 hours before, or no more than 24 hours before the first therapeutically effective amount of the selected nanoparticle is administered.

70. The method of claim 56, wherein the cell attractant is administered at least one hour before, at least 3 hours before, at least 6 hours before, at least 12 hours before, or at least 24 hours before the first therapeutically effective amount of the selected nanoparticle is administered.

71. The method of claim 56, wherein the cell attractant is administered (a) only after the first dose of the first therapeutically effective amount of the selected nanoparticle is administered.

72. The method of claim 8, wherein the subject is in need of treatment for cancer or an infectious disease.

73. The method of claim 72, wherein the cancer is leukemia, prostate cancer, hepatitis B-induced hepatocellular carcinoma, ovarian cancer, glioblastoma, or lung cancer.

74. A method for treating a subject in need thereof, comprising administering to the subject a therapeutically effective amount of a nanoparticle comprising:
(i) a synthetic mRNA encapsulated within a positively-charged carrier matrix, wherein the synthetic mRNA encodes a therapeutic protein, polynucleotide, or combination thereof;
(ii) a neutrally or negatively-charged coating; and
(iii) at least one cell targeting ligand extending from the surface of the coating, which cell targeting ligand is specific for selected cells;
wherein the nanoparticles are selectively incorporated into the selected cells within the subject such that the selected cells express the therapeutic protein, polynucleotide, or combination thereof from the synthetic mRNA, thereby treating the subject in need thereof.

75. The method of claim 74, further comprising administering to the subject an effective amount of a cell attractant.

76. The method of claim 75, wherein the cell attractant is a T cell attractant.

77. The method of claim 76, wherein the T cell attractant is CCL21 or IP10.

78. The method of claim 74, wherein the synthetic mRNA encodes a therapeutic protein, the therapeutic protein comprising at least one disease-specific receptor comprising a cell surface receptor.

79. The method of claim 78, wherein the at least one disease-specific receptor comprises a CAR or a TCR.

80. The method of claim 78, wherein the therapeutic protein comprises a leukemia-specific anti-CD19 CAR with a 1928z or 4-1BBz intracellular domain, a prostate tumor specific anti-ROR1 CAR with a 1928z or 4-1BBz intracellular domain, or a HBV core antigen specific HBcore18-27 TCR.

81. The method of claim 74, wherein the at least one cell targeting ligand selectively binds lymphocytes and initiates receptor-induced endocytosis.

82. The method of claim 74, wherein the expression of the therapeutic protein is for no longer than 14 days, no longer than 12 days, no longer than 10 days, no longer than 9 days, no longer than 8 days, no longer than 7 days, no longer than 6 days, or no longer than 5 days.

83. The method of claim 74, wherein administering the therapeutically effective amount of a nanoparticle to the subject comprises administering two or more doses of the nanoparticle.

84. The method of claim 83, wherein the two or more doses are administered every 5-10 days, or every 6-8 days, or every 7 days.

85. The method of claim 74, wherein the subject is in need of treatment for cancer or an infectious disease.

86. The method of claim 74, wherein administering the therapeutically effective amount of the nanoparticle comprises injection or infusion via catheter (a) into or proximal to a tumor (intratumoral), (b) into a vein (intravenous), or (c) into the peritoneum (intraperitoneally).

87. The method of claim 76, wherein administering the T cell attractant comprises injection or infusion via catheter into or proximal to a tumor (intratumoral), intravenous injection or infusion, or injection or infusion via catheter intraperitoneally.

88. The method of claim 75, wherein the cell attractant is administered to the subject before the therapeutically effective amount of the nanoparticle is administered.

89. The method of claim 88, wherein the cell attractant is administered no more than one hour before, no more than 3 hours before, no more than 6 hours before, no more than 12 hours before, or no more than 24 hours before the therapeutically effective amount of the nanoparticle is administered.

90. The method of claim 75, wherein the cell attractant is administered (a) only after the first dose of the therapeutically effective amount of the nanoparticle; (b) after each of at least two doses of the therapeutically effective amount of the nanoparticle; or (c) after each dose of the therapeutically effective amount of the nanoparticle.

91. The method of claim 74, further comprising administering a macrophage stimulating composition to the subject.

92. The method of claim 91, wherein the macrophage stimulating composition comprises a nanoparticle targeted to macrophage cells and capable of directing expression of transcription factor interferon-regulatory factor 5 (IRF5) in combination with the kinase IKKβ.

93. The method of claim 74, wherein the carrier matrix comprises a positively charged lipid or polymer.

94. The method of claim 93, wherein the positively charged polymer comprises PBAE, poly(L-lysine), PEI, PAMAMs, poly(amine-co-esters), PDMAEMA, chitosan, poly-(L-lactide-co-L-lysine), PAGA, or PHP.

95. The method of claim 74, wherein the coating comprises a neutrally or negatively-charged lipid or polymer.

96. The method of claim 74, wherein the neutrally or negatively-charged coating comprises PGA, poly(acrylic acid), alginic acid, or cholesteryl hemisuccinate/1,2-dioleoyl-sn-glycero-3-phosphoethanolamine.

97. The method of claim 74, wherein the neutrally or negatively-charged coating comprises a zwitterionic polymer.

98. The method of claim 74, wherein the neutrally or negatively-charged coating comprises a liposome.

99. The method of claim 98, wherein the liposome comprises DOTAP, DOTMA, DC-Chol, DOGS, cholesterol, DOPE, or DOPC.

100. The method of claim 74, wherein the at least one cell targeting ligand selectively binds CD4 and/or CD8.

101. The method of claim 74, wherein the at least one cell targeting ligand comprises a binding domain selected from a CD4 antibody and/or a CD8 antibody.

102. The method of claim 74, wherein the at least one cell targeting ligand comprises a binding domain selected from an scFv fragment of a CD4 antibody and/or a CD8 antibody.

103. The method of claim 74, wherein the carrier matrix comprises PBAE.

104. The method of claim 74, wherein the coating comprises PGA.

105. The method of claim 74, wherein the at least one cell targeting ligand comprises a binding domain selected from a CD4 antibody and/or a CD8 antibody; the carrier matrix comprises PBAE; and the coating comprises PGA.

106. A synthetic nanoparticle comprising:
(i) a synthetic mRNA encoding a therapeutic protein and encapsulated within a positively-charged carrier;
(ii) a neutrally or negatively-charged coating; and
(iii) a selected cell targeting ligand extending from the surface of the coating;
wherein the therapeutic protein is a leukemia-specific anti-CD19 CAR with a 1928z or 4-1BBz intracellular domain, a prostate tumor specific anti-ROR1 CAR with a 1928z or 4-1BBz intracellular domain, or a HBV core antigen specific HBcore18-27 TCR.

107. The synthetic nanoparticle of claim 106, wherein the carrier comprises a positively charged lipid or polymer.

108. The synthetic nanoparticle of claim 107, wherein the positively charged lipid or polymer comprises PBAE, poly(L-lysine), PEI, PAMAMs, poly(amine-co-esters), PDMAEMA, chitosan, poly-(L-lactide-co-L-lysine), PAGA, or PHP.

109. The synthetic nanoparticle of claim 106, wherein the coating comprises a neutrally or negatively-charged lipid or polymer.

110. The synthetic nanoparticle of claim 106, wherein the neutrally or negatively-charged coating comprises PGA, poly(acrylic acid), alginic acid, or cholesteryl hemisuccinate/1,2-dioleoyl-sn-glycero-3-phosphoethanolamine.

111. The synthetic nanoparticle of claim 106, wherein the neutrally or negatively-charged coating comprises a zwitterionic polymer.

112. The synthetic nanoparticle of claim 106, wherein the neutrally or negatively-charged coating comprises a liposome.

113. The synthetic nanoparticle of claim 112, wherein the liposome comprises DOTAP, DOTMA, DC-Chol, DOGS, cholesterol, DOPE, or DOPC.

114. The synthetic nanoparticle of claim 106, wherein the selected cell targeting ligand selectively binds CD4 and/or CD8.

115. The synthetic nanoparticle of claim 106, wherein the selected cell targeting ligand comprises a binding domain selected from a CD4 antibody and/or a CD8 antibody.

116. The synthetic nanoparticle of claim 106, wherein the selected cell targeting ligand comprises a binding domain selected from an scFv fragment of a CD4 antibody and/or a CD8 antibody.

117. The synthetic nanoparticle of claim 106, wherein the carrier comprises PBAE.

118. The synthetic nanoparticle of claim 106, wherein the coating comprises PGA.

119. The synthetic nanoparticle of claim 106, wherein the selected cell targeting ligand comprises a binding domain selected from a CD4 antibody and/or a CD8 antibody; the carrier comprises PBAE; and the coating comprises PGA.

120. A composition comprising the synthetic nanoparticle of claim 106.

121. A method of treating a subject in need thereof comprising administering a therapeutically effective amount of the nanoparticle of claim 106, or the composition of claim 120 thereby treating the subject in need thereof.

122. The method of claim 121, further comprising administering to the subject a T cell attractant before administering the therapeutically effective amount of the nanoparticle or the composition.

123. The method of claim 121, wherein the subject is in need of treatment for an infectious disease.

124. The method of claim 123, wherein the infectious disease is an adenovirus, arenavirus, bunyavirus, coronavirusess, flavirvirus, hantavirus, hepadnavirus, herpesvirus, papilomavirus, paramyxovirus, parvovirus, picornavirus, poxvirus, orthomyxovirus, retrovirus, reovirus, rhabdovirus, rotavirus, spongiform virus or togaviruses infectious disease.

125. The method of claim 123, wherein the infectious disease is cytomegalovirus (CMV), cold virus, Epstein-Barr, flu virus, hepatitis virus, herpes simplex, HIV, influenza, Japanese encephalitis, measles, polio, rabies, respiratory syncytial virus, rubella, smallpox, varicella zoster or West Nile virus infectious disease.

126. The method of claim 121, wherein the subject is in need of treatment for cancer.

127. The method of claim 126, wherein the cancer is a leukemia.

128. The method of claim 126, wherein the cancer is a lymphoma.

129. The method of claim 126, wherein the cancer is a stem cell cancer or melanoma.

130. The method of claim 126, wherein the cancer is a solid-organ tumor.

131. The method of claim 130, wherein the solid-organ tumor is prostate cancer.

132. The method of claim 130, wherein the solid-organ tumor is breast cancer, ovarian cancer, mesothelioma, renal cell carcinoma, pancreatic cancer, lung cancer, or HBV-induced hepatocellular carcinoma.

133. The method of claim 121, wherein the method achieves at least 20%, at least 30%, at least 40%, at least 50%, at least 60%, at least 70%, at least 80%, or at least 90% of in vivo T cells expressing the therapeutic protein following the administering.

134. The method of claim 126, wherein the method results in eradication of the cancer in at least 20%, in at least 30%, in at least 40%, in at least 50%, in at least 60%, or in at least 70% of subjects.

135. The method of claim 126, wherein the subject is a relapsing subject and the method results in an average of at least 10 days, at least 15 days, at least 20 days, at least 25 days, at least 30 days, at least 35 days, or at least 37 days improvement in survival of the relapsing subject.

136. The method of claim 121, wherein the method results in at least about the same efficacy as transplantation of T cells contacted with the nanoparticle ex vivo.

137. The method of claim 121, wherein the method results in at least about the same efficacy as transplantation of ex vivo transduced CAR⁺ T cells.

138. A pharmaceutical composition comprising the synthetic nanoparticle of claim 106 and a pharmaceutically acceptable excipient.

139. A pharmaceutical composition comprising the synthetic nanoparticle of claim 106 in lyophilized form.

140. A method of treating a subject, comprising reconstituting the composition of claim 139 into a pharmaceutically acceptable carrier to form a solution and injecting the solution into the subject.

141. A kit comprising the synthetic nanoparticle of claim 106 and instructions for use in treating a disease or disorder.

142. The kit of claim 141 wherein the synthetic nanoparticle is lyophilized.

143. The kit of claim 141 wherein the synthetic nanoparticle is in solution.

144. The kit of claim 141 further comprising a pharmaceutically acceptable carrier.

145. The kit of claim 141 further comprising an injection device.

146. The kit of claim 141 further comprising a cell attractant.

147. A kit comprising a positively-charged carrier matrix, a neutrally or negatively-charged coating, at least one cell targeting ligand, and a synthetic mRNA.
